(19) United States
(12) Patent Application Publication
(10) Pub. No.: US 2021/0347885 A1
(43) Pub. Date: Nov. 11, 2021

(54) SYNTHETIC BINDING AGENTS FOR LIMITING PERMEATION THROUGH MUCUS

(71) Applicants: The University of North Carolina at Chapel Hill, Chapel Hill, NC (US); MUCOMMUNE, LLC, Carrboro, NC (US)

(72) Inventors: Samuel Lai, Carrboro, NC (US); Bhawana Shrestha, Carrboro, NC (US); Alison Schaefer, Chape! Hill, NC (US); Timothy Jacobs, Durham, NC (US); Thomas Moench, Baltimore, MD (US)

(21) Appl. No.: 17/278,217

(22) PCT Filed: Sep. 23, 2019
(86) PCT No.: PCT/US2019/052396

§ 371 Date: Mar. 19, 2021

Related U.S. Application Data
(60) Provisional application No. 62/734,771, filed on Sep. 21, 2018.

(51) Int. Cl. C07K 16/28; C07K 16/12; A61P 15/16; A61P 31/04

(52) U.S. Cl. CPC C07K 16/28; C07K 16/1228; C07K 16/1217; C07K 16/1235; A61P 15/16; C07K 2317/52; C07K 2317/565; C07K 2317/55; C07K 2317/51; C07K 2317/515; A61P 31/04

(57) ABSTRACT

Synthetic binding agents for reducing the fraction of targets that can permeate through mucus and/or freely divide, and methods of reducing mucosal permeation and/or free division of a target using these synthetic binding agents.

### CROSS REFERENCE TO RELATED APPLICATIONS

This patent application claims priority to U.S. provisional patent application No. 62/734,771, filed on Sep. 21, 2018 (titled “SYNTHETIC BINDING AGENTS FOR MUCOSAL TRAPPING”), herein incorporated by reference in its entirety.

### STATEMENT AS TO FEDERALLY SPONSORED RESEARCH

This invention was made with Government support under Grant No. R56HD095629 and U54HD096957 awarded by the National Institutes of Health. The Government has certain rights in the invention.

### INCORPORATION BY REFERENCE

All publications and patent applications mentioned in this specification are herein incorporated by reference in their entirety to the same extent as if each individual publication or patent application was specifically and individually indicated to be incorporated by reference.

### REFERENCE TO SEQUENCE LISTING

The present application includes a listing of sequences. A Sequence Listing in electronic format is submitted with this utility application.

### FIELD

The present disclosure generally relates to methods and compositions for enhancing agglutination of a target, facilitate enchaining of a target, and/or muco-trapping of a target to prevent conception (e.g., for contraception), and/or to prevent or treat infection, including viral, bacterial and/or fungal infections.

### BACKGROUND

The mucosal barrier plays an important potential protective role as a barrier to prevent foreign matter from entering the body. The mucosal barrier may be further enhanced by local immunity that allows a robust immune system response to occur at mucosal membranes of the intestines, the urogenital tract and the respiratory system, i.e., surfaces that are in contact with the external environment. The mucosal immune system may provide protection against pathogens but maintains a tolerance towards non-harmful commensal microbes and benign environmental substances. Since the mucosal membranes are the primary contact point between a host and its environment, a large amount of secondary lymphoid tissue is found here. The mucosa-associated lymphoid tissue, or MALT, provides a critical element of the mucosal immune response. The mucosal immune system provides three main functions: serving as the body's first line defense from antigens and infection, preventing systemic immune responses to commensal bacteria and food antigens (primarily food proteins in the gut-associated lymphoid tissue, so-called oral tolerance), and regulating appropriate immune responses to pathogens encountered on a daily basis.

Unfortunately, the mucosal immune response may be inadequate, and it is often difficult to elicit the necessary immune response for sufficient duration. This is exemplified by the lack of effective vaccines against the majority of sexually transmitted infections, including HIV, Herpes, Chlamydia and Gonorrhea. Consequently, enhancements of the mucus barrier and the mucosal immune system by direct delivery of antibodies have been suggested as one method of treating or preventing infection. See, e.g., US 20150284451, which describes the use of compositions to prevent pathogen infection by applying antibodies that may interact with mucus.

Although some antibodies have been shown to interact with mucins to adhesively crosslink individual antibody-coated pathogens to mucins and thereby immobilizing them in mucus (a process frequently referred to as muco-trapping), it would be beneficial to provide antibodies or antibody constructs having further improved ability to more effectively prevent foreign matter, including viruses and bacteria, from permeating through mucus to reach target cells. In addition, such improved constructs may be beneficially used as a contraceptive, by blocking or limiting passage of sperm to the egg within the female reproductive tract. Beyond crosslinking foreign entities to mucins, it is possible to further enhance the potencies of the antibodies by improving the agglutination and/or enchainment of foreign entities together in a manner that limits their effective permeation through mucus.

Nearly half of all pregnancies in the U.S. are unintended, underscoring the critical need for additional options for contraception. Non-hormonal contraceptives would be of particular use.

Described herein are methods and compositions (including compositions of engineered/synthetic binding agents) for enhancing agglutination, enchainment and/or muco-trapping of a target, i.e., reducing the fraction of target entities that could permeate through mucus, including pathogens and sperm.

### SUMMARY OF THE DISCLOSURE

In general, described herein are synthetic binding agents for enhancing agglutination and/or muco-trapping of a target, and methods of enhancing agglutination and/or muco-trapping of a target using any of these synthetic binding agents. The target typically has one or more epitopes, and may be a virus, bacteria, fungus, sperm or parasite. The synthetic binding agents described herein are multimeric, having multiple epitope-binding regions. All of these epitope-binding regions may be immunoglobulin fragment antigen binding (Fab) regions or fragments, and may include a core of a human or humanized Immunoglobulin G (IgG) having Fab and Fc domains. All of the Fab fragments/domains (including those of the core humanized IgG) may be directed to the same epitope to recognize the foreign body. Thus, the synthetic binding agents for enhancing agglutination and/or muco-trapping described herein may include a human or humanized IgG that is linked to one or more additional Fab domains, wherein the one or more additional Fab domains and the parent IgG Fab domains all specifically bind to the target epitope with high affinity, and may reduce the mobility of the target in mucus to less than about 50% relative to its native mobility in mucus. The synthetic binding agents may be recombinant (e.g., engineered) antibodies. Any of the synthetic binding agents described herein may be further configured (or selected) to enhance mucin crosslinking once bound to the target, but may otherwise be relatively free to diffuse through mucus (e.g., have a low affinity for mucins). As used herein the term “native mobility” refers to the mobility of the target (e.g., sperm, virus, bacteria, etc.) in the same environment (e.g., mucus, saline, etc.) in the absence of a synthetic binding agent or antibody.

Also described herein are also methods and compositions (including compositions of engineered/synthetic binding agents) that could provide bactericidal and/or microbicidal effect by more effectively clumping together pathogens that undergo cell division, which leads to a chain of bacteria and/or other pathogens, and potentially inhibiting replication, triggering cell death, e.g., forming aggregations (including in some variations multi-pathogen aggregations) and/or preventing the spread of the infection through either agglutination or enchained growth.

In particular, described herein are synthetic binding agents configured as recombinant antibodies that may be used as a contraceptive. A contraceptive synthetic binding agent may be referred to herein as a human contraceptive agent (HCA), although these methods may also be used for non-human (e.g., animal) contraception. For example, described herein are contraceptive methods and HCA compositions, including recombinant engineered antibodies (Ab) that can block sperm permeation through mucus. A major effector function for Ab in mucus is to arrest the forward motion of foreign entities such as viruses and highly motile bacteria, and block them from reaching target cells. This function can be accomplished in two ways. First, when concentrations of the foreign entity are high such that the foreign bodies would frequently collide, Ab can crosslink two or more bodies together, resulting not only in an increase in hydrodynamic diameter but also an effective neutralization of the net forward motion of swimming bodies. This process is commonly referred to as agglutination. Second, when concentrations of the foreign entity are modest such that collisions between foreign bodies are relatively infrequent, Ab can immobilize by directly crosslinking the foreign body to the mucin matrix present in mucus via multiple Fc-mucin bonds. This process, which is herein referred to as mucin-crosslinking or muco-trapping, has remained largely unrecognized because the affinity between each Ab molecule and mucin was long thought to be much too weak to effectively bind individual foreign bodies to mucins. However, vaginally dosed antigen-specific IgG that are tuned to possess weak affinity to mucins can trap viruses in mucus by forming multiple weakly adhesive bonds between the virus and the mucin mesh (akin to a VELCRO® patch with individually weak hooks). Finally, for foreign bodies that can divide on its own (e.g. bacteria, fungus, etc.), aggregates of the bacteria may be formed by enchaining the daughter cell of the dividing bacteria with the mother cell i.e. enchained growth. The end result is a clump of foreign bodies (similar to what would be formed by agglutination) but without requiring independent and distinct foreign bodies from colliding with each other.

Sperm concentration varies widely in the female reproductive tract, with the maximum concentration in semen immediately following ejaculation and lower concentrations in more distal sites, such as the cervical canal.

The various HCA constructs described herein are configured to act by blocking sperm permeation through mucus and preventing sperm from reaching the egg and may therefore harness both agglutination and muco-trapping mechanisms. Polyvalent Ig such as sIgA and IgM are markedly more potent agglutinators than IgG (IgM is ˜1000-fold more potent at agglutination than IgG). Unfortunately, large scale manufacturing of IgM or sIgA remains exceptionally challenging, and both IgM and sIgA suffer from stability issues. IgG represents the predominant isotype of Ab under clinical development and has an outstanding track record of safety in humans. Thus, from a translational development perspective, IgG represents the most logical platform for developing more potent HCA. Greater potency not only translates to lower doses of HCA needed but also maximizes the potential effectiveness of HCA-based contraception.

To date, a multimeric Ab construct to create more potent HCA has not been developed. Described herein are engineered multimeric Ab that may radically improve on the current monomeric IgG1-based HCA by more potently agglutinating sperm, thus achieving greater potency in blocking permeation through mucus.

For example, a synthetic binding agent for enhancing agglutination and/or muco-trapping of a target having an epitope may include: a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to the epitope of the target, so that the synthetic binding agent binds to the target with high affinity and reduces the mobility of the target in mucus to less than about 50% relative to its native mobility in mucus (or in some variations, in water). The reduction of mobility in mucus may be due to enhanced agglutination by the synthetic binding agent (construct), and/or due to enhanced enchainment of targets that can divide. In any of the variations described herein the one or more additional Fab domains and the IgG Fab domains may bind different epitopes on the same target (e.g., pathogen).

Any number (preferably an even number) of additional Fab domains may be included. For example, the one or more additional Fab domains may comprise 2, 4, 6 or 8 additional Fab domains.

In some variations, the synthetic binding agent is a contraceptive synthetic binding agent (e.g., a contraceptive antibody), and the target is sperm and all of the one or more Fab domains and the IgG Fab domains specifically bind repeating poly-n-acetyllactosaminyl structures on sperm, an N-linked glycosylated form of SEQ ID: 1 (e.g., an amino acid sequence comprising GQNDTSQTSSPS), where the glycans are poly-n-acetyllactosamine. This target is referred to herein as CD52g.

The additional Fab domains may each comprise: (i) a heavy chain (HC) with a variable region (VH) comprising complementarity determining region(s) (CDRs) having the amino acid sequence of: SEQ ID NO: 4 (e.g., an HC CDR sequence as SEQ ID NO: 4); and/or (ii) a light chain (LC) with a variable region (VL) comprising complementarity determining the amino acid sequence of: SEQ ID NO: 7 (e.g., an LC CDR sequence as SEQ ID NO: 7). In some variations, the at least one additional Fab domain of the synthetic binding agent is linked to a Fab domain of the pair of Fab domains of the IgG; alternatively or additionally, the at least one additional Fab domain may be linked to an Fc region of the IgG. The IgG may comprise at least one Fe region that is a naturally occurring sequence. As described below, in some variations the Fc sequence may also be modified (for instance, to prolong systemic circulation and reduced interactions with other immune cells).

The amino acid sequences of the additional Fab domains do not have to be identical to each other or to the IgG Fab domains, although they may all bind to the same antigen with approximately the same affinity. One of reasonable skill in the art may apply known methods to vary the sequence while retaining substantially all of the binding affinity. For example, conservative amino acid substitutions may be made (e.g., an exchange between two amino acids separated by a small physicochemical distance). For example, in any of the variations described herein, the additional Fab domains may comprise: (i) a heavy chain (HC) with a variable region (VH) comprising complementarity determining region(s) (CDRs) having an amino acid sequence that is between 100% and 75% (e.g., between 100%-80%, between 100%-85%, between 100%-90%, between 100%-95%, etc.) identical to the amino acid sequence of the IgG Fab domain (e.g., for an CD52g synthetic binding peptide, having an amino acid sequence that is between 100% and 75% (e.g., between 100%-80%, between 100%-85%, between 100%-90%, between 100%-95%, etc.) identical to the amino acid sequence of SEQ ID NO: 4, e.g., an HC CDR sequence as SEQ ID NO: 4); and/or (ii) a light chain (LC) with a variable region (VL) comprising complementarity determining the amino acid sequence having an amino acid sequence that is between 100% and 75% (e.g., between 100%-80%, between 100/6-85%, between 100/6-90%, between 100%-95%, etc.) identical to the amino acid sequence of the IgG Fab domain light chain VL (e.g., for a CD52g synthetic binding peptide, having an amino acid sequence that is between 100% and 75% (e.g., between 100%-80%, between 100%-85%, between 100%-90%, between 100%-95%, etc.) identical to the amino acid sequence of: SEQ ID NO: 7, e.g., an LC CDR sequence as SEQ ID NO: 7).

In some variations, the one or more additional Fab domains may be linked to the IgG via a flexible linker comprising an amino acid sequence comprising n pentapeptide repeats consisting of Glycine (G) and Serine (S), wherein n is between 2 and 15 (e.g., between 2 and 14, between 2 and 13, between 2 and 12, between 2 and 11, between 2 and 10, between 2 and 9, between 2 and 8, between 3 and 15, between 3 and 14, between 3 and 13, between 3 and 12, between 3 and 11, between 3 and 10, between 3 and 9, between 3 and 8, etc.). Other linkers, not limited to amino acid/peptide linkers, may be used, for example, non-amino acid polymers such as polynucleotide linkers and other synthetic linkers. In general, the linkers do not need to be identical. Linkers may be one set of glycine serine linkers that are used for connecting one Fab, but another set of linkers uses, e.g., (EAAAK)₃, and/or yet another set of linker uses (Ala-Pro)ₙ (10-34 aa) linker.

Also described herein are isolated nucleic acid molecules encoding any of the synthetic binding agents described herein. Also described herein are vectors comprising any of these nucleic acid molecules and/or an isolated host cell or a non-human organism transformed or transfected with the nucleic acid molecule. Also described herein are compositions comprising any of the synthetic binding agents and a pharmaceutically acceptable carrier.

Although many of the variations described herein are directed specifically to compositions (e.g., synthetic binding agents) and methods for contraception, it should be understood that any of these compositions and methods may be directed to the treatment or prevention of infection by a pathogen (e.g., virus, bacteria, fungi, etc.).

For example, described herein are methods of enhancing agglutination and/or muco-trapping of a target (e.g., sperm, virus, bacteria, fungi, etc.) having an epitope, the method comprising: administering a synthetic binding agent to a patient, the synthetic binding agent comprising a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to the epitope of the target, so that the synthetic binding agent binds to the target with high affinity and enhances agglutination and/or muco-trapping of the target in the patient's mucus to reduce the mobility of the target to less than about 50% (e.g., less than about 40%, 30%, 20%, 10%, etc.) of its native mobility e.g., relative to its native mobility in mucus, or in some variations in water. In some variations, the synthetic binding agents described herein reduce the fraction of progressively motile sperm, e.g. >95% (90% or greater, 85% or greater, 80% or greater, 75% or greater, 70% or greater, 65% or greater, 60% or greater, etc.) as compared to the fraction of progressively motile sperm in control. For example, >50% reduction in progressively motile sperm populations.

As mentioned above, any number (preferably an even number) of additional Fab domains may be included. For example, the one or more additional Fab domains may comprise 2, 4, 6 or 8 additional Fab domains.

In particular, the target may be a sperm and all of the one or more Fab domains and the IgG Fab domains may specifically bind to an epitope of CD52g. The mobility of the sperm is slowed down by at least 50% (e.g., relative to its native mobility in mucus), e.g., less than about 40%, 30%, 20%, or 10% native mobility.

In general, administering comprises administering to the patient vaginally. In some variations (e.g., directed to respiratory or other infection or infectious routes) administering may comprise topical administration, such as, but not limited to, administering via inhalation (e.g., of an aerosol), oral, eye-drop, lavage, etc. In any of these variations, administering may comprise administering systemically to the patient, including systemic delivery for mucosal (e.g. vaginal, respiratory, gastrointestinal) applications.

In some variations, administering comprises delivering from an intravaginal ring (IVR) or vaginal film. In some variations, administering comprises delivering to lung mucosa using a nebulizer.

Any appropriate amount of the synthetic binding agent may be administered. For example, administering may comprise delivering between 0.01 mg and 100 mg/day of the synthetic binding agent. For example, when using a contraceptive synthetic binding agent, administering may comprise administering an amount sufficient to agglutinate the target to enhance overall ability to limit sperm permeation across mucus.

In some variations, the synthetic binding agents described herein are synthetic binding agents for inhibiting sperm mobility through mucus. For example a synthetic binding agents for inhibiting sperm mobility through mucus may include: a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to an epitope of CD52g, so that the synthetic binding agent reduces the mobility of sperm in mucus to less than about 50% relative to its native mobility in mucus and/or reduces the fraction of progressively motile sperm by 50%. In some variations, the one or more additional Fab domains may comprise, for example, 2, 4, 6 or 8 additional Fab domains. The epitope of CD52g may comprise an N-linked glycosylated form of SEQ ID NO: 1.

In any of the variations described herein, the additional Fab domains may each comprise: (i) a heavy chain (HC) with a variable region (VH) comprising complementarity determining regions (CDRs) having the amino acid sequence of: SEQ ID NO: 4; and/or (ii) a light chain (LC) with a variable region (VL) comprising complementarity determining regions (CDRs) having the amino acid sequence of: SEQ ID NO: 7.

The one or more additional Fab domains may be linked to a Fab domain of the pair of Fab domains of the IgG. For example, the at least one additional Fab domain may be linked to an Fc region of the IgG. The IgG may comprise at least one Fc region that is a naturally occurring sequence.

The one or more additional Fab domains may be linked to the IgG via a flexible linker comprising an amino acid sequence comprising n pentapeptide repeats consisting of Glycine (G) and Serine (S), wherein n is between 3 and 8.

Also described herein are isolated nucleic acid molecules encoding the synthetic binding agent, and/or a vector comprising the corresponding nucleic acid molecule, and/or an isolated host cell or a non-human organism transformed or transfected with the nucleic acid molecules. Any of the synthetic binding agents described herein may be part of a composition comprising the synthetic binding agent and a pharmaceutically acceptable carrier.

Any appropriate delivery device may be used with the compositions and/or synthetic binding agents described herein. For example, an intravaginal ring (IVR) or vaginal film may be used.

Described herein are methods of providing contraception in a female subject. These methods may include administering to a mucosa of a reproductive tract of the subject any of the synthetic binding agents (and particularly those that bind a sperm-selective marker, including CD52g, as described herein) in an amount effective to provide contraception. For example, described herein are methods of inhibiting the mobility of sperm in the mucus of a reproductive tract of a female subject that include contacting the mucus (e.g., in the female genital tract) with any of these synthetic binding agents (including via a delivery device) in an amount effective to inhibit the mobility of at least 80% of sperm present in the mucus. In some variations, the mobility of the sperm may be slowed down by at least 50% relative to its normal or native mobility in mucus and/or reduces the fraction of progressively motile sperm by 50%. The synthetic binding agent or composition may be delivered via vaginal administration (e.g., using an intravaginal ring (IVR)); alternatively or additionally, the synthetic binding agent or composition may be delivered via systemic administration. An IVR may be configured to release an effective amount for at least 15 days. In some variations, the composition may be delivered in a film that dissolves intravaginally releasing the synthetic binding agent.

In general, the Fab fragments may be on the N- or C-terminal ends of the core IgG. For example, the additional Fab domain(s) may be inserted at the N-terminus (i.e. extending another Fab arm) or at the C-terminus (i.e. after the Fc domain). In some variations, the synthetic binding agent may include at least two additional Fab domains (also referred to herein as Fab fragments) before and/or after the human or humanized IgG. In particular, the synthetic binding agent for enhancing agglutination, enchainment and/or muco-trapping of a target having an epitope (including an epitope specific to sperm and therefore effective to trap sperm) may include a human or humanized Immunoglobulin G (IgG) having a pair of Fab domains (and a pair of Fc domains), in which the human or humanized IgG is linked to four additional Fab domains, and the IgG Fab domains as well as the additional Fab domains all specifically bind to the epitope of the target, so that the synthetic binding agent binds to the target with high affinity and reduces the mobility of the target in mucus to less than about 50% relative to its native mobility in mucus. In this example, the synthetic binding agent includes additional Fabs on both sides of the core IgG (e.g., 2 on the N terminal and 2 on the C terminal ends, for a total of 6 Fabs on the molecule).

In variations configured as a contraceptive, the potency of the binding agent may be determined in part by the minimum concentration of the synthetic binding agent that is able to effectively agglutinate the sperm and prevent it from freely swimming (i.e. remain as progressively motile sperm). In general, the synthetic binding agents having 6 or more total Fab fragments have been found to have an order of magnitude better potency (e.g., agglutination potency) compared to just the IgG (including the IgG glycosylated to enhance mucosal binding). Specifically, many of the synthetic binding agents configured as contraceptives described herein have been shown to reduce progressively motile sperm (e.g. by 95% vs. untreated control) to a similar extent as native IgG at greater than 10× lower binding agent concentrations. The potency of the binding agent may also be determined in part by enhanced “muco-trapping”, which refers to the synthetic binding agent crosslinking a greater fraction of sperm to mucins compared to native IgG, or crosslinking a similar fraction of sperm to mucins as IgG at lower binding agent concentrations.

The human or humanized IgG forming the core of the synthetic binding agents described herein may include non-native Fc regions (e.g., Fc regions modified to increase stability/half-life in the body, Fc regions modified to decrease immunoreactivity, etc.). For example, the Fc region of the IgG portion of the synthetic binding agent may be modified to include one or more specific mutations whereby specific immune functions are modified. For instance, the Fc region may have enhanced FcRn affinity to extend circulation kinetics. For example, a mutation in human IgG (e.g., IgG1) of T250Q/M428L may increase binding to FcRn, and increase the half-life, and/or a mutation of M252Y/S254T/T256E+H433K/N434F may increase binding to FcRn and increase the half-life. In some variations the synthetic binding agent includes a modified Fc region having reduced FcR affinity which may help ensure that the Ab does not prime the immune system to develop antibodies against sperm. For example, one or more mutations in the human IgG (e.g., IgG1) that decrease binding to FcR (e.g., FcγR) may be included, such as E233P/L234V/L235A/G236+A327G/A330S/P331S, L234A/L235A/P329G, and/or K322A.

For example, as described above, a method of enhancing agglutination and/or muco-trapping of a target having an epitope may include: administering a synthetic binding agent to a subject, the synthetic binding agent comprising a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to the epitope of the target, so that the synthetic binding agent binds to the target with high affinity and enhances the ability for the subject's mucus to limit permeability of the target across mucus, as reflected by a reduction of the mobility of the target to less than about 50% relative to its native mobility in mucus, or the fraction of motile target to less than 50% relative to untreated control. The one or more additional Fab domains comprises 2, 4, 6 or 8 additional Fab domains.

In some variations, and particularly contraceptive methods, target may be sperm. In some variations, the antigen is CD52g. The mobility of the sperm in mucus may be slowed down by at least 50% compared to the native mobility of the sperm in the mucus.

In some variations, the target may be a pathogen, e.g., all of the one or more Fab domains and the IgG Fab domains may specifically bind to a pathogen. For example, the pathogen may be one (or more) of: Acinetobacter baumannii; Bacteroldes fragilis; Burkholderia cepacia, Clostridium difficile; Clostridium sordellii; Carbapenem-resistant Enterobacteriaceae; Enterococcus faecalis; Escherichia coli; Hepatitis A; Hepatitis B; Hepatitis C; human immunodeficiency virus HIV-1 and HIV-2 (HIV, AIDS); Influenza; Klebsiella pneumonia; Methicillin-resistant Staphylococcus aureus; Morganella morganii; Mycobacterium abscessus; Norovirus; Psuedomonas aeruginosa; Staphylococcus aureus; Stenotrophomonas maltophilia; Mycobacterium tuberculosis; Vancomyin-resistant Staphylococcus aureus; Vancomycin-resistant Enterococci; Neisseria gonorrhoeae (gonorrhea); Chlamydia trachomatis (chlamydia, lymphogranuloma venereum); Treponema pallidum (syphilis); Haemophilus ducreyi (chancroid); Klebsiella granulomatis or Calymmatobacterium granulomatis (donovanosis), Mycoplasma genitalium, Ureaplasma urealyticum (mycoplasmas); HTLV-1 (T-lymphotrophic virus type 1); herpes simplex virus type 1 and type 2 (HSV-1 and HSV-2); Epstein-Barr virus; cytomegalovirus; human herpesvirus 6; varicella-zoster virus; human papillomaviruses (genital warts); hepatitis A virus, hepatitis B virus, hepatitis C virus (viral hepatitis); molluscum contagiosum virus (MCV); Trichomona vaginalis (trichomoniasis); and yeasts, such as Candida albicans (vulvovaginal candidiasis). In some variations, the pathogen includes a fungus, such as Aspergillus.

Administering may comprise administering by any appropriate route, or more than one route. For example, administering may comprise administering to the subject vaginally (e.g., from an intravaginal ring, IVR). Administering may comprise administering systemically to the subject. Administering may comprise administering to the subject as a vaginal film. Administering may comprise administering from a nebulizer. Administering may comprise administering by inhalation. Administering may comprise an eye drop. Administering may comprise an oral capsule or pill. Administering may comprise a mouth wash. In some variations, administering comprises delivering between 0.01 mg and 100 mg/day of the synthetic binding agent. Administering may comprise administering in an amount sufficient to agglutinate or form enchainment of the target while maintaining or enhancing muco-trapping, with the overall net effect of reducing the permeability of the target through mucus, and/or reducing the growth or presence of the target.

As mentioned above, the IgG Fab domains may have an amino acid sequence that is not identical to the one or more additional Fab domains. For example, the IgG domains may have an amino acid sequence that is between 100% h (identical) and 75%, 80%, 85%, 90%, 95%, etc. In general, the IgG Fab and the additional Fab domains recognize the same antigen with approximately the same affinity, regardless of their sequence.

For example, described herein are methods of inhibiting fertilization and/or conception by agglutination and/or muco-trapping of sperm that may include: administering a synthetic binding agent to a subject, the synthetic binding agent comprising a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to an epitope of sperm, so that the synthetic binding agent binds to the sperm with high affinity and enhances agglutination and/or muco-trapping of the sperm in mucus. The net effect is either a reduction in the fraction of progressively motile sperm, and/or a reduction in the mobility of motile sperm. As mentioned, the one or more additional Fab domains may comprise 2, 4, 6 or 8 additional Fab domains.

The mobility of the sperm in mucus may be slowed down by at least 50% compared to the native mobility of the sperm in the mucus. In some variations, the mobility of the target (e.g., sperm, pathogen, etc.) may be slowed by at least 40% compared to the native mobility of the target, slowed by at least 30% compared to the native mobility, slowed by at least 20% compared to the native mobility, slowed by at least 15% compared to the native mobility, etc.

In some variations, the synthetic binding agents described herein reduce the fraction of progressively motile sperm among all sperm, e.g. >95% (90% or greater, 85% or greater, 80% or greater, 75% or greater, 70% or greater, 65% or greater, 60% or greater, etc.) than reduction in fraction of progressively motile sperm vs. control. For example, >50% reduction in progressively motile sperm populations.

As mentioned, any appropriate administration route may be used. For example, administering may comprise administering to the subject via one or more of: vaginally (e.g., from an intravaginal ring), topically, systemically, as a vaginal film, from a nebulizer. Administering may comprise administering in an amount sufficient to agglutinate the target, and/or muco-trapping of the target, with the overall effect of reducing target permeation through mucus.

Also described herein are methods of treating or preventing an infection by a pathogen, the method comprising administering a synthetic binding agent to a subject, the synthetic binding agent comprising a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to a single epitope of the pathogen, so that the synthetic binding agent binds to the pathogen with high affinity and enhances agglutination of the target, inducing enchained growth of the target, and/or muco-trapping of the target in the subject's mucus. The one or more additional Fab domains comprises 2, 4, 6 or 8 additional Fab domains. The mobility of the pathogen in mucus is slowed down by at least 10% (at least 50%, at least 40%, at least 30%, at least 20/a, at least 15%, etc.) compared to the native mobility of the pathogen in the mucus.

The pathogen may be one or more of: influenza (including influenza A, B, and C); severe acute respiratory syndrome (SARS); respiratory syncytial virus (RSV); parainfluenza; adenovirus; human rhinovirus; coronavirus; and norovirus. The pathogen may be one or more of: Salmonella and Escherichia coli. The pathogen may be one or more of: Neisseria gonorrhoeae (gonorrhea); Chlamydia trachomatis (chlamydia, lymphogranuloma venereum); Treponema pallidum (syphilis); Haemophilus ducreyi (chancroid); Klebsiella granulomatis or Calymmatobacterium granulomatis (donovanosis), Mycoplasma genitalium, Ureaplasma urealyticum (mycoplasmas); human immunodeficiency virus HIV-1 and HIV-2 (HIV, AIDS); HTLV-1 (T-lymphotrophic virus type 1); herpes simplex virus type 1 and type 2 (HSV-1 and HSV-2); Epstein-Barr virus; cytomegalovirus; human herpesvirus 6; varicella-zoster virus; human papillomaviruses (genital warts); hepatitis A virus, hepatitis B virus, hepatitis C virus (viral hepatitis); molluscum contagiosum virus (MCV); Trichomona vaginalis (trichomoniasis); and yeasts, such as Candida albicans (vulvovaginal candidiasis).

Administering may be any of the types of delivery described herein, including but not limited to: administering systemically, orally, intramuscular injection, intravascular injection, subcutaneous injection, parenteral, inhalation (e.g., from a nebulizer), topical, etc. Administering comprises delivering between 0.01 mg and 100 mg/day of the synthetic binding agent. Administering may comprise administering in an amount sufficient to agglutinate the pathogen and/or preserving or further enhancing muco-trapping of the pathogen. In some variations administering may comprise administering in sufficient amount to cause enchained growth, linking dividing bacteria from the same mother bacteria together into a long chain, which has the effect of creating large clumps too large to permeate through mucus. In some variations administering may comprise administering in sufficient amount to cause enchained growth, linking dividing bacteria from the same mother bacteria together into a long chain, which has the effect of creating large clumps that limit their spread throughout the body and/or limit their growth rate.

As mentioned above, the IgG Fab domains may have an amino acid sequence that is not identical to the one or more additional Fab domains.

Any of the synthetic binding agents described herein for enhancing agglutination, enchained growth and/or muco-trapping of a target having an epitope may include: a human or humanized Immunoglobulin G (IgG) having a pair of Fab domains, wherein the human or humanized IgG is linked to one or more additional immunoglobulin fragment antigen binding (Fab) domains by a linker (e.g., an amino acid/peptide linker), wherein the one or more additional Fab domains and the IgG Fab domains may all specifically bind to the epitope of the target, so that the synthetic binding agent binds to the target with high affinity and reduces the mobility of the target in mucus (e.g., to less than about x % relative to its native mobility in mucus, such as less than about 15%, 20%, 30%, 40%, 50%, etc.). The one or more additional Fab domains may comprise 2, 4, 6 or 8 additional Fab domains.

The target may be sperm and all of the one or more Fab domains and the IgG Fab domains may specifically bind to and epitope of CD52g (e.g., a repeating poly-n-acetyllactosaminyl structures on sperm, an N-linked glycosylated form of SEQ ID: 1). As mentioned, the additional Fab domains of the synthetic binding agent targeting CD52g may each comprise: (i) a heavy chain (H) with a variable region (VH) comprising complementarity determining regions (CDRs) having an amino acid sequence that is between 100% and 80% identical to the amino acid sequence of: SEQ ID NO: 4; and/or (ii) a light chain (LC) with a variable region (V_{L}) comprising complementarity determining regions (CDRs) having an amino acid sequence that is between 100% and 80% identical to the amino acid sequence of: SEQ ID NO: 7.

For example, a synthetic binding agent for inhibiting sperm mobility through mucus, may include: a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to an epitope of CD52g, so that the synthetic binding agent reduces the mobility of sperm in mucus to less than about 50% relative to its native mobility in mucus. The one or more additional Fab domains may comprise 2, 4, 6 or 8 additional Fab domains. The epitope of CD52g may be repeating poly-n-acetyllactosaminyl structures, an N-linked glycosylated form of SEQ ID NO: 1. The additional Fab domains may each comprise: (i) a heavy chain (HC) with a variable region (V_{H}) comprising complementarity determining regions (CDRs) having an amino acid sequence that is between 100% and 80% identical to the amino acid sequence of: SEQ ID NO: 4; and/or (ii) a light chain (LC) with a variable region (V_{L}) comprising complementarity determining regions (CDRs) having an amino acid sequence that is between 100% and 80% identical to the amino acid sequence of: SEQ ID NO: 7.

Also described herein are specific examples of synthetic binding agents that target bacterial pathogens, such as, in one non-limiting example, Klebsiella bacillus. For example, the IgG may be directed to an antigen of Klebsiella, e.g., an example of which is provided in SEQ ID NO: 39 to SEQ ID NO: 45, and the additional Fab domains may each comprise: (i) a heavy chain (HO) with a variable region (V_{H}) comprising complementarity determining regions (CDRs) having the amino acid sequences that is identical or similar to the HC V_{H} region of the IgG (e.g., in relation to the example of SEQ ID NO: 39 to SEQ ID NO: 45, SEQ ID NO: 41); and (ii) a light chain (LC) with a variable region (V_{L}) comprising complementarity determining regions (CDRs) having the amino acid sequences that is identical or similar to that of the IgG (e.g., in relation to the example of SEQ ID NO: 39 to SEQ ID NO: 45, SEQ ID NO: 44). For example, an synthetic binding agent directed to an antigen of Klebsiella may have additional Fab domains each comprise: (i) a heavy chain (HC) with a variable region (V_{H}) comprising complementarity determining regions (CDRs) having an amino acid sequence that is between 100% and 80% identical to the amino acid sequence of: SEQ ID NO: 41; and/or (ii) a light chain (LC) with a variable region (V_{L}) comprising complementarity determining regions (CDRs) having an amino acid sequence that is between 100% and 80% identical to the amino acid sequence of: SEQ ID NO: 44.

For example, a synthetic binding agent for treating or preventing infection by a Klebsiella bacillus pathogen may include: a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to an epitope specific to Klebsiella bacillus, so that the synthetic binding agent reduces the mobility of Klebsiella bacillus in mucus. The one or more additional Fab domains may comprise 2, 4, 6 or 8 additional Fab domains. The additional Fab domains may each comprise: (i) a heavy chain (HC) with a variable region (VH) comprising complementarity determining regions (CDRs) having an amino acid sequence that is between 100% and 80% identical to the amino acid sequence of: SEQ ID NO: 41; and/or (ii) a light chain (LC) with a variable region (V_{L}) comprising complementarity determining regions (CDRs) having an amino acid sequence that is between 100% and 80% identical to the amino acid sequence of: SEQ ID NO: 44.

Another non-limiting example of a synthetic binding agent that targets a bacterial pathogen are synthetic binding agents that target Salmonella bacillus. The IgG portion of the synthetic binding agent may be directed to an antigen of Salmonella (such as described in SEQ ID NO: 67-73), and the additional Fab domains (which are directed to the same target antigen) may have a similar or identical amino acid sequence as the Fab domain of the IgG. For example, the additional Fab domains may each comprise: (i) a heavy chain (HC) with a variable region (V_{H}) comprising complementarity determining regions (CDRs) having the amino acid sequences of the IgG (e.g., SEQ ID NO: 69); and/or (ii) a light chain (LC) with a variable region (V_{L}) comprising complementarity determining regions (CDRs) having the amino acid sequences of the IgG (e.g., SEQ ID NO: 72). In some variations, the synthetic binding agent directed to an antigen of Salmonella includes additional Fab domains that each comprise: (i) a heavy chain (HC) with a variable region (V_{H}) comprising complementarity determining regions (CDRs) having an amino acid sequence that is between 100% and 80% identical to the amino acid sequence of: SEQ ID NO: 69; and/or (ii) a light chain (LC) with a variable region (V_{L}) comprising complementarity determining regions (CDRs) having an amino acid sequence that is between 100% and 80% identical to the amino acid sequence of: SEQ ID NO: 72.

For example, a synthetic binding agent for treating or preventing infection by a Salmonella bacillus pathogen may include: a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to an epitope specific to Salmonella bacillus, so that the synthetic binding agent reduces the mobility of Salmonella bacillus in mucus. The one or more additional Fab domains may comprise 2, 4, 6 or 8 additional Fab domains. The additional Fab domains may each comprise: (i) a heavy chain (HC) with a variable region (V_{H}) comprising complementarity determining regions (CDRs) having an amino acid sequence that is between 100% and 80% identical to the amino acid sequence of: SEQ ID NO: 69; and/or (ii) a light chain (LC) with a variable region (V_{L}) comprising complementarity determining regions (CDRs) having an amino acid sequence that is between 100% and 80% identical to the amino acid sequence of: SEQ ID NO: 72.

Another non-limiting example of a synthetic binding agent that targets a bacterial pathogen are synthetic binding agents that target Neisseria gonorrhoeae. The IgG portion of the synthetic binding agent may be directed to an antigen of Neisseria gonorrhoeae (such as described in SEQ ID NO: 102-108), and the additional Fab domains (which are directed to the same target antigen) may have a similar or identical amino acid sequence as the Fab domain of the IgG. For example, the additional Fab domains may each comprise: (i) a heavy chain (HC) with a variable region (V_{H}) comprising complementarity determining regions (CDRs) having the amino acid sequence that is similar or identical to the HC V_{H} of the IgG (e.g., SEQ ID NO: 104); and/or (ii) a light chain (LC) with a variable region (V_{L}) comprising complementarity determining regions (CDRs) having the amino acid sequence that is similar or identical to that of the LC V_{L} of the IgG (e.g., SEQ ID NO: 107). For example, a synthetic binding agent directed against Neisseria gonorrhoeae may include an IgG against an antigen of Neisseria gonorrhoeae and additional Fab domains that each comprise: (i) a heavy chain (HC) with a variable region (V_{H}) comprising complementarity determining regions (CDRs) having an amino acid sequence that is between 100% and 80% identical to the amino acid sequence of the IgG (e.g., SEQ ID NO: 104); and/or (ii) a light chain (LC) with a variable region (V_{L}) comprising complementarity determining regions (CDRs) having an amino acid sequence that is between 100% and 80% identical to the amino acid sequence of that of the IgG (e.g., SEQ ID NO: 107).

For example, a synthetic binding agent for treating or preventing infection by a Neisseria gonorrhoeae may include: a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to an epitope specific to Neisseria gonorrhoeae, so that the synthetic binding agent reduces the mobility of Neisseria gonorrhoeae in mucus. The one or more additional Fab domains may comprise 2, 4, 6 or 8 additional Fab domains. The additional Fab domains may each comprise: (i) a heavy chain (HC) with a variable region (V_{H}) comprising complementarity determining regions (CDRs) having an amino acid sequence that is between 100% and 80% identical to the amino acid sequence of: SEQ ID NO: 104; and/or (ii) a light chain (LC) with a variable region (V_{L}) comprising complementarity determining regions (CDRs) having an amino acid sequence that is between 100% and 80% identical to the amino acid sequence of: SEQ ID NO: 107.

Any of the synthetic binding agents described herein may include at least one additional Fab domains is linked to a Fab domain of the pair of Fab domains of the IgG; alternatively or additionally, any of the synthetic binding agents described herein may include at least one addition additional Fab domain that is linked to an Fc region of the IgG. The IgG may comprise at least one Fc region that is a naturally occurring sequence. The IgG may comprise at least one Fc region comprising one or more mutations that enhance or decrease binding to Fc receptors.

The one or more additional Fab domains may be linked to the IgG via a linker, as described here, such as a flexible peptide linker comprising an amino acid sequence comprising n pentapeptide repeats consisting of Glycine (G) and Serine (S), wherein n is between 3 and 8

In general, the IgG Fab domains may have an amino acid sequence that is not identical to the one or more additional Fab domains, while still recognizing the same antigen with the same (or nearly equivalent) affinity.

Also described herein are isolated nucleic acid molecules encoding any of these synthetic binding agents, and/or a vector comprising such isolated nucleic acid molecules. In some variations, the nucleotide sequence encoding the additional IgG (e.g., HV and/or LC) may be different from the nucleotide sequence encoding the region of the IgG having corresponding binding affinity; the resulting amino acid sequence may be identical or nearly identical (e.g., having 75% homology or more, 80% homology or more, 85% homology or more, 90% homology or more, 95% homology or more, etc., including corresponding substitutions). Also described herein are isolated host cells or a non-human organisms transformed or transfected with these nucleic acid molecules.

Also described herein are compositions of any of these synthetic binding agents and a pharmaceutically acceptable carrier.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a schematic example of a mAb directed against an epitope that may form a core of a synthetic binding agent (e.g., recombinant mAb) having enhanced agglutination and/or muco-trapping. In some variations the core antibody is directed to an antigen associated with sperm, and particularly human sperm. Alternatively, other epitopes, including viral or bacterial epitopes, may be used.

FIGS. 1B-1F illustrate examples of synthetic, engineered, constructs against an epitope of a target having enhanced agglutination and/or muco-trapping, using the core shown in FIG. 1A. FIG. 1B is an example of a Fab-IgG variation (e.g., duplicate Fab domain(s) linked to the amino ends of the core IgG). FIG. 1C is an example of an IgG-Fab variation (e.g., duplicate Fab domain(s) linked to the carboxyl ends of the core IgG). FIG. 1D is an example of a Fab-IgG-Fab variation (e.g., duplicate Fab domains linked to both the carboxyl ends and the amino ends of the core IgG). FIG. 1E is an example of a Fab-IgG-Fab-Fab variation (e.g., duplicate Fab domains linked to both the carboxyl ends and the amino ends of the core IgG). FIG. 1F is an example of a Fab-Fab-IgG-Fab-Fab variation of a synthetic binding agent as described herein (e.g., a 10-mer, having 4 additional pairs of Fab domains, two linked to the carboxyl ends and two to the amino ends of the core IgG).

FIG. 1G is a graphical table further illustrating structural properties of the core IgG and synthetic constructs shown in FIGS. 1A-1E.

FIGS. 2A-2B schematically illustrate muco-trapping. In this example, the target is a virus; other targets may include bacteria and sperm. In FIG. 2A the schematic shows how a target (e.g., virus) may readily diffuse across native mucus (in the absence of any virus-specific Ab). FIG. 2B shows how an anti-viral Ab, and particularly one that only weakly (in an unbound state) interacts with mucins and therefore freely diffuses through the mucus, may trap the target in mucus by adhesive interactions. Arrows indicate the small fraction of free (not virus-bound) Ab that will interact with mucins at any given time.

FIG. 3A illustrates agglutination of human sperm.

FIG. 3B illustrates trapping of sperm in mucus (e.g., cervicovaginal mucus (CVM) and endocervical mucus (CM)).

FIG. 4A illustrates an SDS-PAGE analysis of one example synthetic binding agent having enhanced agglutination and/or muco-trapping potencies (referred to as MM-006) compared to unmodified IgG. In this example, the synthetic binding agent is configured as an Fab-IgG-Fab construct and is compared to HCA (e.g., IgG).

FIG. 4B shows a color-matched SEC/MALS analysis of MM-006 from FIG. 4A. The SEC curves (solid curves, right y-axis) shows homogenous expression profile and MALS data (thicker line, left y-axis) confirms desired molecular weight (MW).

FIGS. 5A and 5B illustrate the application of the construct (MM-006) for FIG. 4A-4B to reduce the mobility/motility of sperm. FIG. 5A is a brightfield image of sperm 30 s after exposure to MM-006. FIG. 5B shows the sperm agglutination potency of MM-006 and HCA relative to PBS. * indicates p<0.05; **p<0.01; ***p<0.001.

FIGS. 6A-6B illustrate examples of an apparatus for delivering a construct (e.g., a contraceptive synthetic binding agent having multiple Fab repeats). FIG. 6A shows a transparent view of a ring with four capsules inserted into ring cavities. FIG. 6B shows exploded and assembled views of sustained release capsules: coated antibody pellet (center region, top), closed end capsule piece (right end of capsule), and capsule cap with release window (left end of capsule).

FIG. 6C shows daily (top) and cumulative (bottom) release of human IgG over at least 28 days with different sustained release capsule formulations.

FIG. 7 shows a silver stained gel showing human IgG recovered from capsules remained intact even after up to 4 weeks of exposure to human CVM (replaced every 5 days to maintain the degradative environment). Pure IgG band and PBS are shown as controls; all incubations were at 37° C. Each band represents IgG recovered from an individual capsule.

FIGS. 8A-8B show sperm agglutination in the presence of one of the synthetic binding agents having multiple Fab repeats described herein (configured as an HCA). FIG. 8A shows a light microscopy image of agglutinated sperm with a round cell (shown by arrow). FIG. 8B is a fluorescent image of the same field; the construct (HCA) in this example was conjugated to Dylight633 (red) and labeled the entire length of all spermatozoa. CMFDA (Live CellTracker green) labeled seminal leukocyte was also positive for HCA as indicated by the co-labeling (white arrow).

FIG. 9 is a graph illustrating differential scanning calorimetry of an exemplary synthetic binding agent having multiple Fab repeats (e.g., Fab-IgG, bottom) and an scFv-IgG (top) construct. The scFv construct shows unfolding at a much lower temperature than the synthetic binding agent having multiple Fab repeats (Fab-IgG) due to the lack of a CH1/CL domain.

FIG. 10 shows an Analytical Size Exclusion Chromatography graph of the core alone (IgG, bottom), an scFv-IgG construct (middle), and an exemplary synthetic binding agent having multiple Fab repeats (Fab-IgG, top) after single-step protein A purification. Both the IgG and Fab-IgG construct showed a single sharp peak at their expected molecular weight. The scFv-IgG construct shows formation of high molecular-weight aggregates.

FIG. 11A shows an SDS-PAGE analysis of multimeric synthetic binding agent having multiple Fab repeats. In this example the multimeric constructs are HCA (e.g., contraceptive) synthetic binding agents having multiple Fab repeats, where all of the Fabs are directed against the same epitope of CD52g (e.g., all share the same targeting sequence).

FIG. 11B is a color-matched SEC/MALS analysis of the different multimeric HCA constructs from FIG. 11A. SEC curves for each construct (solid curves, right y-axis) shows homogenous expression profile and MALS data (dotted lines, left y-axis) confirms desired molecular weight of each construct (also compared to the core IgG).

FIG. 12A shows a whole-sperm ELISA verifying that different multimeric synthetic binding agents having multiple Fab repeats (configured as HCA constructs) possess functional Fab that binds sperm.

FIGS. 12B and 12C show brightfield images of sperm 1 minute after treatment with PBS (FIG. 12B), or after 30 seconds after treatment with a multimeric synthetic binding agent having multiple Fab repeats (e.g., configured as a Fab-IgG-Fab HCA construct) (FIG. 12C).

FIG. 13 shows the muco-trapping potency of a Fab-IgG, as reflected by the ensemble geometric average of effective diffusivity (Deff) of PEGylated nanoparticles (PS-PEG) in human CVM with no Ab, control IgG (Ctrl IgG) or a multimeric synthetic binding agent having multiple Fab repeats (e.g., an anti-PEG Fab-IgG construct), compared to uncoated nanoparticles (PS-COOH) in untreated CVM. Data is plotted for distinct samples (indicated with different color circles) with averages indicated by solid lines. * indicates a statistically significant difference (p<0.05).

FIGS. 14A and 14B show an amino acid sequence comparison between portions of the heavy (FIG. 14A; the VH portion of SEQ ID NO: 3 is compared to a VH portion of a germine sequence) and light (FIG. 14B; the VL portion of SEQ ID NO: 7 is compared to a VL portion of a germline sequence) chain sequences of a core IgG against an epitope on human sperm, CD52g, compared to a germline sequence (e.g., native IgG).

FIGS. 15A-15D illustrate one example of the production and characterization of multimeric anti-sperm IgG antibodies. FIG. 15A schematically illustrates examples of anti-sperm IgG, Fab, Fc, Fab-IgG, and IgG-Fab. FIG. 15B is a gel showing non-reducing IgG, Fab-IgG, and IgG-Fab. FIG. 15C shows a reducing SDS-Page analysis comparing IgG, Fab-IgG, and IgG-Fab after expression in Expi293 cells and purification by protein A/G chromatography. FIG. 15D is a graph illustrating the purity and homogeneity of the purified multimeric antibodies (IgG compared to Fab-IgG and IgG-Fab) via analytical SEC-MALS analysis.

FIGS. 16A-16C illustrate the characterization of multimeric anti-sperm IgG antibodies. FIG. 16A shows the molar mass versus time of the IgG, Fab-IgG and IgG-Fab respectively as determined by SEC-MALS. FIG. 16B shows the values of melting temperature (Tm) and aggregating temperature (Tagg) of as determined by nanoDSF by measuring intrinsic fluorescence of a protein and changes in back-reflection respectively. FIG. 16C is a graph showing whole sperm ELISA analysis to assess the binding potency of indicated antibodies. Motavizumab (anti-RSV IgG1) is used as the isotype control. ELISA was performed in triplicates and repeated three times using 3 unique specimens. Lines indicate arithmetic mean concentration and standard error of mean.

FIGS. 17A-17B illustrate sperm agglutination potency of parent IgG, Fab-IgG and IgG-Fab using purified motile sperm (10×10⁶ progressively motile sperm per mL). FIG. 17A graphically illustrates measured sperm agglutination potency of the parent and multimeric anti-sperm IgGs by quantifying the percentage of sperm that remains progressively motile post Ab-treatment at different concentrations compared to pre-treatment condition. FIG. 17B shows the percentage of agglutination-escaped progressive sperm post-treatment normalized to the negative control for further comparison. Data represents 6 unique sperm specimens. Lines indicate arithmetic mean concentration and standard error of mean.

FIGS. 18A-18B show sperm agglutination kinetics of parent IgG, Fab-IgG and IgG-Fab using purified motile sperm (10×10⁶ progressively motile sperm per mL). FIG. 18A is a graph showing quantification of the time required to achieve 90% agglutination of progressive sperm compared to untreated control. The CASA analysis was obtained every 30 s post-treatment until 90 s. FIG. 18B shows the rate of sperm agglutination of indicated anti-sperm antibodies by quantifying the percentage of agglutinated sperm post Ab-treatment at three different time points compared to pre-treatment. Data represents 6 unique sperm specimens. Lines indicate arithmetic mean concentration and standard error of mean.

FIG. 19 illustrates the muco-trapping potency of parent IgG, Fab-IgG and IgG-Fab using pH-neutralized female CVM and purified motile sperm (1×10⁶ progressively motile sperm/mL). Muco-trapping potency of indicated antibodies was assessed by performing real-time video microscopy on fluorescently labelled sperm suspended in Ab-treated (25 ug/ml) CVM. A neural network tracker customized with standardized sperm motility parameters was used in all recorded videos to quantify the percentage of progressively motile sperm present in the mucus specimen.

FIGS. 20A-20D illustrate production and characterization of multimeric anti-sperm IgG antibodies. FIG. 20A schematically illustrates anti-sperm Fab, Fc, IgG, Fab-IgG-Fab (FIF), Fab-IgG-Fab-Fab (FIFF) and Fab-Fab-IgG-Fab-Fab (FFIFF). In this example, N-terminal and C-terminal Fab/s of FIF, FIFF and FFIFF contains fully intact anti-sperm Fab/s with V_{H}, V_{L}, C_{H}1, and C_{L}. FIG. 20B is a gel showing non-reducing FIF, FIFF and FFIFF (compared to IgG). FIG. 20C is a reducing SDS-Page analysis of the indicated antibodies (IgG, FIF, FIFF, and FFIFF) after expression in Expi293 cells and purification by protein A/G chromatography. FIG. 20D is a graph demonstrating the purity and homogeneity of the purified multimeric antibodies via analytical SEC-MALS analysis.

FIGS. 21A-21C illustrate additional characterization of multimeric anti-sperm IgG antibodies. FIG. 21A illustrates the molar mass versus time of the IgG, FIF, FIFF and FFIFF respectively as determined by SEC-MALS. FIG. 21B graphically illustrates the value of melting temperature (Tm) and aggregating temperature (Tagg) of indicated antibodies as determined by nanoDSF by measuring intrinsic fluorescence of a protein and changes in back-reflection respectively. FIG. 21C is a graph showing a whole Sperm ELISA analysis to assess the binding potency of the indicated antibodies. Motavizumab (anti-RSV IgG1) is used as the isotype control. ELISA was performed in in triplicates and repeated three times using 3 unique specimens. Lines indicate arithmetic mean concentration and standard error of mean.

FIG. 22A-22D illustrates sperm agglutination potency of parent IgG and multimeric constructs using purified motile sperm (10×10⁶ progressively motile sperm per mL) and whole semen. FIG. 22A is a graph showing sperm agglutination potency of the parent IgG, FIF, FIFF and FFIFF by quantifying the percentage of sperm that remains progressively motile post Ab-treatment compared to pre-treatment condition using purified motile sperm (10×10⁶ progressively motile sperm per mL). FIG. 22B shows the percentage of agglutination-escaped progressive sperm post Ab-treatment using purified motile sperm that was normalized to the negative control for further comparison. FIG. 22C is a graph showing measured sperm agglutination potency of the parent IgG and FFIFF by quantifying the percentage of sperm that remains progressively motile post Ab-treatment compared to pre-treatment condition using whole semen. FIG. 22D shows the percentage of agglutination-escaped progressive sperm post Ab-treatment using whole semen that was normalized to the negative control for further comparison. Data represents 6 unique sperm specimens. Lines indicate arithmetic mean concentration and standard error of mean.

FIGS. 23A-23D shows sperm agglutination kinetics of parent IgG and multimeric constructs using purified motile sperm (10×10⁶ progressively motile sperm per mL) and whole semen. FIG. 23A illustrates agglutination kinetics of parent IgG, FIF, FIFF and FFIFF determined by quantifying the time required to achieve 90% agglutination of progressive sperm compared to untreated control using purified motile sperm (10×10⁶ progressively motile sperm per mL). CASA analysis was obtained every 30 s post-treatment until 90 s. FIG. 23B illustrates the rate of sperm agglutination of parent IgG, FIF, FIFF and FFIFF by quantifying the percentage of agglutinated sperm post Ab-treatment at three different time points compared to pre-treatment using purified motile sperm (10×10⁶ progressively motile sperm per mL). FIG. 23C shows the agglutination kinetics of parent IgG and FFIFF by quantifying the time required to achieve 90% agglutination of progressive sperm compared to untreated control using whole semen. The CASA analysis was obtained every 30 s post-treatment until 90 s. FIG. 23D is a graph showing the rate of sperm agglutination of parent IgG and FFIFF by quantifying the percentage of agglutinated sperm post Ab-treatment at three different time points compared to pre-treatment using whole semen. Data represents 6 unique sperm specimens. Lines indicate arithmetic mean concentration and standard error of mean.

FIGS. 24A-24D show sperm agglutination kinetics of parent IgG and FFIFF using low and high concentration of purified motile sperm (2×10⁶ and 50×10⁶ progressive sperm/mL). FIG. 24A shows the agglutination kinetics of IgG and FFIFF by quantifying the time required to achieve 90% agglutination of progressive sperm compared to untreated control. The CASA analysis was obtained every 30 s post-treatment until 90 s using purified motile sperm (2×10⁶ progressive sperm/mL). FIG. 24B shows the rate of sperm agglutination of IgG and FFIFF by quantifying the percentage of agglutinated sperm post Ab-treatment at three different time points compared to pre-treatment using purified motile sperm (2×10⁶ progressive sperm/mL). FIG. 24C shows the agglutination kinetics of IgG and FFIFF by quantifying the time required to achieve 90% agglutination of progressive sperm compared to untreated control using purified motile sperm (50×10⁶ progressive sperm/mL). The CASA analysis was obtained every 30 s post-treatment until 90 s. FIG. 24D shows the rate of sperm agglutination of IgG and FFIFF by quantifying the percentage of agglutinated sperm post Ab-treatment at three different time points compared to pre-treatment using purified motile sperm (50×10⁶ progressive sperm/mL). Data represents 6 unique sperm specimens. Lines indicate arithmetic mean concentration and standard error of mean.

FIG. 25 is a graph showing sperm agglutination potency of parent IgG, FIF and FFIFF using whole semen in sheep study. Agglutination potency of IgG, FIF and FFIFF was measured in vivo by instilling Abs into sheep vagina, followed by human semen and simulated intercourse. The sperm motility was assessed immediately in the fluids from the sheep vagina. Data represents 3 unique sheep studies for FIF and FFIFF, and 1 sheep study for IgG at both 33 ug/ml and 333 ug/ml. Lines indicate arithmetic mean concentration and standard error of mean.

FIGS. 26 and 27A illustrate agglutination potency of exemplary films (e.g., Nicotiana-produced films) of parent IgG and FIF using purified motile sperm (10×10⁶ progressive sperm/mL) and whole semen. FIG. 26 shows sperm agglutination potency of the parent IgG-Film and FIF-Film by quantifying the percentage of sperm that remains progressively motile post Ab-treatment compared to pre-treatment condition using purified motile sperm (10×10⁶ progressively motile sperm per mL). FIG. 27A illustrates the percentage of agglutination-escaped progressive sperm post Ab-treatment using purified motile sperm; the data is normalized to the negative control.

FIG. 27B illustrates sperm agglutination potency of the parent IgG-Film and FIF-Film by quantifying the percentage of sperm that remains progressively motile post Ab-treatment compared to pre-treatment condition using whole semen.

FIG. 27C shows the percentage of agglutination-escaped progressive sperm post Ab-treatment using whole semen, normalized to the negative control. Data represents 6 unique sperm specimens. Lines indicate arithmetic mean concentration and standard error of mean.

FIGS. 28A-28D show agglutination kinetics of exemplary films (e.g., Nicotiana-produced films) of parent IgG and FIF using purified motile sperm (10×10⁶ progressively motile sperm/mL) and whole semen. FIG. 28A illustrates agglutination kinetics of indicated antibodies by quantifying the time required to achieve 90% agglutination of progressive sperm compared to untreated control using purified motile sperm (10×10⁶ progressively motile sperm/mL). The CASA analysis was obtained every 30 s post-treatment until 90 s. FIG. 28B illustrates the rate of sperm agglutination of indicated anti-sperm antibodies by quantifying the percentage of agglutinated sperm post Ab-treatment at three different time points compared to pre-treatment using purified motile sperm (10×10⁶ progressively motile sperm/mL). FIG. 28C is a graph showing agglutination kinetics of indicated antibodies by quantifying the time required to achieve 90% agglutination of progressive sperm compared to untreated control using whole semen. The CASA analysis was obtained every 30 s post-treatment until 90 s. FIG. 28D shows the measured rate of sperm agglutination of indicated anti-sperm antibodies (quantifying the percentage of agglutinated sperm post Ab-treatment at three different time points compared to pre-treatment using whole semen). Data represents 6 unique sperm specimens. Lines indicate arithmetic mean concentration and standard error of mean.

FIGS. 29A-29D illustrate agglutination kinetics exemplary films (e.g., Nicotiana-produced films) of parent IgG and FIF using low and high concentration of purified motile sperm (2×10⁶ and 50×10⁶ progressively motile sperm/mL). FIG. 29A shows the agglutination kinetics of indicated antibodies based on quantification of the time required to achieve 90% agglutination of progressive sperm compared to untreated control using purified motile sperm (2×10⁶ progressively motile sperm/mL). The CASA analysis was obtained every 30 s post-treatment until 90 s. FIG. 29B shows the rate of sperm agglutination of indicated anti-sperm antibodies as determined by quantifying the percentage of agglutinated sperm post Ab-treatment at three different time points compared to pre-treatment using purified motile sperm (2×10⁶ progressively motile sperm/mL). FIG. 29C shows the agglutination kinetics of indicated antibodies by quantifying the time required to achieve 90% agglutination of progressive sperm compared to untreated control using purified motile sperm (50×10⁶ progressively motile sperm/mL). The CASA analysis was obtained every 30 s post-treatment until 90 s. FIG. 29D shows the rate of sperm agglutination of indicated anti-sperm antibodies by quantifying the percentage of agglutinated sperm post Ab-treatment at three different time points compared to pre-treatment using purified motile sperm (50×10⁶ progressively motile sperm/mL). Data represents 6 unique sperm specimens. Lines indicate arithmetic mean concentration and standard error of mean.

FIG. 30 illustrates the agglutination kinetics of exemplary films (e.g., Nicotiana-produced films) of parent IgG and FIF in an acidic environment using purified motile sperm (20×10⁶ progressively motile sperm/mL) and 24 hr treatment with lactic acid. The agglutination kinetics of lactic acid-treated antibodies were assessed by quantifying the time required to achieve 90% agglutination of progressive sperm compared to untreated control. The CASA analysis was obtained every 30 s post-treatment until 90 s. Data represents 2 unique sperm specimens. Note: Lactic acid (LA) treated antibodies were neutralized with seminal plasma (SP) and then followed by dilution with SP or saline media i.e. MHM.

FIGS. 31A-31D illustrate characterization of a Nicotiana-produced FIF-Film (film of Fab-IgG-Fab synthetic binding agent) and Expi293-produced FFIFF (e.g., Fab-Fab-IgG-Fab-Fab) synthetic binding agent post-nebulization. FIG. 31A shows a non-reducing SDS-Page analysis of the indicated antibodies before and after nebulization using mesh-nebulizer, and FIG. 31B shows a reducing SDS-Page analysis of the indicated antibodies before and after nebulization using mesh-nebulizer. In FIGS. 31C and 31D, whole-sperm ELISA analysis was used to assess the binding potency of the FIF-Film (shown in FIG. 31A) and FFIFF post-nebulization (FIG. 31D). ELISA was performed in triplicates and repeated twice using the same donor specimens. Lines indicate arithmetic mean concentration and standard error of mean.

FIGS. 32A-32B illustrate the production and characterization of multimeric anti-RSV IgG antibodies. FIG. 32A shows a non-reducing gel and reducing SDS-Page analysis of a multimeric IgG against RSV (Motavizumab as parent IgG) after expression in Expi293 cells and purification by protein A/G chromatography. FIG. 32B is an RSV ELISA analysis to assess the binding potency of the indicated antibodies. Synagis/Palivizumab (anti-RSV IgG1) is used as the positive control. ELISA was performed in triplicates.

### DETAILED DESCRIPTION

The methods and compositions (including the multimeric synthetic binding agent having multiple Fab repeats for enhancing agglutination, facilitating enchained growth and/or improving muco-trapping) described herein are based, in part, on the discovery that foreign bodies, including pathogens such as virus and bacteria, as well as sperm, may be more strongly trapped by mucus following binding with a multimeric antibody-based constructs. The constructs may be engineered to stop the penetration of target (e.g., pathogen, sperm, etc.) through mucus by improving the agglutination potency, facilitating enchained growth of the pathogen and/or enabling muco-trapping, and may prevent and/or treat infection, and/or provide contraception.

The present invention is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following specification is intended to illustrate some particular embodiments of the invention, and not to exhaustively specify all permutations, combinations and variations thereof.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Except as otherwise indicated, standard methods known to those skilled in the art may be used for production of recombinant and synthetic polypeptides, antibodies or antigen-binding fragments thereof, manipulation of nucleic acid sequences, and production of transformed cells. Such techniques are known to those skilled in the art. See, e.g., SAMBROOK et al., MOLECULAR CLONING: A LABORATORY MANUAL 2nd Ed. (Cold Spring Harbor, N.Y., 1989); F. M. AUSUBEL et al. CURRENT PROTOCOLS IN MOLECULAR BIOLOGY (Green Publishing Associates, Inc. and John Wiley & Sons, Inc., New York).

All publications, patent applications, patents, nucleotide sequences, amino acid sequences and other references mentioned herein are incorporated by reference in their entirety.

As used in the description of the invention and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

Moreover, the present invention also contemplates that in some embodiments of the invention, any feature or combination of features set forth herein can be excluded or omitted.

The term “about,” as used herein when referring to a measurable value such as an amount of a compound or agent of this invention, dose, time, temperature, and the like, is meant to encompass variations of ±10%, ±5%, ±1%, ±0.5%, or even ±0.1% of the specified amount.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

The transitional phrase “consisting essentially of” means that the scope of a claim is to be interpreted to encompass the specified materials or steps recited in the claim, and those that do not materially affect the basic and novel characteristic(s) of the claimed invention.

As used herein, the term “polypeptide” encompasses both peptides and proteins, unless indicated otherwise.

A “nucleic acid” or “nucleotide sequence” is a sequence of nucleotide bases, and may be RNA, DNA or DNA-RNA hybrid sequences (including both naturally occurring and non-naturally occurring nucleotide), but is preferably either single or double stranded DNA sequences.

As used herein, an “isolated” antibody means an antibody separated or substantially free from at least some of the other components of the naturally occurring organism or virus, for example, the cell structural components or other polypeptides or nucleic acids commonly found associated with the antibody. The term also encompasses antibodies that have been prepared synthetically.

By the terms “treat,” “treating,” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is a delay in the progression of the condition.

As used herein, the terms “prevent,” “prevents,” or “prevention” and “inhibit,” “inhibits,” or “inhibition” (and grammatical equivalents thereof) are not meant to imply complete abolition of disease and encompasses any type of prophylactic treatment that reduces the incidence of the condition, delays the onset of the condition, and/or reduces the symptoms associated with the condition after onset.

An “effective,” “prophylactically effective,” or “therapeutically effective” amount as used herein is an amount that is sufficient to provide some improvement or benefit to the subject. Alternatively stated, an “effective,” “prophylactically effective,” or “therapeutically effective” amount is an amount that will provide some delay, alleviation, mitigation, or decrease in at least one clinical symptom in the subject. Those skilled in the art will appreciate that the effects need not be complete or curative, as long as some benefit is provided to the subject.

As used herein, the term “trapping potency” refers to the ability of an antibody that specially binds to a target pathogen or sperm to inhibit movement of the pathogen or sperm through mucus. Trapping potency can be measured by methods known in the art and as disclosed herein. Trapping potency can be quantitated, e.g., as the amount of antibody (e.g., concentration of antibody in mucus) needed to reduce the mobility of at least 50% (e.g., at least 55%, at least 60%, at least 65%, at least 70%, at least 75%, etc.) of the pathogen or sperm within the mucus gel to at least one-half (e.g., one-quarter, one-tenth, etc.) of its native mobility in solution (e.g., saline) and/or in mucus. For sperm, trapping potency can also be quantitated, e.g., as the amount of antibody (e.g., concentration of antibody in mucus) needed to reduce the fraction of progressively motile sperm by at least 50% (e.g. at least 55%, at least 60%, at least 65%, at least 70%, at least 75%, etc.) as determined by Computer Assisted Sperm Analysis (CASA). Mobility in mucus can be measured using techniques well known in the art and described herein. Alternatively, trapping potency can be quantitated as the reduction in percentage of pathogens or sperm that penetrate mucus.

The term “enhances trapping potency” refers to enhancement compared to the core antibody (e.g., core IgG). Further, any of the multimeric synthetic binding agents having multiple Fab repeats described herein may be selected or further configured to enhance mucin-crosslinking by including a glycosylation pattern comprising the biantennary core glycan structure Manα1-6(Manα1-3)Manβ1-4GlcNAcβ1-4GlcNAcβ1 with terminal N-acetylglucosamine on each branch. This glycosylation pattern may be on the Fc region of the core Ab (e.g., the core IgG). Alternatively or additionally, a composition of the synthetic binding agent having multiple Fab repeats described herein may be selected or configured such that at least x % of the synthetic binding agent having multiple Fab repeats have a glycosylation pattern comprising the biantennary core glycan structure Manα1-6(Manα1-3)Manβ1-4GlcNAcβ1-4GlcNAcβ1 with terminal N-acetylglucosamine on each branch, where x % is 20%, 25%, 30%, 35%, 40%, 45%, 50%, 55%, 60%, 65%, 70%, 75%, 80%, 85%, 90%, 95%, or substantially all). A composition in which, for example, greater than 40% of the synthetic binding agent having multiple Fab repeats described herein (to enhance agglutination potency) while also possessing an oligosaccharide that provides increased mucin crosslinking, may be particularly beneficial for muco-trapping of a target once bound to the target, compared to core IgG as found in nature prior to any modification and/or selection.

As used herein, the term “bind specifically” or “specifically binds” in reference to an antibody of the presently-disclosed subject matter means that the antibody of the invention will bind with an epitope (including one or more epitopes) of a target pathogen or sperm, but does not substantially bind to other unrelated epitopes or molecules. In certain embodiments, the term refers to an antibody that exhibits at least about 60% binding, e.g., at least about 70%, 80%, 90%, or 95% binding, to the target epitope relative to binding to other unrelated epitopes or molecules.

The antibodies, compositions, and methods described herein may include methods for inhibiting and/or treating pathogen infection, eliminating pathogen from a mucosal surface, and providing contraception. In particular, the presently-disclosed subject matter relates to synthetic binding agents having multiple Fab repeats and compositions of these that are capable of facilitating aggregation and/or enchained growth of pathogens and sperm, trapping pathogens and sperm in mucus, thereby inhibiting transport of pathogens or sperm across or through mucus secretions, which may lead to the destruction and/or natural elimination of these pathogens and/or sperm.

Any of the synthetic binding agents having multiple Fab repeats described herein be directed to non-neutralizing epitopes of pathogens; in some variations, the synthetic binding agents having multiple Fab repeats described herein be directed to neutralizing epitopes of pathogens.

Antibodies are naturally found in mucus. The synthetic binding agent having multiple Fab repeats described herein may generally diffuse rapidly through mucus, slowed only slightly by weak, transient adhesive interactions with mucins within the mucus. This rapid diffusion allows the synthetic binding agent having multiple Fab repeats to accumulate rapidly on pathogen or sperm surfaces. When a plurality of synthetic binding agents have accumulated on the surface of a pathogen or sperm, the adhesive interactions between the plurality of antibodies and the mucus become sufficient to trap the bound pathogen or sperm in the mucus, thereby preventing infection/providing contraception. Moreover, and somewhat surprisingly, binding multiple pathogens using the same synthetic binding agent having multiple Fab repeats may more effectively trap the complex formed by the multiple pathogens/sperm and the synthetic binding agent, either by aggregation of distinct pathogens/sperm together or facilitating enchained growth of pathogens. Pathogens or sperm trapped in CVM cannot reach their target cells in the mucosal surface, and will instead be shed with post-coital discharge and/or inactivated by spontaneous thermal degradation as well as additional protective factors in mucus, such as defensins (Cole, Curr. Top. Microbiol. Immunol. 306:199 (2006); Doss et al., J. Leukoc. Biol. 87:79 (2010)). As disclosed herein, this pathogen agglutination and/or trapping activity provides for protection without neutralization, and can effectively inhibit infection at sub-neutralization doses and/or using antibodies to non-neutralizing epitopes of a pathogen. The low-affinity interactions that the synthetic binding agent having multiple Fab repeats described herein may form with mucins are not only Fc-dependent, but may also influenced by antibody glycosylation.

Accordingly, the synthetic binding agent having multiple Fab repeats described herein may include an oligosaccharide at a glycosylation site, the oligosaccharide comprising, consisting essentially of, or consisting of a pattern correlating with (providing) enhanced trapping potency of the antibody in mucus, and wherein the antibody specifically binds an epitope of a target (e.g., pathogen or sperm). The unique glycosylation pattern/unique oligosaccharide component of the antibody may maximize trapping potency of the synthetic binding agent once a synthetic binding agent forms a complex with one or more target (e.g., pathogen or sperm), without unduly hindering the ability of the unbound synthetic binding agent to diffuse readily through mucus to rapidly bind a target. In certain embodiments, the synthetic binding agent having multiple Fab repeats described herein is one that exhibits a mobility in mucus that is reduced no more than about 50%, e.g., no more than about 40%, 30%, 20%, 10%, or 5%, relative to its native mobility in solution (e.g., mucus, saline or water) and effectively traps a target pathogen or sperm in mucus once complexed with one or more targets (e.g., at least 50% of target slowed by at least on half). In some embodiments, the synthetic binding agent having multiple Fab repeats described herein reduces the mobility of at least 50% of the target, e.g., at least 50%, 60%, 70%, 80%, or 90% or more of the target, by at least 50% (e.g., 60%, 70%, 75%, 80%, 85%, 90%, 95%, 96%, 97%, 98%, or 99%, etc.) or more. In other embodiments, the synthetic binding agent having multiple Fab repeats described herein reduces the percentage of target (e.g., pathogens or sperm) that can penetrate mucus by at least 10%, e.g., at least 20%, 30%, 40%, 50%, 60%, 70%, 80%, 90%, or more. For example, the synthetic binding agent having multiple Fab repeats described herein may have a sufficient binding rate to an epitope of the target to trap the target pathogen or sperm in mucus within one hour (e.g., within 30 minutes or 15 minutes) at a synthetic binding agent concentration in the mucus of less than 10 mg/ml (e.g., less than 5 mg/ml, less than 1 mg/ml, less than 0.1 mg/ml, less than 50 μg/ml, less than 30, less than 20, less than 10, less than 5, less than 2.5, less than 1, less than 0.5, less than 0.1 μg/ml, etc.).

In some embodiments, the synthetic binding agent having multiple Fab repeats described herein may include an oligosaccharide component that is bound to an N-linked glycosylation site in an Fc region of the synthetic binding agent (e.g., the core IgG portion of the synthetic binding agent). The N-linked glycosylation site can be an asparagine residue on the Fc region of the core, for example, the Asn 297 asparagine residue. The amino acid numbering is with respect to the standard amino acid structure of a human IgG molecule.

The N-glycan structure may be G0/G0F form, or a pure GnGn form (e.g., with terminal N-acetylglucosamine on each branch without terminal galactose or sialic acid). In some embodiments, the oligosaccharide component, i.e., the glycan, attached to the antibody comprises, consists essentially of, or consists of a core structure without any fucose residue. In other embodiments, the glycan does not contain any galactose residues. In some embodiments the glycan does not include galactose.

The synthetic binding agent having multiple Fab repeats described herein may include a mixture of synthetic binding agents having different oligosaccharide components. In some embodiments, the mixture comprises at least about 30% synthetic binding agent having multiple Fab repeats described herein having the G0/G0F core glycan structure (e.g., with or without the fucose residue), e.g., at least about 40%, 50%, 60%, 70%, 80%, 90% or more.

In some embodiments, the synthetic binding agent having multiple Fab repeats described herein are generated in a human cell line, e.g., a 293 cell line, e.g., a 293T cell line, other mammalian cell lines (e.g. CHO), in plants (e.g. Nicotiana), or in other microorganisms (e.g. Trichoderma).

The synthetic binding agent having multiple Fab repeats described herein may be useful for binding target to trap the target in mucus to inhibit infection or impregnation by the target. In variations in which the target is a pathogen, the synthetic binding agent having multiple Fab repeats described herein can be directed to any pathogen that can infect a subject through a mucus membrane. Pathogens can be in the categories of algae, bacteria, fungi, parasites (helminths, protozoa), viruses, and subviral agents. Target pathogens further include synthetic systems comprising an antigen having an epitope, for example particles or particulates (e.g., polystyrene beads) comprising attached proteins, e.g., as might be used for bioterrorism. Pathogens include those that cause sexually-transmitted diseases (listed with the diseases caused by such pathogens), including, without limitation, Neisseria gonorrhoeae (gonorrhea); Chlamydia trachomatis (chlamydia, lymphogranuloma venereum); Treponema pallidum (syphilis); Haemophilus ducreyi (chancroid); Klebsiella granulomatis or Calymmatobacterium granulomatis (donovanosis), Mycoplasma genitalium, Ureaplasma urealyticum (mycoplasmas); human immunodeficiency virus HIV-1 and HIV-2 (HIV, AIDS); HTLV-1 (T-lymphotrophic virus type 1); herpes simplex virus type 1 and type 2 (HSV-1 and HSV-2); Epstein-Barr virus; cytomegalovirus; human herpesvirus 6; varicella-zoster virus; human papillomaviruses (genital warts); hepatitis A virus, hepatitis B virus, hepatitis C virus (viral hepatitis); molluscum contagiosum virus (MCV); Trichomona vaginalis (trichomoniasis); and yeasts, such as Candida albicans (vulvovaginal candidiasis). The antibodies and compositions may also be active against other diseases that are transmitted by contact with bodily fluids that may also be transmissible by sexual contact and are capable of being prevented by administration of the compositions according to this invention. Accordingly, the phrase, “sexually transmitted diseases (STDs),” is to be interpreted herein as including any disease that is capable of being transmitted in the course of sexual contact, whether or not the genital organs are the site of the resulting pathology. Pathogens also include those that cause respiratory diseases, including, without limitation, influenza (including influenza A, B, and C); severe acute respiratory syndrome (SARS); respiratory syncytial virus (RSV); parainfluenza; adenovirus; human rhinovirus; coronavirus; and norovirus. Other pathogens include, without limitation, Salmonella and Escherichia coli. Other pathogens include Klebsiella bacillus.

Pathogens may include those that affect non-human animals, such as livestock, e.g., swine (e.g., porcine epidemic diarrhea virus (PEDV), transmissible gastroenteritis virus (TGEV), rotavirus, classical swine fever virus (CSFV), porcine circovirus type 2 (PCV2), encephalomyocarditis virus (EMCV), porcine reproductive and respiratory syndrome virus (PRRSV), porcine parvovirus (PPV), pseudorabies virus (PRV), Japanese encephalitis virus (JEV), Brucella, Leptospira, Salmonella, and Lawsonia intracellularis, Pasteurella mulocida, Brachyspira hyodysenteriae, Mycoplasma hyopneumoniae), ruminants (e.g., bovine virus diarrhoea virus (BVDV), border disease virus (BDV), bovine papular stomatitis virus (BPSV), pseudocowpox virus (PCPV), Pasteurella haemolytica, Pasteurella multocida, Haemophilus somnus, Haemophilus agnii, Moraxella bovis, Mycoplasma mycoides, Theileria annulata, Mycobacterium avium paratuberculosis), ungulates (e.g., Brucella abortus, Mycobacterium bovis, Theileria parva, Rift Valley fever virus, foot-and-mouth disease virus, lumpy skin disease virus), horses (e.g., Rhodococcus equi, Salmonella choleraesuls, Pasteurella multocida, equine herpesvirus-1, equine herpesvirus-4, equine influenza virus, Streptococcus equi), poultry (e.g., fowl pox virus, Newcastle disease virus, Marek's disease virus, avian influenza virus, infectious bursal disease virus (IBDV), avian infectious bronchitis virus (IBV)), and the like.

The terms virus and viral pathogen are used interchangeably herein, and further refer to various strains of virus, e.g., influenza is inclusive of new strains of influenza, which would be readily identifiable to one of ordinary skill in the art. The terms bacterium, bacteria, and bacterial pathogen are used interchangeably herein, and further refer to antibiotic-resistant or multidrug resistant strains of bacterial pathogens. As used herein when referring to a bacterial pathogen, the term “antibiotic-resistant strain” or “multidrug resistant strain” refers to a bacterial pathogen that is capable of withstanding an effect of an antibiotic or drug used in the art to treat the bacterial pathogen (i.e., a non-resistant strain of the bacterial pathogen).

In some embodiments, it is contemplated that a synthetic binding agent having multiple Fab repeats described herein is capable of broadly binding to viruses containing lipid envelopes, which are not necessarily specific to one virus.

In variations when the synthetic binding agent having multiple Fab repeats described herein specifically binds a neutralizing epitope of the target pathogen, a sub-neutralization dose can be used. A sub-neutralization doses is a dose below that which would be needed to achieve effective neutralization. For example, in the case of polyclonal anti-HSV gG antibodies targeting HSV, as described hereinbelow, an effective neutralization dose is approximately 5 μg/ml. However, effective agglutination and/or trapping using the synthetic binding agent having multiple Fab repeats described herein can be achieved at a dose below 5 μg/ml, and even below a dose of 1 μg/ml.

As will be recognized by one of skill in the art, doses appropriate for agglutination and/or trapping bacterial pathogens can be higher in some embodiments than the doses appropriate for trapping viral pathogens. It will further be recognized that appropriate doses may differ between pathogens, between mucosal surfaces, and also between individuals. It will also be recognized that different subjects and different mucosal surfaces may have different optimal glycan patterns and optimal antibody-mucin affinities, contributing to different optimal doses.

It is further proposed herein that synthetic binding agent having multiple Fab repeats described herein that selectively bind non-neutralizing epitopes of a target pathogen can be used to effectively trap the target pathogen in mucus. As such, in some embodiments, the synthetic binding agent having multiple Fab repeats specifically binds a non-neutralizing epitope, e.g., one or more non-neutralizing epitopes.

The presently disclosed subject matter further includes synthetic binding agent having multiple Fab repeats that selectively binds a conserved epitope of a target. A benefit of targeting a conserved epitope would be to preserve efficacy of the synthetic binding agent having multiple Fab repeats as against new strains of the pathogen. Targeting such epitopes has been avoided at times in the past because they were viewed as being ineffective targets; however, in view of the disclosure herein such epitopes can serve as effective targets.

The synthetic binding agent having multiple Fab repeats described herein may be particularly useful for binding sperm to trap the sperm in mucus to inhibit fertilization of an egg by the sperm. Sperm specific antigens that can be used as antibody targets are well known in the art. See, e.g., U.S. Pat. Nos. 8,211,666, 8,137,918, 8,110,668, 8,012,932, 7,339,029, 7,230,073, and 7,125,550, each incorporated by reference in its entirety. As will be described herein one particular epitope region for human sperm may include the N-linked glycan of sperm CD52 glycoform. See also U.S. Pat. Nos. 5,227,160 and 6,355,235, herein incorporated by reference in their entirety.

The low-affinity binding interactions that the synthetic binding agent having multiple Fab repeats described herein forms with mucins may be influenced by glycosylation, and may also be Fc-dependent. As such, the synthetic binding agent having multiple Fab repeats described herein may have a preserved and/or engineered Fc region in the core IgG region. Such synthetic binding agents may be one or more subclasses of IgG, e.g., IgG₁, IgG₂, IgG₃, IgG₄, or any combination thereof.

In some embodiments, the synthetic binding agent having multiple Fab repeats described herein has a sufficient binding rate and/or binding affinity to an epitope of the target to accumulate on the surface of the target at sufficient levels to trap the target within one hour after administration of the synthetic binding agent having multiple Fab repeats described herein at a concentration of less than about 10 mg/mL (e.g., less than about 5 mg/mL, less than 2 mg/mL, less than about 1 mg/mL, less than about 0.1 mg/mL, less than about 50 μg/ml, less than about 40 μg/ml, less than about 30 μg/ml, less than about 20 μg/ml, less than about 10 μg/ml, less than about 5 μg/ml, less than about 1 μg/ml, less than about 0.5 μg/ml, less than about 0.1 μg/ml, etc.). The term “trap” in this instance refers to reduction of further movement through the mucus. In some embodiments, the target (e.g., pathogen or sperm) may be trapped within about 30 minutes, e.g., about 25, 20, 15, 10, 5 or 1 minutes after administration of the synthetic binding agent having multiple Fab repeats described herein. In some embodiments, the synthetic binding agent traps the target at a synthetic binding agent concentration of less than about 5 mg/ml, 2.5 mg/ml, 1 mg/ml, 100 μg/ml, 50 μg/ml, 10 μg/ml, 5 μg/ml, 4 μg/ml, 3 μg/ml, 2 μg/ml, or 1 μg/ml.

The following discussion is presented as a general overview of the techniques available for the production of synthetic binding agent having multiple Fab repeats; however, one of skill in the art will recognize that many variations upon the following methods are known.

The term “antibody” or “antibodies” as used herein refers to all types of immunoglobulins, including IgG, IgM, IgA, IgD, and IgE. The antibody can be monoclonal or polyclonal and can be of any species of origin, including (for example) mouse, rat, rabbit, horse, goat, sheep, camel, or human, or can be a chimeric or humanized antibody. See, e.g., Walker et al., Molec. Immunol. 26:403 (1989). The antibodies can be recombinant monoclonal antibodies produced according to the methods disclosed in U.S. Pat. No. 4,474,893 or U.S. Pat. No. 4,816,567, the antibodies can also be chemically constructed according to the method disclosed in U.S. Pat. No. 4,676,980.

Antibody fragments included within the scope of the present invention include, for example, Fab, Fab′, F(ab)₂, and Fv fragments; domain antibodies, diabodies; nanobodies; vaccibodies, linear antibodies; single-chain antibody molecules, scFv; and multispecific antibodies formed from antibody fragments. Such fragments can be produced by known techniques. For example, F(ab′)₂ fragments can be produced by pepsin digestion of the antibody molecule, and Fab fragments can be generated by reducing the disulfide bridges of the F(ab′)₂ fragments. Alternatively, Fab expression libraries can be constructed to allow rapid and easy identification of monoclonal Fab fragments with the desired specificity (Huse et al., Science 254:1275 (1989)). In some embodiments, the term “antibody fragment” as used herein may also include any protein construct that is capable of binding a target.

Antibodies, including the core Ab forming part of the synthetic binding agent having multiple Fab repeats described herein may be humanized or camelized. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementarity determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework (FR) regions (i.e., the sequences between the CDR regions) are those of a human immunoglobulin consensus sequence. The humanized antibody optimally also will comprise at least a portion of an immunoglobulin constant region (Fe), typically that of a human immunoglobulin (Jones et al., Nature 321:522 (1986); Riechmann et al., Nature, 332:323 (1988); and Presta, Curr. Op. Struct. Biol. 2:593 (1992)).

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can essentially be performed following the method of Winter and co-workers (Jones et al., Nature 321:522 (1986); Riechmann et al., Nature 332:323 (1988); Verhoeyen et al., Science 239:1534 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues (e.g., all of the CDRs or a portion thereof) and possibly some FR residues are substituted by residues from analogous sites in rodent antibodies.

Human antibodies and synthetic binding agent having multiple Fab repeats based on human or humanized IgG as described herein can also be produced using various techniques known in the art, including phage display libraries (Hoogenboom and Winter, J. Mol. Biol. 227:381 (1991); Marks et al., J. Mol. Biol. 222:581 (1991)). The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol. 147:86 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10:779 (1992); Lonberg et al., Nature 368:856 (1994); Morrison, Nature 368:812 (1994); Fishwild et al., Nature Biotechnol. 14:845 (1996); Neuberger, Nature Biotechnol. 14:826 (1996); Lonberg and Huszar, Intern. Rev. Immunol. 13:65 (1995).

Immunogens (antigens) are used to produce antibodies specifically reactive with target polypeptides. Recombinant or synthetic polypeptides and peptides, e.g., of at least 5 (e.g., at least 7 or 10) amino acids in length, or greater, are the preferred immunogens for the production of monoclonal or polyclonal antibodies. In one embodiment, an immunogenic polypeptide conjugate is also included as an immunogen. The peptides are used either in pure, partially pure or impure form. Suitable polypeptides and epitopes for target pathogens and sperm are well known in the art. Polynucleotide and polypeptide sequences are available in public sequence databases such as GENBANK®/GENPEPT®. Large numbers of neutralizing and non-neutralizing antibodies that specifically bind to target pathogens and sperm have been described in the art and can be used as starting material to prepare the antibodies of the present invention. Alternatively, new antibodies can be raised against target pathogens and sperm using the techniques described herein and well known in the art.

Recombinant polypeptides are expressed in eukaryotic or prokaryotic cells and purified using standard techniques. The polypeptide, or a synthetic version thereof, is then injected into an animal capable of producing antibodies. Either monoclonal or polyclonal antibodies can be generated for subsequent use in immunoassays to measure the presence and quantity of the polypeptide.

Methods of producing polyclonal antibodies are known to those of skill in the art. In brief, an immunogen, e.g., a purified or synthetic peptide, a peptide coupled to an appropriate carrier (e.g., glutathione-S-transferase, keyhole limpet hemanocyanin, etc.), or a peptide incorporated into an immunization vector such as a recombinant vaccinia virus is optionally mixed with an adjuvant and animals are immunized with the mixture. The animal's immune response to the immunogen preparation is monitored by taking test bleeds and determining the titer of reactivity to the peptide of interest. When appropriately high titers of antibody to the immunogen are obtained, blood is collected from the animal and antisera are prepared. Further fractionation of the antisera to enrich for antibodies reactive to the peptide is performed where desired. Antibodies, including binding fragments and single chain recombinant versions thereof, against the polypeptides are raised by immunizing animals, e.g., using immunogenic conjugates comprising a polypeptide covalently attached (conjugated) to a carrier protein as described above. Typically, the immunogen of interest is a polypeptide of at least about 10 amino acids, in another embodiment the polypeptide is at least about 20 amino acids in length, and in another embodiment, the fragment is at least about 30 amino acids in length. For example, the polypeptide can comprise amino acids acid residues 1 through 200 from the N-terminal of the papillomavirus L2 protein. The immunogenic conjugates are typically prepared by coupling the polypeptide to a carrier protein (e.g., as a fusion protein) or, alternatively, they are recombinantly expressed in an immunization vector.

Monoclonal antibodies are prepared from cells secreting the desired antibody. These antibodies are screened for binding to normal or modified peptides, or screened for agonistic or antagonistic activity. Specific monoclonal and polyclonal antibodies will usually bind with a K_{D} of at least about 50 mM, e.g., at least about 1 mM, e.g., at least about 0.1 mM or better. In some instances, it is desirable to prepare monoclonal antibodies from various mammalian hosts, such as mice, rodents, primates, humans, etc. Description of techniques for preparing such monoclonal antibodies are found in Kohler and Milstein 1975 Nature 256:495-497. Summarized briefly, this method proceeds by injecting an animal with an immunogen, e.g., an immunogenic peptide either alone or optionally linked to a carrier protein. The animal is then sacrificed and cells taken from its spleen, which are fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Alternative methods of immortalization include transformation with Epstein Barr Virus, oncogenes, or retroviruses, or other methods known in the art. Colonies arising from single immortalized cells are screened for production of antibodies of the desired specificity and affinity for the antigen, and yield of the monoclonal antibodies produced by such cells is enhanced by various techniques, including injection into the peritoneal cavity of a vertebrate (preferably mammalian) host. The polypeptides and antibodies of the present invention are used with or without modification, and include chimeric antibodies such as humanized murine antibodies. Other suitable techniques involve selection of libraries of recombinant antibodies in phage or similar vectors. See, Huse et al. 1989 Science 246:1275-1281; and Ward et al. 1989 Nature 341:544-546.

Antibodies specific to the target polypeptide can also be obtained by phage display techniques known in the art.

Synthetic binding agent having multiple Fab repeats as described herein can be labeled by joining, either covalently or noncovalently, a substance which provides a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Synthetic binding agent having multiple Fab repeats as described herein may be useful for detecting or diagnosing the presence of a target on which an antigen is found.

Method of making synthetic binding agent having multiple Fab repeats as described herein with a glycosylation pattern of interest can be achieved by any method known to those or skill in the art. For example, in some embodiments, mammalian cells can be used, such as, Chinese hamster ovary (CHO) cells, baby hamster kidney (BHK) cells, and NS0- and SP2/0-mouse myeloma cells, to produce antibodies having the desired glycosylation pattern. In certain embodiments, human cell lines can be used, e.g., 293 cells. In some embodiments, non-mammalian cells can be used. The cell line can be genetically engineered to produce the antibodies with the desired oligosaccharide. Such cell lines can have altered expression, for example, of one or more enzymes affecting glycosylation patterns, e.g., glycosyltransferases. Glycosyltransferases include, without limitation, a galactosyltransferase, a fucosyltransferase, a glucosyltransferase, an N-acetylgalactosaminyltransferase, an N-acetylglucosaminyltransferase, a glucuronyltransferase, a sialyltransferase, a mannosyltransferase, a glucuronic acid transferase, a galacturonic acid transferase, an oligosaccharyltransferase, or any combination thereof. Specific examples include, without limitation, oligosaccharyltransferase, UDP-N-acetyl-D-galactosamine:polypeptide N-acetylgalactosaminyltransferase, GDP-fucose protein:O-fucosyltransferase 1, GDP-fucose protein:O-fucosyltransferase 2, protein:O-glucosyltransferase, UDP-N-acetylglucosamine:peptide N-aeetylglucosaminyltransferase, protein:O-mannosyltransferase, β1,4 galactosyltransferase, and any combination thereof. Enzymes involved in glycosylation of proteins are well known in the art and can be manipulated using routine techniques. See, for example, U.S. Pat. Nos. 8,383,106, 8,367,374, 8,080,415, 8,025,879, 8,021,856, 7,906,329, and 7,846,434, each incorporated herein by reference in its entirety. In other embodiments, glycans can be synthesized in specific patterns and linked to the synthetic binding agent having multiple Fab repeats described herein. In further embodiments, synthetic binding agent having multiple Fab repeats described herein with mixed glycosylation patterns can be separated to isolate antibodies with the desired glycosylation pattern.

As would be recognized by one skilled in the art, the synthetic binding agent having multiple Fab repeats described herein can also be formed into suitable compositions, e.g., pharmaceutical compositions for administration to a subject in order to act as a contraceptive and/or to treat or prevent an infection caused by a target pathogen or a disease or disorder caused by infection by a target pathogen. A composition may comprise, consist essentially of, or consist of a synthetic binding agent having multiple Fab repeats described herein in a prophylactically or therapeutically effective amount and a pharmaceutically-acceptable carrier.

Pharmaceutical compositions containing the synthetic binding agent having multiple Fab repeats described herein can be formulated in combination with any suitable pharmaceutical vehicle, excipient or carrier that would commonly be used in this art, including such conventional materials for this purpose, e.g., saline, dextrose, water, glycerol, ethanol, and combinations thereof. As one skilled in this art would recognize, the particular vehicle, excipient or carrier used will vary depending on the subject and the subject's condition, and a variety of modes of administration would be suitable for the compositions of synthetic binding agent having multiple Fab repeats described herein. Suitable methods of administration of any pharmaceutical composition disclosed in this application include, but are not limited to, topical, oral, intranasal, buccal, inhalation, anal, and vaginal administration, wherein such administration achieves delivery of the antibody to a mucus membrane of interest.

The composition can be any type of composition suitable for delivering a synthetic binding agent having multiple Fab repeats described herein to a mucosal surface and can be in various forms known in the art, including solid, semisolid, or liquid form or in lotion form, either oil-in-water or water-in-oil emulsions, in aqueous gel compositions. Compositions include, without limitation, gel, paste, suppository, douche, ovule, foam, film, spray, ointment, pessary, capsule, tablet, jelly, cream, milk, dispersion, liposomes, powder/talc or other solid, suspension, solution, emulsion, microemulsion, nanoemulsion, liquid, aerosol, microcapsules, time-release capsules, controlled release formulation, sustained release formulation or bioadhesive gel (e.g., a mucoadhesive thermogelling composition) or in other forms embedded in a matrix for the slow or controlled release of the antibody to the surface onto which it has been applied or in contact.

If topical administration is desired, the composition may be formulated as needed in a suitable form, e.g., an ointment, cream, gel, lotion, drops (such as eye drops and ear drops), or solution (such as mouthwash). The composition may contain conventional additives, such as preservatives, solvents to promote penetration, and emollients. Topical formulations may also contain conventional carriers such as cream or ointment bases, ethanol, or oleyl alcohol. Other formulations for administration, including intranasal administration, etc., are contemplated for use in connection with the presently-disclosed subject matter. All formulations, devices, and methods known to one of skill in the art which are appropriate for delivering the synthetic binding agent having multiple Fab repeats described herein or a composition containing the synthetic binding agent having multiple Fab repeats described herein to one or more mucus membranes of a subject can be used in connection with the presently-disclosed subject matter.

Any of the compositions described herein may include mixtures of the synthetic binding agent having multiple Fab repeats described herein, including mixtures having different numbers of Fab repeats (e.g., some with 4 Fab repeats, some with 6 Fab repeats, etc.).

The compositions used in the methods described herein may include other agents that do not negatively impact or otherwise affect the inhibitory and/or contraceptive effectiveness of the components of the composition, including antibodies, antimicrobial agents, and/or sperm-function inhibitors. For example, solid, liquid or a mixture of solid and liquid pharmaceutically acceptable carriers, diluents, vehicles, or excipients may be employed in the pharmaceutical compositions. Suitable physiologically acceptable, substantially inert carriers include water, a polyethylene glycol, mineral oil or petrolatum, propylene glycol, hydroxyethylcellulose, carboxymethyl cellulose, cellulosic derivatives, polycarboxylic acids, linked polyacrylic acids, such as carbopols; and other polymers such as poly(lysine), poly(glutamic acid), poly(maleic acid), polylactic acid), thermal polyaspartate, and aliphatic-aromatic resin; glycerin, starch, lactose, calcium sulphate dihydrate, terra alba, sucrose, talc, gelatin, pectin, acacia, magnesium stearate, stearic acid, syrup, peanut oil, olive oil, saline solution, and the like.

The pharmaceutical compositions described herein useful in the methods of the present invention may further include diluents, fillers, binding agents, colorants, stabilizers, perfumes, gelling agents, antioxidants, moisturizing agents, preservatives, acids, and other elements known to those skilled in the art. For example, suitable preservatives are well known in the art, and include, for example, methyl paraben, propyl paraben, butyl paraben, benzoic acid and benzyl alcohol.

For injection, the carrier may typically be a liquid, such as sterile pyrogen-free water, pyrogen-free phosphate-buffered saline solution, bacteriostatic water, or Cremophor EL® (BASF, Parsippany, N.J.). For other methods of administration, the carrier can be either solid or liquid.

For oral administration, the synthetic binding agent having multiple Fab repeats described herein can be administered in solid dosage forms, such as capsules, tablets, and powders, or in liquid dosage forms, such as elixirs, syrups, and suspensions. Compositions can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that can be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain coloring and flavoring to increase patient acceptance.

Compositions suitable for buccal (sub-lingual) administration include tablets or lozenges comprising the antibody in a flavored base, usually sucrose and acacia or tragacanth; and pastilles comprising the antibody in an inert base such as gelatin and glycerin or sucrose and acacia. The composition can comprise an orally dissolvable or degradable composition. Alternately, the composition can comprise a powder or an aerosolized or atomized solution or suspension comprising the antibody. Such powdered, aerosolized, or atomized compositions, when dispersed, preferably have an average particle or droplet size in the range from about 0.1 to about 200 nanometers

Compositions of the synthetic binding agent having multiple Fab repeats described herein that are suitable for parenteral administration comprise sterile aqueous and non-aqueous injection solutions of the synthetic binding agent having multiple Fab repeats described herein, which preparations are preferably isotonic with the blood of the intended recipient. These preparations can contain anti-oxidants, buffers, bacteriostats and solutes which render the composition isotonic with the blood of the intended recipient. Aqueous and non-aqueous sterile suspensions can include suspending agents and thickening agents. The compositions can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

Extemporaneous injection solutions and suspensions can be prepared from sterile powders, granules and tablets of the kind previously described. For example, in one aspect, there is provided an injectable, stable, sterile composition comprising a synthetic binding agent having multiple Fab repeats described herein, in a unit dosage form in a sealed container. The synthetic binding agent having multiple Fab repeats described herein may be provided in the form of a lyophilizate which is capable of being reconstituted with a suitable pharmaceutically acceptable carrier to form a liquid composition suitable for injection thereof into a subject.

Compositions suitable for rectal administration may be presented as unit dose suppositories. These can be prepared by admixing the synthetic binding agent having multiple Fab repeats described herein with one or more conventional solid carriers, for example, cocoa butter, and then shaping the resulting mixture.

The synthetic binding agent having multiple Fab repeats described herein can alternatively be formulated for nasal administration or otherwise administered to the lungs of a subject by any suitable means, e.g., administered by an aerosol suspension of respirable particles comprising the synthetic binding agent having multiple Fab repeats described herein, which the subject inhales. The respirable particles can be liquid or solid. The term “aerosol” includes any gas-borne suspended phase, which is capable of being inhaled into the bronchioles or nasal passages. Specifically, aerosol includes a gas-borne suspension of droplets, as can be produced in a metered dose inhaler or nebulizer, or in a mist sprayer. Aerosol also includes a dry powder composition suspended in air or other carrier gas, which can be delivered by insufflation from an inhaler device, for example. See Ganderton & Jones, Drug Delivery to the Respiratory Tract, Ellis Harwood (1987); Gonda (1990) Critical Reviews in Therapeutic Drug Carrier Systems 6:273-313; and Raeburn et al., J. Pharmacol. Toxicol. Meth. 27:143 (1992). Aerosols of liquid particles comprising the synthetic binding agent having multiple Fab repeats described herein can be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the synthetic binding agent having multiple Fab repeats described herein can likewise be produced with any solid particulate medicament aerosol generator, by techniques known in the pharmaceutical art.

Alternatively, one can administer the synthetic binding agent having multiple Fab repeats described herein in a local rather than systemic manner, for example, in a depot or sustained-release formulation.

The synthetic binding agent having multiple Fab repeats described herein may be coated or impregnated on a device (or a composition including the synthetic binding agent having multiple Fab repeats described herein may be coated or impregnated). The device can be for delivery of the synthetic binding agent having multiple Fab repeats described herein and compositions of the synthetic binding agent to a mucus membrane, e.g., to the vagina or uterus. In one embodiment, a device includes a solid support adapted to be inserted into the vagina. The support can be impregnated with or coated with a composition of the synthetic binding agent having multiple Fab repeats described herein. The release of synthetic binding agent from the devices may be controlled by the material composing these devices, such as silicone elastomers, ethylene vinyl acetate and polyurethane polymers. Devices, such as cervicovaginal and rectal devices, include, without limitation, a ring, rod, applicator, sponge, cervical cap, tampon, diaphragm, or intrauterine device. Applicators can be those currently used commercially to deliver spermicidal gels or anti-yeast compounds and include, without limitation, plunger-type applicators, pessaries, sprays, squeezable tubes, vaginal rings, cervical rings, sponges, and the like. All such means for delivery are intended to be encompassed by the present invention.

As noted herein, synthetic binding agent having multiple Fab repeats described herein is capable of diffusing through mucus when it is unbound, to allow the synthetic binding agent having multiple Fab repeats to bind a target (e.g., pathogen or sperm) at a desirable rate. It is also desirable that, when synthetic binding agent having multiple Fab repeats described herein is bound to the target, the cumulative effect of the antibody-mucin interactions effectively traps the pathogen or sperm in the mucus and/or agglutinates the target. To facilitate this goal, in some embodiments, it can be desirable to provide a composition that includes more than one synthetic binding agent having multiple Fab repeats described herein, wherein each synthetic binding agent specifically binds a different epitope of the pathogen or sperm. Such a composition may provide the ability for an increased number of synthetic binding agents having multiple Fab repeats to become bound to the pathogen or sperm, thereby strengthening the antibody-mucin interactions that serve to trap the pathogen or sperm in the mucus.

In some embodiments, a composition includes a first synthetic binding agent having multiple Fab repeats described herein and a second synthetic binding agent having multiple Fab repeats described herein, wherein the first synthetic binding agent specifically binds a first epitope of the target and the second binding agent specifically binds a second epitope of the target, wherein the first epitope is distinct from the second epitope. In certain embodiments, the composition includes three or more different synthetic binding agents having multiple Fab repeats described herein, e.g., 3, 4, 5, 6, 7, 8, 9, 10, or more different synthetic binding agents having multiple Fab repeats described herein, wherein each synthetic binding agent specifically binds a different epitope of the target.

It is also desirable to provide a composition that can provide treatment or prevention of infection due to more than one target pathogen. In some embodiments of the presently-disclosed subject matter, a composition includes a first synthetic binding agent having multiple Fab repeats and a second synthetic binding agent having multiple Fab repeats, wherein the first synthetic binding agent specifically binds an epitope of a first target pathogen and the second synthetic binding agent specifically binds an epitope of second target pathogen. In certain embodiments, the composition includes three or more different synthetic binding agents having multiple Fab repeats, e.g., 3, 4, 5, 6, 7, 8, 9, 10, or more different synthetic binding agents, wherein each synthetic binding agent specifically binds an epitope of a different target. As discussed above, in some variations the target may be the same, but the synthetic binding agents having multiple Fab repeats may have different numbers of Fab repeats.

In other embodiments, a composition provides both contraception and treatment or prevention of infection by one or more target pathogens. In some embodiments, a composition includes a first synthetic binding agent having multiple Fab repeats and a second synthetic binding agent having multiple Fab repeats, wherein the first synthetic binding agent specifically binds an epitope of sperm and the second synthetic binding agent specifically binds an epitope of a target pathogen. In certain embodiments, the composition includes three or more different synthetic binding agents having multiple Fab repeats described herein, e.g., 3, 4, 5, 6, 7, 8, 9, 10, or more different synthetic binding agents having multiple Fab repeats, wherein one or more synthetic binding agents having multiple Fab repeats bind different epitopes of sperm and one or more synthetic binding agent having multiple Fab repeats specifically binds an epitope of a target pathogen or multiple target pathogens.

In some embodiments, the pharmaceutical composition can further include an additional active agent, e.g., a prophylactic or therapeutic agent. For example, the additional active agent can be an antimicrobial agent, as would be known to one of skill in the art. The antimicrobial agent may be active against algae, bacteria, fungi, parasites (helminths, protozoa), viruses, and subviral agents. Accordingly, the antimicrobial agent may be an antibacterial, antifungal, antiviral, antiparasitic, or antiprotozoal agent. The antimicrobial agent is preferably active against infectious diseases. Suitable antiviral agents include, for example, virus-inactivating agents such as nonionic, anionic and cationic surfactants, and C31 G (amine oxide and alkyl betaine), polybiguanides, docosanol, acylcarnitine analogs, octyl glycerol, and antimicrobial peptides such as magainins, gramicidins, protegrins, and retrocyclins. Mild surfactants, e.g., sorbitan monolaurate, may advantageously be used as antiviral agents in the compositions described herein. Other antiviral agents that may advantageously be utilized in the compositions described herein include nucleotide or nucleoside analogs, such as tenofovir, acyclovir, amantadine, didanosine, foscarnet, ganciclovir, ribavirin, vidarabine, zalcitabine, and zidovudine. Further antiviral agents that may be used include non-nucleoside reverse transcriptase inhibitors, such as UC-781 (thiocarboxanilide), pyridinones, TIBO, nevaripine, delavirdine, calanolide A, capravirine and efavirenz. From these reverse transcriptase inhibitors, agents and their analogs that have shown poor oral bioavailability are especially suitable for administration to mucosal tissue, in combination with antibodies and compositions of the invention, to prevent sexual transmission of HIV. Other antiviral agents that may be used are those in the category of HIV entry blockers, such as cyanovirin-N, cyclodextrins, carregeenans, sulfated or sulfonated polymers, mandelic acid condensation polymers, monoclonal antibodies, chemokine receptor antagonists such as TAK-779, SCH-C/D, and AMD-3100, and fusion inhibitors such as T-20 and 1249.

Suitable antibacterial agents include antibiotics, such as aminoglycosides, cephalosporins, including first, second and third generation cephalosporins; macrolides, including erythromycins, penicillins, including natural penicillins, penicillinase-resistant penicillins, aminopenicillins, extended spectrum penicillins; sulfonamides, tetracyclines, fluoroquinolones, metronidazole and urinary tract antiseptics.

Suitable antifungal agents include amphotericin B, nystatin, griseofulvin, flucytosine, fluconazole, potassium iodide, intraconazole, clortrimazole, miconazole, ketoconazole, and tolnaftate. Suitable antiprotozoal agents include antimalarial agents, such as chloroquine, primaquine, pyrimethamine, quinine, fansidar, and mefloquine; amebicides, such as dioloxamide, emetine, iodoquinol, metronidazole, paromomycine and quinacrine; pentamidine isethionate, atovaquone, and eflornithine.

In certain embodiments, the additional active agent can be a sperm-function inhibitor, e.g., an agent that has the ability to inhibit the function of sperm, to otherwise inhibit fertilization of an egg by sperm and/or to otherwise prevent pregnancy, such as by killing and/or functionally inactivating sperm or by other effects on the activity of the sperm. In some embodiments, the active agent may have at least dual functions, such as acting as a sperm-function inhibitor and as an antimicrobial agent.

Sperm-function inhibitors include, without limitation, surfactants, including nonionic surfactants, cationic surfactants, and anionic surfactants; spermicides, such as nonoxynol-9 (α-(4-Nonylphenyl)-ω-hydroxynona(oxyethylene); other sperm-inactivators such as sulfated or sulfonated polymers such as polystyrene sulfonate, mandelic acid condensation polymers, cyclodextrins; antimicrobial peptides such as gramicidins, magainins, indolicidin, and melittin; and acid-buffering compositions, such as BufferGel and AcidForm. Nonionic surfactants include, for example, sorbitan monolaurate, nonylphenoxypolyethoxy ethanol, p-diisobutyphenoxypolyethoxy ethanol, polyoxyethylene (10) oleyl ether and onyx-ol. Suitable anionic surfactants include, without limitation, sodium alkyl sulfonates and the sodium alkylbenzene sulfonates. Cationic surfactants include, for example, the quaternary ammonium surfactants, such as cetyl pyrimidinium chloride and benzalkonium chlorides. Zwitterionic surfactants such as acylcamitine analogs and C31G are especially suitable for their mild skin and mucosal irritation properties.

The presently-disclosed subject matter further includes a kit including the synthetic binding agent having multiple Fab repeats described herein or a composition comprising the synthetic binding agent having multiple Fab repeats as described herein; and optionally a device for administering the synthetic binding agent or composition. In some embodiments, the kit can include multiple synthetic binding agents having multiple Fab repeats and/or compositions containing such synthetic binding agents. In some embodiments, each of the multiple synthetic binding agents provided in such a kit can specifically bind to a different epitope of the target, e.g., pathogen or sperm. In other embodiments, each of the multiple synthetic binding agents having multiple Fab repeats as described herein provided in such a kit can specifically bind to an epitope of a different target pathogen or to an epitope of sperm. In some embodiments, the kit can further include an additional active agent, e.g., antimicrobial, such as an antibiotic, an antiviral, or other antimicrobial, or a sperm-function inhibitor as would be known to one of skill in the art.

### Synthetic Binding Agents Having Multiple Fab Repeats

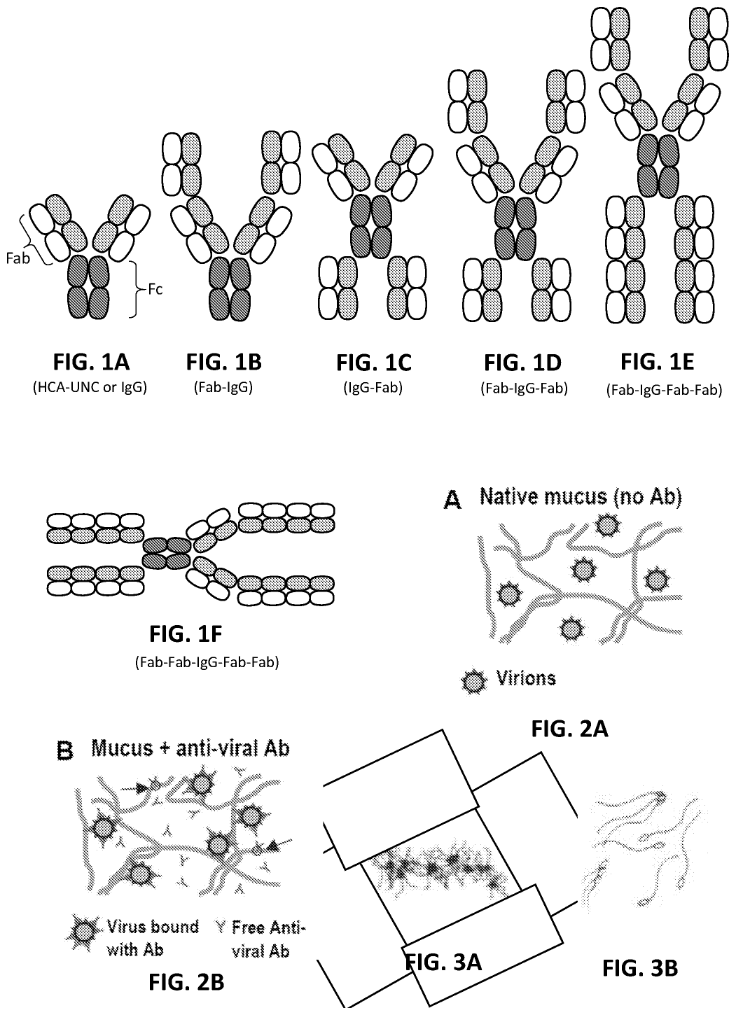

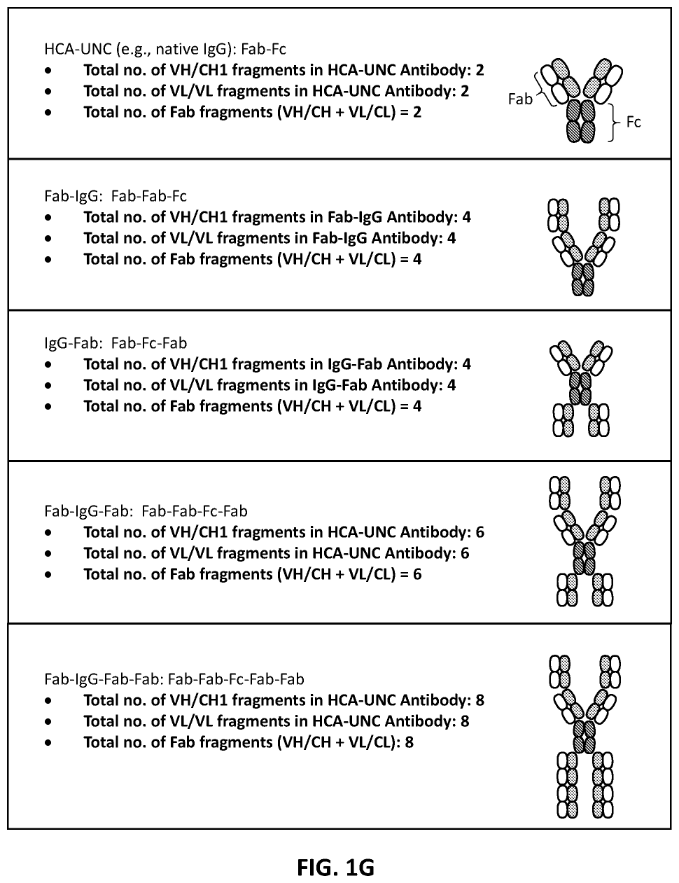

In general, the synthetic binding agent having multiple Fab repeats described herein may include a core IgG that is directed to an epitope of a target. The synthetic binding agent having multiple Fab repeats may be constructed by coupling multiple additional copies of the same (or a portion of the same) Fab domain of the IgG core. In some embodiments, the additional Fab may not be identical to the Fab domain of the IgG core but still bind the same epitope. The additional copies may be added to the amino and/or carboxyl ends of the IgG core. This is schematically illustrated in FIGS. 1A-1G. In addition, the core IgG includes an Fc region that may be glycosylated (or a composition including the synthetic binding agent having multiple Fab repeats may be selected to enrich for glycosylation) in a pattern that enhances muco-trapping, such as the G0 glycosylation form.

For example, FIGS. 1A-1E illustrate different multimeric constructs (“synthetic binding agent having multiple Fab repeats”) that may be produced and characterized, and the agglutination and muco-trapping potencies measured. The one or more constructs, e.g., having the greatest potency, may be combined and used.

FIG. 1A shows an example of a core IgG including a pair of Fab and Fc regions. As shown in FIGS. 1B-1E, the same Fab regions may then be combined in pairs (e.g., 2 additional, 4 additional, 6 additional, 8 additional, 10 additional, etc.) to the core IgG to form synthetic binding agents having multiple Fab repeats.

### Contraceptive Synthetic Binding Agents Having Multiple Fab Repeats

There is a strong unmet demand for non-hormonal contraceptives: Per the CDC, roughly half of ˜30 million women between the ages of 20-35 in the U.S. seek some form of reversible contraceptive method (e.g. pills, IUDs, condoms, rings, etc.). Socio-societal issues no doubt contribute to the limited uptake and adherence of common contraceptive methods; nevertheless, numerous studies have also demonstrated the need for alternative contraceptive methods, particularly non-hormonal options. The vast majority of women start with hormonal contraceptives, which is readily available and highly effective. However, many women are naturally averse to exogenous hormones despite counselling. Over half of women quit (majority within 3-6 months) due to reasons including real and perceived side effects associated with hormonal contraceptives (such as weight gain, mood swings and depression, headaches and nausea), strongly underscoring the need for non-hormonal contraception. Also, both oral and IUD-based hormonal contraception frequently lead to intermenstrual “spotting” (light bleeding in weeks prior to the period). Although this may be viewed as mere inconvenience in western societies, many women/couples find it seriously objectionable. Spotting can significantly limit use of hormonal contraception among certain populations, since men contacting women's menstrual blood can be a serious taboo for religious reasons (e.g., Muslims and orthodox Jews).

The synthetic binding agent having multiple Fab repeats described herein may be non-hormonal contraceptives that can block sperm permeation through mucus. A major effector function for Ab in mucus is to arrest the forward motion of foreign entities such as viruses and highly motile bacteria, and block them from reaching target cells. This function can be accomplished in two ways. First, when concentrations of the foreign entity are high such that the foreign bodies would frequently collide, Ab can crosslink two or more bodies together, resulting not only in an increase in hydrodynamic diameter but more importantly an effective neutralization of the net forward motion of swimming bodies. This process is commonly referred to as agglutination (see, e.g., FIG. 3A). Second, when concentrations of the foreign entity are modest such that collisions between foreign bodies are relatively infrequent, Ab can immobilize by directly crosslinking the foreign body to the mucin matrix present in mucus via multiple Fc-mucin bonds (see, e.g., FIG. 3B). This process, which is referred to herein as muco-trapping, remained largely unrecognized because the affinity between each Ab molecule and mucin was long thought to be much too weak to effectively bind individual foreign bodies to mucins. Vaginally dosed antigen-specific IgG can trap viruses in mucus by forming multiple weakly adhesive bonds between the virus and the mucin mesh (akin to a Velcro® patch with individually weak hooks). IgG-mediated trapping effectively reduced the flux of viruses arriving at target cells and directly blocked vaginal Herpes transmission in mice.

Sperm concentration varies widely in the female reproductive tract, with the maximum concentration in semen immediately following ejaculation and lower concentrations in more distal sites, such as the cervical canal. The ideal human contraceptive Ab (HCA, i.e. an Ab molecule that can block sperm permeation through mucus and prevent sperm from reaching the egg) should therefore harness one or both agglutination and trapping in mucus. Polyvalent Ig such as sIgA and IgM are markedly more potent agglutinators than IgG (IgM is ˜1000-fold more potent at agglutination than IgG). Unfortunately, large scale manufacturing of IgM or sIgA remains exceptionally challenging, and IgG represents the predominant isotype of Ab under clinical development. However, it may be beneficial to use IgG.

Described herein are synthetic binding agents having multiple Fab repeats with greater agglutination potency as compared to current monomeric IgG1-based HCA (see, e.g., JPS638400A) by engineering multimeric HCA that can more potently agglutinate sperm.

Thus, described herein are multimeric HCA constructs (e.g., synthetic binding agent having multiple Fab repeats) with enhanced agglutination potency. These synthetic binding agents may include a Fab from a human IgM that binds a unique antigen restricted to only sperm and cells in the male reproductive tract, CD52g, and appears to be universal in all men (see, e.g., Norton et al., Tissue Antigens 2002, 60:354-364, Aug. 14, 2002). This Fab may serve as the basis for the HCA molecule (e.g., the synthetic binding agent). As described above, different synthetic binding agents having multiple Fab repeats constructs may be formed, comprised of increasing valency of Fab domains relative to traditional IgG, while maintaining its native muco-trapping potency. The different synthetic binding agents may include: Fab-IgG, IgG-Fab, Fab-IgG-Fab, and Fab-IgG-Fab-Fab; the core IgG may be used as a control. See FIG. 1A-1G, discussed above. All of these synthetic binding agent constructs were tested for expression and activity, including (i) ELISA and biolayer interferometry for antigen affinity, (ii) gel electrophoresis and SEC to assess incorporation of additional Fab domains, and (iii) verification of the ability to purify different HCA constructs using conventional Protein A/G columns.

Described herein are synthetic binding agents having multiple Fab repeats that may be formed as discussed above in relation to FIGS. 1A-1G. For example, in FIG. 1A, the core antibody may be an IgG form of the HCA-UNC antibody targeting the CD52g glycan, as described in greater detail below. In some variations the synthetic binding agent having multiple Fab repeats includes the core IgG and a pair of additional copies of the Fab (copied from the IgG core) attached to the amino terminal ends, as shown in FIG. 1B, or the carboxyl ends, as shown in FIG. 1C. In some variations, additional Fab copies are attached to either or both the amino and/or carboxyl ends, as shown in FIG. 1D (additional Fab copies at both amino and carboxyl ends) and FIG. 1E (two additional Fab copies at the amino end and four additional Fab copies at the carboxyl end). FIG. 1G shows examples of these structures. The attached sequence listing provides examples of sequences for each of these five structures. For example, SEQ ID NO: 9 is an exemplary listing of a full-length Fab-IgG heavy chain portion, while SEQ ID No: 13 is the corresponding full length Fab-IgG light chain amino acid sequence. Similarly, SEQ ID NO: 15 and SEQ ID NO: 19 illustrate an example of amino acid sequences of heavy chain and light chain, respectively, of an IgG-Fab synthetic binding agent having multiple Fab repeats. SEQ ID NO: 21 and SEQ ID NO: 25 illustrate an example of amino acid sequences of heavy chain and light chain, respectively, of a Fab-IgG-Fab synthetic binding agent having multiple Fab repeats. Further, SEQ ID NO: 27 and SEQ ID NO: 31 illustrate an example of amino acid sequences of heavy chain and light chain, respectively, of a Fab-IgG-Fab-Fab synthetic binding agent having multiple Fab repeats.

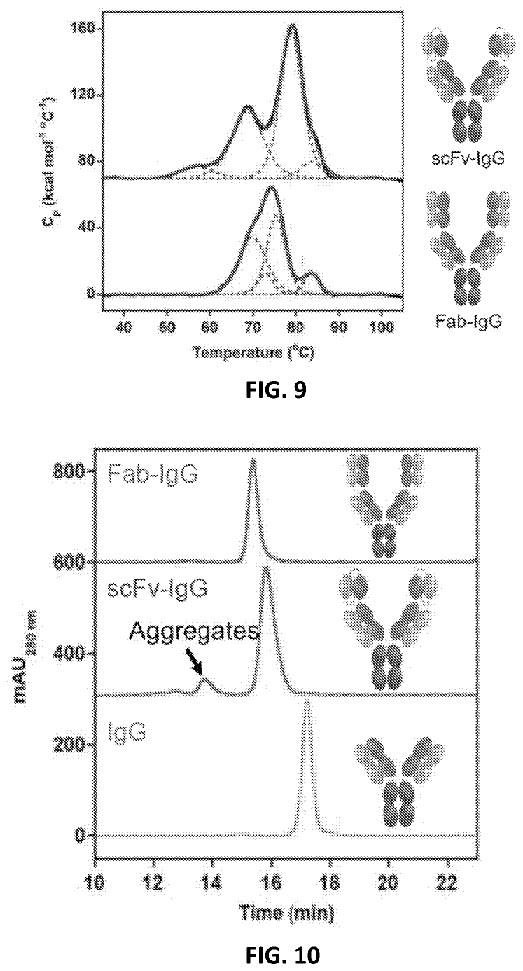

These various synthetic binding agents having multiple Fab repeats were examined against each other, as well as against the core IgG (e.g., as encoded by the amino acid sequences of SEQ ID NO: 3 and SEQ ID NO: 7). These synthetic binding agents were also examined against single-chain variable fragment (scFv) moieties or camel-derived nanobodies. scFv-based multimeric Ab constructs frequently suffer from low stability, heterogeneous expression, and decreased affinity and specificity stemming from the removal of the CH1/CL interface present in a full-length Fab (FIG. 9). In contrast, the synthetic binding agent having multiple Fab repeats exhibit enhanced agglutination potential compared to HCA-UNC target the same antigen. These synthetic binding agents having multiple Fab repeats have increased valency, span, and directional separation of the Fabs (diametrically opposed orientation). Because all Fabs bind the same target epitope and have essentially the same amino acids, the synthetic binding agents are not limited by incorrect heavy and light chain pairing that necessitates the use of scFv-based designs typical for engineering multimeric bispecific antibodies that target two distinct epitopes. Instead, these multimeric Ab constructs (synthetic binding agent having multiple Fab repeats) link full Fab domains to IgG. These Fab-based HCA constructs will likely result in improved stability and manufacturability compared to scFv-based constructs, preserved binding affinity to specific epitopes, and preserved Fc-mucin affinity.

For example, initial studies with Fab-IgG (one form of synthetic binding agent having multiple Fab repeats) showed that these molecules can be expressed and purified using industry-standard techniques while avoiding the formation of aggregates commonly observed with scFv-based multimeric constructs, as shown in FIG. 10, showing an analytical size exclusion chromatography result for IgG (bottom), scFv-IgG (middle), and Fab-IgG (top) after single-step protein A purification. Both IgG and Fab-IgG formats show a single sharp peak at their expected molecular weight, while scFv-IgG shows formation of high molecular-weight aggregates.

Thus, a synthetic binding agent having multiple Fab repeats may be used for IgG-based HCA for contraception. There is at least 10-fold more IgG present in CVM than IgA, which suggests IgG is the optimal mAb for vaginal protection in humans.

Undiluted, physiological human genital secretions were used in an ex vivo investigation of sperm and STI trapping of the synthetic binding agents having multiple Fab repeats targeting a CD52g epitope. Trapping of sperm in fresh, minimally perturbed ex vivo samples of CVM and CM was observed to ensure that our observations reflect physiological conditions as closely as possible.

Synthetic binding agents having multiple Fab repeats configured as multimeric HCA constructs (e.g., Fab-IgG, IgG-Fab, Fab-IgG-Fab) were created using standard cloning methods. Briefly, genes encoding HCA VH/VL domains and flexible linkers (GSSSS×3 (SEQ ID NO: 32) were synthesized and cloned into an in-house HCA IgG1 mammalian expression vector.

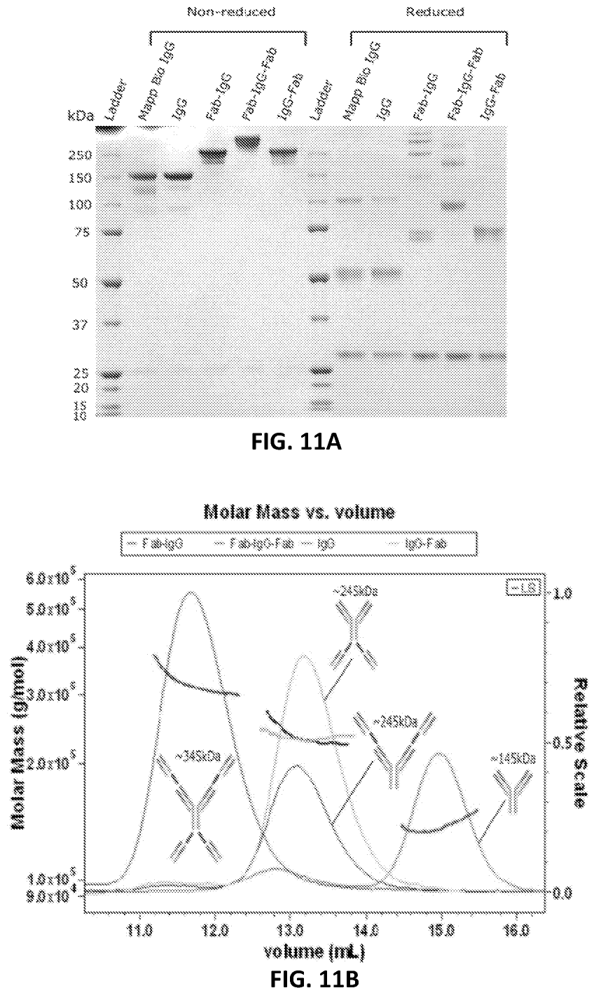

Constructs were expressed by transient expression in 30 mL cultures of Expi293 cells, and the corresponding HCA constructs were purified using protein-A affinity chromatography. The purity was verified by SDS-PAGE (FIG. 11A; note: we compared both an in-house IgG HCA and an IgG HCA that was independently prepared by Mapp Biopharmaceutical). We also performed size-exclusion chromatography/multi-angle light scattering (SEC/MALS) on each multimeric HCA construct to ensure homogenous expression of the desired antibody construct (FIG. 11B). In all cases, constructs expressed at high yield with minimal (<10%) aggregation.

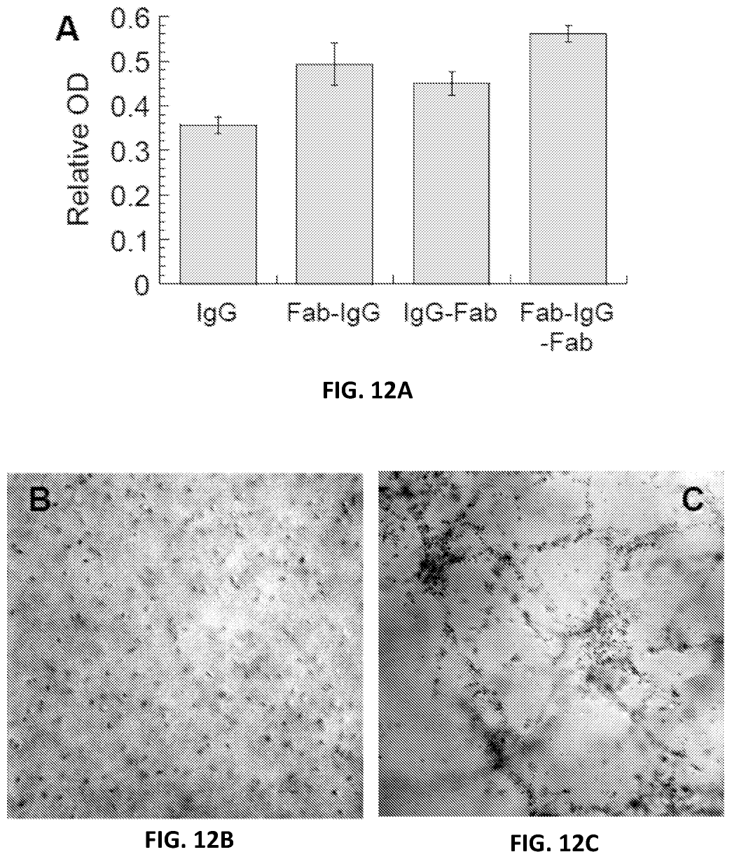

Synthetic binding agent having multiple Fab repeats configured as multimeric HCA binds and agglutinates sperm. The multimeric HCA was shown to bind sperm. We first performed a whole-sperm ELISA assay using equimolar quantities of each construct (1.5 nM), followed by detection using anti-Fc HRP secondary. Our pilot assay showed that each of the multimeric constructs tested (Fab-IgG, IgG-Fab, Fab-IgG-Fab) had comparable if not superior binding to sperm than the native IgG (FIG. 12A), demonstrating that the new HCA constructs (the synthetic binding agents having multiple Fab repeats directed to CD52g) can indeed bind sperm. To further verify that the HCA constructs can mediate agglutination, we mixed IgG-Fab HCA and PBS into semen and imaged the distribution of cells in the semen after 1 minute, and found that IgG-Fab HCA potently agglutinated cells (FIGS. 12B-12C). FIG. 12B shows the PBS control, while FIG. 12C shows the agglutination due to a synthetic binding agent having multiple Fab repeats against CD52g (IgG-Fab) after 1 minute, as quantified by the fraction of progressively motile sperm.

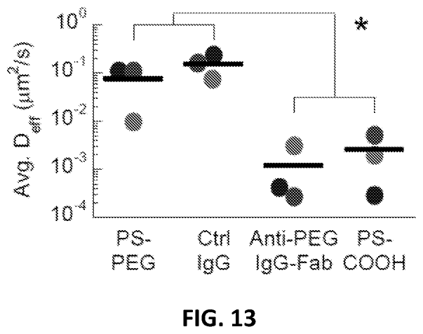

Muco-trapping was shown with IgG-Fab constructs. The synthetic binding agent having multiple Fab domains retained its muco-trapping potency, as confirmed by microscopy studies using anti-HER2×anti-PEG IgG-Fab, showing that the construct can immobilize ˜100 nm PEG-coated nanoparticles (PS-PEG) in human CVM. The anti-PEG Fab portion is functional and binds the antigen of interest (PEG), and the IgG-Fab structure retains adequate muco-affinity to trap virus-sized particles in mucus. This is illustrated in FIG. 13. PS-PEG in native CVM (no Ab) as well as in CVM treated with control IgG-Fab (anti-HER2×anti-VSVG) both exhibited rapid diffusion only a few fold slower than their theoretical rates in pure water. In contrast, addition of PEG-binding IgG-Fab to CVM resulted in extensive trapping of PS-PEG, with the fraction of mobile particles reduced from 71% to only 3%, comparable to muco-adhesive uncoated nanoparticles (PS-COOH). The degree of trapping was similar to the level we demonstrated previously with native anti-PEG IgG. These results underscore the potential of IgG-Fab and other synthetic binding agents with multiple Fabs to facilitate trapping of foreign bodies in mucus, including virus, bacteria and sperm.

Sperm trapping and agglutination observed in fresh, minimally perturbed ex vivo samples of CVM and CM provide evidence of physiological relevance.

As discussed herein, agglutination potential of the native IgG1 HCA (e.g., IgG-UNC) has been shown in various synthetic binding agents having multiple Fab repeats directed against an epitope of CD52g. HCA constructs with different polyvalency (i.e. number of Fab domains per molecule), as shown in FIGS. 1B-1F, were constructed and examined. Preliminary results suggest a greater agglutination potency will be achieved by increasing the number of CD52g-binding Fab domains along an IgG backbone, and that we may achieve agglutination potency comparable to native IgM molecules with our Fab-IgG-Fab-Fab and Fab-Fab-IgG-Fab-Fab constructs. The evaluation of both Fab-IgG and IgG-Fab formats, which differ only in the location of the appended Fab fragment, provide additional information regarding the importance of geometric orientation and sterics (particularly separation of Fabs, or “spanning” capability) in sperm agglutination and trapping potency. By using whole Fab domains rather than scFv, we expect the multimeric HCA constructs to remain stable, and that each Fab domain possess comparably high affinity to the CD52g antigen. We anticipate that HCA constructs with identical Fc-N-glycan profiles as native IgG molecules would possess comparable muco-trapping potency.

A baseline IgG1 HCA construct has been used to incorporate additional identical Fab domains against CD52g at different locations along the heavy chain. The heavy- and light-chain gene sequences for IgG control antibodies and each of the Fab-based multimeric antibodies may be codon-optimized, synthesized, and cloned into mammalian expression vectors (Integrated DNA Technologies). For each format, Fab-components may be separated by a flexible peptide linker (e.g., a flexible linker comprising an amino acid sequence comprising n pentapeptide repeats consisting of Glycine (G) and Serine (S), wherein n is between 3 and 8 amino acids, such as 6 repeated units of GSSSS (SEQ ID NO: 33), GGGGS (SEQ ID NO:34), etc.).

Upon verification of the cloning, small batches (30-60 mL) of each HCA construct will be expressed by transient transfection in Expi293 mammalian cells. After three days of cell growth, the various HCA constructs will be purified from culture supernatant by protein A affinity chromatography. Expression yield will be quantified using absorbance at 280 nm and BCA assay using human IgG as standard, and purified products will be assessed for purity using SDS-PAGE electrophoresis under both reducing and non-reducing conditions. The correct assembly, thermal stability and binding kinetics for each of the multimeric HCA formats may be verified; for example, correct assembly may be determined by molecular weight evaluation by size-exclusion chromatography/multi-angle light scattering (Wyatt DAWN HELEOS II; see, e.g., FIG. 10). Thermal stability (Tm) may be measured using differential scanning calorimetry (MicroCal VP-DSC; see FIG. 9). Antibodies that express and purify with less than 95% correct assembled product, or that are destabilized >10° C. from the parental sequence will not be further tested without optimization.

Whole-sperm ELISA may be used to quantify different HCA mAbs. First, high-affinity 96-well half-area plates (Thermo Scientific, Rockford, Ill.) may be coated overnight at 4° C. with 50 uL per well of sperm at 10′/ml (measured using cell counter). Plates are washed three times with 0.05% Tween in PBS (PBS-T), blocked with 5% milk for at least 1 hr, and incubated for at least 2 hr with serial dilutions of each HCA mAbs. Following three PBS-T washes, plates are incubated with F(ab′)2 anti-human IgG Fc (Goat)-HRP conjugate (709-1317; Rockland, Gilbertsville, Pa.) for 1 hr. 1-Step Ultra TMB substrate (Thermo Scientific, Rockford, Ill.) is used to develop the HRP conjugated IgG for 15 min followed by quenching with 2N sulfuric acid. Absorbance is measured at 450 nm using a BioTek Synergy 2 plate reader. Binding kinetics to sperm will also be evaluated by bio-layer interferometry (Octet Red384) by using anti-hIgG Fc Capture biosensors dipped into TritonX-100 treated sperm lysates (which serve as source of HCA antigen). We anticipate the HCA will be structurally intact, stable and bind sperm.

The synthetic binding agents having multiple Fab repeats described herein are derived from fully human Ab from an immune infertile but otherwise healthy woman (Isojim et al.). The epitope may include the glycosylation structure; and may specifically recognize a poly-n-acetyllactosamine region (e.g., repeating poly-n-acetyllactosaminyl structures) an antibody such as H6-3C4 may bind to an internal stretch of N-acetyllactosamines, and unlike antibodies against blood group i, it is not affected by terminal sialylation. This glycoform (referred to as CD52g) is believed to be specific to male-derived cells (e.g., sperm). Thus, a Fab may binds this CD52g (see, SEQ ID NO: 1) glycoprotein that is unique to the male genital tract and present on the surface of all sperm and other cells in semen. Although CD52g shares a short peptide backbone with leukocyte CD52, the HCA-UNC used as the core IgG does NOT bind CD52, and only binds the unique form of CD52g that is produced and secreted only by epithelial cells lining the lumen of the epididymis, vas deferens and seminal vesicles. CD52g contains a glycosylphosphatidylinositol (GPI) anchor, and is transferred to the plasma membrane of sperm as they mature in the epididymis. As shown in FIGS. 8A-8B, HCA also co-agglutinates leukocytes in semen; these cells are potential HIV-infected “Trojan Horse leukocytes” that may act as motile vectors for HIV transmission, implying that HCA may also afford some protection against cell-mediated transmissions. A WHO-sponsored anti-sperm vaccine workshop has identified CD52g as a promising antifertility vaccine candidate due to its unique expression in the male reproductive tract, potent antigenicity, and its ability to induce infertility in otherwise healthy individuals. Importantly, this HCA target appears to be ubiquitous in men: we have tested fresh semen samples from 100 men (73% Caucasian, 26% African American, and 1% Asian), and all specimens had >90% of sperm agglutinated within seconds by the prototype HCA.

The synthetic binding agents having multiple Fab repeats described herein may be produced in CHO cells, in Nicotiana plants, and in Trichoderma (for the latter two, they can be produced in modified plants or yeast containing the human glycosylation pathway, and are capable of making fully human mAb, such as ZMapp in Nicotiana).

The total dose of synthetic binding agents having multiple Fab repeats for contraception (e.g., HCA) may be, e.g., ˜20-80 mg to maintain ˜400 μg/mL of HCA in CVM for 28 days. Improving agglutination potency by just 10-fold over, e.g., HCA-UNC in the synthetic binding agents having multiple Fab repeats described herein may allow substantially lower concentrations to be delivered.

These synthetic binding agents having multiple Fab repeats were then tested to measure sperm agglutination and trapping potency in vitro. Sperm agglutination and trapping potency for different synthetic binding agents having multiple Fab repeats (referred to in this example as HCA constructs) were tested. Fresh human cervicovaginal mucus (CVM) and mid-cycle endocervical mucus (CM) was used to measure the real-time mobility for thousands of individual sperm cells in mucus treated with different HCA constructs to determine the precise extent the mobility and mobile fraction of spermatozoa in mucus is reduced by agglutination and muco-trapping over time.

A sheep vagina model may be further used to evaluate the potency of HCA in reducing free motile sperm by agglutinating and/or trapping human sperm in vaginal mucus. The anatomy of the sheep vagina is similar to the human vagina, and is the best available animal model for preclinical assessment of vaginal products. To examine potential in vivo efficacy, agglutination and trapping of fresh human semen in the sheep vagina may be assessed at different times after dosing semen, such as 2 min after deposition.

Sperm must swim through mucus to reach the egg. In some infertile women without other known causes of infertility, Ab have been isolated that bind the surface of living sperm and block sperm from penetrating mucus, with IgM offering the most potent combination of agglutination and trapping among naturally occurring Ab (see, e.g., Isojima et al., “Establishment and characterization of a human hybridoma secreting monoclonal antibody with high titers of sperm immobilizing and agglutinating activities against human seminal plasma.” J Reprod Immunol, 1987. 10(1): p. 67-78; and Tsuji et al., “Human sperm carbohydrate antigens defined by an antisperm human monoclonal antibody derived from an infertile woman bearing antisperm antibodies in her serum.” J Exp Med, 1988. 168(1): p. 343-56. PMCID: 2188971). In good agreement with human studies, animal studies have also shown that vaginal sperm-binding IgG, sIgA and IgM can provide contraception. This natural mechanism of infertility may be used to design a synthetic binding agent having multiple Fab repeats to enable non-hormonal contraception. A fully human mAb, termed HCA-UNC (or “HCA original”), which binds a highly validated and well characterized antigen target ubiquitously present only on the surface of sperm and cells in the male reproductive tract was used to form the core IgG of the synthetic binding agent having multiple Fab repeats for use as a contraceptive.

Multimeric HCA constructs (e.g., synthetic binding agents having multiple Fab repeats) were constructed having multiple Fab domains linked to a parent IgG molecule, with the overall goal of engineering an HCA that possesses IgM-like agglutination potency, while still amenable to commercial IgG purification process using, e.g., Protein A/G, to enable a potent, topical, non-hormonal contraceptive via an HCA that is cost effective and sorely needed by women around the world.

Antibodies can bind antigen on the sperm surface in the context of immune infertility. Immune infertility broadly refers to immune mechanisms that can contribute to infertility, and can be mediated by a variety of antibodies, including anti-phospholipid, anti-thyroid and anti-sperm antibodies (ASA). ASA refers to a broad spectrum of antibodies that can bind any sperm-associated antigens. The vast majority of naturally occurring ASA bind cytoplasmic antigens only accessible after sperm die, and are thus irrelevant for contraception. However, some Ab isolated from women who are immune infertile can cause infertility even without directly blocking sperm-egg interactions, including the IgM molecule isolated by Isojima from an infertile woman that serves as the basis for our current HCA. The HCA-UNC that may be used as the basis for the synthetic binding agent having multiple Fab repeats described herein binds an accessible surface antigen unique to sperm and cells in the male reproductive tract, and can prevent sperm from reaching the egg by agglutinating and/or immobilizing sperm in mucus. Indeed, it is partially because of the increased barrier function imparted by sperm-binding Ab in mucus that the motility of sperm in CM is often measured in clinical evaluation of infertility. Other ASAs may form the basis (e.g., core IgG) for other HCAs using the principles described herein.

Vaginally delivered HCA is likely to provide highly effective and safe contraception. Sperm must swim through mucus to reach and fertilize the egg. Not surprisingly, poor sperm motility in cervical mucus is generally a good correlate to infertility, and sperm motility in mucus remains a gold standard test in diagnosing infertility. By extension, arresting sperm motility in mucus through antibodies that can agglutinate and immobilize individual sperm in mucus, by directly reducing the number of sperm that reach the egg, should provide an effective form of contraception. Indeed, such sperm-binding Ab have been isolated from the cervicovaginal secretions of infertile women. Studies have shown that these sperm-binding Ab (IgG, IgA or IgM) can trap vigorously motile sperm in cervical mucus without interfering with the sperm motility apparatus (Ab-coated sperm will swim freely in buffer), and trapped sperm shake in place for hours in mucus until they die. This “shaking phenomenon” was and continues to be a standard clinical diagnosis for a cause of infertility in humans, Local delivery of sperm-binding Ab is highly effective in vivo, reducing egg fertilization by at least 95% in a highly fertile rabbit model.

The female reproductive tract is coated with far smaller volumes of mucus (˜1-2 mL) than the volume of blood in circulation (˜5,000 mL). Thus, by delivering HCA locally, contraceptive concentrations may be achieved with far lower amounts of HCA than with systemic delivery. Vaginally delivered mAb are poorly absorbed into the systemic circulation, further reducing the HCA amount needed to sustain contraceptive levels in the female reproductive tract.

HCA delivered into the vagina is highly unlikely to generate systemic toxicity, because: HCA is a fully human IgG; HCA is unlikely to be absorbed into the systemic circulation, the vagina is poorly responsive to immunization, and the target antigen of HCA is found exclusively in cells originating from the male reproductive tract, and is not present in females. The exceptionally limited systemic uptake could lead to a sufficient safety profile for HCA. Vaginal secretions possess very low complement activity, and have exceedingly few, if any, live leukocytes due to continuous acidification of the vagina to pH ˜4 by lactic acid from commensal Lactobacilli (leukocytes are effectively immobilized or killed at pH<6). Thus, HCA, especially if delivered at doses below total IgG present in CVM, is unlikely to trigger toxicity or inflammation in local vaginal tissues, yet remain effective at vaginal pH.

Weak and transient mucin bonds with the synthetic binding agent having multiple Fab repeats may allow the synthetic binding agent to freely diffuse in mucus most of the time and rapidly accumulate on the pathogens. In turn, the array of bound Ab on Ab/pathogen complex can form a sufficient number of weak crosslinks with the mucin mesh to trap pathogen with permanent avidity. Interactions between IgG and mucins appear to occur through N-glycans on IgG-Fe. IgG can be harnessed to trap even highly motile bacterial pathogens and enable pathogen trapping in different mucus secretions, including from the airways as well as GI and female reproductive tracts, underscoring pathogen trapping by IgG-mucin affinity as a universal mucosal protective mechanism. Our multimeric HCA constructs retain the muco-trapping potencies relative to IgG and effectively immobilize individual sperm in mucus.

### Release Device Example: Intravaginal Ring

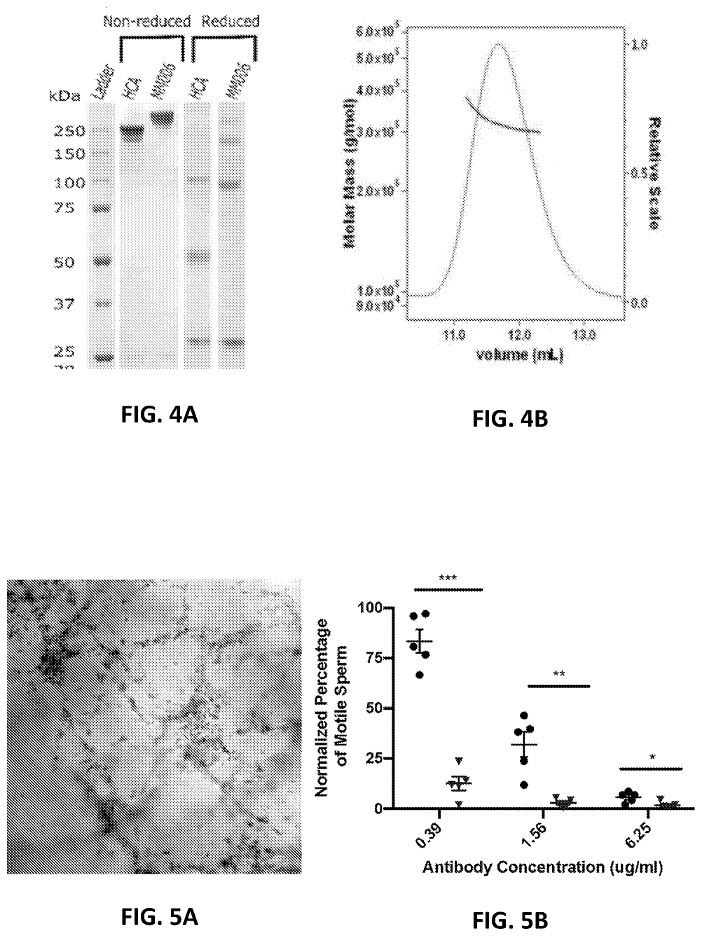

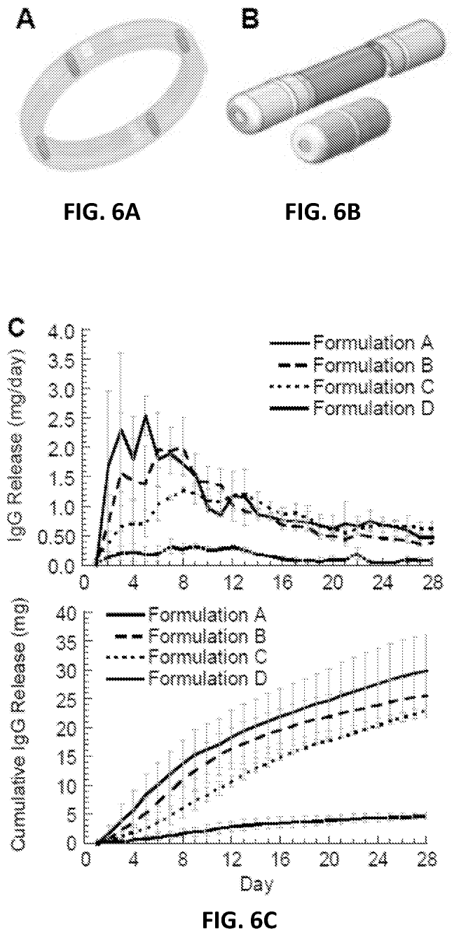

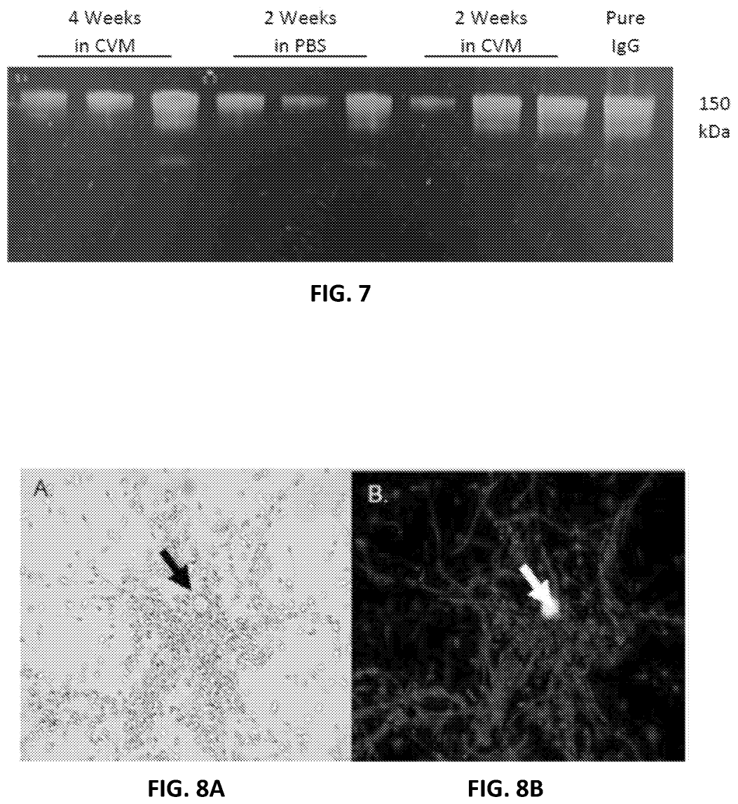

FIGS. 6A-6C describe a capsule-IVR (intravaginal ring) system that may be used with the synthetic binding agent having multiple Fab repeats described herein. This delivery apparatus makes use of conventional pill-processing to fabricate capsules that can be embedded in IVR and facilitate sustained release of the synthetic binding agent. In FIG. 6A the device is a ring that may be placed in the vagina (e.g., intrauterine) and multiple time-release capsules (FIG. 6B) may be loaded thereon. The capsules have been shown to maintain structural stability of the synthetic binding agent having multiple Fab repeats for at least 4 weeks when immersed in human CVM at 37° C. (CVM replaced every 3-4 days). This is illustrated in FIG. 6C showing both daily and cumulative release over one month. The release rates can be readily tuned over a wide range of release rates, including as low as in the 0.1-0.3 mg/day range (FIG. 4C; Formulation D) for 28 days or more. The capsules can also be formulated to provide greater release rates during the Days 2-6 window (Formulation A & B); it may be desirable to have greater dose of HCA delivered immediately before the fertility window. The synthetic binding agent having multiple Fab repeats configured as HCA described herein, when loaded into suitable IVR systems, will enable a reliable and safe contraceptive product that is not only non-hormonal but also economically feasible, and does not require daily or coitally-associated administration.

### Examples: Sequences

The synthetic binding agents described herein typically include multiple additional copies of Fab regions, as described above. As describe above in FIGS. 1A-1G, these synthetic binding agents may be arranged in in a variety of different configurations of a core IgG (including Fc and Fab domains), in which the duplicated copies of the Fab domains (directed to the same target epitope as the Fab domains on the core IgG) are attached at either or both the NH2 and/or the COOH ends. Any Fab domain may be used, and may be linked to the amino or carboxyl ends via a flexible linker comprising an amino acid sequence comprising n pentapeptide repeats consisting of Glycine (G) and Serine (S), wherein n is between 3 and 8.

As an example, a synthetic binding agent may be directed to an N-linked glycan of an epitope specific to sperm, referred to as CD52 glycoform (“CD52g”). SEQ ID NO: 1 shows one example of an amino acid sequence corresponding to CD52g (see, e.g., Diekman et al., FASEB Journal, vol. 13: 1303-1313, August 1999). The variable domains (heavy and/or light) of any antibody directed against a protein including this sequence may be used, and configured as a synthetic binding agent as described herein. The exemplary synthetic binding agents described by SEQ ID NOS: 2-31 illustrate examples of such antibodies directed against a sperm-specific epitope. For example, a synthetic binding agent that is directed to an epitope specific to sperm, such as CD52g (e.g., an n-glycosylated form of CD52) includes both heavy chain and light chain. SEQ ID NO: 2 is an exemplary DNA sequence for a heavy chain domain of a core IgG directed to an epitope of CD52g, and SEQ ID NO: 3 is an example of an amino acid sequence for a heavy chain portion of the IgG. SEQ ID NO: 4 is an example of an amino acid sequence of a Fab fragment for a heavy chain. SEQ ID NO: 5 is an example of an amino acid sequence of an Fc fragment of a heavy chain. SEQ ID NO: 6 is an example of an exemplary DNA sequence for a light chain domain of a core IgG directed to an epitope of CD52g. SEQ ID NO: 7 is an example of an amino acid sequence of a light chain domain of a core IgG directed to an epitope of CD52g.

SEQ ID NO: 8 to SEQ ID NO: 13 show exemplary DNA and amino acid sequences for heavy and light chain portions of a synthetic binding agent (e.g., recombinant mAb) that may reduce sperm mobility in mucus having a structure similar to that shown in FIG. 1B (e.g., Fab-IgG). SEQ ID NO: 8 is an exemplary DNA sequence for a heavy chain domain of a Fab-IgG synthetic binding agent directed to an epitope of CD52g, and SEQ ID NO: 9 is an example of an amino acid sequence for a heavy chain portion of a Fab-IgG. SEQ ID NO: 10 is an example of an amino acid sequence of a Fab fragment for a heavy chain of a synthetic binding agent including a Fab extending from the N-terminal end of a core IgG. SEQ ID NO: 11 is an example of an amino acid sequence of an Fc fragment of a heavy chain. SEQ ID NO: 12 is an example of an exemplary DNA sequence for a light chain domain of a core IgG directed to an epitope of CD52g. SEQ ID NO: 13 is an example of an amino acid sequence of a light chain domain of a synthetic binding agent including a Fab extending from the N-terminal end of a core IgG directed to an epitope of CD52g.

SEQ ID NO: 14 to SEQ ID NO: 19 show exemplary DNA and amino acid sequences for heavy and light chain portions of a synthetic binding agent (e.g., recombinant mAb) that may reduce sperm mobility in mucus having a structure similar to that shown in FIG. 1C (e.g., IgG-Fab). SEQ ID NO: 14 is an exemplary DNA sequence for a heavy chain domain of an IgG-Fab synthetic binding agent directed to an epitope of CD52g, and SEQ ID NO: 15 is an example of an amino acid sequence for a heavy chain portion of such an IgG-Fab. SEQ ID NO: 16 is an example of an amino acid sequence of a Fab fragment for a heavy chain of a synthetic binding agent including a Fab extending from the N-terminal end of a core IgG. SEQ ID NO: 17 is an example of an amino acid sequence of an Fc fragment of a heavy chain. SEQ ID NO: 18 is an example of an exemplary DNA sequence for a light chain domain directed to an epitope of CD52g. SEQ ID NO: 19 is an example of an amino acid sequence of a light chain domain of a synthetic binding agent directed to an epitope of CD52g.

SEQ ID NO: 20 to SEQ ID NO: 25 show exemplary DNA and amino acid sequences for heavy and light chain portions of a synthetic binding agent (e.g., recombinant mAb) that may reduce sperm mobility in mucus having a structure similar to that shown in FIG. 1D (e.g., Fab-IgG-Fab). SEQ ID NO: 20 is an exemplary DNA sequence for a heavy chain domain of an Fab-IgG-Fab synthetic binding agent directed to an epitope of CD52g, and SEQ ID NO: 21 is an example of an amino acid sequence for a heavy chain portion of such a Fab-IgG-Fab. SEQ ID NO: 22 is an example of an amino acid sequence of a Fab fragment for a heavy chain of a synthetic binding agent directed to an epitope of CD52g. SEQ ID NO: 23 is an example of an amino acid sequence of an Fc fragment of a heavy chain of a synthetic binding agent directed to an epitope of CD52g. SEQ ID NO: 24 is an example of an exemplary DNA sequence for a light chain domain directed to an epitope of CD52g. SEQ ID NO: 25 is an example of an amino acid sequence of a light chain domain of a synthetic binding agent directed to an epitope of CD52g.

SEQ ID NO: 26 to SEQ ID NO: 31 show exemplary DNA and amino acid sequences for heavy and light chain portions of a synthetic binding agent (e.g., recombinant mAb) that may reduce sperm mobility in mucus having a structure similar to that shown in FIG. 1E (e.g., Fab-IgG-Fab-Fab). SEQ ID NO: 26 is an exemplary DNA sequence for a heavy chain domain of an Fab-IgG-Fab-Fab synthetic binding agent directed to an epitope of CD52g, and SEQ ID NO: 27 is an example of an amino acid sequence for a heavy chain portion of such a Fab-IgG-Fab-Fab. SEQ ID NO: 28 is an example of an amino acid sequence of a Fab fragment for a heavy chain of a synthetic binding agent directed to an epitope of CD52g. SEQ ID NO: 29 is an example of an amino acid sequence of an Fc fragment of a heavy chain of a synthetic binding agent directed to an epitope of CD52g. SEQ ID NO: 30 is an example of an exemplary DNA sequence for a light chain domain directed to an epitope of CD52g. SEQ ID NO: 31 is an example of an amino acid sequence of a light chain domain of a synthetic binding agent directed to an epitope of CD52g.

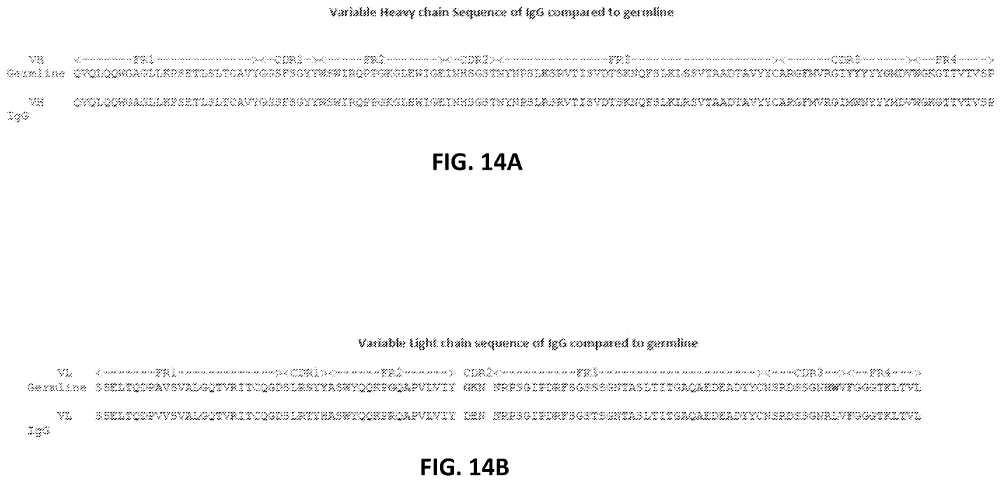

FIGS. 14A and 14B illustrate a comparison between the amino acid sequences of the heavy (FIG. 14A) and light (FIG. 14B) chain sequences described in the sequence listing, as compared to a germline sequence (e.g., native IgG). The notation of the different constructs are listed from NH2 to COOH ends, with IgG implying Fab-Fc.

SEQ ID NO: 32 to SEQ ID NO: 38 show an exemplary DNA and amino acid sequences for heavy and light chain portions of a synthetic binding agent (e.g., recombinant mAb) that may reduce sperm mobility in mucus having a structure of Fab-Fab-IgG-Fab-Fab. SEQ ID NO: 32 is an exemplary DNA sequence for a heavy chain domain of a Fab-Fab-IgG-Fab-Fab synthetic binding agent directed to an epitope of CD52g, and SEQ ID NO: 33 is an example of an amino acid sequence for a heavy chain portion of such a Fab-Fab-IgG-Fab-Fab. SEQ ID NO: 34 is an example of a DNA sequence for a light chain of the anti-CD53g Fab-Fab-IgG-Fab-Fab synthetic protein. SEQ ID NO: 35 is an example of an amino acid sequence of the anti-CD52g Fab-Fab-IgG-Fab-Fab synthetic binding agent. SEQ ID NO: 36 is an amino acid sequence of the Fab fragment of the Fab-Fab-IgG-Fab-Fab (heavy chain) portion, while SEQ ID NO: 37 is the amino acid sequence of anti-CD52g Fab fragment of Fab-Fab-IgG-Fab-Fab. SEQ ID NO: 38 is an example of an amino acid sequence of an Fc fragment of a heavy chain of a synthetic binding agent directed to an epitope of CD52g, including configured as a Fab-Fab-IgG-Fab-Fab.

In another example, a synthetic binding agent, which in particular may reduce the fraction of pathogen that can permeate through mucus and/or freely divide as described herein, may be directed against Klebsiella (e.g., having anti-Klebsiella activity). For example, a human or humanized IgG (mAb) that specifically recognizes an epitope of Klebsiella pneumonia O1 may be used. For example, the anti-Klebsiella mAb illustrated by SEQ ID NO: 39 to SEQ ID NO: 45 is directed against the D-galactan-II antigen of Klebsiella pneumonia; other epitopes or other anti-Klebsiella mAbs may be used instead. For example, SEQ ID NO: 39 is a polynucleotide (DNA) sequence of the heavy chain of an anti-Klebsiella IgG. SEQ ID NO: 40 is an amino acid sequence of an anti-Klebsiella heavy chain. SEQ ID NO: 43 is a polynucleotide (DNA) sequence of a light chain of the anti-Klebsiella IgG; SEQ ID NO: 44 is an amino acid sequence of the anti-Klebsiella light chain. SEQ ID NO: 45 is an amino acid sequence of a Fab fragment of this anti-Klebsiella IgG light chain, while SEQ ID NO: 41 is an amino acid sequence of a Fab fragment of an anti-Klebsiella heavy chain and SEQ ID NO: 42 is an amino acid sequence of an Fc fragment of the heavy chain of this anti-Klebsiella antibody.

An example of a synthetic binding agent specific to Klebsiella constructed as described herein as a Fab-IgG construct (similar to FIG. 1B) is described by SEQ ID NO: 46 to SEQ ID NO: 52. SEQ ID NO: 46 is the DNA sequence of the anti-Klebsiella Fab-IgG heavy chain, while SEQ ID NO: 47 is an amino acid sequence of a heavy chain of a Fab-IgG. SEQ ID NO: 48 is an amino acid sequence of this anti-Klebsiella Fab fragment of a Fab-IgG heavy chain. SEQ ID NO: 49 is an amino acid sequence of the Fc fragment of an IgG-Fab. SEQ ID NO: 50 is a DNA sequence of the light chain of the Fab-IgG, and SEQ ID NO: 51 is an amino acid sequence of the light chain of the Fab-IgG. SEQ ID NO: 52 shows an amino acid sequence of a Fab fragment of Fab-IgG Light Chain.

Another example of a synthetic binding agent specific to Klebsiella, constructed as an IgG-Fab construct (similar to FIG. 1C) is described by SEQ ID NO: 53 to SEQ ID NO: 59. SEQ ID NO: 53 is the DNA sequence of the anti-Klebsiella IgG-Fab heavy chain, while SEQ ID NO: 54 is an amino acid sequence of a heavy chain of a IgG-Fab. SEQ ID NO: 55 is an amino acid sequence of an anti-Klebsiella Fab fragment of a IgG-Fab heavy chain. SEQ ID NO: 56 is an amino acid sequence of an Fc fragment of an IgG-Fab. SEQ ID NO: 57 is a DNA sequence of the light chain of this IgG-Fab synthetic binding agent, and SEQ ID NO: 58 is an amino acid sequence of the light chain of the IgG-Fab. SEQ ID NO: 59 shows an amino acid sequence of a Fab fragment of IgG-Fab Light Chain.

An example of a synthetic binding agent specific to Klebsiella constructed as described herein as a Fab-IgG-Fab construct (similar to FIG. 1D) is described by SEQ ID NO: 60 to SEQ ID NO: 66. SEQ ID NO: 60 is the DNA sequence of the anti-Klebsiella Fab-IgG-Fab heavy chain, and SEQ ID NO: 61 is an amino acid sequence of a heavy chain of a Fab-IgG-Fab. SEQ ID NO: 62 is an amino acid sequence of this anti-Klebsiella Fab fragment of a Fab-IgG-Fab heavy chain. SEQ ID NO: 63 is an amino acid sequence of the Fc fragment of an Fab-IgG-Fab. SEQ ID NO: 64 is a DNA sequence of the light chain of the Fab-IgG-Fab, and SEQ ID NO: 65 is an amino acid sequence of the light chain of the Fab-IgG-Fab. SEQ ID NO: 66 shows an amino acid sequence of a Fab fragment of Fab-IgG-Fab Light Chain.

An example of a synthetic binding agent specific to Klebsiella constructed as described herein as a Fab-Fab-IgG-Fab-Fab construct (similar to FIG. 1F) is described by SEQ ID NO: 160 to SEQ ID NO: 166. SEQ ID NO: 160 is the DNA sequence of the anti-Klebsiella Fab-Fab-IgG-Fab-Fab heavy chain, and SEQ ID NO: 161 is an amino acid sequence of a heavy chain of a Fab-Fab-IgG-Fab-Fab. SEQ ID NO: 162 is an amino acid sequence of this anti-Klebsiella Fab fragment of a Fab-Fab-IgG-Fab-Fab heavy chain. SEQ ID NO: 163 is an amino acid sequence of the Fc fragment of a Fab-Fab-IgG-Fab-Fab. SEQ ID NO: 164 is a DNA sequence of the light chain of the Fab-Fab-IgG-Fab-Fab, and SEQ ID NO: 165 is an amino acid sequence of the light chain of the Fab-Fab-IgG-Fab-Fab. SEQ ID NO: 166 shows an amino acid sequence of a Fab fragment of Fab-Fab-IgG-Fab-Fab Light Chain.

In another example, a synthetic binding agent, which in particular may reduce the fraction of pathogen that can permeate through mucus and/or freely divide as described herein, may be directed against Salmonella (e.g., having anti-Salmonella activity). For example, a human or humanized IgG (mAb) that specifically recognizes an epitope of Salmonella may be used. For example, the anti-Salmonella mAb illustrated by SEQ ID NO: 67 to SEQ ID NO: 73 is directed against an antigen of Salmonella. Any appropriate epitope or other anti-Salmonella mAbs may be used. For example, SEQ ID NO: 67 is a polynucleotide (DNA) sequence of the heavy chain of an anti-Salmonella IgG. SEQ ID NO: 68 is an amino acid sequence of an anti-Salmonella heavy chain. SEQ ID NO: 71 is a polynucleotide (DNA) sequence of a light chain of the anti-Salmonella IgG; SEQ ID NO: 72 is an amino acid sequence of the anti-Salmonella light chain. SEQ ID NO: 69 is an amino acid sequence of a Fab fragment of this anti-Salmonella IgG heavy chain, while SEQ ID NO: 73 is an amino acid sequence of a Fab fragment of an anti-Salmonella light chain and SEQ ID NO: 70 is an amino acid sequence of an Fc fragment of the heavy chain of this anti-Klebsiella antibody.

An example of a Fab-IgG synthetic anti-Salmonella LP binding agent is described by SEQ ID NO: 74 to SEQ ID NO: 80, including the DNA sequence of a synthetic Fab-IgG Heavy Chain in SEQ ID NO: 74 (the amino acid sequence of this heavy chain is shown in SEQ ID NO: 75). The amino acid residue of the Fab fragment of Fab-IgG Heavy Chain is provided in SEQ ID NO: 76 and the amino acid residues of the Fc fragment of Fab-IgG is provided in SEQ ID NO: 77. SEQ ID NO: 78 is a DNA sequence of Fab-IgG (Light Chain) portion, and the amino acid sequence is in SEQ ID NO: 79. SEQ ID NO: 80 lists the amino acid residues of the Fab fragment of Fab-IgG Light Chain.

An example of an IgG-Fab synthetic anti-Salmonella LPS binding agent is described by SEQ ID NO: 81 to SEQ ID NO: 87, including the DNA sequence of a synthetic Fab-IgG Heavy Chain in SEQ ID NO: 81 (the amino acid sequence of this heavy chain is shown in SEQ ID NO: 82). The amino acid residue of the Fab fragment of Fab-IgG Heavy Chain is provided in SEQ ID NO: 83 and the amino acid residues of the Fc fragment of Fab-IgG is provided in SEQ ID NO: 84. SEQ ID NO: 85 is a DNA sequence of Fab-IgG (Light Chain) portion, and the amino acid sequence is in SEQ ID NO: 86. SEQ ID NO: 87 lists the amino acid residues of the Fab fragment of Fab-IgG Light Chain.

An example of a Fab-IgG-Fab synthetic anti-Salmonella LPS binding agent is described by SEQ ID NO: 88 to SEQ ID NO: 94, including the DNA sequence of a synthetic Fab-IgG Heavy Chain in SEQ ID NO: 88 (the amino acid sequence of this heavy chain is shown in SEQ ID NO: 89). The amino acid residue of the Fab fragment of Fab-IgG Heavy Chain is provided in SEQ ID NO: 90 and the amino acid residues of the Fc fragment of Fab-IgG is provided in SEQ ID NO: 91. SEQ ID NO: 92 is a DNA sequence of Fab-IgG (Light Chain) portion, and the amino acid sequence is in SEQ ID NO: 93. SEQ ID NO: 94 lists the amino acid residues of the Fab fragment of Fab-IgG Light Chain.

An example of a Fab-Fab-IgG-Fab-Fab synthetic anti-Salmonella LPS binding agent is described by SEQ ID NO: 95 to SEQ ID NO: 101, including the DNA sequence of a synthetic Fab-IgG Heavy Chain in SEQ ID NO: 95 (the amino acid sequence of this heavy chain is shown in SEQ ID NO: 96). The amino acid residue of the Fab fragment of Fab-IgG Heavy Chain is provided in SEQ ID NO: 97 and the amino acid residues of the Fc fragment of Fab-IgG is provided in SEQ ID NO: 98. SEQ ID NO: 99 is a DNA sequence of Fab-IgG (Light Chain) portion, and the amino acid sequence is in SEQ ID NO: 100. SEQ ID NO: 101 lists the amino acid residues of the Fab fragment of Fab-IgG Light Chain.

In another example, a synthetic binding agent, which in particular may reduce the fraction of pathogen that can permeate through mucus and/or freely divide as described herein, may be directed against Neisseria gonorrhoeae (e.g., having anti-Gonorrhea activity). For example, a human or humanized IgG (mAb) that specifically recognizes an epitope of Neisseria gonorrhoeae may be used. For example, the anti-Gonorrhea mAb (2C7) illustrated by SEQ ID NO: 102 to SEQ ID NO: 108 is directed against an antigen of Neisseria gonorrhoeae. Any appropriate epitope or other anti-gonorrhoeae mAbs may be used. For example, SEQ ID NO: 102 is a polynucleotide (DNA) sequence of the heavy chain of an anti-gonorrhoeae IgG. SEQ ID NO: 103 is an amino acid sequence of an anti-gonorrhoeae heavy chain. SEQ ID NO: 106 is a polynucleotide (DNA) sequence of a light chain of the anti-gonorrhoeae IgG; SEQ ID NO: 107 is an amino acid sequence of the anti-gonorrhoeae light chain. SEQ ID NO: 104 is an amino acid sequence of a Fab fragment of this anti-gonorrhoeae IgG heavy chain, while SEQ ID NO: 108 is an amino acid sequence of a Fab fragment of an anti-gonorrhoeae light chain and SEQ ID NO: 105 is an amino acid sequence of an Fc fragment of the heavy chain of this anti-gonorrhoeae antibody.

An example of a Fab-IgG synthetic anti-gonorrhoeae (2C7) binding agent is described by SEQ ID NO: 109 to SEQ ID NO: 115, including the DNA sequence of a synthetic Fab-IgG Heavy Chain in SEQ ID NO: 109 (the amino acid sequence of this heavy chain is shown in SEQ ID NO: 110). The amino acid residue of the Fab fragment of Fab-IgG Heavy Chain is provided in SEQ ID NO: 111 and the amino acid residues of the Fc fragment of Fab-IgG is provided in SEQ ID NO: 112. SEQ ID NO: 113 is a DNA sequence of Fab-IgG (Light Chain) portion, and the amino acid sequence is in SEQ ID NO: 114. SEQ ID NO: 115 lists the amino acid residues of the Fab fragment of Fab-IgG Light Chain.

An example of an IgG-Fab synthetic anti-gonorrhoeae binding agent is described by SEQ ID NO: 116 to SEQ ID NO: 122, including the DNA sequence of a synthetic IgG-Fab Heavy Chain in SEQ ID NO: 116 (the amino acid sequence of this heavy chain is shown in SEQ ID NO: 117). The amino acid residue of the Fab fragment of IgG-Fab Heavy Chain is provided in SEQ ID NO: 118 and the amino acid residues of the Fc fragment of IgG-Fab is provided in SEQ ID NO: 119. SEQ ID NO: 120 is a DNA sequence of IgG-Fab (Light Chain) portion, and the amino acid sequence is in SEQ ID NO: 121. SEQ ID NO: 122 lists the amino acid residues of the Fab fragment of IgG-Fab Light Chain.

An example of a Fab-IgG-Fab synthetic anti-gonorrhoeae binding agent is described by SEQ ID NO: 123 to SEQ ID NO: 129, including the DNA sequence of a synthetic Fab-IgG-Fab Heavy Chain in SEQ ID NO: 123 (the amino acid sequence of this heavy chain is shown in SEQ ID NO: 124). The amino acid residue of the Fab fragment of Fab-IgG-Fab Heavy Chain is provided in SEQ ID NO: 125 and the amino acid residues of the Fc fragment of Fab-IgG-Fab is provided in SEQ ID NO: 126. SEQ ID NO: 127 is a DNA sequence of Fab-IgG-Fab (Light Chain) portion, and the amino acid sequence is in SEQ ID NO: 128. SEQ ID NO: 129 lists the amino acid residues of the Fab fragment of Fab-IgG-Fab Light Chain.

An example of a Fab-Fab-IgG-Fab-Fab synthetic anti-gonorrhoeae binding agent is described by SEQ ID NO: 153 to SEQ ID NO: 159, including the DNA sequence of a synthetic Fab-Fab-IgG-Fab-Fab Heavy Chain in SEQ ID NO: 153 (the amino acid sequence of this heavy chain is shown in SEQ ID NO: 154). The amino acid residue of the Fab fragment of Fab-Fab-IgG-Fab-Fab Heavy Chain is provided in SEQ ID NO: 155 and the amino acid residues of the Fc fragment of Fab-Fab-IgG-Fab-Fab is provided in SEQ ID NO: 156. SEQ ID NO: 157 is a DNA sequence of Fab-Fab-IgG-Fab-Fab (Light Chain) portion, and the amino acid sequence is in SEQ ID NO: 158. SEQ ID NO: 159 lists the amino acid residues of the Fab fragment of Fab-Fab-IgG-Fab-Fab Light Chain.

In another example, a synthetic binding agent, which in particular may reduce the fraction of pathogen that can permeate through mucus as described herein, may be directed against Respiratory Syncytial Virus (RSV). For example, a human or humanized IgG (mAb) that specifically recognizes an epitope of RSV may be used. For example, an anti-RSV mAb (modeled after published Motavizumab) is illustrated by SEQ ID NO: 132 to SEQ ID NO: 138 and is directed against an antigen of RSV. Any appropriate epitope or other anti-RSV mAbs may be used. SEQ ID NO: 132 is a polynucleotide (DNA) sequence of the heavy chain of an anti-RSV IgG. SEQ ID NO: 133 is an amino acid sequence of an anti-RSV heavy chain. SEQ ID NO: 136 is a polynucleotide (DNA) sequence of a light chain of the anti-RSV IgG; SEQ ID NO: 137 is an amino acid sequence of the anti-RSV light chain. SEQ ID NO: 134 is an amino acid sequence of a Fab fragment of this anti-RSV IgG heavy chain, while SEQ ID NO: 138 is an amino acid sequence of a Fab fragment of an anti-RSV light chain and SEQ ID NO: 135 is an amino acid sequence of an Fc fragment of the heavy chain of this anti-RSV antibody.

An example of a Fab-IgG synthetic anti-RSV binding agent is described by SEQ ID NO: 139 to SEQ ID NO: 145, including the DNA sequence of a synthetic Fab-IgG Heavy Chain in SEQ ID NO: 139 (the amino acid sequence of this heavy chain is shown in SEQ ID NO: 140). The amino acid residue of the Fab fragment of Fab-IgG Heavy Chain is provided in SEQ ID NO: 141 and the amino acid residues of the Fc fragment of Fab-IgG is provided in SEQ ID NO: 142. SEQ ID NO: 143 is a DNA sequence of Fab-IgG (Light Chain) portion, and the amino acid sequence is in SEQ ID NO: 144. SEQ ID NO: 145 lists the amino acid residues of the Fab fragment of Fab-IgG Light Chain.

An example of an IgG-Fab synthetic anti-RSV binding agent is described by SEQ ID NO: 146 to SEQ ID NO: 152, including the DNA sequence of a synthetic Fab-IgG Heavy Chain in SEQ ID NO: 146 (the amino acid sequence of this heavy chain is shown in SEQ ID NO: 147). The amino acid residue of the Fab fragment of Fab-IgG Heavy Chain is provided in SEQ ID NO: 148 and the amino acid residues of the Fc fragment of Fab-IgG is provided in SEQ ID NO: 149. SEQ ID NO: 150 is a DNA sequence of Fab-IgG (Light Chain) portion, and the amino acid sequence is in SEQ ID NO: 151. SEQ ID NO: 152 lists the amino acid residues of the Fab fragment of Fab-IgG Light Chain.

Other synthetic binding agents (e.g., multimeric constructs) may be directed against Psuedomonas aeruginosa, Methicillin-resistant Staphylococcus aureus, Acinetobacter baumannii, and Clostridium difficile. Sequences for IgG mAbs against surface antigens for these (and other pathogens) are published and synthetic binding agents may be formed as described herein. Thus, although specific sequences of exemplary synthetic binding agents that may reduce the fraction of pathogen that can permeate through mucus and/or freely divide are described above, one of skill in the art, may understand that the specification generally teaches the method of making and using synthetic binding agents from an IgG, particularly IgGs directed against surface antigens.

### Examples

The synthetic binding agents described herein are synthetic human or humanized Immunoglobulin G (IgG) having a pair of Fab domains to which additional Fab domains directed to the same antigen are linked by a flexible linker at either or both the end(s) of the Fab domains of the IgG and/or the Fc region of the IgG, in tandem. The resulting synthetic binding agent has been found to dramatically reduce the mobility of the target (e.g., pathogen, such as bacteria, virus, yeast, etc. and/or sperm, etc.) in mucus. The synthetic binding agents were found to be stable across a variety of delivery forms, including nebulized forms, and can be readily produced using the methods and techniques described herein.

For example, studies were performed to demonstrate that virtually any starting IgG (e.g., IgG1 mAb) having specific binding for an antigen (or antigen region) of a target, such as sperm or a pathogen (virus, bacteria, yeast, mold, etc.), a synthetic binding agent as described herein may be generated. In some variations the variable heavy chain and light chain, in some cases as well as constant heavy and light chain sequences, of a starting IgG1 mAb were codon-optimized for Homo sapiens using the optimization tool, such as that provided by GeneArt (ThermoFisher Scientific). Codon-optimized sequences of VH, CH1, VL and CL may be used to design the gene fragments required to assemble the synthetic binding agents described herein (e.g., using software such as Benchling software).

For example, to assemble a Fab-IgG synthetic binding agent, a gene fragment comprised of VH-CH1-6×G4S-Linkers-VH was designed to be cloned into mammalian expression vector comprised of CH1-CH2-CH3 DNA sequences. Similarly, to assemble Fab-Fab-IgG-Fab-Fab, the gene fragments comprised of VH-CH1-6×G4SLinkers-VH-CH1-6×G4SLinkers-VH and 6×G4SLinkers-VH-CH1-6×G4SLinkers-VH-CH1 were designed to be further cloned into an IgG1 expression vector. In some examples, to minimize synthesis problems that could occur due to repeated sequences, the DNA sequences for repeated fragments may be, e.g., manually, codon-optimized resulting in increased variability of DNA sequences and subsequent reduced complexity for gene synthesis. After codon-optimization, gene sequences may be further processed through a complexity-analyzing tool provided (e.g., such as that provided by IDT (Integrated DNA Technologies) to obtain a complexity score. Gene fragments with complexity scores<25 are known to be easily and successfully synthesized via GeneArt Gene synthesis.

Expression vectors encoding the synthetic binding agent may be generated. For example, an expression plasmid encoding the light chain, the gene fragment consisting of V_{L} and CL (C.) DNA sequences may be synthesized using custom gene-synthesis service (e.g., Integrated DNA Technologies) and cloned into an empty mammalian expression vector using, e.g., KpnI (5′) and EcoRI (3′) restriction sites. For the construction of expression plasmids encoding heavy chains (HC) for the synthetic binding agent, in some examples four cloning vectors comprising of VH-CH1-6×G4SLinkers-VH, VH-CH1-6×G4SLinkers-VH-CH1-6×G4SLinkers-VH, 6×G4SLinkers-VH-CH1 and 6×G4SLinkers-VH-CH1-6×G4SLinkers-VH-CH1 DNA sequences were synthesized using GeneArt® gene synthesis service (ThermoFisher Scientific). In some examples, for the construction of expression plasmid encoding HC for IgG, VH fragment was amplified from the cloning vector comprising of VH-CH1-6×G4SLinkers-VH vector using forward primer, 5′-TAAGCAGGTACCGCCACCATGAAGTG-3′ (SEQ ID NO: 130), and reverse primer, 5′-TGCTTAGCTAGCTGGAGAAACTGTC-3′ (SEQ ID NO: 131), and then cloned into the mammalian expression vector comprised of CH1-CH2-CH3 DNA sequences using KpnI (5′) and NheI (3′) restriction sites. In some examples, for the construction of expression plasmid encoding HC for Fab-IgG, VH-CH1-6×G4SLinkers-VH fragment was cloned into the same mammalian expression vector using KpnI (5′) and NheI (3′) restriction sites. For example, the construction of an expression plasmid encoding HC for IgG-Fab may include using a 6×G4SLinkers-VH-CH1 fragment that is cloned into the IgG mammalian expression vector using BamHI (5′) and MluI (3′) restriction sites. For the construction of expression plasmid encoding HC for Fab-IgG-Fab, VH-CH1-6×G4SLinkers-VH fragment may be first cloned into the mammalian expression vector using KpnI (5′) and NheI (3′) restriction sites followed by the cloning of 6×G4SLinkers-VH-CH1 fragment using BamHI (5′) and MluI (3′) restriction sites. For the construction of expression plasmid encoding HC for Fab-IgG-Fab-Fab, VH-CH1-6×G4SLinkers-VH fragment may be first cloned into the mammalian expression vector using KpnI (5′) and NheI (3′) restriction sites followed by the cloning of 6×G4SLinkers-VH-CH1-6×G4SLinkers-VH-CH1 fragment using BamHI (5′) and MluI (3′) restriction sites. For the construction of expression plasmid encoding HC for Fab-Fab-IgG-Fab-Fab, VH-CH1-6×G4SLinkers-VH-CH1-6×G4SLinkers-VH fragment may be first cloned into the mammalian expression vector using KpnI (5′) and NheI (3′) restriction sites followed by the cloning of 6×G4SLinkers-VH-CH1-6×G4SLinkers-VH-CH1 fragment using BamHI (5′) and MluI (3′) restriction sites. For the ligation of all heavy chains as well as a light chain into the expression vectors, quick ligation kit (New England Biolabs, Ipswich, Mass.) may be used. All ligated DNA constructs may be transformed into chemically competent TOP10 E. coli cells (Life Technologies) and plated on ampicillin plates for selection. Bacterial colonies may be picked, cultured, and the plasmids prepped (e.g., Qiagen MiniPrep Kit). Correct assembly of the constructs into the expression vector may be confirmed by Sanger sequencing (e.g., Eurofins Genomics).

In some of the experiments described herein, expression plasmids encoding the heavy chain (HC) and light chain (LC) for IgG, Fab-IgG, IgG-Fab, FIF, FIFF and FFIFF antibodies were scaled up by transforming the sequencing-confirmed expression plasmids in chemically competent TOP10 E. coli, inoculating the transformation mix into 100 mL Luria broth in a 250 mL baffled flask and overnight shaking at 220 r.p.m at 37° C. Midi-prep plasmid purifications were done using NucleoBond® Xtra Midi EF Kits (Macherey-Nagel) according to the manufacturer's protocols. Proteins were expressed in Expi293 cells using ExpiFectamine™ 293 Transfection reagents and protocols provided by the manufacturer (ThermoFisher Scientific). For IgG, one HC and one LC plasmid were co-transfected using a 1:1 ratio at 1 μg total DNA per 1 mL of culture. For both Fab-IgG and IgG-Fab, one HC and one LC plasmid were co-transfected using a 1:2 ratio at 1 μg total DNA per 1 mL culture. For Fab-IgG-Fab, one HC and one LC plasmid were co-transfected using a 1:3 ratio at 1 μg total DNA per 1 mL culture. For Fab-IgG-Fab-Fab, one HC and one LC plasmid were co-transfected using a 1:4 ratio at 1 μg total DNA per 1 mL culture. For Fab-Fab-IgG-Fab-Fab, one HC plasmid and one LC plasmid were co-transfected using a 1:5 ratio at 1 μg total DNA per 1 mL culture. Transfected cells were grown at 37° C. in a 5% CO₂ incubator while shaking at 125 r.p.m. for 5 days. Supernatants were harvested by centrifugation at 5000 g for 10 min and passed through 0.22-μm filters for purification using standard protein A affinity chromatography. Briefly, 30 mL of transfected supernatant was incubated with 400 μL PBS-washed Pierce™ Protein A Plus Agarose Resin (ThermoFisher Scientific) overnight at 4° C. Next, the resin-supernatant solution was flown through the gravity columns followed by the washing of resin. Protein was eluted by adding 900 μL of Pierce™ IgG Elution Buffer (ThermoFisher Scientific) into PBS-washed resin and was immediately neutralized by adding 100 μL of UltraPure™ 1 M Tris-HCl Buffer, pH 7.5 (ThermoFisher Scientific). Eluted proteins were further dialyzed into PBS using Amicon® Ultra Centrifugal Filters (Millipore Sigma).

In some variations the synthetic binding agent may be delivered to a mucosa, as described herein. Delivery may be via topical delivery, including aerosol, liquid, or gel (including dissolvable gel). For example in some variations a film may be used to deliver the synthetic binding agent. In some cases, a vaginal film may be used.

To examine this in the context of a synthetic anti-sperm binding agent, genes containing the complete heavy chain and light chain sequences of IgG and Fab-IgG-Fab were cloned into plant expression vectors (TMV and PVX; Icon Genetics) followed by transformation into Agrobacterium tumefaciens strain ICF320 (Icon Genetics). Next, the transformation mixture was infiltrated into the 4 wk old N. benthamiana plants that were genetically modified to produce highly homogenous mammalian N-glycans of the GnGn glycoform. Seven days later, anti-sperm antibodies were extracted from the leaf tissue and purified using protein A chromatography. To remove the endotoxins, purified mAbs were passed through an Acrodise Units with Mustang Q Membrane (Pall Life Sciences). Endosafe PTS (Charles River) was utilized to measure the endotoxin level, which was found to less than 150 EU/mg. A film, such as a Nicotiana-produced HCA film, may then be made. In some experiments, the HCA films were formulated as a 2-inch by 2-inch polyvinyl alcohol (PVA) film casts and dried from an aqueous wet blend. The aqueous wet blend was composed of approximately 38.5 mL formulated antibody concentrate (200 mg/mL maltitol, 10.0 mM histidine, 0.05 mg/mL, polysorbate 20) mixed with an aqueous polymer concentrate (17.88 g PVA 8-88 dissolved in 53.6 g WFI). IgG-Film and FIF-Film (e.g., Fab-IgG-Fab) were composed with 20 mg and 10 mg of mAbs per film respectively. The films were dissolved in ultra-pure water to perform characterization studies followed by sperm potency and agglutination kinetics assays.

The synthetic binding agents described herein may also be nebulized for delivery without significantly reducing their efficacy. For example, nebulization of FIF (Fab-IgG-Fab) constructs and FFIFF (Fab-Fab-IgG-Fab-Fab) constructs were examined. FIF and FFIFF antibodies were nebulized using a PARI eRapid vibrating mesh nebulizer system. The nebulized solutions were collected into a 50 mL conical tube, and the stability of the nebulized antibodies was assessed using SDS-PAGE, and the affinity of the antibody to its antigen assessed using whole-sperm ELISA assay.

In some of the examples described herein, biophysical characterization of the synthetic binding agents (e.g., Fab-IgG, IgG-Fab, Fab-IgG-Fab, Fab-Fab-IgG, IgG-Fab-Fab, Fab-Fab-IgG-Fab, Fab-IgG-Fab-Fab, Fab-Fab-IgG-Fab-Fab) were done using SDS-PAGE, SEC-MALS and nano-DSF. SDS-PAGE experiments were performed using 4-12% NuPage Bis-Tris gels (ThermoFisher Scientific) in 1× NuPage MOPS buffer under both reducing and non-reducing conditions to confirm the correct assembly of all HCA protein constructs. For each protein sample, 1 μg of protein was diluted in 3.75 μL LDS sample buffer followed by the addition of 11.25 μL nuclease-free water. Proteins were then denatured at 70° C. for 10 min in a thermocycler. Next, 0.3 μL of 0.5 M TCEP was added as a reducing agent to the denatured protein for reduced samples and incubated at room temperature for 5 min. Bio-Rad Precision Protein Plus Unstained Standard and Novex™ Sharp Pre-stained Protein Standard were used as ladders. After loading the samples, the gel was run for 50 min at a constant voltage of 200 V and washed 3 times with Milli-Q water. Then, the protein bands were visualized by staining with Imperial Protein Stain (ThermoFisher Scientific) for 1 hr followed by overnight de-staining with Milli-Q water.

SEC-MALS experiments were performed at room temperature using a GE Superdex 200 10/300 column connected to an Agilent FPLC system, a Wyatt DAWN HELEOS II multi-angle light-scattering instrument, and a Wyatt T-rEX refractometer. The flow rate was maintained at 0.5 mL/min. The column was equilibrated with 1×PBS, pH 7.4 containing 200 mg/liter of NaN3 prior to sample loading. 50-100 ug of each sample was injected onto the column, and data were collected for 50 min. The MALS data were collected and analyzed using Wyatt ASTRA software (Ver. 6).

NanoDSF experiments were performed using a Nanotemper Prometheus NT.48 system. Samples were diluted to 0.5 mg/mL in 1×PBS at pH 7.4 and loaded into Prometheus NT.48 capillaries. Thermal denaturation experiments were performed from 25° C. to 95° C. at the rate of 1° C./min, measuring the intrinsic tryptophan fluorescence at 330 nm and at 350 nm. The melting temperature (Tm) for each experiment was calculated automatically by Nanotemper PR Thermcontrol software by plotting ratiometric measurement of the fluorescent signal against increasing temperature. The aggregation temperature (Tagg) for each experiment was also calculated automatically by Nanotemper PR Thermcontrol software via the detection of the back-reflection intensity of a light beam that passes the sample.

The anti-sperm synthetic binding agents described herein were examined to determine sperm agglutination potency as well as muco-trapping potency. Fresh semen was examined. For example, male subjects were asked to refrain from sexual activity for 24 hrs prior to semen collection. Semen was collected by masturbation into sterile 50 mL sample cups and incubated for a minimum of 15 min post-ejaculation at room temperature to allow liquefaction. Semen volume was measured, and density gradient sperm separation procedure was used to extract motile sperm from liquefied ejaculates. Briefly, 1.5 mL of liquefied semen was carefully layered over 1.5 mL of Isolate (90% density gradient medium, Irvine Scientific) at room temperature, and centrifuged at 300 g for 20 min. Following centrifugation, the upper layer containing dead cells and seminal plasma was carefully removed without disturbing the motile sperm pellet in the lower layer. The sperm pellet was then washed twice with sperm washing medium (Irvine Scientific) by centrifugation at 300 g for 10 min. Finally, the purified motile sperm pellet was resuspended in sperm washing medium, and an aliquot was taken for determination of sperm count and motility using computer-assisted sperm analysis (CASA). All semen samples used in the functional assays exceeded lower reference limits for sperm count (15×10⁶ spermatozoa per mL) and total motility (40%) as indicated by WHO guidelines. A Hamilton-Thorne computer-assisted sperm analyzer, 12.3 version, was used for the sperm count and motility analysis in all experiments unless stated otherwise. This device consists of a phase-contrast microscope (Olympus CX41), a camera, an image digitizer and a computer with a Hamilton-Thome Ceros 12.3 software to save and analyze the procured data. For each analysis, 4.4 μL of semen sample was inserted into MicroTool counting chamber slides (Cytonix). Then, six randomly selected microscopic fields, near the center of the slide, were imaged and analyzed for progressive and non-progressive motile sperm count. The parameters that were assessed by CASA for motility analysis were as follows: average pathway velocity (VAP: the average velocity of the spermatozoa through a smoothed cell path in μm/sec), the straight-line velocity (VSL: the average velocity measured in a straight line from the beginning to the end of track in μm/sec), the curvilinear velocity (VCL: the average velocity measured over the actual point-to-point track of the cell in μm/sec), the lateral head amplitude (ALH: amplitude of lateral head displacement in μm), the beat cross-frequency (BCF: frequency of sperm head crossing the sperm average path in Hz), the straightness (STR: the average value of the ratio VSL/VAP in %), and the linearity (LIN: the average value of the ratio VSL/VCL in %). Progressive motile sperm were defined as having a minimum of 25 μm/sec VAP and 80% of STR.

Whole Sperm ELISA was also performed. Briefly, half-area polystyrene plates (CLS3690, Corning) were coated with 2.0×10⁵ sperm per well in 50 μL of NaHCO₃ buffer (pH 9.6). After overnight incubation at 4° C., the plates were centrifuged at the speed of 300 g for 20 min. The supernatant was discarded, and the plates were air-dried for 1 hr at 45° C. The plates were washed once with 1×PBS. 100 μL of 5% milk was incubated at room temperature for 1 hr to prevent non-specific binding of an antibody to the microwells. The serial dilution of monoclonal antibodies in 1% milk were added to the microwells and incubated overnight at 4° C. Motavizumab, a mAb against respiratory syncytial virus, was constructed and expressed in the laboratory by accessing the published sequence and used as a negative control for this assay. After primary incubation, the plates were washed three times using PBS. Then, the secondary antibody, goat anti-human IgG F(ab′)2 antibody HRP-conjugated (1:50,000 dilutions in 1% milk, 209-1304, Rockland Inc.) was added to the wells and incubated for 1 hr at room temperature. The washing procedure was repeated and 50 uL of the buffer containing substrate (1-Step Ultra TMB ELISA Substrate, ThermoFisher Scientific) was added to develop the colorimetric reaction for 15 min. The reaction was quenched using 50 uL of 2N H₂SO₄, and the absorbance at 450 nm (signal) and 570 nm (background) was measured using SpectraMax M2 Microplate Reader (Molecular Devices). Each experiment was done with samples in triplicates and repeated at least twice as a measure assay variability.

Sperm escape assays were also performed with purified motile sperm and whole semen. Purified motile sperm were diluted in sperm washing medium to a final concentration of 10×10⁶ progressively motile sperm per mL. Next, 40 μL aliquots of diluted sperm or whole semen were transferred to individual 0.2 mL PCR tubes, and sperm count, and motility was again performed on each 40 μL aliquot using CASA. This count serves as the original (untreated) concentration of sperm for evaluating the agglutination potencies of respective HCA constructs. Following CASA, 30 μL of sperm were added to 30 μL of HCA constructs, and gently mixed by pipetting. The tubes were then fixed at 45° angles in a custom 3D printed tube holder for 5 min at room temperature. Following this incubation period, 4.4 μL was extracted from the top layer of the mixture with minimal perturbation of the tube and transferred to the CASA instrument to quantify the number of progressively motile sperm. The percentage of the progressively motile sperm that escaped agglutination was computed by dividing the sperm count obtained after treatment with HCA constructs by the original sperm count in each respective tube, correcting for the 2-fold dilution with antibody. Each experimental condition was evaluated in duplicates on each semen specimen, and the average from the two experiments was used in the analysis. At least 5 independent experiments were done per assay, each using a single semen donor.

Agglutination kinetics assays with purified motile sperm and whole semen were also performed to characterize the sperm (“anti-sperm”) synthetic binding agent. Purified motile sperm were diluted in sperm washing medium to a final concentration of 10×10⁶ progressively motile sperm per mL or 50×10⁶ progressively motile sperm per mL or 2×10⁶ progressively motile sperm per mL. Next, 4.4 μL of diluted sperm or whole semen was added to 4.4 μL of HCA constructs in 0.2 mL PCR tubes, and mixed by gently pipetting up and down three times over 3 s. A timer was started immediately by a second person while 4.4 μL of the mixture was transferred to chamber slides with a depth of 20 μM (Cytonix, Beltsville, Md.), and video microscopy (Olympus CKX41) using a 10× objective lens focused on the center of chamber slide was captured up to 90 s at 60 frames/s. Progressive sperm count was measured by CASA every 30 s up to 90 s. The percentage of the agglutinated sperm at each time point was computed by normalizing the progressive sperm count obtained after treatment with HCA constructs to the progressive sperm count obtained after treatment with sperm washing media. Each experimental condition was evaluated in duplicates on each semen specimen, and the average from the two experiments used in the analysis. At least 6 independent experiments were done per assay, each using a single semen donor.

Acidic pH stability of IgG- and FIF-Film via agglutination kinetics assay were performed using IgG-Film and FIF-Film constructs that were incubated in 0.5% Lactic Acid (LA) or sperm washing medium (MHM; control) for 24 hrs at 37° C. HCA constructs incubated in LA were neutralized using equal volume of seminal plasma (SP). Next, neutralized HCA were diluted further using either SP or MHM media. Purified motile sperm were diluted in sperm washing medium to a final concentration of 20×10⁶ progressively motile sperm per mL. Next, 4.4 μL of diluted sperm was added to 4.4 μL of HCA constructs (HCA-LA/SP or HCA-LA/MHM or HCA-MHM/MHM) in 0.2 mL PCR tubes, and mixed by gently pipetting up and down three times over 3 s. A timer was started immediately by a second person while 4.4 μL of the mixture was transferred to chamber slides with a depth of 20 μM (Cytonix, Beltsville, Md.), and video microscopy (Olympus CKX41) using a 10× objective lens focused on the center of chamber slide was captured up to 90 s at 60 frames/s. Progressive sperm count was measured by CASA every 30 s up to 90 s. The percentage of the agglutinated sperm at each time point was computed by normalizing the progressive sperm count obtained after treatment with HCA constructs to the progressive sperm count obtained after treatment with sperm washing media.

In some cases sperm were fluorescently labeled. Purified motile sperm were fluorescently labelled using Live/Dead Sperm Viability Kit (Invitrogen Molecular Probes), which stains live sperm with SYBR 14 dye, a membrane-permeant nucleic acid stain, and dead sperm with propidium iodide, a membrane impermeant nucleic acid stain. For labelling of 1 mL of washed semen, final concentration of 200 nM and 12 μM were respectively used for SYBR 14 and Propidium Iodide dye. Once labelled, the sperm solution was washed twice to remove unbound fluorophores by centrifuging at 300 g for 10 min using Sperm Washing Media (Irvine Scientific). Next, the labelled motile sperm pellet was resuspended in sperm washing medium, and an aliquot was taken for determination of sperm count and motility using CASA.

In some cases cervicovaginal mucus (CVM) was used. Briefly, undiluted CVM secretions, averaging 0.5 g per sample, were obtained from women of reproductive age, ranging from 20 to 32 years old (27.4±0.9 years, mean±SD), by using a self-sampling menstrual collection device (Instead Softcup). Participants inserted the device into the vagina for at least 30 s, removed it, and placed it into a 50 mL centrifuge tube. Samples were centrifuged at 230 g for 5 min to collect the secretions. Aliquots of CVM for lactic acid and Ab measurements (diluted 1:5 with 1×PBS and stored at −80° C.) and slides for gram staining were prepared immediately, and the remainder of the sample was stored at 4° C. until microscopy, typically within a few hours.

Multiple particle tracking of fluorescently labelled sperm in CVM was used to mimic the dilution and neutralization of CVM by alkaline seminal fluid. CVM was first diluted three-fold using sperm washing media and titrated to pH 6.8-7.1 using small volumes (˜3% v/v) of 3 N NaOH. The pH was confirmed using a micro pH electrode (Microelectrodes, Inc., Bedford, N.H.) calibrated to pH 4, 7 and 10 buffers. Next, 4 μL of HCA constructs or control (anti-RSV IgG1) was added to 60 μL of diluted and pH adjusted CVM and mixed well in a CultureWell™ chamber slides (Invitrogen #C37000, ThermoFisher Scientific) followed by addition of 4 μL of 7.5×10⁵ per mL of fluorescently labelled sperm. Once mixed, sperm, antibody and CVM were incubated for 15 min at room temperature. Then, translational motions of the sperm were recorded using an electron multiplying charge-coupled-device camera (Evolve 512; Photometrics, Tucson, Ariz.) mounted on an inverted epifluorescence microscope (AxioObserver D1; Zeiss, Thornwood, N.Y.) equipped with an Alpha Plan-Apo 20/0.4 objective, an environmental (temperature and C02) control chamber, and light-emitting diode (LED) light source (Lumencor Light Engine DAPI/GFP/543/623/690). 6 videos (512×512 pixels, 16-bit image depth) were captured for each antibody condition with MetaMorph imaging software (Molecular Devices, Sunnyvale, Calif.) at a temporal resolution of 66.7 ms and spatial resolution of 50 nm (nominal pixel resolution, 0.78 μm/pixel) for 20 s. Microscopy videos obtained for this trapping were run through a Neural Net Tracker to determine the percentage of progressively motile sperm after incubation with different concentrations of HCA constructs.

In some experiments, HCA constructs were instilled to the sheep's vagina followed by human semen and simulated intercourse (˜30 s) with a vaginal dilator. Two minutes later, the fluids from the sheep vagina were recovered and immediately assessed for progressive sperm motility. The condition with multimeric HCA constructs was repeated two more times in the same sheep with at least 7 days in between experiments. For each experiment, semen samples were pooled from 3-4 donors.

In one example described herein, native IgG sequences and synthetic anti-RSV binding agents based on the anti-RSV IgG were generated and characterized. The codon-optimized sequences of VH, CH1, VL and CL were utilized to design the sequences for anti-RSV IgG, Fab-IgG and IgG-Fab antibodies. The complete sequence of IgG, IgG-Fab and Fab-IgG were ordered using GeneArt Gene Synthesis. To minimize synthesis problems that could occur due to repeated sequences in IgG-Fab and Fab-IgG, the DNA sequences for repeated fragments were manually codon-optimized resulting in increased variability of DNA sequences and subsequent reduced complexity for gene synthesis. After manual codon-optimization, gene sequences were further processed through the complexity-analyzing tool provided by IDT to obtain a complexity score. Gene fragments with complexity scores<25 had been easily and successfully synthesized via GeneArt Gene synthesis.

Plasmids encoding native anti-RSV mAbs and anti-RSV synthetic binding agents were generated as described above. The variable heavy (VH) and variable light (VL) DNA sequences for anti-RSV antibodies were obtained from the publication of Motavizumab. For the construction of expression plasmid encoding the light chain, the gene fragment consisting of VL and CL (Ck) DNA sequences was synthesized using GeneArt® gene synthesis service and cloned into the empty mammalian expression vector (pAH) using KpnI (5′) and EcoRI (3′) restriction sites. For the construction of expression plasmids encoding heavy chains (HC) for IgG, Fab-IgG and IgG-Fab, the complete gene sequences of all Abs were synthesized using GeneArt® gene synthesis service (ThermoFisher Scientific) and cloned into empty mammalian expression vector (pAH) sequences using KpnI (5′) and MluI (3′) restriction sites. For the ligation of all heavy chains as well as a light chain into the expression vectors, quick ligation kit (New England Biolabs, Ipswich, Mass.) was used. All ligated DNA constructs were transformed into chemically competent TOP10 E. coli cells (Life Technologies) and plated on ampicillin plates for selection. Bacterial colonies were picked, cultured, and the plasmids were prepped (Qiagen MiniPrep Kit). Correct assembly of the constructs into the expression vector were confirmed by Sanger sequencing (Eurofins Genomics).

The expression plasmids encoding the heavy chain (HC) and light chain (LC) for IgG, Fab-IgG and IgG-antibodies were scaled up by transforming the sequencing-confirmed expression plasmids in chemically competent TOP10 E. coli, inoculating the transformation mix into 100 mL Luria broth in a 250 mL baffled flask and overnight shaking at 220 r.p.m at 37° C. Midi-prep plasmid purifications were done using NucleoBond® Xtra Midi EF Kits (Macherey-Nagel) according to the manufacturer's protocols. Proteins were expressed in Expi293 cells using ExpiFectamine® 293 Transfection reagents and protocols provided by the manufacturer (ThermoFisher Scientific). For IgG, one HC and one LC plasmid were co-transfected using a 1:1 ratio at 1 μg total DNA per 1 mL of culture. For both Fab-IgG and IgG-Fab, one HC and one LC plasmid were co-transfected using a 1:2 ratio at 1 μg total DNA per 1 mL culture. Transfected cells were grown at 37° C. in a 5% CO₂ incubator while shaking at 125 r.p.m. for 5 days. Supernatants were harvested by centrifugation at 5000 g for 10 min and passed through 0.22 μm filters for purification using standard protein A affinity chromatography. Briefly, 30 mL of transfected supernatant was incubated with 400 μL PBS-washed Pierce™ Protein A Plus Agarose Resin (ThermoFisher Scientific) overnight at 4° C. Next, the resin-supernatant solution was flown through the gravity columns followed by the washing of resin. Protein was eluted by adding 900 μL of Pierce™ IgG Elution Buffer (ThermoFisher Scientific) into PBS-washed resin and was immediately neutralized by adding 100 μL of UltraPure™ 1 M Tris-HCl Buffer, pH 7.5 (ThermoFisher Scientific). Eluted proteins were further dialyzed into PBS using Amicon® Ultra Centrifugal Filters (Millipore Sigma).

SDS-PAGE experiments were performed using 4-12% NuPage Bis-Tris gels (ThermoFisher Scientific) in 1× NuPage MOPS buffer under both reducing and non-reducing conditions to confirm the correct assembly of all anti-RSV antibody constructs. For each protein sample, 1 μg of protein was diluted in 3.75 μL LDS sample buffer followed by the addition of 11.25 μL nuclease-free water. Proteins were then denatured at 70° C. for 10 min in a thermocycler. Next, 0.3 μL of 0.5 M TCEP was added as a reducing agent to the denatured protein for reduced samples and incubated at room temperature for 5 min. Bio-Rad Precision Protein Plus Unstained Standard was used as protein ladder. After loading the samples, the gel was run for 50 min at a constant voltage of 200 V and washed 3 times with Milli-Q water. Then, the protein bands were visualized by staining with Imperial Protein Stain (ThermoFisher Scientific) for 1 hr followed by overnight de-staining with Milli-Q water.

Anti-RSV synthetic binding agents were examined using ELISA. Briefly, half-area polystyrene plates (CLS3690, Corning) were coated with 50 μL of 10 μg/mL of human RSV (ATCC® VR-1540P) per well using NaHCO₃ buffer (pH 9.6). After overnight incubation at 4° C., the plates were incubated under the UV for 1 hr to inactivate the virus. The plates were washed twice with 1×PBS. 100 μL of 5% milk was incubated at room temperature for 1 hr to prevent non-specific binding of an antibody to the microwells. The serial dilution of monoclonal antibodies in 1% milk were added to the microwells and incubated overnight at 4° C. Palivizumab (Synagis), an FDA-approved mAb against RSV, was used as a positive control for this assay. After primary incubation, the plates were washed three times using 1×PBS. Then, the secondary antibody, goat anti-human IgG F(ab′)2 antibody HRP-conjugated (1:50,000 dilutions in 1% milk, 209-1304, Rockland Inc.) was added to the wells and incubated for 1 hr at room temperature. The washing procedure was repeated and 50 μL of the buffer containing substrate (1-Step Ultra TMB ELISA Substrate, ThermoFisher Scientific) was added to develop the colorimetric reaction for 15 min. The reaction was quenched using 50 μL of 2N H₂SO₄, and the absorbance at 450 nm (signal) and 570 nm (background) was measured using SpectraMax M2 Microplate Reader (Molecular Devices). Each experiment was done with samples in triplicates and repeated at least twice as a measure assay variability.

In general, the anti-RSV synthetic binding agents were synthetized as described herein to form Fab-IgG, IgG-Fab, Fab-IgG-Fab, Fab-Fab-IgG, Fab-Fab-IgG-Fab, IgG-Fab-Fab, Fab-IgG-Fab-Fab, and Fab-Fab-IgG-Fab-Fab. Both the synthesis and characterization of these anti-RSV synthetic binding agents were similar to those described for the other synthetic binding agents described, resulting in improved muco-trapping. In particular, as compared to IgG, the synthetic binding agents described herein had a superior muco-trapping and agglutination effect on the target. In particular, motile targets, such as sperm, bacteria, and other pathogens, may show an increase in mucosal tracking as compared to native antibodies. Preliminary evidence also suggests that the synthetic binding agents described herein may specifically inhibit growth of bacteria. Surface antigens on targets (e.g., virus) were used in all of the examples described herein.

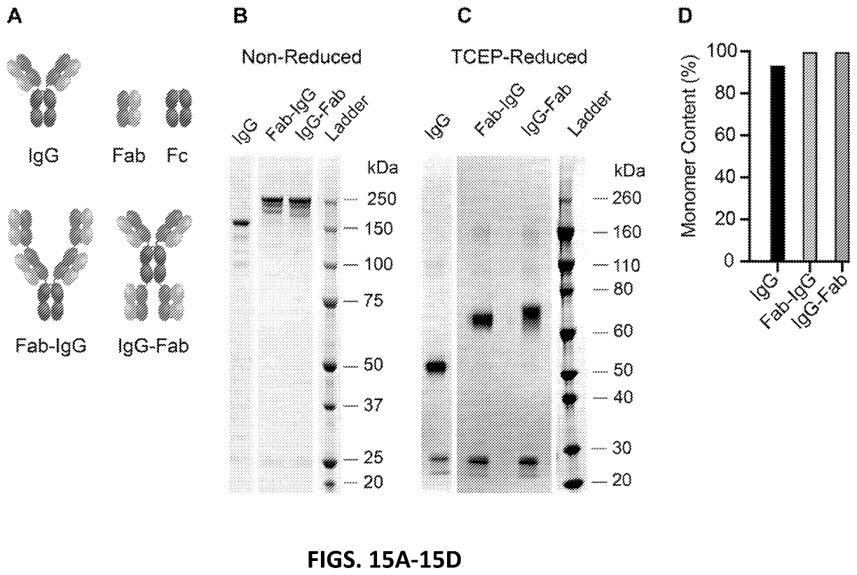

For example, FIG. 15A illustrates native (IgG, top, including component Fab and Fc regions), and FI (Fab-IgG) and IF (IgG-Fab) synthetic binding agents (bottom). For example, the diagrams shown in FIG. 15A may be of anti-sperm IgG, Fab-IgG, and IgG-Fab; the N-terminal Fab of Fab-IgG and C-terminal Fab of IgG-Fab may contain a fully intact anti-sperm Fab with VH, VL, CH1, and CL, as described above. In FIG. 15B a non-reducing gel shows that the synthesized Fab-IgG and IgG-Fab were produced as expected, and were expressed with high efficiency. FIG. 15C shows a reducing SDS-Page analysis of the indicated antibodies after expression in Expi293 cells and purification by protein A/G chromatography. FIG. 15D is a demonstration of the purity and homogeneity of the purified multimeric antibodies via analytical SEC-MALS analysis.

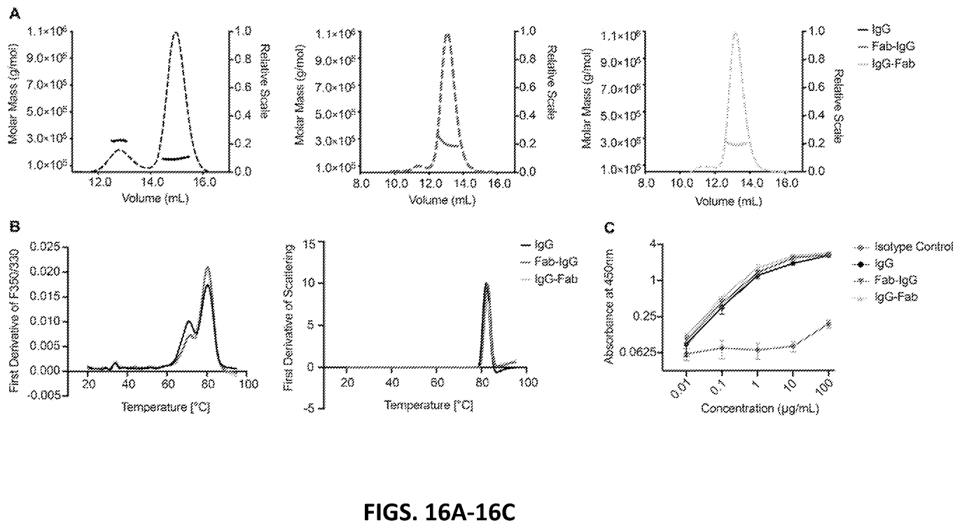

FIGS. 16A-16C further characterize the synthetic binding agents described herein. In this example, anti-sperm synthetic binding agents were compared to anti-sperm IgG antibodies. As shown in FIG. 16A, the molar mass versus time of the IgG, Fab-IgG and IgG-Fab respectively as determined by SEC-MALS showed that the synthetic binding agents described herein resembled the native mAb from which they originated. This was also apparent in FIG. 16B. The value of melting temperature (Tm) and aggregating temperature (Tagg) of as determined by nanoDSF by measuring intrinsic fluorescence of a protein and changes in back-reflection respectively were nearly the same. In FIG. 16C, whole Sperm ELISA analysis was used to assess the binding potency of indicated antibodies. Motavizumab (e.g., anti-RSV IgG1) was used as the isotype control. ELISA was performed in triplicates and repeated three times using 3 unique specimens. Lines indicate arithmetic mean concentration and standard error of mean.

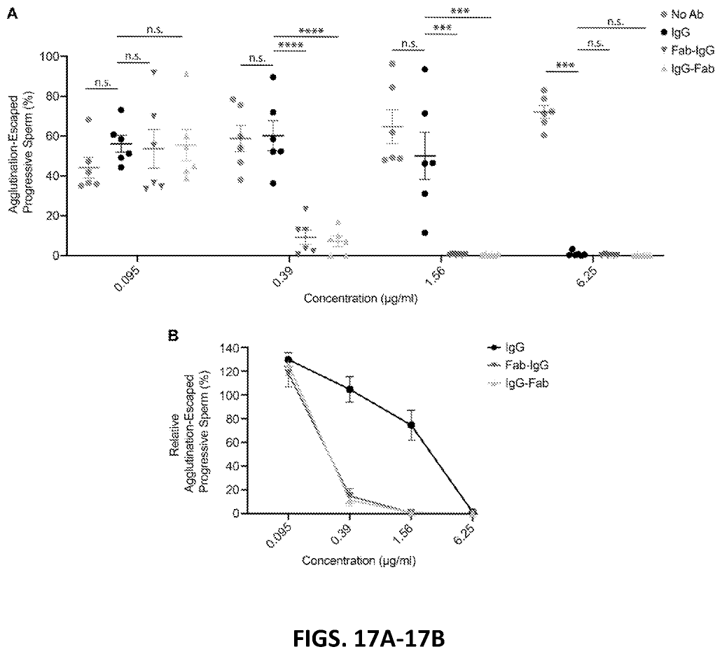

FIGS. 17A-17B show the effect of anti-sperm synthetic binding agents as described herein (e.g., with additional FAB regions linked by flexible linkers to either or both the native variable (Fab) or constant ends). In FIG. 17A, the sperm agglutination potency of parent IgG, Fab-IgG and IgG-Fab was examined using purified motile sperm (10×10⁶ progressively motile sperm per mL). The sperm agglutination potency of the parent and multimeric anti-sperm IgGs (e.g., the synthetic binding agents) were measured by quantifying the percentage of sperm that remains progressively motile post Ab-treatment at different concentrations compared to pre-treatment condition. In FIG. 17B, the percentage of agglutination-escaped progressive sperm post-treatment was normalized to the negative control for further comparison. Data represents 6 unique sperm specimens. Lines indicate arithmetic mean concentration and standard error of mean. There was little appreciable difference between the Fab-IgG and the IgG-Fab synthetic binding agents, however a substantial improvement was seen over the native IgG in both the relative extent of agglutination (i.e. fraction agglutinated), and the minimum concentrations of Fab-IgG or IgG-Fab needed to agglutinate a comparable fraction of sperm.

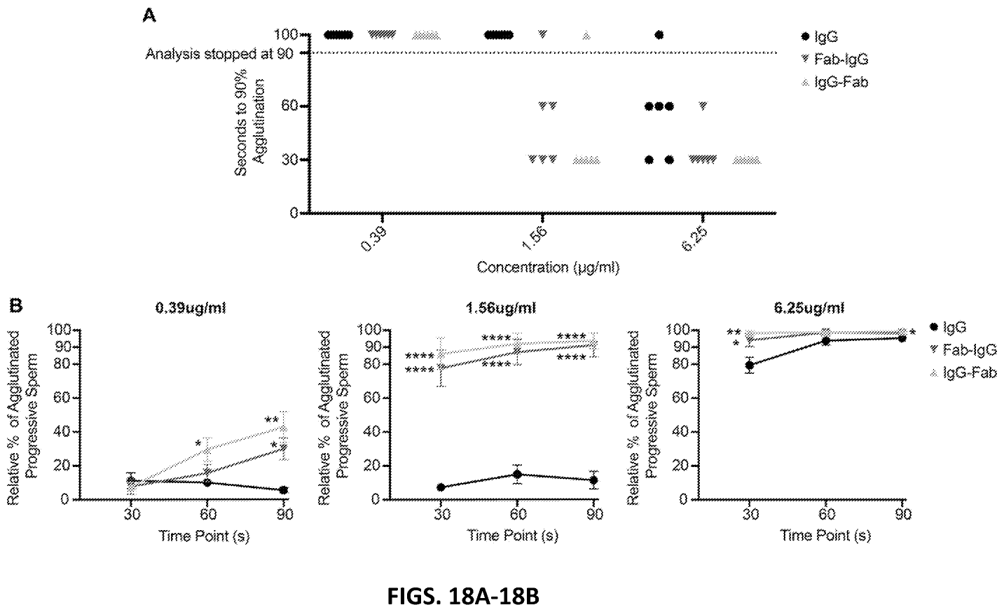

FIGS. 18A-18B show the effect of sperm agglutination kinetics for parent IgG, Fab-IgG and IgG-Fab using purified motile sperm (10×10⁶ progressively motile sperm per mL). In FIG. 18A, the agglutination kinetics of indicated antibodies was assayed by quantifying the time required to agglutinate 90% of progressively motile sperm compared to untreated control. The CASA analysis was obtained every 30 s post-treatment until 90 s. In FIG. 18B the rate of sperm agglutination of indicated anti-sperm antibodies was measured by quantifying the percentage of agglutinated sperm post Ab-treatment at three different time points compared to pre-treatment. Data represents 6 unique sperm specimens. Lines indicate arithmetic mean concentration and standard error of mean. Again, the synthetic binding agents has far superior sperm-agglutination potential compared to IgG alone, particularly at lower concentrations (e.g., less than about 2 ug/ml).

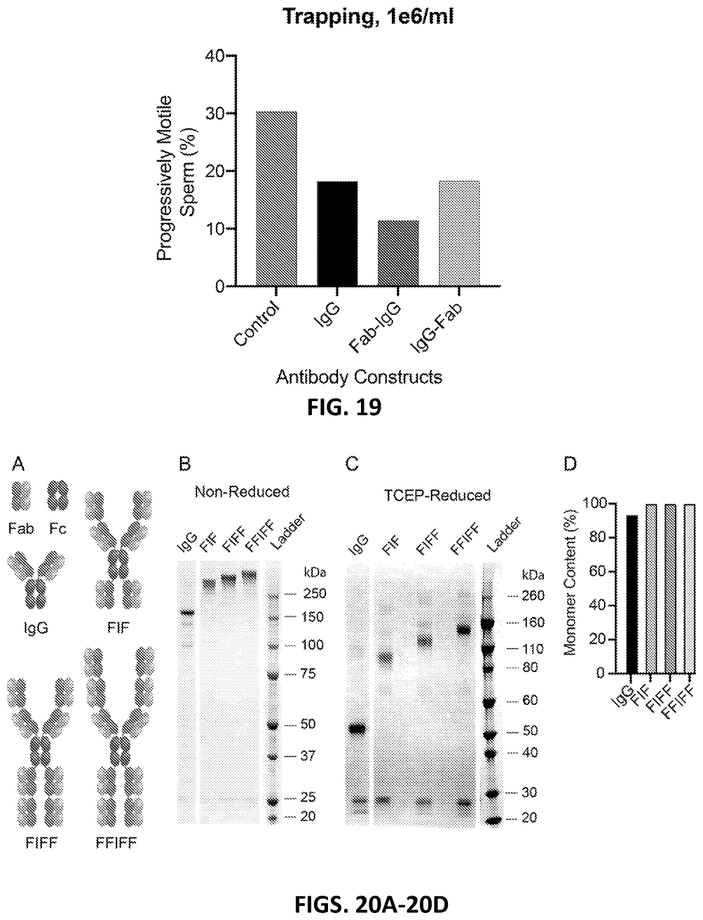

FIG. 19 shows the muco-trapping potency of parent IgG, Fab-IgG and IgG-Fab using pH-neutralized female CVM and purified motile sperm (1.5×10⁶ progressively motile sperm/mL). The muco-trapping potency of indicated antibodies was assessed by performing real-time video microscopy on fluorescently labelled sperm suspended in Ab-treated (25 ug/ml) CVM. We employed neural network tracker customized with standardized sperm motility parameters in all recorded videos to quantify the percentage of progressively motile sperm.

In general there is further improvement with additional Fab groups, up to 10 Fab groups. The 10-mer (e.g., Fab-Fab-IgG-Fab-Fab, having a total of 10 Fabs) performed slightly better than 6 mer (Fab-IgG-Fab, Fab-Fab-IgG, IgG-Fab-Fab) or 8-mer (Fab-Fab-IgG-Fab, Fab-IgG-Fab-Fab) and 4 mer (Fab-IgG, IgG-Fab). FIGS. 20A-20D show the characterization of multimeric anti-sperm IgG antibodies. FIG. 20A is a schematic diagrams of anti-sperm IgG, Fab-IgG-Fab (FIF), Fab-IgG-Fab-Fab (FIFF) and Fab-Fab-IgG-Fab-Fab (FFIFF). N-terminal and C-terminal Fabs of FIF, FIFF and FFIFF contains fully intact anti-sperm Fabs with VH, VL, CH1, and CL. FIGS. 20B and 20C show non-reducing and reducing SDS-Page analysis, respectively, of the indicated antibodies after expression in Expi293 cells and purification by protein A/G chromatography. FIG. 20D shows a demonstration of the purity and homogeneity of the purified multimeric antibodies via analytical SEC-MALS analysis.

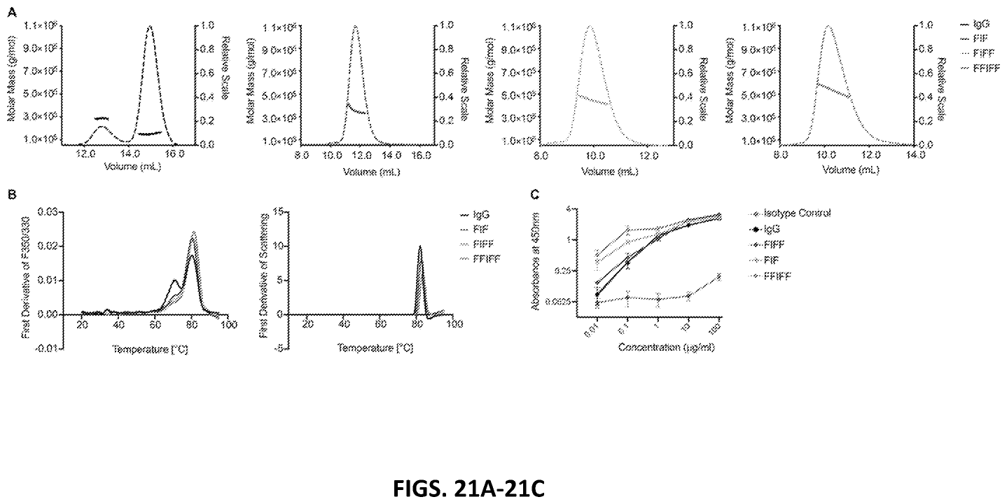

FIGS. 21A-21C also illustrate that the synthetic binding agents (e.g., multimeric anti-sperm IgG antibodies) described herein have comparable properties as compared to each other and to the native IgG. In FIG. 21A, molar mass versus time of the IgG, FIF, FIFF and FFIFF respectively is determined by SEC-MALS. FIG. 21B shows the value of melting temperature (Tm) and aggregating temperature (Tagg) of indicated antibodies as determined by nanoDSF by measuring intrinsic fluorescence of a protein and changes in back-reflection respectively. In FIG. 21C, the whole Sperm ELISA analysis is used to assess the binding potency of the indicated antibodies. Motavizumab (anti-RSV IgG1) is used as the isotype control. ELISA was performed in in triplicates and repeated three times using 3 unique specimens. Lines indicate arithmetic mean concentration and standard error of mean.

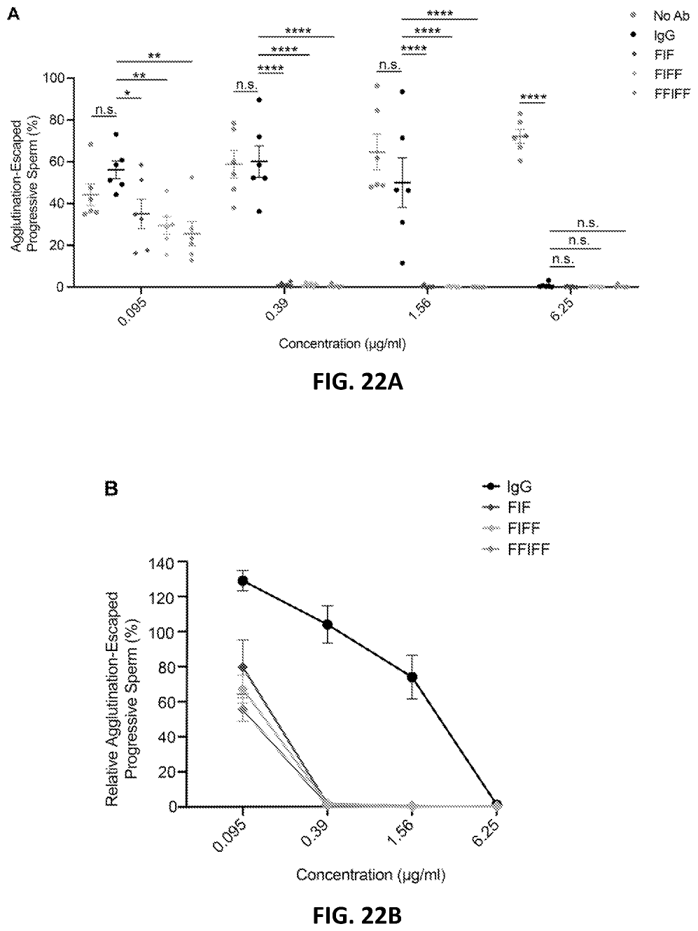

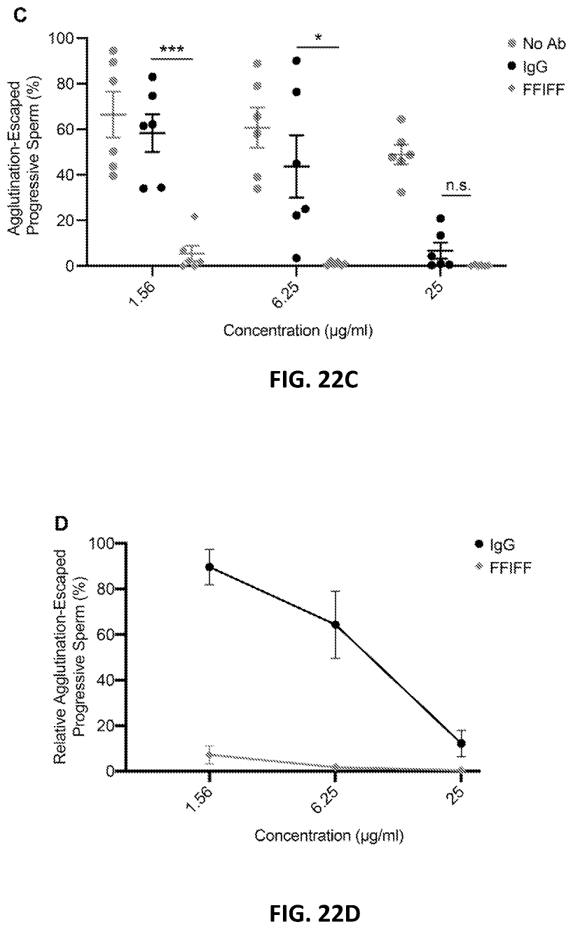

As mentioned, the synthetic binding agents, including the high-Fab number (e.g., 8 mer, 10 mer) were highly effective at enhancing sperm agglutination. FIG. 22A-22B show sperm agglutination potency of parent IgG and multimeric constructs using purified motile sperm (10×10⁶ progressively motile sperm per mL) and whole semen. In FIG. 22A, sperm agglutination potency of the parent IgG, FIF, FIFF and FFIFF was measured by quantifying the percentage of sperm that remains progressively motile post Ab-treatment compared to pre-treatment condition using purified motile sperm (10×10⁶ progressively motile sperm per mL). In FIG. 22B, the percentage of agglutination-escaped progressive sperm post Ab-treatment using purified motile sperm was normalized to the negative control for further comparison. As mentioned, the 10 mer (Fab-Fab-IgG-Fab-Fab) was slightly better than the lower Fab constructs, particularly at lower concentrations, and faster times to agglutination. In FIG. 22C, the sperm agglutination potency of the parent IgG and FFIFF was measured by quantifying the percentage of sperm that remains progressively motile post Ab-treatment compared to pre-treatment condition using whole semen. In FIG. 22D, the percentage of agglutination-escaped progressive sperm post Ab-treatment using whole semen was normalized to the negative control for further comparison. Data represents 6 unique sperm specimens. Lines indicate arithmetic mean concentration and standard error of mean.

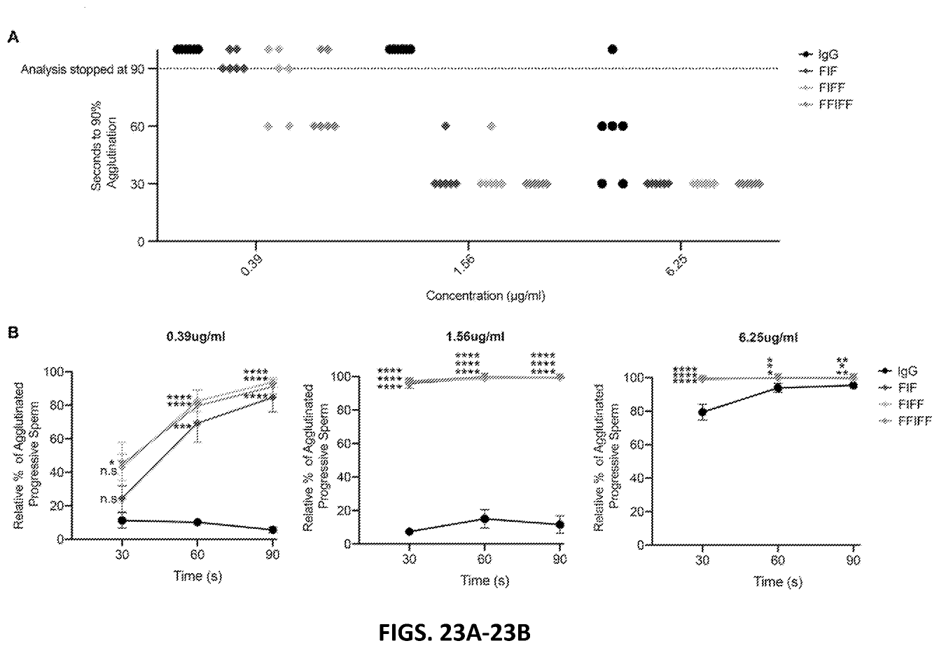

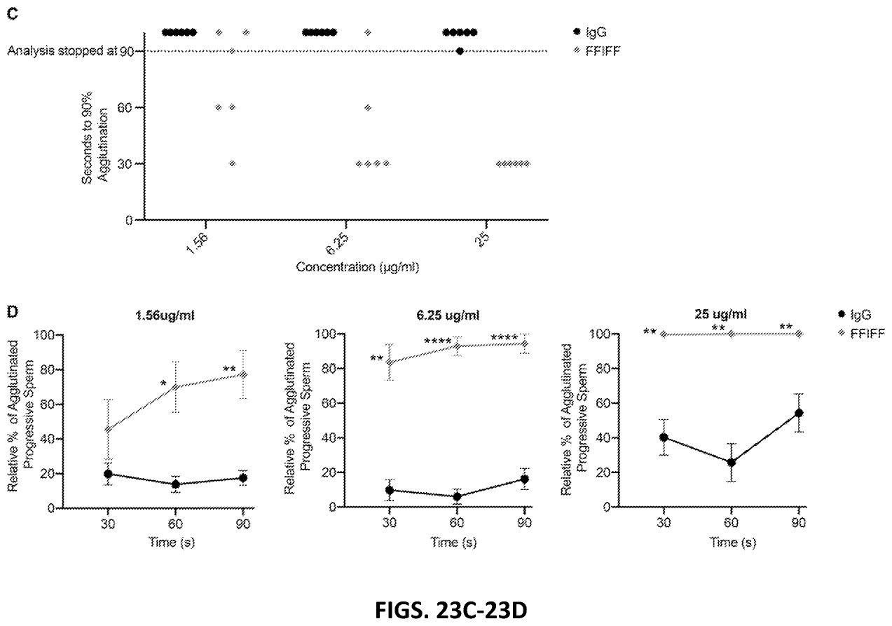

Sperm agglutination kinetics of parent IgG and multimeric constructs using purified motile sperm (10×10⁶ progressively motile sperm per mL) and whole semen is also illustrated in FIGS. 23A-23B. In FIG. 23A, the agglutination kinetics of parent IgG, FIF, FIFF and FFIFF was examined by quantifying the time required to achieve 90% agglutination of progressive sperm compared to untreated control using purified motile sperm (10×10⁶ progressively motile sperm per mL). The CASA analysis was obtained every 30 s post-treatment until 90 s. In FIG. 23B the rate of sperm agglutination of parent IgG, FIF, FIFF and FFIFF was measured by quantifying the percentage of agglutinated sperm post Ab-treatment at three different time points compared to pre-treatment using purified motile sperm (10×10⁶ progressively motile sperm per mL). The agglutination kinetics of parent IgG and FFIFF was assessed (in FIG. 23C) by quantifying the time required to achieve 90% agglutination of progressive sperm compared to untreated control using whole semen. The CASA analysis was obtained every 30 s post-treatment until 90 s. In FIG. 23D, the rate of sperm agglutination of parent IgG and FFIFF was measured by quantifying the percentage of agglutinated sperm post Ab-treatment at three different time points compared to pre-treatment using whole semen. Data represents 6 unique sperm specimens. Lines indicate arithmetic mean concentration and standard error of mean.

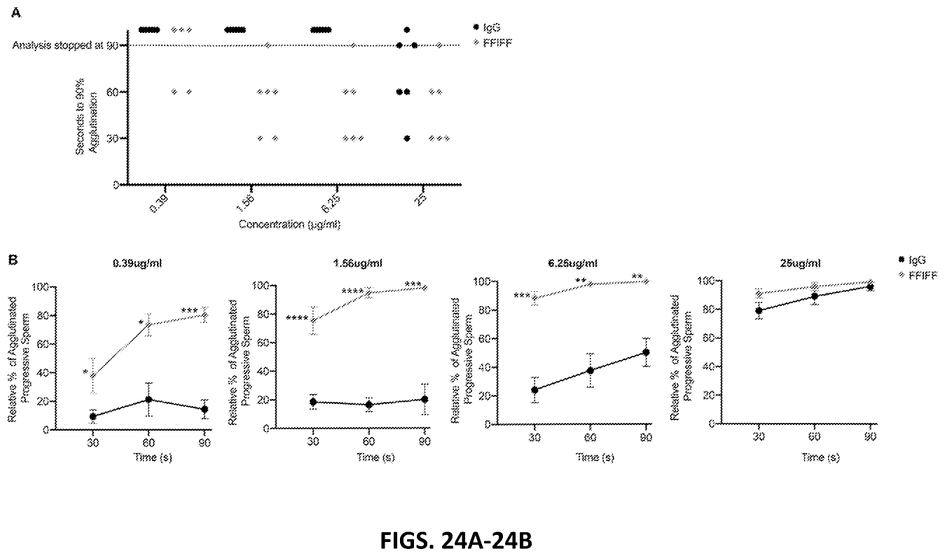

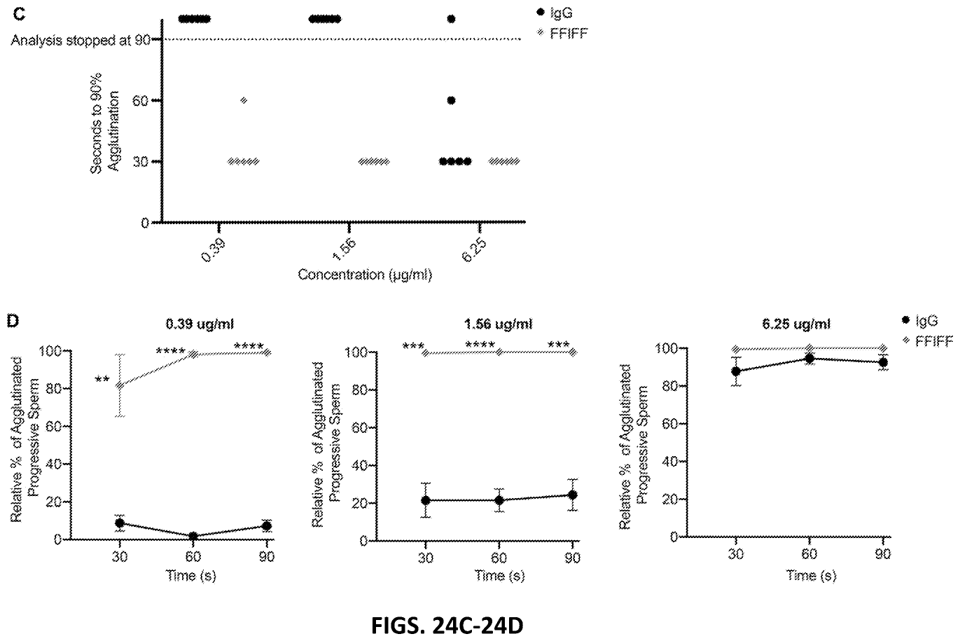

Sperm agglutination kinetics of parent IgG and FFIFF was examined using low and high concentration of purified motile sperm (2×10⁶ and 50×10⁶ progressive sperm/mL), as shown in FIGS. 24A-24B. In FIG. 24A, the agglutination kinetics of IgG and FFIFF was examined by quantifying the time required to achieve 90% agglutination of progressive sperm compared to untreated control. The CASA analysis was obtained every 30 s post-treatment until 90 s using purified motile sperm (2×10⁶ progressive sperm/mL). The rate of sperm agglutination of IgG and FFIFF was measured by quantifying the percentage of agglutinated sperm post Ab-treatment at three different time points compared to pre-treatment using purified motile sperm (2×10⁶ progressive sperm/mL), as shown in FIG. 24B. The agglutination kinetics of IgG and FFIFF were examined in FIG. 24C by quantifying the time required to achieve 90% agglutination of progressive sperm compared to untreated control using purified motile sperm (50×10⁶ progressive sperm/mL). The CASA analysis was obtained every 30 s post-treatment until 90 s. In FIG. 24D, the rate of sperm agglutination of IgG and FFIFF was measured by quantifying the percentage of agglutinated sperm post Ab-treatment at three different time points compared to pre-treatment using purified motile sperm (50×10⁶ progressive sperm/mL). Data represents 6 unique sperm specimens. Lines indicate arithmetic mean concentration and standard error of mean.

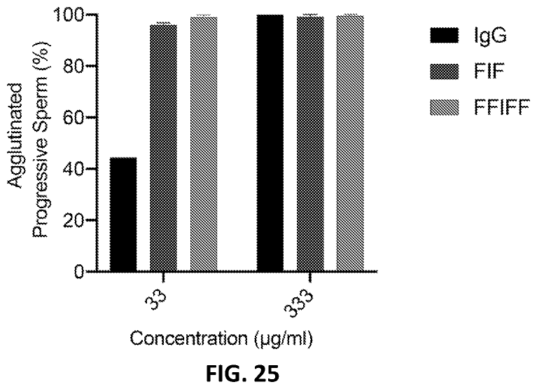

FIG. 25 shows the sperm agglutination potency of parent IgG, FIF and FFIFF using whole semen in a sheep study. The agglutination potency of IgG, FIF and FFIFF was measured in vivo by instilling Abs into sheep vagina, followed by human semen and simulated intercourse. Sperm motility was examined immediately in the fluids from the sheep vagina. Data represents 3 unique sheep studies for FIF and FFIFF, and 1 sheep study for IgG at both 33 ug/ml and 333 ug/ml. Lines indicate arithmetic mean concentration and standard error of mean.

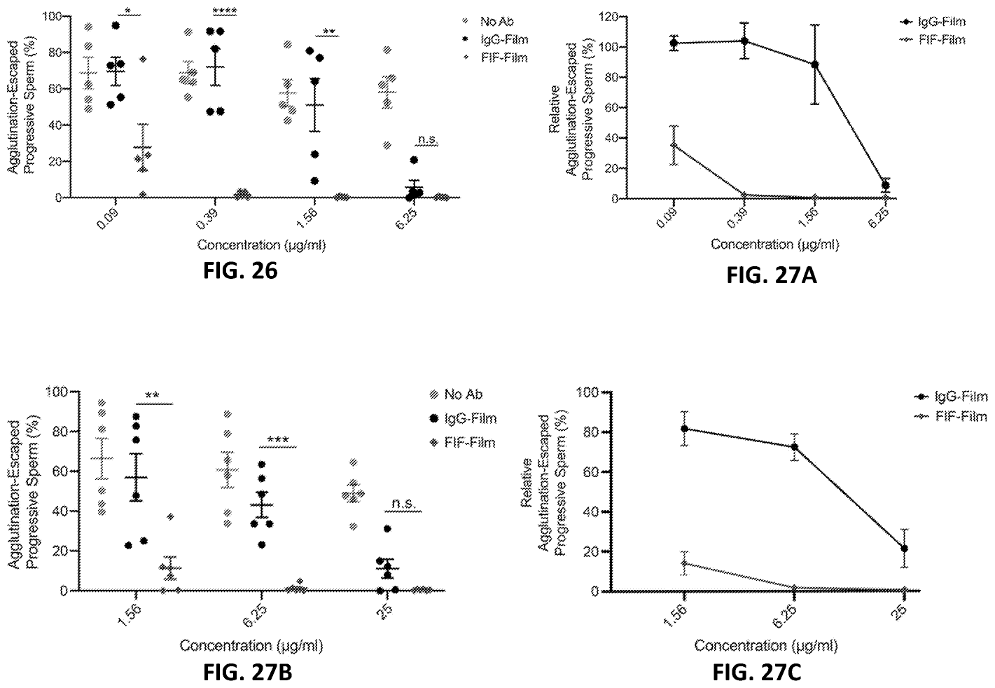

As mentioned above, films may also be used to deliver the synthetic binding agents described herein. For example, FIGS. 26 and 27A-27C illustrate agglutination potency of a Nicotiana-produced film of parent IgG and FIF using purified motile sperm (10×10⁶ progressive sperm/mL) and whole semen. FIG. 26 shows measured sperm agglutination potency of the parent IgG-Film and FIF-Film by quantifying the percentage of sperm that remains progressively motile post Ab-treatment compared to pre-treatment condition using purified motile sperm (10×10⁶ progressively motile sperm per mL). FIG. 27A shows the percentage of agglutination-escaped progressive sperm post Ab-treatment using purified motile sperm was normalized to the negative control for further comparison. The sperm agglutination potency of the parent IgG-Film and FIF-Film was measured (in FIG. 27B) by quantifying the percentage of sperm that remains progressively motile post Ab-treatment compared to pre-treatment condition using whole semen. In FIG. 27C, the percentage of agglutination-escaped progressive sperm post Ab-treatment using whole semen was normalized to the negative control for further comparison. Data represents 6 unique sperm specimens. Lines indicate arithmetic mean concentration and standard error of mean.

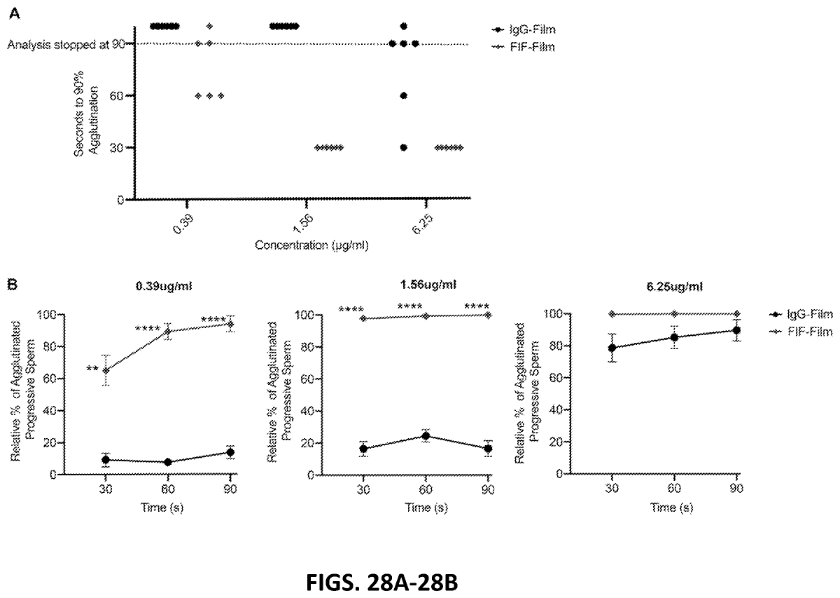

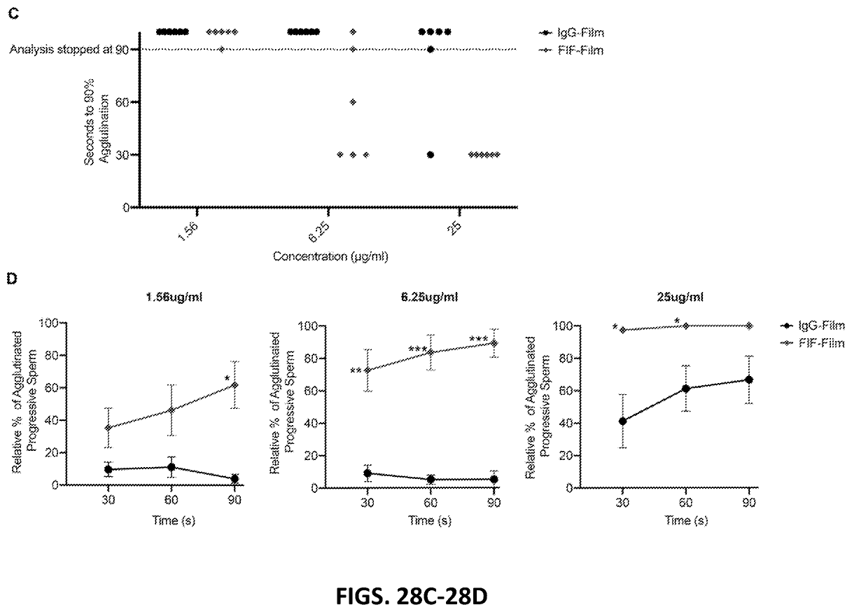

FIGS. 28A-28B illustrate agglutination kinetics of a Nicotiana-produced films of parent IgG and FIF using purified motile sperm (10×10⁶ progressively motile sperm/mL) and whole semen. FIG. 28A shows the agglutination kinetics of indicated antibodies by quantifying the time required to achieve 90% agglutination of progressive sperm compared to untreated control using purified motile sperm (10×10⁶ progressively motile sperm/mL). The CASA analysis was obtained every 30 s post-treatment until 90 s. The rate of sperm agglutination of indicated anti-sperm antibodies was assessed (in FIG. 28B) by quantifying the percentage of agglutinated sperm post Ab-treatment at three different time points compared to pre-treatment using purified motile sperm (10×10⁶ progressively motile sperm/mL).

Agglutination kinetics of the Nicotiana-produced films of parent IgG and FIF was assessed using purified motile sperm (10×10⁶ progressively motile sperm/mL) and whole semen, as shown in FIGS. 28C and 28D. The agglutination kinetics of indicated antibodies was assessed by quantifying the time required to achieve 90% agglutination of progressive sperm compared to untreated control using whole semen. The CASA analysis was obtained every 30 s post-treatment until 90 s. The rate of sperm agglutination of indicated anti-sperm antibodies was measured by quantifying the percentage of agglutinated sperm post Ab-treatment at three different time points compared to pre-treatment using whole semen. Data represents 6 unique sperm specimens. Lines indicate arithmetic mean concentration and standard error of mean.

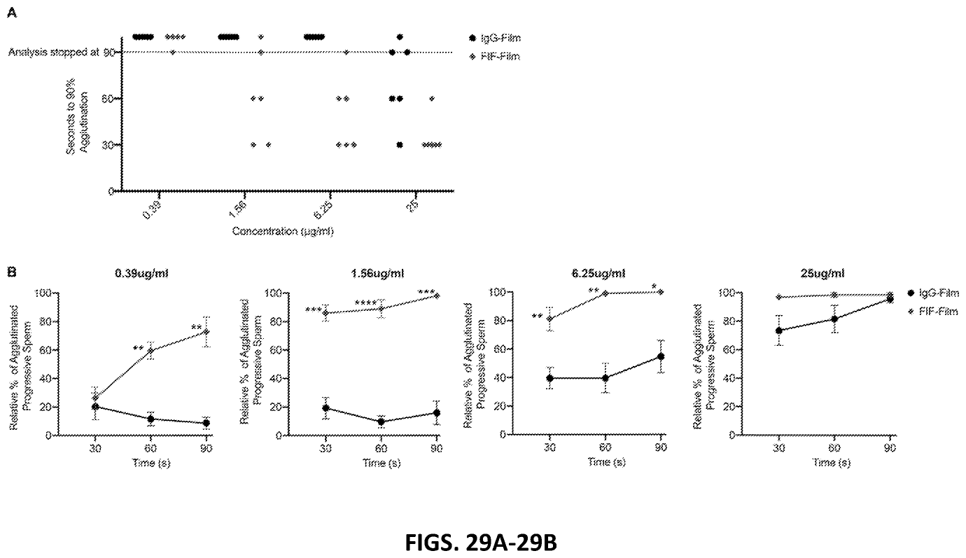

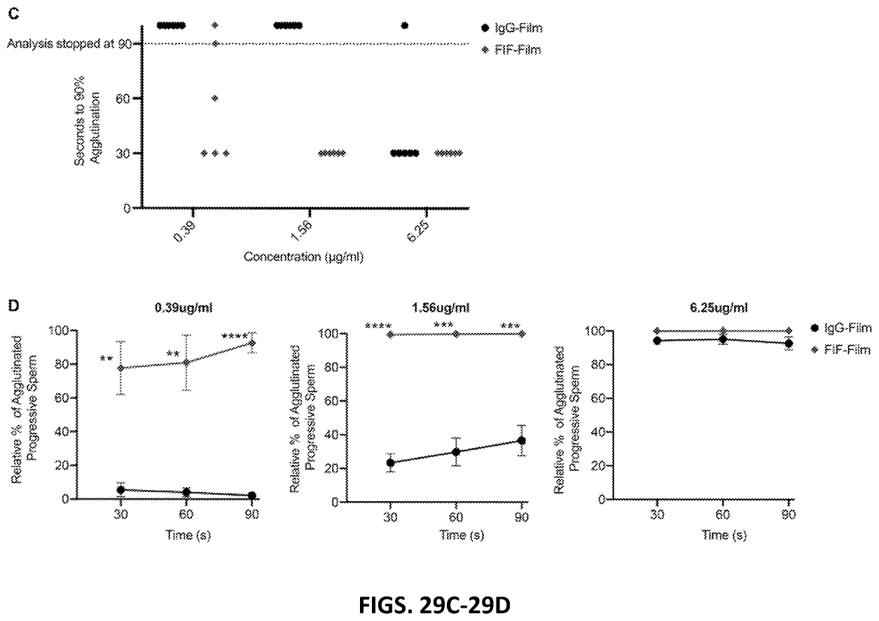

FIGS. 29A-28B illustrate agglutination kinetics of the Nicotiana-produced films of parent IgG and FIF using low and high concentration of purified motile sperm (2×10⁶ and 50×10⁶ progressively motile sperm/mL). The agglutination kinetics of indicated antibodies was assessed by quantifying the time required to achieve 90% agglutination of progressive sperm compared to untreated control using purified motile sperm (2×10⁶ progressively motile sperm/mL). The CASA analysis was obtained every 30 s post-treatment until 90 s. We also measured the rate of sperm agglutination of indicated anti-sperm antibodies by quantifying the percentage of agglutinated sperm post Ab-treatment at three different time points compared to pre-treatment using purified motile sperm (2×10⁶ progressively motile sperm/mL) (FIG. 29B). The agglutination kinetics of indicated antibodies was assessed by quantifying the time required to achieve 90% agglutination of progressive sperm compared to untreated control using purified motile sperm (50×10⁶ progressively motile sperm/mL) as shown in FIG. 29C. The CASA analysis was obtained every 30 s post-treatment until 90 s. We also measured the rate of sperm agglutination of indicated anti-sperm antibodies by quantifying the percentage of agglutinated sperm post Ab-treatment at three different time points compared to pre-treatment using purified motile sperm (50×106 progressively motile sperm/mL) (FIG. 29D). Data represents 6 unique sperm specimens. Lines indicate arithmetic mean concentration and standard error of mean.

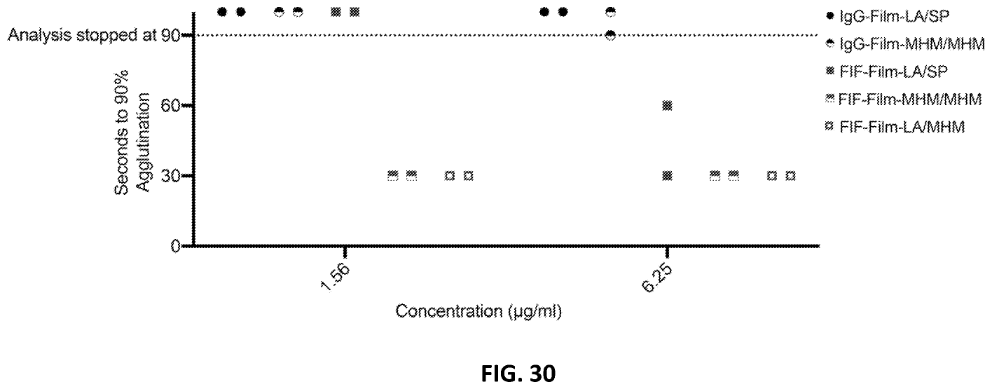

FIG. 30 shows agglutination kinetics of the Nicotiana-produced films of parent IgG and FIF in the acidic environment using purified motile sperm (20×10⁶ progressively motile sperm/mL) and 24 hr treatment with lactic acid. The agglutination kinetics of lactic acid-treated antibodies was assessed by quantifying the time required to achieve 90% agglutination of progressive sperm compared to untreated control. The CASA analysis was obtained every 30 s post-treatment until 90 s. Data represents 2 unique sperm specimens. Note: Lactic acid (LA) treated antibodies were neutralized with seminal plasma (SP) and then followed by dilution with SP or saline media i.e. MHM.

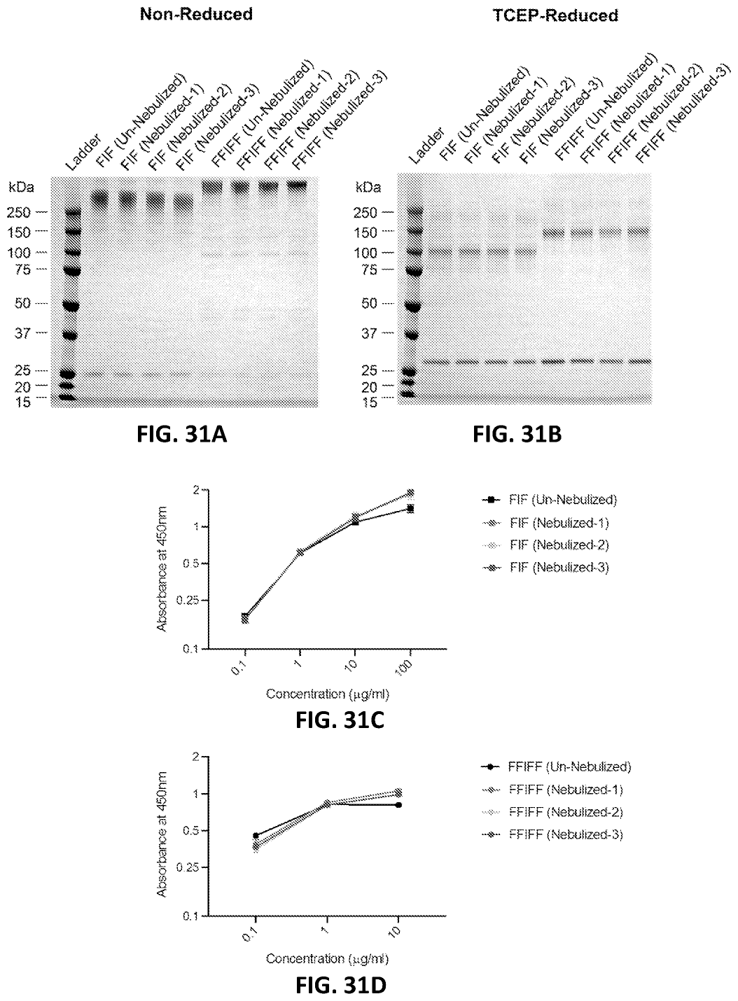

FIGS. 31A-31D show the characterization of Nicotiana-produced FIF-Film and Expi293-produced FFIFF antibodies post-nebulization. Non-reducing (FIG. 31A) and reducing (FIG. 31B) SDS-Page analysis of the indicated antibodies before and after nebulization using mesh-nebulizer. Whole Sperm ELISA analysis to assess the binding potency of (FIG. 31C) FIF-Film and (FIG. 31D) FFIFF post-nebulization. ELISA was performed in triplicates and repeated twice using the same donor specimens. Lines indicate arithmetic mean concentration and standard error of mean. Thus, nebulizing the synthetic binding agents described herein did not damage or disrupt them, and they may be effectively delivered by nebulization (e.g., by aerosolization).

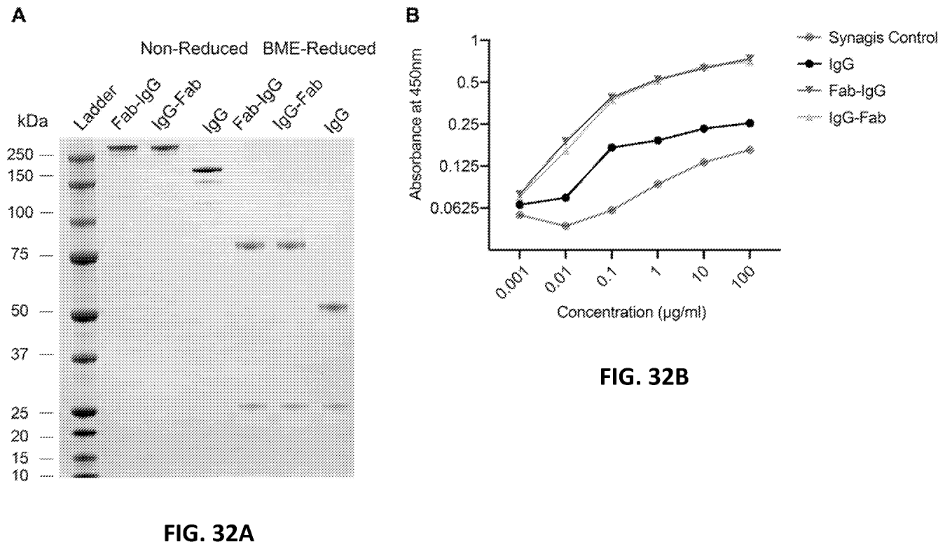

FIGS. 32A-32B illustrate the production and characterization of multimeric anti-RSV IgG antibodies. FIG. 32A shows non-reducing and reducing SDS-Page analysis, respectively, of ant-RSV synthetic binding agents (e.g., formed from a Motavizumab parent IgG) after expression in Expi293 cells and purification by protein A/G chromatography. FIG. 32B shows RSV ELISA analysis to assess the binding potency of the indicated antibodies. Synagis/Palivizumab (anti-RSV IgG1) was used as the positive control. ELISA was performed in triplicates.

As compared to control, the anti-RSV synthetic binding agents (in this case Fab-IgG and IgG-Fab, however Fab-Fab-IgG-Fab-Fab and Fab-IgG-Fab may have similar results as described above), were superior to native IgG in binding the same target, which will result in substantially greater muco-trapping potency and therefore therapeutic efficacy, particularly at lower concentrations and faster times.

As mentioned above, in some variations a synthetic binding agent may be made with multiple scFvs or nanobodies in the same manner as described with Fabs. Further, in some variations multimeric antibodies similar to those described herein may be made without Fc regions.

SEQUENCE LISTING

SEQ ID NO: 1
Amino Acid sequence of CD52g
GQNDTSQTSSPS
SEQ ID NO: 2
Annotated DNA sequence of HCA-UNC (anti-CD52g) (Heavy Chain)
CAGGTTCAGCTGCAGCAATGGGGAGCCGGACTGCTGAAGCCTAGCGAGACACTGTCTCTGAC
CTGTGCCGTGTACGGCGGCAGCTTCAGCGGCTACTACTGGTCCTGGATCAGACAGCCTCCTG
GCAAAGGCCTGGAATGGATCGGCGAGATCAATCACAGCGGCAGCACCAACTACAACCCCAGC
CTGAGAAGCAGAGTGACCATCAGCGTGGACACCAGCAAGAACCAGTTCAGCCTGAAGCTGAG
AAGCGTGACAGCCGCCGATACCGCCGTGTACTATTGCGCCAGAGGCTTTATGGTCCGAGGCA
TCATGTGGAACTACTACTACATGGACGTGTGGGGCAAGGGCACCACCGTGACAGTTTCTCCA
GCTAGCACCAAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAAAGTTGAGCCCAAATCTTGTG
ACAAAACTCACACATGCCCACCGTGCCCAGCACCTGAACTCCTGGGGGGACCGTCAGTCTTC
CTCTTCCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACGGCGTGG
AGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAG
AACCACAGGTGTACACCCTGCCCCCATCCCGGGACGAGCTGACCAAGAACCAGGTCAGCCTG
ACCTGCCTGGTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCTTCTTCCTCT
ACAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTAAA
SEQ ID NO: 3
Annotated Amino Acid sequence of HCA-UNC (anti-CD52g)(Heavy
Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGFMVRGIMWNYYYMDVWGKGTTVTVSP
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVF
LFPPKPMDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 4
Annotated Amino Acid sequence of Fab fragment of HCA-UNC
(anti-CD52g) (Heavy Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGEMVRGIMWNYYYMDVWGKGTTVTVSP
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHT
SEQ ID NO: 5
Annotated Amino Acid sequence of Fc fragment of HCA-UNC (anti-
CD52g) (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 6
DNA sequence of HCA-UNC (anti-CD52g) (Light Chain)
AGGAGCGAGCTGACACAGGATCCAGTGGTGTCTGTGGCCCTGGGCCAGACAGTGCGGATTAC
TTGTCAGGGCGACAGCCTGAGAACCTACCACGCCTCTTGGTATCAGCAGAAGCCCAGACAGG
CCCCTGTGCTGGTCATCTACGACGAGAACAACAGACCCAGCGGCATCCCCGATAGATTCAGC
GGCAGCACATCTGGCAATACCGCCAGCCTGACAATCACTGGCGCCCAGGCTGAAGATGAGGC
CGACTACTACTGCAACAGCAGAGACAGCAGCGGCAACCGGCTGGTTTTTGGCGGAGGCACAA
AGCTGACAGTGCTGGGCCAACCTAAGGCCGCTCCTAGCGTGACACTGTTCCCTCCAAGCAGC
GAAGAACTGCAGGCCAACAAGGCCACACTCGTGTGCCTGATCAGCGACTTTTATCCTGGCGC
CGTGACCGTGGCCTGGAAGGCTGATAGTTCTCCTGTGAAGGCCGGCGTGGAAACCACCACAC
CTAGCAAGCAGAGCAACAACAAATACGCCGCCAGCAGCTACCTGAGCCTGACACCTGAGCAG
TGGAAGTCCCACAGATCCTACAGCTGCCAAGTGACCCACGAGGGCAGCACCGTGGAAAAAAC
AGTGGCCCCTACCGAGTGCAGC
SEQ ID NO: 7
Annotated Amino Acid sequence of HCA-UNC (anti-CD52g) (Light
Chain)
SSELTQDPVVSVALGQTVRITCQGDSLRTYHASWYQQKPRQAPVLVIYDENNRPSGIPDRES
GSTSGNTASLTITGAQAEDEADYYCNSRDSSGNRLVFGGGTKLTVLGQPKAAPSVTLFPPSS
EELQANKATLVCLISDFYPGAVTVAWKADSSPVKAGVETTTPSKQSNNKYAASSYLSLTPEQ
WKSHRSYSCQVTHEGSTVEKTVAPTECS
SEQ ID NO: 8
Annotated DNA sequence of Synthetic anti-CD52g Fab-IgG (Heavy
Chain)
CAGGTTCAGCTGCAGCAATGGGGAGCCGGACTGCTGAAGCCTAGCGAGACACTGTCTCTGAC
CTGTGCCGTGTACGGCGGCAGCTTCAGCGGCTACTACTGGTCCTGGATCAGACAGCCTCCTG
GCAAAGGCCTGGAATGGATCGGCGAGATCAATCACAGCGGCAGCACCAACTACAACCCCAGC
CTGAGAAGCAGAGTGACCATCAGCGTGGACACCAGCAAGAACCAGTTCAGCCTGAAGCTGAG
AAGCGTGACAGCCGCCGATACCGCCGTGTACTATTGCGCCAGAGGCTTTATGGTCCGAGGCA
TCATGTGGAACTACTACTACATGGACGTGTGGGGCAAGGGCACCACCGTGACAGTTTCTCCA
GCCTCTACCAAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAAAGTTGAGCCCAAATCTTGTG
ACAAAACTCACACAGGTGGCGGAGGATCTGGCGGAGGGGGGAGCGGAGGGGGTGGGTCCGGC
GGGGGGGGTTCCGGGGGTGGTGGATCAGGCGGCGGAGGAAGTCAGGTTCAGCTGCAGCAATG
GGGAGCCGGACTGCTGAAGCCTAGCGAGACACTGTCTCTGACCTGTGCCGTGTACGGCGGCA
GCTTCAGCGGCTACTACTGGTCCTGGATCAGACAGCCTCCTGGCAAAGGCCTGGAATGGATC
GGCGAGATCAATCACAGCGGCAGCACCAACTACAACCCCAGCCTGAGAAGCAGAGTGACCAT
CAGCGTGGACACCAGCAAGAACCAGTTCAGCCTGAAGCTGAGAAGCGTGACAGCCGCCGATA
CCGCCGTGTACTATTGCGCCAGAGGCTTTATGGTCCGAGGCATCATGTGGAACTACTACTAC
ATGGACGTGTGGGGCAAGGGCACCACCGTGACAGTTTCTCCAGCTAGCACCAAGGGCCCATC
GGTCTTCCCCCTGGCACCCTCCTCCAAGAGCACCTCTGGGGGCACAGCGGCCCTGGGCTGCC
TGGTCAAGGACTACTTCCCCGAACCGGTGACGGTGTCGTGGAACTCAGGCGCCCTGACCAGC
GGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCA
GCAACACCAAGGTGGACAAGAAAGTTGAGCCCAAATCTTGTGACAAAACTCACACATGCCCA
CCGTGCCCAGCACCTGAACTCCTGGGGGGACCGTCAGTCTTCCTCTTCCCCCCAAAACCCAA
GGACACCCTCATGATCTCCCGGACCCCTGAGGTCACATGCGTGGTGGTGGACGTGAGCCACG
AAGACCCTGAGGTCAAGTTCAACTGGTACGTGGACGGCGTGGAGGTGCATAATGCCAAGACA
AAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTGTACACCCTG
CCCCCATCCCGGGACGAGCTGACCAAGAACCAGGTCAGCCTGACCTGCCTGGTCAAAGGCTT
CTATCCCAGCGACATCGCCGTGGAGTGGGAGAGCAATGGGCAGCCGGAGAACAACTACAAGA
CCACGCCTCCCGTGCTGGACTCCGACGGCTCCTTCTTCCTCTACAGCAAGCTCACCGTGGAC
AAGAGCAGGTGGCAGCAGGGGAACGTCTTCTCATGCTCCGTGATGCATGAGGCTCTGCACAA
CCACTACACGCAGAAGAGCCTCTCCCTGTCTCCGGGTAAA
SEQ ID NO: 9
Annotated Amino Acid sequence of Synthetic anti-CD52g Fab-IgG
(Heavy Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGEMVRGIMWNYYYMDVWGKGTTVTVSP
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTGGGGSGGGGSGGGGSG
GGGSGGGGSGGGGSQVQLQQWGAGLLKPSETLSLTCAVYGGSFSGYYWSWIRQPPGKGLEWI
GEINHSGSTNYNPSLRSRVTISVDTSKNQFSLKLRSVTAADTAVYYCARGFMVRGIMWNYYY
MDVWGKGTTVTVSPASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTHTCP
PCPAPELLGGPSVFLFETKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTFPVLDSDGSFFLYSKLTVD
KSRWQQGNVFSCSVMHEALHNHYTQKSLSLSPGK
SEQ ID NO: 10
Annotated Amino Acid sequence of Synthetic anti-CD52g Fab
fragment of Fab-IgG (Heavy Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGFMVRGIMWNYYYMDVWGKGTTVTVSP
ASTKGPSVFPLAPSSKSTSGGTAALGCLVKDYFPEPVTVSWNSGALTSGVHTFRAVLQSSGL
YSLSSVVTVPSSSLGTQTYICNVNHKPSNTKVDKKVEPKSCDKTHT
SEQ ID NO: 11
Annotated Amino Acid sequence of Synthetic anti-CD52g Fc
fragment of Fab-IgG (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPTEKTISKAKGQPREPTFYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 12
DNA sequence of Synthetic anti-CD52g Fab-IgG (Light Chain)
AGCAGCGAGCTGACACAGGATCCAGTGGTGTCTGTGGCCCTGGGCCAGACAGTGCGGATTAC
TTGTCAGGGCGACAGCCTGAGAACCTACCACGCCTCTTGGTATCAGCAGAAGCCCAGACAGG
CCCCTGTGCTGGTCATCTACGACGAGAACAACAGACCCAGCGGCATCCCCGATAGATTCAGC
GGCAGCACATCTGCCAATACCGCCAGCCTGACAATCACTGGCGCCCAGGCTGAAGATGAGGC
CGACTACTACTGCAACAGCAGAGACAGCAGCGGCAACCGGCTGGTTTTTGGCGGAGGCACAA
AGCTGACAGTGCTGGGCCAACCTAAGGCCGCTCCTAGCGTGACACTGTTCCCTCCAAGCAGC
GAAGAACTGCAGGCCAACAAGGCCACACTCGTGTGCCTGATCAGCGACTTTTATCCTGGCGC
CGTGACCGTGGCCTGGAAGGCTGATAGTTCTCCTGTGAAGGCCGGCGTGGAAACCACCACAC
CTAGCAAGCAGAGCAACAACAAATACGCCGCCAGCAGCTACCTGAGCCTGACACCTGAGCAG
TGGAAGTCCCACAGATCCTACAGCTGCCAAGTGACCCACGAGGGCAGCACCGTGGAAAAAAC
AGTGGCCCCTACCGAGTGCAGC
SEQ ID NO: 13
Annotated Amino Acid sequence of Synthetic anti-CD52g Fab-IgG
(Light Chain)
SSELTQDPVVSVALGQTVRITCQGDSLRTYHASWYQQKPRQAPVLVIYDENNRTSGIPDRFS
GSTSGNTASLTITGAQAEDEADYYCNSRDSSGNRLVFGGGTKLTVLGQPKAAPSVTLFPPSS
EELQANKATINCLISDFYPGAVTVAWKADSSPVKAGVETTTPSKQSNNKYAASSYLSLTPEQ
WKSHRSYSCQVTHEGSTVEKTVAPTECS
SEQ ID NO: 14
Annotated DNA sequence of Synthetic anti-CD52g IgG-Fab (Heavy
Chain)
CAGGTTCAGCTGCAGCAATGGGGAGCCGGACTGCTGAAGCCTAGCGAGACACTGTCTCTGAC
CTGTGCCGTGTACGGCGGCAGCTTCAGCGGCTACTACTGGTCCTGGATCAGACAGCCTCCTG
GCAAAGGCCTGGAATGGATCGGCGAGATCAATCACAGCGGCAGCACCAACTACAACCCCAGC
CTGAGAAGCAGAGTGACCATCAGCGTGGACACCAGCAAGAACCAGTTCAGCCTGAAGCTGAG
AAGCGTGACAGCCGCCGATACCGCCGTGTACTATTGCGCCAGAGGCTTTATGGTCCGAGGCA
TCATGTGGAACTACTACTACATGGACGTGTGGGGCAAGGGCACCACCGTGACAGTTTCTCCA
GCTAGCACCAAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAAAGTTGAGCCCAAATCTTGTG
ACAAAACTCACACATGCCCACCGTGCCCAGCACCTGAACTCCTGGGGGGACCGTCAGTCTTC
CTCTTCCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACGGCGTGG
AGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAG
AACCACAGGTGTACACCCTGCCCCCATCCCGGGACGAGCTGACCAAGAACCAGGTCAGCCTG
ACCTGCCTGGTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCTTCTTCCTCT
ACAGCAAGCTCACCGTGGACAAGAGCAGGTGGCAGCAGGGGAACGTCTTCTCATGCTCCGTG
ATGCATGAGGCTCTGCACAACCACTACACGCAGAAGAGCCTCTCCCTGTCTCCGGGTAAAGG
ATCCGGTGGCGGAGGATCTGGCGGAGGGGGGAGCGGAGGGGGTGGGTCCGGCGGGGGGGGTT
CCGGGGGTGGTGGATCAGGCGGCGGAGGAAGTCAGGTTCAGCTGCAGCAATGGGGAGCCGGA
CTGCTGAAGCCTAGCGAGACACTGTCTCTGACCTGTGCCGTGTACGGCGGCAGCTTCAGCGG
CTACTACTGGTCCTGGATCAGACAGCCTCCTGGCAAAGGCGTGGAATGGATCGGCGAGATCA
ATCACAGCGGCAGCACCAACTACAACCCCAGCCTGAGAAGGAGAGTGACCATCAGCGTGGAC
ACCAGCAAGAACCAGTTCAGCCTGAAGCTGAGAAGCGTGACAGCCGCCGATACCGCCGTGTA
CTATTGCGCCAGAGGCTTTATGGTCCGAGGCATCATGTGGAACTACTACTACATGGACGTGT
GGGGCAAGGGCACCACCGTGACAGTTTCTCCAGCCTCTACCAAGGGCCCATCGGTCTTCCCC
CTGGCACCCTCCTCCAAGAGCACCTCTGGGGGCACAGCGGCCCTGGGCTGCCTGGTCAAGGA
CTACTTCCCCGAACCGGTGACGGTGTCGTGGAACTCAGGCGCCCTGACCAGCGGCGTGCACA
CCTTCCCGGCTGTCCTACAGTCCTCAGGACTCTACTCCCTCAGCAGCGTGGTGACCGTGCCC
TCCAGCAGCTTGGGCACCCAGACCTACATCTGCAACGTGAATCACAAGCCCAGCAACACCAA
GGTGGACAAGAAAGTTGAGCCCAAATCTTGTGACAAAACTCACACA
SEQ ID NO: 15
Annotated Amino Acid sequence of Synthetic anti-CD52g IgG-Fab
(Heavy Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGFMVRGIMWNYYYMDVWGKGTTVTVSP
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPPSRDELTKNQVSL
TUNKGFYPSDIAVEWESNGQPENNYKTTETVLDSIDGSFFLYSKLTVDKSRWQQGNVFSCSV
MHEALHNHYTQKSLSLSPGKGSGGGGSGGGGSGGGGSGGGGSGGGGSGGGGSQVQLQQWGAG
LLKPSETLSLTCAVYGGSFSGYYWSWIRQPPGKGLEWIGEINESGSTNYNPSLRSRVTISVD
TSKNQFSLKLRSVTAADTAVYYCARGFMVRGIMWNYYYMDVWGKGTTVTVSPASTKGPSVFP
LAPSSKSTSGGTAALGCLVKDYFPEPVTVSWNSGALTSGVHTFPAVLQSSGLYSLSSVVTVP
SSSLGTQTYICNVNHKPSNTKVDKKVEPKSCDKTHT
SEQ ID NO: 16
Annotated Amino Amid sequence of Synthetic anti-CD52g Fab
fragment of IgG-Fab (Heavy Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGEMVRGIMWNYYYMDVWGKGTTVTVSP
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHT
SEQ ID NO: 17
Annotated Amino Acid sequence of Synthetic anti-CD52g Fc
fragment of IgG-Fab (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 18
DNA sequence of Synthetic anti-CD52g IgG-Fab (Light Chain)
AGCAGCGAGCTGACACAGGATCCAGTGGTGTCTGTGGCCCTGGGCCAGACAGTGCGGATTAC
TTGTCAGGGCGACAGCCTGAGAACCTACCACGCCTCTTGGTATCAGCAGAAGCCCAGACAGG
CCCCTGTGCTGGTCATCTACGACGAGAACAACAGACCCAGCGGCATCCCCGATAGATTCAGC
GGCAGCACATCTGGCAATACCGCCAGCCTGACAATCACTGGCGCCCAGGCTGAAGATGAGGC
CGACTACTACTGCAACAGCAGAGACAGCAGCGGCAACCGGCTGGTTTTTGGCGGAGGCACAA
AGCTGACAGTCCTGGGCCAACCTAAGGCCGCTCCTAGCGTGACACTGTTCCCTCCAAGCAGC
GAAGAACTGCAGGCCAACAAGGCCACACTCGTGTGCCTGATCAGCGACTTTTATCCTGGCGC
CGTGACCGTGGCCTGGAAGGCTGATAGTTCTCCTGTGAAGGCCGGCGTGGAAACCACCACAC
CTAGCAAGCAGAGCAACAACAAATACGCCGCCAGCAGCTACCTGAGCCTGACACCTGAGCAG
TGGAAGTCCCACAGATCCTACAGCTGCCAAGTGACCCACGAGGGCAGCACCGTGGAAAAAAC
AGTGGCCCCTACCGAGTGCAGC
SEQ ID NO: 19
Annotated Amino Acid sequence of Synthetic anti-CD52g IgG-Fab
(Light Chain)
SSELTQDPVVSVALGQTVRITCQGDSLRTYHASWYQQKPRQAPVLVIYDENNRPSGIPDRFS
GSTSGNTASLTITGAQAEDEADYYCNSRDSSGNRLVFGGGTKLTVLGQPKAAPSVTLFPPSS
EELQANKATLVCLISDFYPGAVTVAWKADSSPVKAGVETTTPSKQSNNKYAASSYLSLTPEQ
WKSHRSYSCQVTHEGSTVEKTVAPTECS
SEQ ID NO: 20
Annotated DNA sequence of Synthetic anti-CD52g Feb-IgG-Fab
(Heavy Chain)
CAGGTTCAGCTCCAGCAATGGGGAGCCGGACTGCTGAAGCCTAGCGAGACACTGTCTCTGAC
CTGTGCCGTGTACGGCGGCAGCTTCAGCGGCTACTACTGGTCCTGGATCAGACAGCCTCCTG
GCAAAGGCCTGGAATGGATCGGCGAGATCAATCACAGCGGCAGCACCAACTACAACCCCAGC
CTGAGAAGGAGAGTGACCATCAGCGTGGACACCAGCAAGAACCAGTTCAGCCTGAAGCTGAG
AAGCGTGACAGCCGCCGATACCGCCGTGTACTATTGCGCCAGAGGCTTTATGGTCCGAGGCA
TCATGTGGAACTACTACTACATGGACGTGTGGGGCAAGGGCACCACCGTGACAGTTTCTCCA
GCCTCTACCAAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAAAGTTGAGCCCAAATCTTGTG
ACAAAACTCACACAGGTGGCGGAGGATCTGGCGGAGGGGGGAGCGGAGGGGGTGGGTCCGGC
GGGGGGGGTTCCGGGGGTGGTGGATCAGGCGGCGGAGGAAGTCAGGTTCAGCTGCAGCAATG
GGGAGCCGGACTGCTGAAGCCTAGCGAGACACTGTCTCTGACCTGTGCCGTGTACGGCGGCA
GCTTCAGCGGCTACTACTGGTCCTGGATCAGACAGCCTCCTGGCAAAGGCCTGGAATGGATC
GGCGAGATCAATCACAGCGGCAGCACCAACTACAACCCCAGCCTGAGAAGCAGAGTGACCAT
CAGCGTGGACACCAGCAAGAACCAGTTCAGCCTGAAGCTGAGAAGCGTGACAGCCGCCGATA
CCGCCGTGTACTATTGCGCCAGAGGCTTTATGGTCCGAGGCATCATGTGGAACTACTACTAC
ATGGACGTGTGGGGCAAGGGCACCACCGTGACAGTTTCTCCAGCTAGCACCAAGGGCCCATC
GGTCTTCCCCCTGGCACCCTCCTCCAAGAGCACCTCTGGGGGCACAGCGGCCCTGGGCTGCC
TGGTCAAGGACTACTTCCCCGAACCGGTGACGGTGTCGTGGAACTCAGGCGCCCTGACCAGC
GGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCA
GCAACACCAAGGTGGACAAGAAAGTTGAGCCCAAATCTTGTGACAAAACTCACACATGCCCA
CCGTGCCCAGCACCTGAACTCCTGGGGGGACCGTCAGTCTTCCTCTTCCCCCCAAAACCCAA
GGACACCCTCATGATCTCCCGGACCCCTGAGGTCACATGCGTGGTGGTGGACGTGAGCCACG
AAGACCCTGAGGTCAAGTTCAACTGGTACGTGGACGGCGTGGAGGTGCATAATGCCAAGACA
AAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTGTACACCCTG
CCCCCATCCCGGGACGAGCTGACCAAGAACCAGGTCAGCCTGACCTGCCTGGTCAAAGGCTT
CTATCCCAGCGACATCGCCGTGGAGTGGGAGAGCAATGGGCAGCCGGAGAACAACTACAAGA
CCACGCCTCCCGTGCTGGACTCCGACGGCTCCTTCTTCCTCTACAGCAAGCTCACCGTGGAC
AAGAGCAGGTGGCAGCAGGGGAACGTCTTCTCATGCTCCGTGATGCATGAGGCTCTGCACAA
CCACTACACGCAGAAGAGCCTCTCCCTGTCTCCGGGTAAAGGATCCGGTGGCGGAGGATCTG
GCGGAGGGGGGAGCGGAGGGGGTGGGTCCGGCGGGGGGGGTTCCGGGGGTGGTGGATCAGGC
GGCGGAGGAAGTCAGGTTCAGCTGCAGCAATGGGGAGCCGGACTGCTGAAGCCTAGCGAGAC
ACTGTCTCTGACCTGTGCCGTGTACGGCGGCAGCTTCAGCGGCTACTACTGGTCCTGGATCA
GACAGCCTCCTGGCAAAGGCCTGGAATGGATCGGCGAGATCAATCACAGCGGCAGCACCAAC
TACAACCCCAGCCTGAGAAGCAGAGTGACCATCAGCGTGGACACCAGCAAGAACCAGTTCAG
CCTGAAGCTGAGAAGCGTGACAGCCGCCGATACCGCCGTGTACTATTGCGCCAGAGGCTTTA
TGGTCCGAGGCATCATGTGGAACTACTACTACATGGACGTGTGGGGCAAGGGCACCACCGTG
ACAGTTTCTCCAGCCTCTACCAAGGGCCCATCGGTCTTCCCCCTGGCACCCTCCTCCAAGAG
CACCTCTGGGGGCACAGCGGCCCTGGGCTGCCTGGTCAAGGACTACTTCCCCGAACCGGTGA
CGGTGTCGTGGAACTCAGGCGCCCTGACCAGCGGCGTGCACACCTTCCCGGCTGTCCTACAG
TCCTCAGGACTCTACTCCCTCAGCAGCGTGGTGACCGTGCCCTCCAGCAGCTTGGGCACCCA
GACCTACATCTGCAACGTGAATCACAAGCCCAGCAACACCAAGGTGGACAAGAAAGTTGAGC
CCAAATCTTGTGACAAAACTCACACA
SEQ ID NO: 21
Annotated Amino Acid sequence of Synthetic anti-CD52g Fab-IgG-
Fab (Heavy Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGFMVRGIMWNYYYMDVWCKGTTVTVSP
ASTKGPSVFPLAPSSKSTSGGTAALGCLVKDYFPEPVTVSWNSGALTSGVHTFPAVLQSSGL
YSLSSVVTVPSSSLGTQTYICNVNEKPSNTKVDKKVEPKSCDKTHTGGGGSGGGGSGGGGSG
GGGSGGGGSGGGGSQVQLQQWGAGLLKPSETLSLTCAVYGGSFSGYYWSWIRQPPGKGLEWI
GEINHSGSTNYNPSLRSRVTISVDTSKNQFSLKLRSVTAADTAVYYCARGFMVRGIMWNYYY
MDVWGKGTTVTVSPASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTIPPVLDSDGSFFLYSKLTVD
KSRWQQGNVFSCSVMHEALHNHYTQKSLSLSPGKGSGGGGSGGGGSGGGGSGGGGSGGGGSG
GGGSQVQLQQWGAGLLKPSETLSLTCAVYGGSFSGYYWSWIRQPPGKGLEWIGEINHSGSTN
YNPSLRSRVTISVDTSKNQFSLKLRSVIAADTAVYYCARGFMVRGIMWNYYYMDVWGKGTTV
TVSPASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHT
SEQ ID NO: 22
Annotated Amino Acid sequence of Synthetic anti-CD52g Fab
fragment of Fab-IgG-Fab (Heavy Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGFMVRGIMWNYYYMDVWGKGTIVTVSP
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHT
SEQ ID NO: 23
Annotated Amino Acid sequence of Synthetic anti-CD52g Fc
fragment of Fab-IgG-Fab (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFELYSKLTVD
KSRWQQGNVFSCSVMHEALHNHYTQKSLSLSPGK
SEQ ID NO: 24
DNA sequence of Synthetic anti-CD52g Fab-IgG-Fab (Light Chain)
AGCAGCGAGCTGACACAGGATCCAGTGGTGTCTGTGGCCCTGGGCCAGACAGTGCGGATTAC
TTGTCAGGGCGACAGCCTGAGAACCTACCACGCCTCTTGGTATCAGCAGAAGCCCAGACAGG
CCCCTGTGCTGGTCATCTACGACGAGAACAACAGACCCAGCGGCATCCCCGATAGATTCAGC
GGCAGCACATCTGGCAATACCGCCAGCCTGACAATCACTGGCGCCCAGGCTGAAGATGAGGC
CGACTACTACTGCAACAGCAGAGACAGCAGCGGCAACCGGCTGGTTTTTGGCGGAGGCACAA
AGCTGACAGTGCTGGGCCAACCTAAGGCCGCTCCTAGCGTGACACTGTTCCCTCCAAGCAGC
GAAGAACTGCAGGCCAACAAGGCCACACTCGTGTGCCTGATCAGCGACTTTTATCCTGGCGC
CGTGACCGTGGCCTGGAAGGCTGATAGTTCTCCTGTGAAGGCCGGCGTGGAAACCACCACAC
CTAGCAAGCAGAGCAACAACAAATACGCCGCCAGCAGCTACCTGAGCCTGACACCTGAGGAG
TGGAAGTCCCACAGATCCTACAGCTGCCAAGTGACCCACGAGGGCAGCACCGTGGAAAAAAC
AGTGGCCCCTACCGAGTGCAGC
SEQ ID NO: 25
Annotated Amino Acid sequence of Synthetic anti-CD52g Fab-IgG-
Fab (Light Chain)
SSELTQDPVVSVALGQTVRITCQGDSLRTYHASWYQQKPRQAPVLVIYDENNRPSGIPDRES
GSTSGNTASLTITGAQAEDEADYYCNSRDSSGNRLVEGGGTKLTVLGQPKAAPSVTLEPPSS
EELQANKATLVCLISDFYPGAVTVAWKADSSPVKAGVETTTPSKQSNNKYAASSYLSLTPEQ
WKSHRSYSCQVTHEGSTVEKTVAPTECS
SEQ ID NO: 26
Annotated DNA sequence of Synthetic anti-CD52g Fab-IgG-Fab-Fab
(Heavy Chain)
CAGGTTCAGCTGCAGCAATGGGGAGCCGGACTGCTGAAGCCTAGCGAGACACTGTCTCTGAC
CTGTGCCGTGTACGGCGGCAGCTTCAGCGGCTACTACTGGTCCTGGATCAGACAGCCTCCTG
GCAAAGGCCTGGAATGGATCGGCGAGATCAATCACAGCGGCAGCACCAACTACAACCCCAGC
CTGAGAAGCAGAGTGACCATCAGCGTGGACACCAGCAAGAACCAGTTCAGCCTGAAGCTGAG
AAGCGTGACAGCCGCCGATACCGCCGTGTACTATTGCGCCAGAGGCTTTATGGTCCGAGGCA
TCATGTGGAACTACTACTACATGGACGTGTGGGGCAAGGGCACCACCGTGACAGTTTCTCCA
GCCTCTACCAAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGGAGCTTGGGCACCCAGACCTACATCTG
CAACGTGAATCACAAGCCCAGCAACACCAAGGTGGACAAGAAAGTTGAGCCCAAATCTTGTG
ACAAAACTCACACAGGTGGCGGAGGATCTGGCGGAGGGGGGAGCGGAGGGGGTGGGTCCGGC
GGGGGGGGTTCCGGGGGTGGTGGATCAGGCGGCGGAGGAAGTCAGGTTCAGCTGCAGCAATG
GGGAGCCGGACTGCTGAAGCCTAGCGAGACACTGTCTCTGACCTGTGCCGTGTACGGCGGCA
GCTTCAGCGGCTACTACTGGTCCTGGATCAGACAGCCTCCTGGCAAAGGCCTGGAATGGATC
GGCGAGATCAATCACAGCGGCAGCACCAACTACAACCCCAGCCTGAGAAGCAGAGTGACCAT
CAGCGTGGACACCAGCAAGAACCAGTTCAGCCTGAAGCTGAGAAGCGTGACAGCCGCCGATA
CCGCCGTGTACTATTGCGCCAGAGGCTTTATGGTCCGAGGCATCATGTGGAACTACTACTAC
ATGGACGTGTGGGGCAAGGGCACCACCGTGACAGTTTCTCCAGCTAGCACCAAGGGCCCATC
GGTCTTCCCCCTGGCACCCTCCTCCAAGAGCACCTCTGGGGGCACAGCGGCCCTGGGCTGCC
TGGTCAAGGACTACTTCCCCGAACCGGTGACGGTGTCGTGGAACTCAGGCGCCCTGACCAGC
GGCGTGCACACCTTCCCGGCTGTCCTACAGTCCTCAGGACTCTACTCCCTCAGCAGCGTGGT
GACCGTGCCCTCCAGCAGCTTGGGCACCCAGACCTACATCTGCAACGTGAATCACAAGCCCA
GCAACACCAAGGTGGACAAGAAAGTTGAGCCCAAATCTTGTGACAAAACTCACACATGCCCA
CCGTGCCCAGCACCTGAACTCCTGGGGGGACCGTCAGTCTTCCTCTTCCCCCCAAAACCCAA
GGACACCCTCATGATCTCCCGGACCCCTGAGGTCACATGCGTGGTGGTGGACGTGAGCCACG
AAGACCCTGAGGTCAAGTTCAACTGGTACGTGGACGGCGTGGAGGTGCATAATGCCAAGACA
AAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCC
CCATCGAGAAAACCATCTCCAAAGCCAAAGGGCAGCCCCGAGAACCACAGGTGTACACCCTG
CCCCCATCCCGGGACGAGCTGACCAAGAACCAGGTCAGCCTGACCTGCCTGGTCAAAGGCTT
CTATCCCAGCGACATCGCCGTGGAGTGGGAGAGCAATGGGCAGCCGGAGAACAACTACAAGA
CCACGCCTCCCGTGCTGGACTCCGACGGCTCCTTCTTCCTCTACAGCAAGCTCACCGTGGAC
AAGAGCAGGTGGCAGGAGGGGAACGTCTTCTCATGCTCCGTGATGCATGAGGCTCTGCACAA
CCACTACACGCAGAAGAGCCTCTCCCTGTCTCCGGGTAAAGGATCCGGTGGCGGAGGCTCTG
GTGGAGGAGGCAGTGGCGGAGGCGGTTCTGGTGGTGGTGGCTCTGGTGGCGGCGGTTCAGGC
GGTGGCGGATCTCAAGTTCAGCTGCAGCAATGGGGAGCCGGCCTGCTGAAGCCTTCTGAGAC
ACTGTCTCTGACCTGCGCCGTGTACGGCGGCAGCTTTAGCGGCTACTACTGGTCCTGGATCA
GACAGCCTCCTGGCAAAGGCCTGGAATGGATCGGCGAGATCAATCACAGCGGCAGCACCAAC
TACAACCCCAGCCTGAGAAGCAGAGTGACCATCAGCGTGGACACCAGCAAGAACCAGTTCAG
CCTGAAGCTGCGGAGCGTGACAGCCGCTGATACAGCCGTGTACTACTGCGCCAGAGGCTTTA
TGGTCCGAGGCATCATGTGGAACTACTACTACATGGACGTGTGGGGCAAGGGCACCACCGTG
ACAGTTTCTCCAGCCAGCACAAAGGGCCCCAGCGTTTTCCCACTGGCTCCTAGGAGCAAGAG
CACATCTGGTGGAACAGCCGCTCTGGGCTGCCTGGTCAAGGATTACTTTCCCGAGCCTGTGA
CCGTGTCCTGGAATTCTGGCGCTCTGACAAGCGGCGTGCACACCTTTCCAGCTGTGCTGCAA
AGGAGCGGCCTGTACTCTCTGAGGAGCGTGGTCACAGTGCCAAGCTCTAGCCTGGGCACCCA
GACCTACATCTGCAATGTGAACCACAAGCCTAGCAACACCAAGGTGGACAAGAAGGTGGAAC
CCAAGAGCTGCGACAAGACCCATACAGGGGGCGGTGGAAGCGGAGGCGGGGGTAGCGGTGGT
GGCGGCAGCGGCGGAGGCGGATCAGGGGGCGGCGGAAGTGGTGGCGGTGGTTCTCAGGTTCA
ACTCCAACAGTGGGGCGCTGGACTGCTGAAACCTAGCGAAACCCTGAGCCTGACATGTGCTG
TGTATGGCGGCTCCTTCTCCGGCTACTATTGGAGCTGGATTCGGCAGCCACCAGGCAAGGGA
CTCGAGTGGATTGGAGAGATCAACCACTCCGGCTCCACCAATTACAATCCATCTCTGCGGTC
CCGCGTGACCATCTCCGTGGATACCTCTAAGAATCAGTTCTCACTGAAGCTGAGATCCGTGA
CCGCTGCCGACACTGCCGTGTATTATTGTGCCCGGGGATTCATGGTTCGAGGGATTATGTGG
AATTACTATTATATGGATGTCTGGGGAAAAGGGACGACCGTGACTGTGTCCCCTGCCTCTAC
AAAGGGACCCTCCGTGTTTCCTCTGGCTCCCAGCTCTAAGTCTACCAGCGGAGGAACAGCTG
CCCTGGGATGTCTCGTGAAAGACTACTTCCCCGAACCAGTGACAGTCAGCTGGAACAGCGGA
GCCCTGACTTCTGGGGTGCACACATTCCCTGCCGTCCTGCAATCTTCTGGCCTGTACAGCCT
GTCCAGCGTCGTGACCGTTCCTTCTAGCTCTCTGGGAACACAGACATATATCTGTAATGTCA
ATCACAAACCCTCCAATACGAAGGTCGACAAAAAGGTCGAGCCTAAGTCCTGTGATAAGACC
CACACC
SEQ ID NO: 27
Annotated Amino Acid sequence of Synthetic anti-CD52g Fab-IgG-
Fab-Fab (Heavy Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGFMVRGIMWNYYYMDVWGKGTTVTVSP
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTGGGGSGGGGSGGGGSG
GGGSGGGGSGGGGSQVQLQQWGAGLLKPSETLSLTCAVYGGSFSGYYWSWIRQPPGKGLEWI
GEINHSGSTNYNPSLRSRVTISVDTSKNQFSLKLRSVTAADTAVYYCARGFMVRGIMWNYYY
MDVWGKGTTVTVSPASTKGPSVFPLAPSSKSTSGGTAALGCLVKDYFPEPVTVSWNSGALTS
GVHTFPAVLQSSGLYSLSSVVTVPSSSLGTQTYICNVNHKPSNTKVDKKVEPKSCDKTHTCP
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVESCSVMHEALHNHYTQKSLSLSPGKGSGGGGSGGGGSGGGGSGGGGSGGGGSG
GGGSQVQLQQWGAGLLKPSETLSLTCAVYGGSFSGYYNSWIRQPPGKGLEWIGEINHSGSTN
YNPSLRSRVTISVDTSKNQFSLKLRSVTAADTAVYYCARGFMVRGIMWNYYYMDVWGKGTTV
TVSPASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTGGGGSGGGGSGG
GGSGGGGSGGGGSGGGGSQVQLQQWGAGLLKPSETLSLTCAVYGGSFSGYYWSWIRQPPGKG
LEWIGEINHSGSTNYNPSLRSRVTISVDTSKNQFSLKLRSVTAADTAVYYCARGFMVRGIMW
NYYYMDVWGKGTTVTVSPASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKSCDKT
HT
SEQ ID NO: 28
Annotated Amino Acid sequence of Synthetic anti-CD52g Fab
fragment of Fab-IgG-Fab-Fab (Heavy Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGFMVRGIMWNYYYMDVWGKGTTVTVSP
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHT
SEQ ID NO: 29
Annotated Amino Acid sequence of Synthetic anti-CD52g Fc
fragment of Fab-IgG-Fab-Fab (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 30
DNA sequence of Synthetic anti-CD52g Fab-IgG-Fab-Fab (Light
Chain)
AGGAGCGAGCTGACACAGGATCCAGTGGTGTCTGTGGCCCTGGGCCAGACAGTGCGGATTAC
TTGTCAGGGCGACAGCCTGAGAACCTACCACGCCTCTTGGTATCAGGAGAAGCCCAGACAGG
CCCCTGTGCTGGTCATCTACGACGAGAACAACAGACCCAGCGGCATCCCCGATAGATTCAGC
GGCAGCACATCTGGCAATACCGCCAGCCTGACAATCACTGGCGCCCAGGCTGAAGATGAGGC
CGACTACTACTGCAACAGGAGAGACAGGAGCGGCAACCGGCTGGTTTTTGGCGGAGGCACAA
AGCTGACAGTGCTGGGCCAACCTAAGGCCGCTCCTAGCGTGACACTGTTCCCTCCAAGGAGC
GAAGAACTGCAGGCCAACAAGGCCACACTCGTGTGCCTGATCAGCGACTTTTATCCTGGCGC
CGTGACCGTGGCCTGGAAGGCTGATAGTTCTCCTGTGAAGGCCGGCGTGGAAACCACCACAC
CTAGCAAGGAGAGCAACAACAAATACGCCGCCAGCAGCTACCTGAGCCTGACACCTGAGGAG
TGGAAGTCCCACAGATCCTACAGCTGCCAAGTGACCCACGAGGGCAGCACCGTGGAAAAAAC
AGTGGCCCCTACCGAGTGCAGC
SEQ ID NO: 31
Annotated Amino Acid sequence of Synthetic anti-CD52g Fab-IgG-
Fab-Fab (Light Chain)
SSELTQDPVVSVALGQTVRITCQGDSLRTYHASWYQQKPRQAPVLVIYDENNRPSGIPDRFS
GSTSGNTASLTITGAQAEDEADYYCNSRDSSGNRLVFGGGTKLTVL
GQPKAAPSVTLFPPSSEELQANKATLVCLISDFYPGAVTVAWKADSSPVKAGVETTTPSKQS
NNKYAASSYLSLTPEQWKSHRSYSCQVTHEGSTVEKTVAPTECS
SEQ ID NO: 32
Annotated DNA sequence of Synthetic anti-CD52g Fab-Fab-IgG-
Fab-Fab (Heavy Chain)
CAAGTTCAGCTGCAGCAATGGGGAGCCGGCCTGCTGAAGCCTTCTGAGACACTGTCTCTGAC
CTGCGCCGTGTACGGCGGCAGCTTTAGCGGCTACTACTGGTCCTGGATCAGACAGCCTCCTG
GCAAAGGCCTGGAATGGATCGGCGAGATCAATCACAGCGGCAGCACCAACTACAACCCCAGC
CTGAGAAGCAGAGTGACCATCAGCGTGGACACCAGCAAGAACCAGTTCAGCCTGAAGCTGCG
GAGCGTGACAGCCGCTGATACAGCCGTGTACTACTGCGCCAGAGGCTTTATGGTCCGAGGCA
TCATGTGGAACTACTACTACATGGACGTGTGGGGCAAGGGCACCACCGTGACAGTTTCTCCA
GCCAGCACAAAGGGCCCCAGCGTTTTCCCACTGGCTCCTAGGAGCAAGAGCACATCTGGTGG
AACAGCCGCTCTGGGCTGCCTGGTCAAGGATTACTTTCCCGAGCCTGTGACCGTGTCCTGGA
ATTCTGGCGCTCTGACAAGCGGCGTGCACACCTTTCCAGCTGTGCTGCAAAGGAGCGGCCTG
TACTCTCTGAGCAGCGTGGTCACAGTGCCAAGCTCTAGCCTGGGCACCCAGACCTACATCTG
CAATGTGAACCACAAGCCTAGCAACACCAAGGTGGACAAGAAGGTGGAACCCAAGAGCTGCG
ACAAGACCCATACAGGGGGCGGTGGAAGCGGAGGCGGGGGTAGCGGTGGTGGCGGCAGCGGC
GGAGGCGGATCAGGGGGCGGCGGAAGTGGTGGCGGTGGTTCTCAGGTTCAACTCCAACAGTG
GGGCGCTGGACTGCTGAAACCTAGCGAAACCCTGAGCCTGACATGTGCTGTGTATGGCGGCT
CCTTCTCCGGCTACTATTGGAGCTGGATTCGGCAGCCACCAGGCAAGGGACTCGAGTGGATT
GGAGAGATCAACCACTCCGGCTCCACCAATTACAATCCATCTCTGCGGTCCCGCGTGACCAT
CTCCGTGGATACCTCTAAGAATCAGTTCTCACTGAAGCTGAGATCCGTGACCGCTGCCGACA
CTGCCGTGTATTATTGTGCCCGGGGATTCATGGTTCGAGGGATTATGTGGAATTACTATTAT
ATGGATGTCTGGGGAAAAGGGACGACCGTGACTGTGTCCCCTGCCTCTACAAAGGGACCCTC
CGTGTTTCCTCTGGCTCCCAGCTCTAAGTCTACCAGCGGAGGAACAGCTGCCCTGGGATGTC
TCGTGAAAGACTACTTCCCCGAACCAGTGACAGTCAGCTGGAACAGCGGAGCCCTGACTTCT
GGGGTGCACACATTCCCTGCCGTCCTGCAATCTTCTGGCCTGTACAGCCTGTCCAGCGTCGT
GACCGTTCCTTCTAGCTCTCTGGGAACACAGACATATATCTGTAATGTCAATCACAAACCCT
CCAATACGAAGGTCGACAAAAAGGTCGAGCCTAAGTCCTGTGATAAGACCCACACCGGTGGC
GGAGGCTCTGGGGGAGGAGGCAGTGGCGGAGGCGGTTCTGGTGGTGGTGGCTCTGGTGGCGG
CGGTTCAGGCGGTGGCGGATCTCAGGTTCAGCTGCAGCAATGGGGAGCCGGACTGCTGAAGC
CTAGCGAGACACTGTCTCTGACCTGTGCCGTGTACGGCGGCAGCTTCAGCGGCTACTACTGG
TCCTGGATCAGACAGCCTCCTGGCAAAGGCCTGGAATGGATCGGCGAGATCAATCACAGCGG
CAGCACCAACTACAACCCCAGCCTGAGAAGGAGAGTGACCATCAGCGTGGACACCAGCAAGA
ACCAGTTCAGCCTGAAGCTGAGAAGCGTGACAGCCGCCGATACCGCCGTGTACTATTGCGCC
AGAGGCTTTATGGTCCGAGGCATCATGTGGAACTACTACTACATGGACGTGTGGGGCAAGGG
CACCACCGTTACAGTCTCACCTGCTAGCACCAAGGGCCCATCGGTCTTCCCCCTGGCACCCT
CCTCCAAGAGCACCTCTGGGGGCACAGCGGCCCTGGGCTGCCTGGTCAAGGACTACTTCCCC
GAACCGGTGACGGTGTCGTGGAACTCAGGCGCCCTGACCAGCGGCGTGCACACCTTCCCGGC
TGTCCTACAGTCCTCAGGACTCTACTCCCTCAGGAGCGTGGTGACCGTGCCCTCCAGCAGCT
TGGGCACCCAGACCTACATCTGCAACGTGAATCACAAGCCCAGCAACACCAAGGTGGACAAG
AAAGTTGAGCCCAAATCTTGTGACAAAACTCACACATGCCCACCGTGCCCAGCACCTGAACT
CCTGGGGGGACCGTCAGTCTTCCTCTTCCCCCCAAAACCCAAGGACACCCTCATGATCTCCC
GGACCCCTGAGGTCACATGCGTGGTGGTGGACGTGAGCCACGAAGACCCTGAGGTCAAGTTC
AACTGGTACGTGGACGGCGTGGAGGTGCATAATGCCAAGACAAAGCCGCGGGAGGAGGAGTA
CAACAGCACGTACCGTGTGGTCAGCGTCCTCACCGTCCTGCACCAGGACTGGCTGAATGGCA
AGGAGTACAAGTGCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATCCCGGGACGAGCT
GACCAAGAACCAGGTCAGCCTGACCTGCCTGGTCAAAGGCTTCTATCCCAGCGACATCGCCG
TGGAGTGGGAGAGCAATGGGCAGCCGGAGAACAACTACAAGACCACGCCTCCCGTGCTGGAC
TCCGACGGCTCCTTCTTCCTCTACAGCAAGCTCACCGTGGACAAGAGCAGGTGGCAGGAGGG
GAACGTCTTCTCATGCTCCGTGATGCATGAGGCTCTGCACAACCACTACACGCAGAAGAGCC
TCTCCCTGTCTCCGGGTAAAGGATCCGGTGGCGGAGGCTCTGGTGGAGGAGGCAGTGGCGGA
GGCGGTTCTGGTGGTGGTGGCTCTGGTGGCGGCGGTTCAGGCGGTGGCGGATCTCAAGTTCA
GCTGCAGCAATGGGGAGCCGGCCTGCTGAAGCCTTCTGAGACACTGTCTCTGACCTGCGCCG
TGTACGGCGGCAGCTTTAGCGGCTACTACTGGTCCTGGATCAGACAGCCTCCTGGCAAAGGC
CTGGAATGGATCGGCGAGATCAATCACAGCGGCAGCACCAACTACAACCCCAGCCTGAGAAG
CAGAGTGACCATCAGCGTGGACACCAGCAAGAACCAGTTCAGCCTGAAGCTGCGGAGCGTGA
CAGCCGCTGATACAGCCGTGTACTACTGCGCCAGAGGCTTTATGGTCCGAGGCATCATGTGG
AACTACTACTACATGGACGTGTGGGGCAAGGGCACCACCGTGACAGTTTCTCCAGCCAGCAC
AAAGGGCCCCAGCGTTTTCCCACTGGCTCCTAGCAGCAAGAGCACATCTGGTGGAACAGCCG
CTCTGGGCTGCCTGGTCAAGGATTACTTTCCCGAGCCTGTGACCGTGTCCTGGAATTCTGGC
GCTCTGACAAGCGGCGTGCACACCTTTCCAGCTGTGCTGCAAAGCAGCGGCCTGTACTCTCT
GAGCAGCGTGGTCACAGTGCCAAGCTCTAGCCTGGGCACCCAGACCTACATCTGCAATGTGA
ACCACAAGCCTAGCAACACCAAGGTGGACAAGAAGGTGGAACCCAAGAGCTGCGACAAGACC
CATACAGGGGGCGGTGGAAGCGGAGGCGGGGGTAGCGGTGGTGGCGGCAGCGGCGGAGGCGG
ATCAGGGGGCGGCGGAAGTGGTGGCGGTGGTTCTCAGGTTCAACTCCAACAGTGGGGCGCTG
GACTGCTGAAACCTAGCGAAACCCTGAGCCTGACATGTGCTGTGTATGGCGGCTCCTTCTCC
GGCTACTATTGGAGCTGGATTCGGCAGCCACCAGGCAAGGGACTCGAGTGGATTGGAGAGAT
CAACCACTCCGGCTCCACCAATTACAATCCATCTCTGCGGTCCCGCGTGACCATCTCCGTGG
ATACCTCTAAGAATCAGTTCTCACTGAAGCTGAGATCCGTGACCGCTGCCGACACTGCCGTG
TATTATTGTGCCCGGGGATTCATGGTTCGAGGGATTATGTGGAATTACTATTATATGGATGT
CTGGGGAAAAGGGACGACCGTGACTGTGTCCCCTGCCTCTACAAAGGGACCCTCCGTGTTTC
CTCTGGCTCCCAGCTCTAAGTCTACCAGCGGAGGAACAGCTGCCCTGGGATGTCTCGTGAAA
GACTACTTCCCCGAACCAGTGACAGTCAGCTGGAACAGCGGAGCCCTGACTTCTGGGGTGCA
CACATTCCCTGCCGTCCTGCAATCTTCTGGCCTGTACAGCCTGTCCAGCGTCGTGACCGTTC
CTTCTAGCTCTCTGGGAACACAGACATATATCTGTAATGTCAATCACAAACCCTCCAATACG
AAGGTCGACAAAAAGGTCGAGCCTAAGTCCTGTGATAAGACCCACACC
SEQ ID NO: 33
Annotated Amino Acid sequence of Synthetic anti-CD52g Fab-Fab-
IgG-Fab-Fab (Heavy Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGFMVRGIMWNYYYMEWWGKGTTVTVSP
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTGGGGSGGGGSGGGGSG
GGGSGGGGSGGGGSQVQLQQWGAGLLKPSETLSLTCAVYGGSFSGYYWSWIRINTGKGLEWI
GEINHSGSTNYNPSLRSRVTISVDTSKNQFSLKLRSVTAADTAVYYCARGFMVRGIMWNYYY
MDVWGKGTTVTVSPASTKGPSVFPLAPSSKSTSGGTAALGCLVKDYFETPVTVSWNSGALTS
GVHTFPAVLQSSGLYSLSSVVTVPSSSLGTQTYICNVNHKPSNTKVDKKVEPKSCDKTHTGG
GGSGGGGSGGGGSGGGGSGGGGSGGGGSQVQLQQWGAGLLKPSETLSLTCAVYGGSFSGYYW
SWIRQPPGKGLEWIGEINHSGSTNYNPSLRSRVTISVDTSKNQFSLKLRSVTAADTAVYYCA
RGFMVRGIMWNYYYMDVWGKGTTVTVSPASTKGPSVFPLAPSSKSTSGGTAALGCLVKDYFP
EPVTVSWNSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSHEDETVKF
NWYVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPVLD
SDGSFFLYSKLTVDKSRWQQGNVFSCSVMHEALHNHYTQKSLSLSPGKGSGGGGSGGGGSGG
GGSGGGGSGGGGSGGGGSQVQLQQWGAGLLKPSETLSLTCAVYGGSFSGYYWSWIRQPPGKG
LEWIGEINHSGSTNYNPSLRSRVTISVDTSKNQFSLKLRSVTAADTAVYYCARGFMVRGIMW
NYYYMDVWGKGTTVTVSPASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKSCDKT
HTGGGGSGGGGSGGGGSGGGGSGGGGSGGGGSQVQLQQWGAGLLKPSETLSLTCAVYGGSFS
GYYWSWIRQPPGKGLEWIGEINHSGSTNYNPSLRSRVTISVDTSKNQFSLKLRSVTAADTAV
YYCARGFMVRGIMWNYYYMDVWGKGTTVTVSPASTKGPSVFPLAPSSKSTSGGTAALGCLVK
DYFPEPVTVSWNSGALTSGVHTFPAVLQSSGLYSLSSVVTVPSSSLGTQTYICNVNHKPSNT
KVDKKVEPKSCDKTHT
SEQ ID NO: 34
Annotated DNA sequence of Synthetic anti-CD52g Fab-Fab-IgG-
Fab-Fab (Light Chain)
AGCAGCGAGCTGACACAGGATCCAGTGGTGTCTGTGGCCCTGGGCCAGACAGTGCGGATTAC
TTGTCAGGGCGACAGCCTGAGAACCTACCACGCCTCTTGGTATCAGCAGAAGCCCAGACAGG
CCCCTGTGCTGGTCATCTACGACGAGAACAACAGACCCAGCGGCATCCCCGATAGATTCAGC
GGCAGCACATCTGGCAATACCGCCAGCCTGACAATCACTGGCGCCCAGGCTGAAGATGAGGC
CGACTACTACTGCAACAGCAGAGACAGGAGCGGCAACCGGCTGGTTTTTGGCGGAGGCACAA
AGCTGACAGTGCTGGGCCAACCTAAGGCCGCTCCTAGCGTGACACTGTTCCCTCCAAGCAGC
GAAGAACTGCAGGCCAACAAGGCCACACTCGTGTGCCTGATCAGCGACTTTTATCCTGGCGC
CGTGACCGTGGCCTGGAAGGCTGATAGTTCTCCTGTGAAGGCCGGCGTGGAAACCACCACAC
CTAGCAAGCAGAGCAACAACAAATACGCCGCCAGCAGCTACCTGAGCCTGACACCTGAGCAG
TGGAAGTCCCACAGATCCTACAGCTGCCAAGTGACCCACGAGGGCAGCACCGTGGAAAAAAC
AGTGGCCCCTACCGAGTGCAGC
SEQ ID NO: 35
Annotated Amino Acid sequence of Synthetic anti-CD52g Fab-Fab-
IgG-Fab-Fab (Light Chain)
SSELTQDPVVSVALGQTVRITCQGDSLRTYHASWYQQKPRQAPVLVIYDENNRPSGIPDRFS
GSTSGNTASLTITGAQAEDEADYYCNSRDSSGNRINFGGGTKLTVLGQPKAAPSVTLFPPSS
EELQANKATLVCLISDFYPGAVTVAWKADSSPVKAGVETTTPSKQSNNKYAASSYLSLTPEQ
WKSHRSYSCQVTHEGSTVEKTVAPTECS
SEQ ID NO: 36
Annotated Amino Acid sequence of Synthetic anti-CD52g Fab
fragment of Fab-Fab-IgG-Fab-Fab (Heavy Chain)
QVQLQQWGAGLLKPSETLSLTCAVYGGSFSGYYWSWIRQPPGKGLEWIGEINHSGSTNYNPS
LRSRVTISVDTSKNQFSLKLRSVTAADTAVYYCARGFMVRGIMWNYYYMDVWGKGTTVTVSP
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHT
SEQ ID NO: 37
Annotated Amino Amid sequence of Synthetic anti-CD52g Fab
fragment of Fab-Fab-IgG-Fab-Fab (Light Chain)
SSELTQDPVVSVALGQTVRITCQGDSLRTYHASWYQQKPRQAPVLVIYDENNRPSGIPDRFS
GSTSGNTASLTITGAQAEDEADYYCNSRDSSGNRLVFGGGTKLTVLGQPKAAPSVTLFPPSS
EELQANKATLVCLISDFYPGAVTVAWKADSSPVKAGVETTTPSKQSNNKYAASSYLSLTPEQ
WKSHRSYSCQVTHEGSTVEKTVAPTECS
SEQ ID NO: 38
Annotated Amino Acid sequence of Synthetic anti-CD52g Fc
fragment of Fab-Fab-IgG-Fab-Fab (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 39
Annotated DNA sequence of anti-klebsiella IgG (Heavy Chain)
GAGGTGCAGCTGGTTGAATCTGGCGGAGGACTGGTTCAGCCTGGCGGATCTCTGAGACTGTC
TTGCGCCGCCAGCTTTAGCCTGACAAGCTACGCCGTGCACATCCACTGGGTTCGACAGGCCC
CTGGCAAAGGCCTTGAATGGGTTGCCAGAGTGATCTGGGCTGGCGGCATCACCCACTACAAT
AGCGCCCTGATGAGCAGATACGCCGACAGCGTGAAGGGCAGATTCACCATCAGCGCCGACAC
CAGCAAGAACACCGCCTACCTGCAGATGAACAGCCTGAGAGCCGAGGACACCGCCGTGTACT
ATTGCGCCAGAGGCAACTGGGCCTTCGACTATTGGGGACAGGGCACCCTGGTCACCGTTAGC
TCTGCCTCTACAAAGGGCCCTAGTGTGTTCCCTCTGGCTCCCAGCAGCAAGTCTACATCTGG
CGGAACAGCCGCTCTGGGCTGCCTGGTCAAGGATTACTTTCCCGAGCCTGTGACCGTGTCCT
GGAATTCTGGCGCTCTGACAAGCGGCGTGCACACCTTTCCAGCTGTGCTGCAAAGCAGCGGC
CTGTACTCTCTGAGCAGCGTGGTCACAGTGCCTAGCTCTAGCCTGGGCACCCAGACCTACAT
CTGCAATGTGAACCACAAGCCTAGCAACACCAAGGTGGACAAGAAGGTGGAACCCAAGAGCT
GCGACAAGACCCACACCTGTCCTCCATGTCCTGCTCCAGAACTGCTCGGCGGACCTTCCGTG
TTCCTGTTTCCTCCAAAGCCTAAGGACACCCTGATGATCAGCAGAACCCCTGAAGTGACCTG
CGTGGTGGTGGATGTGTCCCACGAGGATCCCGAAGTGAAGTTCAATTGGTACGTGGACGGCG
TGGAAGTGCACAACGCCAAGACCAAGCCTAGAGAGGAACAGTACAACAGCACCTACAGAGTG
GTGTCCGTGCTGACCGTGCTGCACCAGGATTGGCTGAACGGCAAAGAGTACAAGTGCAAGGT
GTCCAACAAGGCCCTGCCTGCTCCTATCGAGAAAACCATCAGCAAGGCCAAGGGCCAGCCTA
GGGAACCCCAGGTTTACACACTGCCTCCAAGCCGGGAAGAGATGACCAAGAACCAGGTGTCC
CTGACCTGCCTCGTGAAGGGCTTCTACCCTTCCGATATCGCCGTGGAATGGGAGAGCAATGG
CCAGCCTGAGAACAACTACAAGACAACCCCTCCTGTGCTGGACAGCGACGGCTCATTCTTCC
TGTACAGCAAGCTGACAGTGGACAAGTCCAGATGGCAGCAGGGCAACGTGTTCAGCTGCAGC
GTGATGCACGAGGCCCTGCACAACCACTACACCCAGAAGTCCCTGAGCCTGTCTCCTGGCAA
A
SEQ ID NO: 40
Annotated Amino Acid sequence of anti-klebsiella IgG (Heavy
Chain)
EVQLVESGGGLVQPGGSLRLSCAASFSLTSYAVHIHWVRQAPGKGLEWVARVIWAGGITHYN
SALMSRYADSVKGRFTISADTSKNTAYLQMNSLRAEDTAVYYCARGNWAFDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVFSCS
VMHEALHNHYTQKSLSLSPGK
SEQ ID NO: 41
Annotated Amino Acid sequence of Fab fragment of anti-
klebsiella (Heavy Chain)
EVQLVESGGGLVQPGGSLRLSCAASFSLTSYAVHIHWVRQAPGKGLEWVARVIWAGGITHYN
SALMSRYADSVKGRFTISADTSKNTAYLQMNSLRAEDTAVYYCARGNWAFDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHT
SEQ ID NO: 42
Annotated Amino Acid sequence of Fc fragment of anti-
kiebsiella (Heavy Chain)
PCPAPELLGGPSVFLFETKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 43
DNA sequence of anti-klebsiella (Light Chain)
GACATCCAGATGACACAGAGCCCTAGCAGCCTGTCTGCCAGCGTGGGAGACAGAGTGACCAT
CACCTGTAGAGCCAGCAGCGCCAGATCCAGCGTGTCCTATATTCACGTGGCCTGGTATCAGC
AGAAGCCCGGCAAAGCCCCTAAGCTGCTGATCTACGACACCAGCAAACTGGCCAGCTTCCTG
TACAGCGGCGTGCCCTCTAGATTCAGCGGCAGCAGATCTGGCACCGACTTCACCCTGACCAT
AAGCAGCCTGCAGCCTGAGGACTTCGCCACCTACTACTGCTTTCAAGGCAGCGGCTACCCCT
ACACCTTTGGCCAGGGAACAAAGGTGGAAATCAAGAGAACAGTGGCCGCTCCTAGCGTGTTC
ATCTTCCCACCTTCCGACGAGCAGCTGAAGTCTGGCACAGCCTCTGTCGTGTGCCTGCTGAA
CAACTTCTACCCCAGAGAAGCCAAGGTGCAGTGGAAGGTGGACAACGCCCTGCAGAGCGGCA
ATAGCCAAGAGAGCGTGACCGAGCAGGACAGCAAGGACTCTACCTACAGCCTGAGCAGCACC
CTGACACTGAGCAAGGCCGACTACGAGAAGCACAAAGTGTACGCCTGCGAAGTGACCCACCA
GGGCCTTTCTAGCCCTGTGACCAAGAGCTTCAACCGGGGCGAATGT
SEQ ID NO: 44
Annotated Amino Acid sequence of anti-klebsiella (Light Chain)
DIQMTQSPSSLSASVGDRVTITCRASSARSSVSYIHVAWYQQKPGKAPKLLIYDTSKLASFL
YSGVPSRFSGSRSGTDFTLTISSLQPEDFATYYCFQGSGYPYTFGQGTKVEIKRTVAAPSVF
IFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO: 45
Annotated Amino Acid sequence of Fab fragment of anti-
klebsiella IgG (Light Chain)
DIQMTQSPSSLSASVGDRVTITCRASSARSSVSYTHVAWYQQKPGKAPKLLIYDTSKLASFL
YSGVPSRFSGSRSGTDFTLTISSLQPEDFATYYCFQGSGYPYTFGQGTKVEIKRTVAAPSVF
IFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO: 46
Annotated DNA sequence of Synthetic anti-klebsiella Fab-IgG
(Heavy Chain)
GAGGTGCAGTTGGTTGAATCCGGTGGCGGGTTGGTCCAGCCTGGCGGGAGCCTTCGGCTTAG
TTGTGCCGCATCATTTAGCCTGACATCTTACGCTGTCCATATCCATTGGGTGCGGCAAGCGC
CTGGTAAGGGCCTGGAATGGGTGGCAAGGGTGATATGGGCAGGGGGTATTACGCATTACAAC
TCTGCATTGATGAGTCGGTACGCCGACAGCGTCAAAGGTCGGTTCACCATTTCTGCCGATAC
CTCTAAGAACACAGCCTACCTCCAGATGAACTCACTGCGAGCGGAGGACACTGCTGTGTACT
ATTGCGCCCGCGGCAATTGGGCATTTGACTACTGGGGGCAAGGTACACTCGTAACGGTCTCA
TCTGCCTCTACAAAGGGCCCTAGTGTGTTCCCTCTGGCTCCCAGGAGCAAGTCTACATCTGG
CGGAACAGCCGCTCTGGGCTGCCTGGTCAAGGATTACTTTCCCGAGCCTGTGACCGTGTCCT
GGAATTCTGGCGCTCTGACAAGCGGCGTGCACACCTTTCCAGCTGTGCTGCAAAGGAGCGGC
CTGTACTCTCTGAGGAGCGTGGTCACAGTGCCTAGCTCTAGCCTGGGCACCCAGACCTACAT
CTGCAATGTGAACCACAAGCCTAGCAACACCAAGGTGGACAAGAAGGTGGAACCCAAGAGCT
GCGACAAGACCCACACCGGTGGCGGAGGATCTGGTGGAGGAGGGAGCGGAGGGGGTGGGTCC
GGAGGGGGTGGTTCCGGAGGTGGTGGATCAGGTGGCGGAGGAAGTGAGGTGCAGCTGGTTGA
ATCTGGCGGAGGACTGGTTCAGCCTGGCGGATCTCTGAGACTGTCTTGCGCCGCCAGCTTTA
GCCTGACAAGCTACGCCGTGCACATCCACTGGGTTCGACAGGCCCCTGGCAAAGGCCTTGAA
TGGGTTGCCAGAGTGATCTGGGCTGGCGGCATCACCCACTACAATAGCGCCCTGATGAGGAG
ATACGCCGACAGCGTGAAGGGCAGATTCACCATCAGCGCCGACACCAGCAAGAACACCGCCT
ACCTGCAGATGAACAGCCTGAGAGCCGAGGACACCGCCGTGTACTATTGCGCCAGAGGCAAC
TGGGCCTTCGACTATTGGGGACAGGGCACCCTGGTCACCGTTAGCTCTGCCTCTACAAAGGG
CCCTAGTGTGTTCCCTCTGGCTCCCAGCAGCAAGTCTACATCTGGCGGAACAGCCGCTCTGG
GCTGCCTGGTCAAGGATTACTTTCCCGAGCCTGTGACCGTGTCCTGGAATTCTGGCGCTCTG
ACAAGCGGCGTGCACACCTTTCCAGCTGTGCTGCAAAGGAGCGGCCTGTACTCTCTGAGCAG
CGTGGTCACAGTGCCTAGCTCTAGCCTGGGCACCCAGACCTACATCTGCAATGTGAACCACA
AGCCTAGCAACACCAAGGTGGACAAGAAGGTGGAACCCAAGAGCTGCGACAAGACCCACACC
TGTCCTCCATGTCCTGCTCCAGAACTGCTCGGCGGACCTTCCGTGTTCCTGTTTCCTCCAAA
GCCTAAGGACACCCTGATGATCAGGAGAACCCCTGAAGTGACCTGCGTGGTGGTGGATGTGT
CCCACGAGGATCCCGAAGTGAAGTTCAATTGGTACGTGGACGGCGTGGAAGTGCACAACGCC
AAGACCAAGCCTAGAGAGGAACAGTACAACAGCACCTACAGAGTGGTGTCCGTGCTGACCGT
GCTGCACCAGGATTGGCTGAACGGCAAAGAGTACAAGTGCAAGGTGTCCAACAAGGCCCTGC
CTGCTCCTATCGAGAAAACCATCAGCAAGGCCAAGGGCCAGCCTAGGGAACCCCAGGTTTAC
ACACTGCCTCCAAGCCGGGAAGAGATGACCAAGAACCAGGTGTCCCTGACCTGCCTCGTGAA
GGGCTTCTACCCTTCCGATATCGCCGTGGAATGGGAGAGCAATGGCCAGCCTGAGAACAACT
ACAAGACAACCCCTCCTGTGCTGGACAGCGACGGCTCATTCTTCCTGTACAGCAAGCTGACA
GTGGACAAGTCCAGATGGCAGCAGGGCAACGTGTTCAGCTGCAGCGTGATGCACGAGGCCCT
GCACAACCACTACACCCAGAAGTCCCTGAGCCTGTCTCCTGGCAAA
SEQ ID NO: 47
Annotated Amino Acid sequence of Synthetic anti-klebsiella IgG
Fab-IgG (Heavy Chain)
EVQLVESGGGLVQPGGSLRLSCAASFSLTSYAVHIHWVRQAPGKGLEWVARVIWAGGITHYN
SALMSRYADSVKGRFTISADTSKNTAYLQMNSLRAEDTAVYYCARGNWAFDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTGGGGSGGGGSGGGGS
GGGGSGGGGSGGGGSEVQLVESGGGLVQPGGSLRLSCAASFSLTSYAVHIHWVRQAPGKGLE
WVARVIWAGGITHYNSALMSRYADSVKGRFTISADTSKNTAYLQMNSLRAEDTAVYYCARGN
WAFDYWGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSCDKTHT
CPPCPAPELLGGPSVFLFPPKPECTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFFLYSKLT
VDKSRWQQGNVFSCSVMHEALHNHYTQKSLSLSPGK
SEQ ID NO: 48
Annotated Amino Acid sequence of Synthetic anti-klebsiella Fab
fragment of Fab-IgG (Heavy Chain)
EVQLVESGGGLVQPGGSLRLSCAASFSLTSYAVHIHWVRQAPGKGLEWVARVIWAGGITHYN
SALMSRYADSVKGRFTISADTSKNTAYLQMNSLRAEDTAVYYCARGNWAFDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHT
SEQ ID NO: 49
Annotated Amino Acid sequence of Synthetic anti-klebsiella Fc
fragment of Fab-IgG (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 50
DNA sequence of Synthetic anti-klebsiella Fab-IgG (Light
Chain)
GACATCCAGATGACACAGAGCCCTAGCAGCCTGTCTGCCAGCGTGGGAGACAGAGTGACCAT
CACCTGTAGAGCCAGCAGCGCCAGATCCAGCGTGTCCTATATTCACGTGGCCTGGTATCAGC
AGAAGCCCGGCAAAGCCCCTAAGCTGCTGATCTACGACACCAGCAAACTGGCCAGCTTCCTG
TACAGCGGCGTGCCCTCTAGATTCAGCGGCAGCAGATCTGGCACCGACTTCACCCTGACCAT
AAGCAGCCTGCAGCCTGAGGACTTCGCCACCTACTACTGCTTTCAAGGCAGCGGCTACCCCT
ACACCTTTGGCCAGGGAACAAAGGTGGAAATCAAGAGAACAGTGGCCGCTCCTAGCGTGTTC
ATCTTCCCACCTTCCGACGAGCAGCTGAAGTCTGGCACAGCCTCTGTCGTGTGCCTGCTGAA
CAACTTCTACCCCAGAGAAGCCAAGGTGCAGTGGAAGGTGGACAACGCCCTGCAGAGCGGCA
ATAGCCAAGAGAGCGTGACCGAGCAGGACAGCAAGGACTCTACCTACAGCCTGAGCAGCACC
CTGACACTGAGCAAGGCCGACTACGAGAAGCACAAAGTGTACGCCTGCGAAGTGACCCACCA
GGGCCTTTCTAGCCCTGTGACCAAGAGCTTCAACCGGGGCGAATGT
SEQ ID NO: 51
Annotated Amino Acid sequence of Synthetic anti-klebsiella
Fab-IgG (Light Chain)
DIQMTQSPSSLSASVGDRVTITCRASSARSSVSYTHVAWYQQKPGKAPKLLIYDTSKLASFL
YSGVPSRFSGSRSGTDFTLTISSLQPEDFATYYCFQGSGYPYTFGQGTKVEIKRTVAAPSVF
IFPPSDEQLKSGTASVVCLLNNEYPREAKVQWKVDNALQSGNSQESVTEQDSKDSTYSLSST
LTLSKADYEKHKVYACEVTHQGLSSPVTKSFNRGEC
SEQ ID NO: 52
Annotated Amino Acid sequence of Synthetic anti-klebsiella Fab
fragment of Fab-IgG (Light Chain)
DIQMTQSPSSLSASVGDRVTITCRASSARSSVSYTHVAWYQQKPGKAPKLLTYDTSKLASEL
YSGVPSRFSGSRSGTDFTLTISSLQPEDFATYYCFQGSGYPYTEGQGTKVEIKRTVAAPSVF
IFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO: 53
Annotated DNA sequence of Synthetic anti-klebsiella IgG-Fab
(Heavy Chain)
GAGGTGCAGCTGGTTGAATCTGGCGGAGGACTGGTTCAGCCTGGCGGATCTCTGAGACTGTC
TTGCGCCGCCAGCTTTAGCCTGACAAGCTACGCCGTGCACATCCACTGGGTTCGACAGGCCC
CTGGCAAAGGCCTTGAATGGGTTGCCAGAGTGATCTGGGCTGGCGGCATCACCCACTACAAT
AGCGCCCTGATGAGCAGATACGCCGACAGCGTGAAGGGCAGATTCACCATCAGCGCCGACAC
CAGCAAGAACACCGCCTACCTGCAGATGAACAGCCTGAGAGCCGAGGACACCGCCGTGTACT
ATTGCGCCAGAGGCAACTGGGCCTTCGACTATTGGGGACAGGGCACCCTGGTCACCGTTAGC
TCTGCCTCTACAAAGGGCCCTAGTGTGTTCCCTCTGGCTCCCAGCAGCAAGTCTACATCTGG
CGGAACAGCCGCTCTGGGCTGCCTGGTCAAGGATTACTTTCCCGAGCCTGTGACCGTGTCCT
GGAATTCTGGCGCTCTGACAAGCGGCGTGCACACCTTTCCAGCTGTGCTGCAAAGCAGCGGC
CTGTACTCTCTGAGCAGCGTGGTCACAGTGCCTAGCTCTAGCCTGGGCACCCAGACCTACAT
CTGCAATGTGAACCACAAGCCTAGCAACACCAAGGTGGACAAGAAGGTGGAACCCAAGAGCT
GCGACAAGACCCACACCTGTCCTCCATGTCCTGCTCCAGAACTGCTCGGCGGACCTTCCGTG
TTCCTGTTTCCTCCAAAGCCTAAGGACACCCTGATGATCAGCAGAACCCCTGAAGTGACCTG
CGTGGTGGTGGATGTGTCCCACGAGGATCCCGAAGTGAAGTTCAATTGGTACGTGGACGGCG
TGGAAGTGCACAACGCCAAGACCAAGCCTAGAGAGGAACAGTACAACAGCACCTACAGAGTG
GTGTCCGTGCTGACCGTGCTGCACCAGGATTGGCTGAACGGCAAAGAGTACAAGTGCAAGGT
GTCCAACAAGGCCCTGCCTGCTCCTATCGAGAAAACCATCAGCAAGGCCAAGGGCCAGCCTA
GGGAACCCCAGGTTTACACACTGCCTCCAAGCCGGGAAGAGATGACCAAGAACCAGGTGTCC
CTGACCTGCCTCGTGAAGGGCTTCTACCCTTCCGATATCGCCGTGGAATGGGAGAGCAATGG
CCAGCCTGAGAACAACTACAAGACAACCCCTCCTGTGCTGGACAGCGACGGCTCATTCTTCC
TGTACAGCAAGCTGACAGTGGACAAGTCCAGATGGCAGGAGGGCAACGTGTTCAGCTGCAGC
GTGATGCACGAGGCCCTGCACAACCACTACACCCAGAAGTCCCTGAGCCTGTCTCCTGGCAA
AGGATCCGGTGGCGGAGGATCTGGTGGAGGAGGGAGCGGAGGGGGTGGGTCCGGAGGGGGTG
GTTCCGGAGGTGGTGGATCAGGTGGCGGAGGAAGTGAGGTGCAGTTGGTTGAATCCGGTGGC
GGGTTGGTCCAGCCTGGCGGGAGCCTTCGGCTTAGTTGTGCCGCATCATTTAGCCTGACATC
TTACGCTGTCCATATCCATTGGGTGCGGCAAGCGCCTGGTAAGGGCCTGGAATCGGTGGCAA
GGGTGATATGGGCAGGGGGTATTACGCATTACAACTCTGCATTGATGAGTCGGTACGCCGAC
AGCGTCAAAGGTCGGTTCACCATTTCTGCCGATACCTCTAAGAACACAGCCTACCTCCAGAT
GAACTCACTGCGAGCGGAGGACACTGCTGTGTACTATTGCGCCCGCGGCAATTGGGCATTTG
ACTACTGGGGGCAAGGTACACTCGTAACGGTCTCATCTGCCTCTACAAAGGGCCCTAGTGTG
TTCCCTCTGGCTCCCAGCAGCAAGTCTACATCTGGCGGAACAGCCGCTCTGGGCTGCCTGGT
CAAGGATTACTTTCCCGAGCCTGTGACCGTGTCCTGGAATTCTGGCGCTCTGACAAGCGGCG
TGCACACCTTTCCAGCTGTGCTGCAAAGCAGCGGCCTGTACTCTCTGAGGAGCGTGGTCACA
GTGCCTAGCTCTAGCCTGGGCACCCAGACCTACATCTGCAATGTGAACCACAAGCCTAGCAA
CACCAAGGTGGACAAGAAGGTGGAACCCAAGAGCTGCGACAAGACCCACACC
SEQ ID NO: 54
Annotated Amino Amid sequence of Synthetic anti-klebsiella
IgG-Fab (Heavy Chain)
EVQLVESGGGLVQPGGSLRLSCAASFSLTSYAVHIHWVRQAPGKGLEWVARVIWAGGITHYN
SALMSRYADSVKGRFTISADTSKNTAYLQMNSLRAEDTAVYYCARGNWAFDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTCPPCPAPELLGGPSV
FLFPPKPKDTLMISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLPPSREEMTKNQVS
LTCLVKGFYPSDIAVEWESNGQPENNYKTTPPVLDSDGSFFLYSKLTVDKSRWQQGNVESCS
VMHEALHNHYTQKSLSLSPGKGSGGGGSGGGGSGGGGSGGGGSGGGGSGGGGSEVQLVESGG
GLVQPGGSLRLSCAASPSLTSYAVHIHWVRQAPGKGLEWVARVIWAGGITHYNSALMSRYAD
SVKGRFTISADTSKNTAYLQMNSLRAEDTAVYYCARGNWAFDYWGQGTLVTVSSASTKGPSV
FPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHT
SEQ ID NO: 55
Annotated Amino Acid sequence of Synthetic anti-klebsiella Fab
fragment of IgG-Fab (Heavy Chain)
EVQLVESGGGLVQPGGSLRLSCAASFSLTSYAVHIHWVRQAPGEGLEWVARVIWAGGITHYN
SALMSRYADSVKGRFTISADTSKNTAYLQMNSLRAEDTAVYYCARGNWAFDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHT
SEQ ID NO: 56
Annotated Amino Acid sequence of Synthetic anti-klebsiella Fc
fragment of IgG-Fab (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 57
DNA sequence of Synthetic anti-klebsiella IgG-Fab (Light
Chain)
GACATCCAGATGACACAGAGCCCTAGGAGCCTGTCTGCCAGCGTGGGAGACAGAGTGACCAT
CACCTGTAGAGCCAGGAGCGCCAGATCCAGCGTGTCCTATATTCACGTGGCCTGGTATCAGC
AGAAGCCCGGCAAAGCCCCTAAGCTGCTGATCTACGACACCAGCAAACTGGCCAGCTTCCTG
TACAGCGGCGTGCGCTCTAGATTCAGCGGCAGGAGATCTGGCACCGACTTCACCCTGACCAT
AAGGAGCCTGCAGCCTGAGGACTTCGCCACCTACTACTGCTTTCAAGGCAGCGGCTACCCCT
ACACCTTTGGCCAGGGAACAAAGGTGGAAATCAAGAGAACAGTGGCCGCTCCTAGCGTGTTC
ATCTTCCCACCTTCCGACGAGCAGCTGAAGTCTGGCACAGCCTCTGTCGTGTGCCTGCTGAA
CAACTTCTACCCCAGAGAAGCCAAGGTGCAGTGGAAGGTGGACAACGCCCTGCAGAGCGGCA
ATAGCCAAGAGAGCGTGACCGAGGAGGACAGCAAGGACTCTACCTACAGCCTGAGGAGCACC
CTGACACTGAGCAAGGCCGACTACGAGAAGCACAAAGTGTACGCCTGCGAAGTGACCCACCA
GGGCCTTTCTAGCCCTGTGACCAAGAGCTTCAACCGGGGCGAATGT
SEQ ID NO: 58
Annotated Amino Acid sequence of Synthetic anti-klebsiella
IgG-Fab (Light Chain)
DIQMTQSPSSLSASVGDRVTITCRASSARSSVSYTHVAWYQQKPGKAPKLLTYDTSKLASFL
YSGVPSRFSGSRSGTDFTLTISSLQPEDFATYYCFQGSGYPYTFGQGTKVEIKRTVAAPSVF
IFPPSDEQLKSGTASVVCLLNNFYPREAKVQWKVDNALQSGNSQESVTEQDSKDSTYSLSST
LTLSKADYEKHKVYACEVTHQGLSSPVTKSENRGEC
SEQ ID NO: 59
Annotated Amino Acid sequence of Synthetic anti-klebsiella Fab
fragment of IgG-Fab (Light Chain)
DIQMTQPPSSLSASVGDRVTITCRASSARSSVSYIHVAWYQQKPGKAPKLLTYDTSKLASFL
YSGVPSRFSGSRSGTDFTLTISSLQPEDFATYYCFQGSGYPYTEGQGTKVEIKRTVAAPSVF
IFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO: 60
Annotated DNA sequence of Synthetic anti-klebsiella Fab-IgG-
Fab (Heavy Chain)
GAGGTGCAGTTGGTTGAATCCGGTGGCGGGTTGGTCCAGCCTGGCGGGAGCCTTCGGCTTAG
TTGTGCCGCATCATTTAGCCTGACATCTTACGCTGTCCATATCCATTGGGTGCGGCAAGCGC
CTGGTAAGGGCCTGGAATGGGTGGCAAGGGTGATATGGGCAGGGGGTATTACGCATTACAAC
TCTGCATTGATGAGTCGGTACGCCGACAGCGTCAAAGGTCGGTTCACCATTTCTGCCGATAC
CTCTAAGAACACAGCCTACCTCCAGATGAACTCACTGCGAGCGGAGGACACTGCTGTGTACT
ATTGCGCCCGCGGCAATTGGGCATTTGACTACTGGGGGCAAGGTACACTCGTAACGGTCTCA
TCTGCCTCTACAAAGGGCCCTAGTGTGTTCCCTCTGGCTCCCAGGAGCAAGTCTACATCTGG
CGGAACAGCCGCTCTGGGCTGCCTGGTCAAGGATTACTTTCCCGAGCCTGTGACCGTGTCCT
GGAATTCTGGCGCTCTGACAAGCGGCGTGCACACCTTTCCAGCTGTGCTGCAAAGGAGCGGC
CTGTACTCTCTGAGGAGCGTGGTCACAGTGCCTAGCTCTAGCCTGGGCACCCAGACCTACAT
CTGCAATGTGAACCACAAGCCTAGCAACACCAAGGTGGACAAGAAGGTGGAACCCAAGAGCT
GCGACAAGACCCACACCGGTGGCGGAGGATCTGGTGGAGGAGGGAGCGGAGGGGGTGGGTCC
GGAGGGGGTGGTTCCGGAGGTGGTGGATCAGGTGGCGGAGGAAGTGAGGTGCAGCTGGTTGA
ATCTGGCGGAGGACTGGTTCAGCCTGGCGGATCTCTGAGACTGTCTTGCGCCGCCAGCTTTA
GCCTGACAAGCTACGCCGTGCACATCCACTGGGTTCGACAGGCCCCTGGCAAAGGCCTTGAA
TGGGTTGCCAGAGTGATCTGGGCTGGCGGCATCACCCACTACAATAGCGCCCTGATGAGGAG
ATACGCCGACAGCGTGAAGGGCAGATTCACCATCAGCGCCGACACCAGCAAGAACACCGCCT
ACCTGCAGATGAACAGCCTGAGAGCCGAGGACACCGCCGTGTACTATTGCGCCAGAGGCAAC
TGGGCCTTCGACTATTGGGGACAGGGCACCCTGGTCACCGTTAGCTCTGCCTCTACAAAGGG
CCCTAGTGTGTTCCCTCTGGCTCCCAGGAGCAAGTCTACATCTGGCGGAACAGCCGCTCTGG
GCTGCCTGGTCAAGGATTACTTTCCCGAGCCTGTGACCGTGTCCTGGAATTCTGGCGCTCTG
ACAAGCGGCGTGCACACCTTTCCAGCTGTGCTGCAAAGCAGCGGCCTGTACTCTCTGAGCAG
CGTGGTCACAGTGCCTAGCTCTAGCCTGGGCACCCAGACCTACATCTGCAATGTGAACCACA
AGCCTAGCAACACCAAGGTGGACAAGAAGGTGGAACCCAAGAGCTGCGACAAGACCCACACC
TGTCCTCCATGTCCTGCTCCAGAACTGCTCGGCGGACCTTCCGTGTTCCTGTTTCCTCCAAA
GCCTAAGGACACCCTGATGATCAGGAGAACCCCTGAAGTGACCTGCGTGGTGGTGGATGTGT
CCCACGAGGATCCCGAAGTGAAGTTCAATTGGTACGTGGACGGCGTGGAAGTGCACAACGCC
AAGACCAAGCCTAGAGAGGAACAGTACAACAGCACCTACAGAGTGGTGTCCGTGCTGACCGT
GCTGCACCAGGATTGGCTGAACGGCAAAGAGTACAAGTGCAAGGTGTCCAACAAGGCCCTGC
CTGCTCCTATCGAGAAAACCATCAGCAAGGCCAAGGGCCAGCCTAGGGAACCCCAGGTTTAC
ACACTGCCTCCAAGCCGGGAAGAGATGACCAAGAACCAGGTGTCCCTGACCTGCCTCGTGAA
GGGCTTCTACCCTTCCGATATCGCCGTGGAATGGGAGAGCAATGGCCAGCCTGAGAACAACT
ACAAGACAACCCCTCCTGTGCTGGACAGCGACGGCTCATTCTTCCTGTACAGCAAGCTGACA
GTGGACAAGTCCAGATGGCAGGAGGGCAACGTGTTCAGCTGCAGCGTGATGCACGAGGCCCT
GCACAACCACTACACCCAGAAGTCCCTGAGCCTGTCTCCTGGCAAAGGATCCGGTGGCGGAG
GATCTGGTGGAGGAGGGAGCGGAGGGGGTGGGTCCGGAGGGGGTGGTTCCGGAGGTGGTGGA
TCAGGTGGCGGAGGAAGTGAGGTGCAGTTGGTTGAATCCGGTGGCGGGTTGGTCCAGCCTGG
CGGGAGCCTTCGGCTTAGTTGTGCCGCATCATTTAGCCTGACATCTTACGCTGTCCATATCC
ATTGGGTGCGGCAAGCGCCTGGTAAGGGCCTGGAATGGGTGGCAAGGGTGATATGGGCAGGG
GGTATTACGCATTACAACTCTGCATTGATGAGTCGGTACGCCGACAGCGTCAAAGGTCGGTT
CACCATTTCTGCCGATACCTCTAAGAACACAGCCTACCTCCAGATGAACTCACTGCGAGCGG
AGGACACTGCTGTGTACTATTGCGCCCGCGGCAATTGGGCATTTGACTACTGGGGGCAAGGT
ACACTCGTAACGGTCTCATCTGCCTCTACAAAGGGCCCTAGTGTGTTCCCTCTGGCTCCCAG
CAGCAAGTCTACATCTGGCGGAACAGCCGCTCTGGGCTGCCTGGTCAAGGATTACTTTCCCG
AGCCTGTGACCGTGTCCTGGAATTCTGGCGCTCTGACAAGCGGCGTGCACACCTTTCCAGCT
GTGCTGCAAAGGAGCGGCCTGTACTCTCTGAGGAGCGTGGTCACAGTGCCTAGCTCTAGCCT
GGGCACCCAGACCTACATCTGCAATGTGAACCACAAGCCTAGCAACACCAAGGTGGACAAGA
AGGTGGAACCCAAGAGCTGCGACAAGACCCACACC
SEQ ID NO: 61
Annotated Amino Acid sequence of Synthetic anti-klebsiella
Fab-IgG-Fab (Heavy Chain)
EVQLVESGGGLVQPGGSLRLSCAASFSLTSYAVHIHWVRQAPGKGLEWVARVIWAGGITHMT
SALMSRYADSVKGRFTISADTSKNTAYLQMNSLRAEDTAVYYCARGNWAFDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTGGGGSGGGGSGGGGS
GGGGSGGGGSGGGGSEVQLVESGGGLVQPGGSLRLSCAASFSLTSYAVHIHWVRQAPGKGLE
WVARVIWAGGITHYNSALMSRYADSVKGRFTISADTSKNTAYLQMNSLRAEDTAVYYCARGN
WAFDYWGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEPKSCDKTHT
CPPCPAPELLGGPSVFLFETKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLDSDGSFELYSKLT
VDKSRWQQGNVFSCSVMHEALHNHYTQKSLSLSPGKGSGGGGSGGGGSGGGGSGGGGSGGGG
SGGGGSEVQLVESGGGLVQPGGSLRLSCAASFSLTSYAVHIHWVRQAPGKGLEWVARVIWAG
GITHYNSALMSRYADSVKGRFTISADTSKNTAYLQMNSLRAEDTAVYYCARGNWAFDYWGQG
TLVTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHT
SEQ ID NO: 62
Annotated Amino Acid sequence of Synthetic anti-klebsiella Fab
fragment of Fab-IgG-Fab (Heavy Chain)
EVQLVESGGGLVQPGGSLRLSCAASFSLTSYAVHIRWVRQAPGKGLEWVARVIWAGGITHYN
SALMSRYADSVKGRFTISADTSKNTAYLQMNSLRAEDTAVYYCARGNWAFDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHT
SEQ ID NO: 63
Annotated Amino Acid sequence of Synthetic anti-klebsiella Fc
fragment of Fab-IgG-Fab (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 64
DNA sequence of Synthetic anti-klebsiella Fab-IgG-Fab (Light
Chain)
GACATCCAGATGACACAGAGCCCTAGCAGCCTGTCTGCCAGCGTGGGAGACAGAGTGACCAT
CACCTGTAGAGCCAGCAGCGCCAGATCCAGCGTGTCCTATATTCACGTGGCCTGGTATCAGC
AGAAGCCCGGCAAAGCCCCTAAGCTGCTGATCTACGACACCAGCAAACTGGCCAGCTTCCTG
TACAGCGGCGTGCCCTCTAGATTCAGCGGCAGCAGATCTGGCACCGACTTCACCCTGACCAT
AAGCAGCCTGCAGCCTGAGGACTTCGCCACCTACTACTGCTTTCAAGGCAGCGGCTACCCCT
ACACCTTTGGCCAGGGAACAAAGGTGGAAATCAAGAGAACAGTGGCCGCTCCTAGCGTGTTC
ATCTTCCCACCTTCCGACGAGCAGCTGAAGTCTGGCACAGCCTCTGTCGTGTGCCTGCTGAA
CAACTTCTACCCCAGAGAAGCCAAGGTGCAGTGGAAGGTGGACAACGCCCTGCAGAGCGGCA
ATAGCCAAGAGAGCGTGACCGAGCAGGACAGCAAGGACTCTACCTACAGCCTGAGCAGCACC
CTGACACTGAGCAAGGCCGACTACGAGAAGCACAAAGTGTACGCCTGCGAAGTGACCCACCA
GGGCCTTTCTAGCCCTGTGACCAAGAGCTTCAACCGGGGCGAATGT
SEQ ID NO: 65
Annotated Amino Acid sequence of Synthetic anti-klebsiella
Fab-IgG-Fab (Light Chain)
DIQMTQSPSSLSASVGDRVTITCRASSARSSVSYTHVAWYQQKPGKAPKLLIYDTSKLASFL
YSGVPSRFSGSRSGTDFTLTISSLQPEDFATYYCFQGSGYPYTEGQGTKVEIKRTVAAPSVF
IFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO: 66
Annotated Amino Acid sequence of Synthetic anti-klebsiella Fab
fragment of Fab-IgG-Fab (Light Chain)
DIQMTQPPSSLSASVGDRVTITCRASSARSSVSYIHVAWYQQKPGKAPKLLIYDTSKLASFL
YSGVPSRFSGSRSGTDFTLTISSLQPEDFATYYCFQGSGYPYTFGQGTKVEIKRTVAAPSVF
IFPPSDEQLKSGTASVVCLLNNFYPREAKVQWKVDNALQSGNSQESVTEQDSKDSTYSLSST
LTLSKADYEKHKVYACEVTHQGLSSPVTKSFNRGEC
SEQ ID NO: 67
Annotated DNA sequence of anti-Salmonella LPS IgG (Heavy
Chain)
GAAGTGAAGCTGGTGGAATCTGGCGGCGGACTGGTTCAACCTGGCGGATCTCTGTCTCTGAG
CTGTGCCGCCAGCGGCTTCACCTTCAGCGACTACTACATGACCTGGGTCCGACAGGCCCCTG
GAAAAGCTCCTGAATGGCTGGCCCTGATCCGGAACAAGAGAAACGGCGATACCGCCGAGTAC
AGCGCCTCTGTGAAGGGCAGATTCACCATCAGCCGGGACTACAGCCGGTCCATCCTGCACCT
TCAGATGAACGCCCTGAGAACCGAGGACTCCGCCACCTACTATTGCGTGCGACAAGGCAGAG
GCTACACCCTGGATTATTGGGGCCAGGGCACAAGCGTGACAGTGTCTAGCGCTAGCACCAAG
GGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGC
AGCGTGGTGACCGTGCCCTCCAGCAGCTTGGGCACCCAGACCTACATCTGCAACGTGAATCA
CAAGCCCAGCAACACCAAGGTGGACAAGAAAGTTGAGCCCAAATCTTGTGACAAAACTCACA
CATGCCCACCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGTGGTGGACGT
GAGCCACGAAGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGCGTCCTCACC
GTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGT
ACACCCTGCCCCCATCCCGGGACGAGCTGACCAAGAACCAGGTCAGCCTGACCTGCCTGGTC
AAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTACAGCAAGCTCA
CCGTGGACAAGAGCAGGTGGCAGCAGGGGAACGTCTTCTCATGCTCCGTGATGCATGAGGCT
CTGCACAACCACTACACGCAGAAGAGCCTCTCCCTGTCTCCGGGTAAA
SEQ ID NO: 68
Annotated Amino Acid sequence of anti-Salmonella LPS IgG
(Heavy Chain)
EVKLVESGGGLVQPGGSLSLSCAASGFTFSDYYMTWVRQAPGKAPEWLALIRNKRNGDTAEY
SASVKGRFTISRDYSRSILHLQMNALRTEDSATYYCVRQGRGYTLDYNGQGTSVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEA
LHNHYTQKSLSLSPGK
SEQ ID NO: 69
Annotated Amino Acid sequence of Fab fragment of anti-
Salmonella LPS IgG (Heavy Chain)
EVKLVESGGGLVQPGGSLSLSCAASGFTFSDYYMTWVRQAPGKAPEWLALIRNKRNGDTAEY
SASVKGRFTISRDYSRSILHLQMNALRTEDSATYYCVRQGRGYTLDYWGQGTSVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHT
SEQ ID NO: 70
Annotated Amino Acid sequence of Fc fragment of anti-
Salmonella LPS (Heavy Chain)
CPAPELLGGPSVFLFPPKPKDTLMISRTPEVTCVVVDVSHEDPEVKFNWYVDGVEVHNAKTK
PREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVDK
SRWQQGNVFSCSVMHEALHNHYTQKSLSLSPGK
SEQ ID NO: 71
DNA sequence of anti-Salmonella LPS (Light Chain)
GACATCCAGATGAATCAGAGCCCCAGCAGCCTGTCTGCCAGCCTGGGAGATACCATCAGCAT
CACCTGTCGGGCCAGCCAGAACATCAACATCTGGCTGAGCTGGTATCAGCAGAAACCCGGCA
ACGTGCCCAAGCTGCTGATCTACAAGGCCAGCAATCTGCACACCGGCGTGCCCAGCAGATTT
TCTGGCTCTGGCAGCGGCACCGACTTCACCCTGATCATATCTAGCCTGCAGCCTGAGGATAT
CGCCACCTACTACTGCCTGCAAGGCCAGAGCTACCCCAGAACATTTGGCGGAGGCACCAAGC
TGGAAATCAAGACGGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGT
ACAGTGGAAGGTGGATAACGCCCTCCAATCGGGTAACTCCCAGGAGAGTGTCACAGAGCAGG
ACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAG
CTTCAACAGGGGAGAGTGT
SEQ ID NO: 72
Annotated Amino Acid sequence of anti-Salmonella LPS (Light
Chain)
DIQMNQSPSSLSASLGDTISITCRASQNINIWLSWYQQKPGNVPKLLIYKASNLHTGVPSRF
SGSGSGTDFTLIISSLQPEDIATYYCLQGQSYPRTFGGGTKLEIKTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 73
Annotated Amino Acid sequence of Fab fragment of Synthetic
anti-Salmonella LPS IgG (Light Chain)
DIQMNQSPSSLSASLGDTISITCRASQNINIWLSWYQQKPGNVPKLLIYKASNLHTGVPSRF
SGSGSGTDFTLIISSLQPEDIATYYCLQGQSYPRTFGGGTKLEIKTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 74
Annotated DNA sequence of Synthetic anti-Salmonella LPS Fab-
IgG (Heavy Chain)
GAAGTGAAGCTGGTGGAATCTGGCGGCGGACTGGTTCAACCTGGCGGATCTCTGTCTCTGAG
CTGTGCCGCCAGCGGCTTCACCTTCAGCGACTACTACATGACCTGGGTCCGACAGGCCCCTG
GAAAAGCTCCTGAATGGCTGGCCCTGATCCGGAACAAGAGAAACGGCGATACCGCCGAGTAC
AGCGCCTCTGTGAAGGGCAGATTCACCATCAGCCGGGACTACAGCCCGTCCATCCTGCACCT
TCAGATGAACGCCCTGAGAACCGAGGACTCCGCCACCTACTATTGCGTGCGACAAGGCAGAG
GCTACACCCTGGATTATTGGGGCCAGGGCACAAGCGTGACAGTGTCTAGCGCCAGCACAAAG
GGCCCCTCTGTTTTTCCACTGGCTCCCAGCAGCAAGAGCACAAGCGGAGGAACAGCTGCCCT
GGGATGCCTCGTGAAGGACTACTTCCCTGAACCAGTGACCGTGTCCTGGAACTCTGGCGCTC
TGACTTCTGGGGTCCACACTTTCCCAGCTGTCCTGCAGTCTAGCGGACTGTACTCTCTGAGC
AGCGTGGTCACAGTGCCTAGCTCTAGCCTGGGCACCCAGACCTACATCTGCAATGTGAACCA
CAAGCCTAGCAACACGAAGGTCGACAAAAAGGTGGAACCCAAGAGCTGCGACAAGACCCATA
CAGGCGGCGGAGGATCTGGCGGAGGTGGAAGCGGAGGCGGAGGAAGCGGTGGCGGCGGTAGT
GGCGGTGGTGGTTCAGGCGGTGGCGGATCTGAAGTGAAACTGGTTGAAAGCGGCGGAGGCCT
GGTTCAGCCAGGTGGAAGTCTCTCTCTGTCTTGTGCCGCCTCTGGCTTTACCTTCTCTGATT
ACTATATGACGTGGGTTCGCCAAGCTCCTGGCAAGGCACCAGAATGGCTCGCTCTGATTAGA
AACAAGCGGAATGGCGACACAGCCGAGTATTCCGCCAGCGTGAAAGGCCGGTTCACCATCTC
CAGAGACTACTCCCGCAGCATCCTGCATCTGCAAATGAATGCTCTGCGGACCGAGGACAGCG
CTACCTATTACTGCGTTAGGCAAGGCCGGGGATACACACTGGACTACTGGGGACAAGGCACC
TCCGTGACTGTGTCCTCCGCTAGCACCAAGGGCCCATCGGTCTTCCCCCTGGCACCCTCCTC
CAAGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGG
CACCCAGACCTACATCTGCAACGTGAATCACAAGCCCAGCAACACCAAGGTGGACAAGAAAG
TTGAGCCCAAATCTTGTGACAAAACTCACACATGCCCACCGTGCCCAGCACCTGAACTCCTG
GGGGGACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCAGTACAAC
AGCACGTACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGACGAGCTGACC
AAGAACCAGGTCAGCCTGACCTGCCTGGTCAAAGGCTTCTATCCCAGCGACATCGCCGTGGA
GTGGGAGAGCAATGGGCAGCCGGAGAACAACTACAAGACCACGCCTCCCGTGCTGGACTCCG
ACGGCTCCTTCTTCCTCTACAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTCTC
CCTGTCTCCGGGTAAA
SEQ ID NO: 75
Annotated Amino Acid sequence of Synthetic anti-Salmonella LPS
IgG Fab-IgG (Heavy Chain)
EVKLVESGGGLVQPGGSLSLSCAASGFTFSDYYMTWVRQAPGRAPEWLALIRNKRNGDTAEY
SASVKGRFTISRDYSRSILHLQMNALRTEDSATYYCVRQGRGYTLDYWGQGTSVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTGGGGSGGGGSGGGGSGGGGS
GGGGSGGGGSEVKLVESGGGLVQPGGSLSLSCAASGFTFSDYYMTWVRQAPGKAPEWLALIR
NKRNGDTAEYSASVKGRFTISRDYSRSILHLQMNALRTEDSATYYCVRQGRGYTLDYWGQGT
SVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLFTSRDELT
KNQVSLTCLVKGFYPSDIAVEWESNGQPENNYKTTPPVLDSDGSFFLYSKLTVDKSRWQQGN
VFSCSVMHEALHNHYTQKSLSLSPGK
SEQ ID NO: 76
Annotated Amino Acid sequence of Synthetic anti-Salmonella LPS
Fab fragment of Fab-IgG (Heavy Chain)
EVKLVESGGGLVQPGGSLSLSCAASGFTFSDYYMTWVRQAPGKAPEWLALIRNKRNGDTAEY
SASVKGRFTISRDYSRSILHLQMNALRTEDSATYYCVRQGRGYTLDYWGQGTSVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHT
SEQ ID NO: 77
Annotated Amino Acid sequence of Synthetic anti-Salmonella LPS
Fe fragment of Fab-IgG (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKQKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVESCSVMHEALENHYTQKSLSLSPGK
SEQ ID NO: 78
DNA sequence of Synthetic anti-Salmonella LPS Fab-IgG (Light
Chain)
GACATCCAGATGAATCAGAGCCCCAGCAGCCTGTCTGCCAGCCTGGGAGATACCATCAGCAT
CACCTGTCGGGCCAGCCAGAACATCAACATCTGGCTGAGCTGGTATCAGCAGAAACCCGGCA
ACGTGCCCAAGCTGCTGATCTACAAGGCCAGCAATCTGCACACCGGCGTGCCCAGCAGATTT
TCTGGCTCTGGCAGCGGCACCGACTTCACCCTGATCATATCTAGCCTGCAGCCTGAGGATAT
CGCCACCTACTACTGCCTGCAAGGCCAGAGCTACCCCAGAACATTTGGCGGAGGCACCAAGC
TGGAAATCAAGACGGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGT
ACAGTGGAAGGTGGATAACGCCCTCCAATCGGGTAACTCCCAGGAGAGTGTCACAGAGCAGG
ACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAG
CTTCAACAGGGGAGAGTGT
SEQ ID NO: 79
Annotated Amino Acid sequence of Synthetic anti-Salmonella LPS
Fab-IgG (Light Chain)
DIQMNQSPSSLSASLGDTISITCRASQNINIWLSWYQQKPGNVPKLLIYKASNLHTGVPSRF
SGSGSGTDFTLIISSLQPEDIATYYCLQGQSYPRTFGGGTKLEIKTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 80
Annotated Amino Acid sequence of Synthetic anti-Salmonella LPS
Fab fragment of Fab-IgG (Light Chain)
DIQMNQSPSSLSASLGDTISITCRASQNINIWLSWYQQKPGNVPKLLIYKASNLHTGVPSRF
SGSGSGTDFTLIISSLQPEDIATYYCLQGQSYPRTFGGGTKLEIKTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 81
Annotated DNA sequence of Synthetic anti-Salmonella LPS IgG-
Fab (Heavy Chain)
GAAGTGAAGCTGGTGGAATCTGGCGGCGGACTGGTTCAACCTGGCGGATCTCTGTCTCTGAG
CTGTGCCGCCAGCGGCTTCACCTTCAGCGACTACTACATGACCTGGGTCCGACAGGCCCCTG
GAAAAGCTCCTGAATGGCTGGCCCTGATCCGGAACAAGAGAAACGGCGATACCGCCGAGTAC
AGCGCCTCTGTGAAGGGCAGATTCACCATCAGCCGGGACTACAGCCGGTCCATCCTGCACCT
TCAGATGAACGCCCTGAGAACCGAGGACTCCGCCACCTACTATTGCGTGCGACAAGGCAGAG
GCTACACCCTGGATTATTGGGGCCAGGGCACAAGCGTGACAGTGTCTAGCGCTAGCACCAAG
GGCCCATCGGTCTTCCCCCTGGCACCCTCCTCCAAGAGCACCTCTGGGGGCACAGCGGCCCT
GGGCTGCCTGGTCAAGGACTACTTCCCCGAACCGGTGACGGTGTCGTGGAACTCAGGCGCCC
TGACCAGCGGCGTGCACACCTTCCCGGCTGTCCTACAGTCCTCAGGACTCTACTCCCTCAGC
AGCGTGGTGACCGTGCCCTCCAGCAGCTTGGGCACCCAGACCTACATCTGCAACGTGAATCA
CAAGCCCAGCAACACCAAGGTGGACAAGAAAGTTGAGCCCAAATCTTGTGACAAAACTCACA
CATGCCCACCGTGCCCAGCACCTGAACTCCTGGGGGGACCGTCAGTCTTCCTCTTCCCCCCA
AAACCCAAGGACACCCTCATGATCTCCCGGACCCCTGAGGTCACATGCGTGGTGGTGGACGT
GAGCCACGAAGACCCTGAGGTCAAGTTCAACTGGTACGTGGACGGCGTGGAGGTGCATAATG
CCAAGACAAAGCCGCGGGAGGAGCAGTACAACAGCACGTACCGTGTGGTCAGCGTCCTCACC
GTCCTGCACCAGGACTGGCTGAATGGCAAGGAGTACAAGTGCAAGGTCTCCAACAAAGCCCT
CCCAGCCCCCATCGAGAAAACCATCTCCAAAGCCAAAGGGCAGCCCCGAGAACCACAGGTGT
ACACCCTGCCCCCATCCCGGGACGAGCTGACCAAGAACCAGGTCAGCCTGACCTGCCTGGTC
AAAGGCTTCTATCCCAGCGACATCGCCGTGGAGTGGGAGAGCAATGGGCAGCCGGAGAACAA
CTACAAGACCACGCCTCCCGTGCTGGACTCCGACGGCTCCTTCTTCCTCTACAGCAAGCTCA
CCGTGGACAAGAGCAGGTGGCAGGAGGGGAACGTCTTCTCATGCTCCGTGATGCATGAGGCT
CTGCACAACCACTACACGCAGAAGAGCCTCTCCCTGTCTCCGGGTAAAGGATCCGGCGGAGG
CGGATCTGGTGGCGGAGGTAGTGGCGGCGGAGGTTCAGGTGGTGGTGGTAGCGGAGGTGGCG
GTTCTGGCGGTGGTGGATCTGAAGTGAAGCTGGTGGAATCTGGCGGAGGCCTGGTTCAACCT
GGCGGATCTCTGTCTCTGAGCTGTGCCGCCAGCGGCTTCACCTTCAGCGACTACTACATGAC
CTGGGTCCGACAGGCCCCTGGAAAAGCTCCTGAATGGCTGGCCGTGATCCGGAACAAGAGAA
ACGGCGATACCGCCGAGTACAGCGCCTCTGTGAAGGGCAGATTCACCATCAGCCGGGACTAC
AGCCGGTCCATCCTGCACCTTCAGATGAACGCCCTGAGAACCGAGGATAGCGCCACCTACTA
CTGCGTGCGACAAGGCAGAGGCTACACCCTGGATTATTGGGGCCAGGGCACAAGCGTGACAG
TGTCTAGCGCCTCTACAAAGGGCCCCAGCGTTTTCCCACTGGCTCCTAGGAGCAAGAGCACA
AGCGGAGGAACAGCCGCTCTGGGCTGTCTGGTCAAGGACTACTTTCCCGAGCCTGTGACCGT
GTCCTGGAATTCTGGCGCTCTGACAAGCGGCGTGCACACCTTTCCAGCTGTGCTGCAAAGCA
GCGGCCTGTACTCTCTGAGCAGCGTGGTCACAGTGCCAAGCTCTAGCCTGGGCACCCAGACC
TACATCTGCAATGTGAACCACAAGCCTAGCAACACCAAGGTGGACAAGAAGGTGGAACCCAA
GAGCTGCGACAAGACCCACACC
SEQ ID NO: 82
Annotated Amino Acid sequence of Synthetic anti-Salmonella LPS
IgG-Fab (Heavy Chain)
EVKLVESGGGLVQPGGSLSLSCAASGFTFSDYYMTWVRQAPGKAPEWLALIRNKRNGDTAEY
SASVKGRFTISRDYSRSILHLQMNALRTEDSATYYCVRQGRGYTLDYWGQGTSVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEA
LHNHYTQKSLSLSPGKGSGGGGSGGGGSGGGGSGGGGSGGGGSGGGGSEVKLVESGGGLVQP
GGSLSLSCAASGFTESDYYMTWVRQAPGKAPEWLALIRNKRNGDTAEYSASVKGRFTISRDY
SRSILHLQMNALRTEDSATYYCVRQGRGYTLDYWGQGTSVTVSSASTKGPSVFPLAPSSKST
SGGTAALGCLVKDYFPEPVTVSWNSGALTSGVHTFPAVLQSSGLYSLSSVVTVPSSSLGTQT
YICNVNHKPSNTKVDKKVEPKSCDKTHT
SEQ ID NO: 83
Annotated Amino Acid sequence of Synthetic anti-Salmonella LPS
Fab fragment of IgG-Fab (Heavy Chain)
EVKLVESGGGLVQPGGSLSLSCAASGFTFSDYYMTWVRQAPGKAPEWLALIRNKRNGDTAEY
SASVKGRETISRDYSRSILHLQMNALRTEDSATYYCVRQGRGYTLDYWGQGTSVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHT
SEQ ID NO: 84
Annotated Amino Acid sequence of Synthetic anti-Salmonella LPS
Fc fragment of IgG-Fab (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 85
DNA sequence of Synthetic anti-Salmonella LPS IgG-Fab (Light
Chain)
GACATCCAGATGAATCAGAGCCCCAGCAGCCTGTCTGCCAGCCTGGGAGATACCATCAGCAT
CACCTGTCGGGCCAGCCAGAACATCAACATCTGGCTGAGCTGGTATCAGCAGAAACCCGGCA
ACGTGCCCAAGCTGCTGATCTACAAGGCCAGCAATCTGCACACCGGCGTGCCCAGCAGATTT
TCTGGCTCTGGCAGCGGCACCGACTTCACCCTGATCATATCTAGCCTGCAGCCTGAGGATAT
CGCCACCTACTACTGCCTGCAAGGCCAGAGCTACCCCAGAACATTTGGCGGAGGCACCAAGC
TGGAAATCAAGACGGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGT
ACAGTGGAAGGTGGATAACGCCCTCCAATCGGGTAACTCCCAGGAGAGTGTCACAGAGCAGG
ACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAG
CTTCAACAGGGGAGAGTGT
SEQ ID NO: 86
Annotated Amino Acid sequence of Synthetic anti-Salmonella LPS
IgG-Fab (Light Chain)
DIQMNQSPSSLSASLGDTISITCRASQNINIWLSWYQQKPGNVPKLLIYKASNLHTGVPSRF
SGSGSGTDFTLITSSLQPEDIATYYCLQGQSYPRTFGGGTKLEIKTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 87
Annotated Amino Acid sequence of Synthetic anti-Salmonella LPS
Fab fragment of IgG-Fab (Light Chain)
DIQMNQSPSSLSASLGDTISITCRASQNINIWLSWYQQKPGNVPKLLIYKASNLHTGVPSRF
SGSGSGTDFTLIISSLQPEDIATYYCLQGQSYPRTFGGGTKLEIKTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 88
Annotated DNA sequence of Synthetic anti-Salmonella LPS Fab-
IgG-Fab (Heavy Chain)
GAAGTGAAGCTGGTGGAATCTGGCGGCGGACTGGTTCAACCTGGCGGATCTCTGTCTCTGAG
CTGTGCCGCCAGCGGCTTCACCTTCAGCGACTACTACATGACCTGGGTCCGACAGGCCCCTG
GAAAAGCTCCTGAATGGCTGGCCCTGATCCGGAACAAGAGAAACGGCGATACCGCCGAGTAC
AGCGCCTCTGTGAAGGGCAGATTCACCATCAGCCGGGACTACAGCCGGTCCATCCTGCACCT
TCAGATGAACGCCCTGAGAACCGAGGACTCCGCCACCTACTATTGCGTGCGACAAGGCAGAG
GCTACACCCTGGATTATTGGGGCCAGGGCACAAGCGTGACAGTGTCTAGCGCCAGCACAAAG
GGCCCCTCTGTTTTTCCACTGGCTCCCAGCAGCAAGAGCACAAGCGGAGGAACAGCTGCCCT
GGGATGCCTCGTGAAGGACTACTTCCCTGAACCAGTGACCGTGTCCTGGAACTCTGGCGCTC
TGACTTCTGGGGTCCACACTTTCCCAGCTGTCCTGCAGTCTAGCGGACTGTACTCTCTGAGC
AGCGTGGTCACAGTGCCTAGCTCTAGCCTGGGCACCCAGACCTACATCTGCAATGTGAACCA
CAAGCCTAGCAACACGAAGGTCGACAAAAAGGTGGAACCCAAGAGCTGCGACAAGACCCATA
CAGGCGGCGGAGGATCTGGCGGAGGTGGAAGCGGAGGCGGAGGAAGCGGTGGCGGCGGTAGT
GGCGGTGGTGGTTCAGGCGGTGGCGGATCTGAAGTGAAACTGGTTGAAAGCGGCGGAGGCCT
GGTTCAGCCAGGTGGAAGTCTCTCTCTGTCTTGTGCCGCCTCTGGCTTTACCTTCTCTGATT
ACTATATGACGTGGGTTCGCCAAGCTCCTGGCAAGGCACCAGAATGGCTCGCTCTGATTAGA
AACAAGCGGAATGGCGACACAGCCGAGTATTCCGCCAGCGTGAAAGGCCGGTTCACCATCTC
CAGAGACTACTCCCGCAGCATCCTGCATCTGCAAATGAATGCTCTGCGGACCGAGGACAGCG
CTACCTATTACTGCGTTAGGCAAGGCCGGGGATACACACTGGACTACTGGGGACAAGGCACC
TCCGTGACTGTGTCCTCCGCTAGCACCAAGGGCCCATCGGTCTTCCCCCTGGCACCCTCCTC
CAAGAGCACCTCTGGGGGCACAGCGGCCCTGGGCTGCCTGGTCAAGGACTACTTCCCCGAAC
CGGTGACGGTGTCGTGGAACTCAGGCGCCCTGACCAGCGGCGTGCACACCTTCCCGGCTGTC
CTACAGTCCTCAGGACTCTACTCCCTCAGCAGCGTGGTGACCGTGCCCTCCAGCAGCTTGGG
CACCCAGACCTACATCTGCAACGTGAATCACAAGCCCAGCAACACCAAGGTGGACAAGAAAG
TTGAGCCCAAATCTTGTGACAAAACTCACACATGCCCACCGTGCCCAGCACCTGRACTCCTG
GGGGGACCGTCAGTCTTCCTCTTCCCCCCAAAACCCAAGGACACCCTCATGATCTCCCGGAC
CCCTGAGGTCACATGCGTGGTGGTGGACGTGAGCCACGAAGACCCTGAGGTCAAGTTCAACT
GGTACGTGGACGGCGTGGAGGTGCATAATGCCAAGACAAAGCCGCGGGAGGAGCAGTACAAC
AGCACGTACCGTGTGGTCAGCGTCCTCACCGTCCTGCACCAGGACTGGCTGAATGGCAAGGA
GTACAAGTGCAAGGTCTCCAACAAAGCCCTCCCAGCCCCCATCGAGAAAACCATCTCCAAAG
CCAAAGGGCAGCCCCGAGAACCACAGGTGTACACCCTGCCCCCATCCCGGGACGAGCTGACC
AAGAACCAGGTCAGCCTGACCTGCCTGGTCAAAGGCTTCTATCCCAGCGACATCGCCGTGGA
GTGGGAGAGCAATGGGCAGCCGGAGAACAACTACAAGACCACGCCTCCCGTGCTGGACTCCG
ACGGCTCCTTCTTCCTCTACAGCAAGCTCACCGTGGACAAGAGCAGGTGGCAGCAGGGGAAC
GTCTTCTCATGCTCCGTGATGCATGAGGCTCTGCACAACCACTACACGCAGAAGAGCCTCTC
CCTGTCTCCGGGTAAAGGATCCGGCGGAGGCGGATCTGGTGGCGGAGGTAGTGGCGGCGGAG
GTTCAGGTGGTGGTGGTAGCGGAGGTGGCGGTTCTGGCGGTGGTGGATCTGAAGTGAAGCTG
GTGGAATCTGGCGGAGGCCTGGTTCAACCTGGCGGATCTCTGTCTCTGAGCTGTGCCGCCAG
CGGCTTCACCTTCAGCGACTACTACATGACCTGGGTCCGACAGGCCCCTGGAAAAGCTCCTG
AATGGCTGGCCCTGATCCGGAACAAGAGAAACGGCGATACCGCCGAGTACAGCGCCTCTGTG
AAGGGCAGATTCACCATCAGCCGGGACTACAGCCGGTCCATCCTGCACCTTCAGATGAACGC
CCTGAGAACCGAGGATAGCGCCACCTACTACTGCGTGCGACAAGGCAGAGGCTACACCCTGG
ATTATTGGGGCCAGGGCACAAGCGTGACAGTGTCTAGCGCCTCTACAAAGGGCCCCAGCGTT
TTCCCACTGGCTCCTAGCAGCAAGAGCACAAGCGGAGGAACAGCCGCTCTGGGCTGTCTGGT
CAAGGACTACTTTCCCGAGCCTGTGACCGTGTCCTGGAATTCTGGCGCTCTGACAAGCGGCG
TGCACACCTTTCCAGCTGTGCTGCAAAGCAGCGGCCTGTACTCTCTGAGCAGCGTGGTCACA
GTGCCAAGCTCTAGCCTCGGCACCCAGACCTACATCTGCAATGIGAACCACAAGCCIAGCAA
CACCAAGGTGGACAAGAAGGTGGAACCCAAGAGCTGCGACAAGACCCACACC
SEQ ID NO: 89
Annotated Amino Acid sequence of Synthetic anti-Salmonella LPS
Fab-IgG-Fab (Heavy Chain)
EVKLVESGGGLVQPGGSLSLSCAASGFTFSDYYMTWVRQAPGKAPEWLALIRNKRNGDTAEY
SASVKGRFTISRDYSRSILHLQMNALRTEDSATYYCVRQGRGYTLDYWGQGTSVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTGGGGSGGGGSGGGGSGGGGS
GGGGSGGGGSEVKLVESGGGLVQSGGSLSLSCAASGFIFSDYYMTWVRQAPGKAPEWLALIR
NKRNGDTAEYSASVKGRFTTSRDYSRSILHLQMNALRTEDSATTYCVRQGRGYTLDYWGQGT
SVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTETVLDSDGSFFLYSKLTVDKSRWQQGN
VFSCSVMHEALHNHYTQKSLSLSPGKGSGGGGSGGGGSGGGGSGGGGSGGGGSGGGGSEVKL
VESGGGLVQPGGSLSLSCAASGFTFSDYYMTWVRQAPGKAPEWLALIRNKRNGQTAEYSASV
KGRFTISRDYSRSILHLQMNALRTEDSATYYCVRQGRGYTLDYWGQGTSVTVSSASTKGPSV
FPLAPSSKSTSGGIAALGCLVKDYFPEPVTVSWNSGALTSGVHTFPAVLQSSGLYSLSSVVT
VPSSSLGTQTYICNVNHKPSNTKVDKKVEPKSCDKTHT
SEQ ID NO: 90
Annotated Amino Acid sequence of Synthetic anti-Salmonella LPS
Fab fragment of Fab-IgG-Fab (Heavy Chain)
EVKLVESGGGLVQPGGSLSLSCAASGFTFSDYYMTWVRQAPGKAPEWLALIRNKRNGDTAEY
SASVKGRFTISRDYSRSILHLQMNALRTEDSATYYCVRQGRGYTLDYWGQGTSVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHT
SEQ ID NO: 91
Annotated Amino Acid sequence of Synthetic anti-Salmonella LPS
Fc fragment of Fab-IgG-Fab (Heavy Chain)
PCPAPELLGGPSVFLFETKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVESCSVMHEALHNHYTQKSLSLSPGK
SEQ ID NO: 92
DNA sequence of Synthetic anti-Salmonella LPS Fab-IgG-Fab
(Light Chain)
GACATCCAGATGAATCAGAGCCCCAGCAGCCTGTCTGCCAGCCTGGGAGATACCATCAGCAT
CACCTGTCGGGCCAGCCAGAACATCAACATCTGGCTGAGCTGGTATCAGGAGAAACCCGGCA
ACGTGCCCAAGCTGCTGATCTACAAGGCCAGCAATCTGCACACCGGCGTGCCCAGCAGATTT
TCTGGCTCTGGCAGCGGCACCGACTTCACCCTGATCATATCTAGCCTGCAGCCTGAGGATAT
CGCCACCTACTACTGCCTGCAAGGCCAGAGCTACCCCAGAACATTTGGCGGAGGCACCAAGC
TGGAAATCAAGACGGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGT
ACAGTGGAAGGTGGATAACGCCCTCCAATCGGGTAACTCCCAGGAGAGTGTCACAGAGCAGG
ACAGCAAGGACAGCACCTACAGCCTCAGGAGCACCCTGACGCTGAGCAAAGCAGACTACGAG
AAACACAAAGTCTACGCCTGCGAAGTCACCCATCAGGGCCTGAGCTCGCCCGTCACAAAGAG
CTTCAACAGGGGAGAGTGT
SEQ ID NO: 93
Annotated Amino Acid sequence of Synthetic anti-Salmonella LPS
Fab-IgG-Fab (Light Chain)
DIQMNQSPSSLSASLGDTISITCRASQNINIWLSWYQQKPGNVPKLLTYKASNLHTGVPSRF
SGSGSGTDFTLIISSLQPEDIATYYCLQGQSYPRTFGGGTKLEIKTVAApSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 94
Annotated Amino Acid sequence of Synthetic anti-Salmonella LPS
Fab fragment of Fab-IgG-Fab (Light Chain)
DIQMNQSPSSLSASLGDTISITCRASQNINIWLSWYQQKPGNVPKLLIYKASNLHTGVPSRF
SGSGSGTDFTLIISSLQPEDIATYYCLQGQSYPRTFGGGTKLEIKTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSENRGEC
SEQ ID NO: 95
Annotated DNA sequence of Synthetic anti-Salmonella LPS Fab-
Fab-IgG-Fab-Fab (Heavy Chain)
GAAGTGAAGCTGGTGGAAAGCGGCGGAGGACTGGTTCAACCTGGCGGATCTCTGAGCCTGTC
TTGTGCCGCCAGCGGCTTCACCTTCAGCGACTACTACATGACCTGGGTCCGACAGGCCCCTG
GAAAAGCTCCTGAATGGCTGGCCCTGATCCGGAACAAGAGAAACGGCGATACCGCCGAGTAC
AGCGCCTCTGTGAAGGGCAGATTCACCATCAGCCGGGACTACAGCCGGTCCATCCTGCACCT
TCAGATGAACGCCCTGAGAACCGAGGACTCCGCCACCTACTATTGCGTGCGACAAGGCAGAG
GCTACACCCTGGATTATTGGGGCCAGGGCACAAGCGTGACAGTGTCTAGCGCCAGCACAAAG
GGCCCCTCTGTTTTTCCACTGGCTCCCAGGAGCAAGAGCACAAGCGGAGGAACAGCTGCCCT
GGGATGCCTCGTGAAGGACTACTTCCCTGAACCAGTGACCGTGTCCTGGAACTCTGGCGCTC
TGACATCTGGGGTGCACACATTCCCTGCTGTGCTGCAGAGCAGCGGCCTGTATTCTCTGAGC
AGCGTGGTCACAGTGCCCAGCTCTAGTCTGGGCACCCAGACCTACATCTGCAATGTGAACCA
CAAGCCTAGCAACACGAAGGTCGACAAAAAGGTGGAACCCAAGAGCTGCGACAAGACCCATA
CAGGCGGAGGCGGATCTGGTGGTGGTGGATCTGGCGGTGGCGGTTCAGGTGGCGGCGGTAGC
GGAGGTGGTGGTAGTGGTGGTGGCGGCTCTGAAGTGAAACTCGTCGAATCTGGTGGCGGACT
GGTGCAGCCAGGTGGAAGTCTGTCTCTGAGCTGTGCCGCCTCTGGCTTTACCTTCTCTGATT
ACTATATGACGTGGGTTCGCCAAGCTCCTGGCAAGGCACCAGAATGGCTCGCTCTGATTAGA
AACAAGCGGAATGGCGACACAGCCGAGTATTCCGCCAGCGTGAAAGGCCGGTTCACCATCTC
CAGAGACTACTCCCGCAGCATCCTGCATCTGCAAATGAATGCTCTGCGGACCGAGGACAGCG
CTACCTATTACTGCGTTAGGCAAGGCCGGGGATACACACTGGACTACTGGGGACAAGGCACC
TCCGTGACTGTGTCCTCTGCCTCTACCAAGGGACCCAGCGTGTTCCCACTTGCACCTAGCAG
CAAGTCTACCAGCGGCGGAACTGCCGCTCTCGGATGCCTGGTCAAAGATTATTTCCCCGAAC
CTGTCACCGTCAGCTGGAATAGCGGAGCCCTTACCAGCGGAGTGCATACTTTCCCTGCCGTC
CTCCAGTCATCCGGGCTGTATAGTCTGTCCTCCGTGGTTACCGTGCCAAGCAGCTCTCTGGG
AACACAGACATATATCTGTAATGTCAATCACAAACCCTCCAACACAAAAGTGGACAAAAAAG
TCGAGCCGAAGTCCTGTGATAAGACACACACTGGCGGCGGAGGTTCTGGCGGAGGTGGAAGC
GGAGGCGGTGGCTCAGGCGGCGGTGGCAGTGGCGGAGGCGGTAGCGGCGGAGGCGGTTCTGA
AGTTAAGCTGGTTGAGTCCGGCGGTGGCCTTGTGCAGCCTGGTGGTTCTCTCTCTCTGTCCT
GTGCTGCCTCCGGATTCACCTTTTCCGATTATTACATGACATGGGTTCGACAAGCACCAGGG
AAAGCCCCAGAGTGGCTGGCACTCATCAGAAACAAACGCAACGGGGACACCGCCGAATACTC
TGCCAGTGTCAAAGGCAGGTTTACAATCAGCAGGGATTACTCTCGGAGCATTCTCCACCTCC
AAATGAACGCACTCCGCACAGAGGATAGCGCCACTTACTACTGTGTCCGGCAAGGACGGGGC
TATACCCTCGATTACTGGGGTCAAGGGACATCTGTGACCGTCAGTTCTGCTAGCACCAAGGG
CCCATCGGTCTTCCCCCTGGCACCCTCCTCCAAGAGCACCTCTGGGGGCACAGCGGCCCTGG
GCTGCCTGGTCAAGGACTACTTCCCCGAACCGGTGACGGTGTCGTGGAACTCAGGCGCCCTG
ACCAGCGGCGTGCACACCTTCCCGGCTGTCCTACAGTCCTCAGGACTCTACTCCCTCAGCAG
CGTGGTGACCGTGCCCTCCAGCAGCTTGGGCACCCAGACCTACATCTGCAACGTGAATCACA
AGCCCAGCAACACCAAGGTGGACAAGAAAGTTGAGCCCAAATCTTGTGACAAAACTCACACA
TGCCCACCGTGCCCAGCACCTGAACTCCTGGGGGGACCGTCAGTCTTCCTCTTCCCCCCAAA
ACCCAAGGACACCCTCATGATCTCCCGGACCCCTGAGGTCACATGCGTGGTGGTGGACGTGA
GCCACGAAGACCCTGAGGTCAAGTTCAACTGGTACGTGGACGGCGTGGAGGTGCATAATGCC
AAGACAAAGCCGCGGGAGGAGCAGTACAACAGCACGTACCGTGTGGTCAGCGTCCTCACCGT
CCTGCACCAGGACTGGCTGAATGGCAAGGAGTACAAGTGCAAGGTCTCCAACAAAGCCCTCC
CAGCCCCCATCGAGAAAACCATCTCCAAAGCCAAAGGGCAGCCCCGAGAACCACAGGTGTAC
ACCCTGCCCCCATCCCGGGACGAGCTGACCAAGAACCAGGTCAGCCTGACCTGCCTGGTCAA
AGGCTTCTATCCCAGCGACATCGCCGTGGAGTGGGAGAGCAATGGGCAGCCGGAGAACAACT
ACAAGACCACGCCTCCCGTGCTGGACTCCGACGGCTCCTTCTTCCTCTACAGCAAGCTCACC
GTGGACAAGAGCAGGTGGCAGCAGGGGAACGTCTTCTCATGCTCCGTGATGCATGAGGCTCT
GCACAACCACTACACGCAGAAGAGCCTCTCCCTGTCTCCGGGTAAAGGATCCGGCGGAGGCG
GATCTGGCGGCGGAGGTAGTGGCGGCGGAGGTTCAGGTGGTGGTGGTAGCGGAGGTGGCGGT
TCTGGCGGTGGTGGAAGCGAAGTGAAGCTGGTGGAATCTGGCGGAGGCCTGGTTCAACCTGG
CGGATCTCTGTCTCTGAGCTGTGCCGCCAGCGGCTTCACCTTCAGCGACTACTACATGACCT
GGGTCCGACAGGCCCCTGGAAAAGCTCCTGAATGGCTGGCCCTGATCCGGAACAAGAGAAAC
GGCGATACCGCCGAGTACAGCGCCTCTGTGAAGGGCAGATTCACCATCAGCCGGGACTACAG
CCGGTCCATCCTGCACCTTCAGATGAACGCCCTGAGAACCGAGGATAGCGCCACCTACTACT
GCGTGCGACAAGGCAGAGGCTACACCCTGGATTATTGGGGCCAGGGCACAAGCGTGACAGTG
TCTAGCGCCTCTACAAAGGGCCCCAGCGTTTTCCCACTGGCTCCTAGCAGCAAGAGCACAAG
CGGAGGAACAGCCGCTCTGGGCTGTCTGGTCAAGGACTACTTTCCCGAGCCTGTGACCGTGT
CCTGGAATTCTGGCGCTCTGACAAGCGGCGTGCACACCTTTCCAGCTGTGCTGCAAAGCAGC
GGCCTGTACTCTCTGAGCAGCGTGGTCACAGTGCCAAGCTCTAGCCTGGGCACCCAGACCTA
CATCTGCAATGTGAACCACAAGCCTAGCAACACCAAGGTGGACAAGAAGGTGGAACCCAAGA
GCTGCGACAAGACCCATACAGGCGGTGGCGGAAGCGGAGGCGGTGGCTCAGGTGGCGGAGGT
TCTGGCGGAGGCGGCAGTGGTGGTGGTGGCAGTGGTGGCGGAGGATCTGAAGTCAAACTGGT
CGAAAGCGGAGGTGGACTGGTTCAGCCAGGTGGAAGCCTGTCTCTGTCTTGTGCCGCTTCCG
GCTTTACCTTCTCTGATTACTATATGACGTGGGTTCGCCAAGCTCCTGGCAAGGCACCAGAA
TGGCTCGCTCTGATTAGAAACAAGCGGAATGGCGACACAGCCGAGTATTCCGCCAGCGTGAA
AGGCCGGTTCACCATCTCCAGAGACTACTCCCGCAGCATCCTGCATCTGCAAATGAATGCTC
TGCGGACCGAGGACTCCGCCACATATTACTGTGTCAGACAAGGCCGGGGATACACACTCGAC
TACTGGGGACAGGGAACCTCCGTGACTGTGTCCTCTGCCAGCACAAAGGGGCCCTCCGTGTT
TCCTCTGGCTCCAAGCTCCAAGTCTACCAGCGGTGGAACTGCTGCCCTGGGATGCCTCGTGA
AGGATTACTTCCCAGAACCAGTGACAGTCAGCTGGAACAGCGGAGCCCTGACTTCTGGGGTG
CACACATTCCCTGCCGTCCTGCAATCTTCTGGCCTGTACAGCCTGTCCTCCGTCGTGACCGT
TCCTTCTAGCTCTCTGGGAACACAGACATATATCTGTAATGTCAATCACAAACCCTCCAATA
CGAAGGTCGACAAAAAGGTCGAGCCTAAGTCCTGTGATAAGACCCACACC
SEQ ID NO: 96
Annotated Amino Acid sequence of Synthetic anti-Salmonella LPS
Fab-Fab-IgG-Fab-Fab (Heavy Chain)
EVKLVESGGGLVQPGGSLSLSCAASGFTFSDYYMTWVRQAPGKAPEWLALIRNKRNGDTAEY
SASVKGRFTISRDYSRSILHLQMNALRTEDSATYYCVRQGRGYTLDYNGQGTSVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTGGGGSGGGGSGGGGSGGGGS
GGGGSGGGGSEVKLVESGGGLVQSGGSLSLSCAASGFTFSDYYMTWVRQAPGKAPEWLALIR
NKRNGDTAEYSASVKGRFTISRDYSRSILHLQMNALRTEDSATYYCVRQGRGYTLDYWGQCT
SVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTGGGGSGGGGS
GGGGSGGGGSGGGGSGGGGSEVKLVESGGGLVQSGGSLSLSCAASGFTFSDYYMTWVRQAPG
KAPEWLALIRNKRNGDTAEYSASVKGRFTISRDYSRSILHLQMNALRTEDSATYYCVRQGRG
YTLDYWGQGTSVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEAKSCDKTHT
CPPCPAPELLGGPSVFLFPPKPKDTLMISRTPEVTCVVVDVSHEDPEVKFNWYVDGVEVHNA
KTKPREEQYNSTYRVVSVLTVLHQDWLNGKEYKCKVSNKALPAPIEKTISKAKGQPREPQVY
TLPPSRDELTKNQVSLTCLVKGEYPSDIAVEWESNGQPENNYKTTPPVLDSDGSFFLYSKLT
VDKSRWQQGNVFSCSVMHEALHNHYTQKSLSLSPGKGSGGGGSGGGGSGGGGSGGGGSGGGG
SGGGGSEVKLVESGGGLVQPGGSLSLSCAASGFTFSDYYMTWVRQAPGKAPEWLALIRNKRN
GDTAEYSASVKGRFTISRDYSRSILHLQMNALRTEDSATYYCVRQGRGYTLDYWGQGTSVTV
SSASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTGGGGSGGGGSGGGG
SGGGGSGGGGSGGGGSEVKLVESGGGLVQSGGSLSLSCAASGFTFSDYYMTWVRQAPGKAPE
WLALIRNKRNGDTAEYSASVKGRFTISRDYSRSILHLQMNALRTEDSATYYCVRQGRGYTLD
YWGQGTSVTVSSASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEMSCDKTHT
SEQ ID NO: 97
Annotated Amino Acid sequence of Synthetic anti-Salmonella LPS
Fab fragment of Fab-Fab-IgG-Fab-Fab (Heavy Chain)
EVKLVESGGGLVQPGGSLSLSCAASGFTFSDYYMTWVRQAPGKAPEWLALIRNKRNGDTAEY
SASVKGRFTISRDYSRSILHLQMNALRTEDSATYYCVRQGRGYTLDYWGQGTSVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHT
SEQ ID NO: 98
Annotated Amino Acid sequence of Synthetic anti-Salmonella LPS
Fc fragment of Fab-Fab-IgG-Fab-Fab (Heavy Chain)
PCPAPELLGGPSVFLFETKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVESCSVMHEALHNHYTQKSLSLSPGK
SEQ ID NO: 99
DNA sequence of Synthetic anti-Salmonella LPS Fab-Fab-IgG-Fab-
Fab (Light Chain)
GACATCCAGATGAATCAGAGCCCCAGCAGCCTGTCTGCCAGCCTGGGAGATACCATCAGCAT
CACCTGTCGGGCCAGCCAGAACATCAACATCTGGCTGAGCTGGTATCAGCAGAAACCCGGCA
ACGTGCCCAAGCTGCTGATCTACAAGGCCAGCAATCTGCACACCGGCGTGCCCAGCAGATTT
TCTGGCTCTGGCAGCGGCACCGACTTCACCCTGATCATATCTAGCCTGCAGCCTGAGGATAT
CGCCACCTACTACTGCCTGCAAGGCCAGAGCTACCCCAGAACATTTGGCGGAGGCACCAAGC
TGGAAATCAAGACGGTGGCTGCACCATCTGTCTTCATCTTCCCGCCATCTGATGAGCAGTTG
AAATCTGGAACTGCCTCTGTTGTGTGCCTGCTGAATAACTTCTATCCCAGAGAGGCCAAAGT
ACAGTGGAAGGTGGATAACGCCCTCCAATCGGGTAACTCCCAGGAGAGTGTCACAGAGCAGG
ACAGCAAGGACAGCACCTACAGCCTCAGCAGCACCCTGACGCTGAGCAAAGCAGACTACGAG
AAACACAAAGTCTACGCCTGCGAAGTCACCCATCAGGGCCTGAGCTCGCCCGTCACAAAGAG
CTTCAACAGGGGAGAGTGT
SEQ ID NO: 100
Annotated Amino Amid sequence of Synthetic anti-Salmonella LPS
Fab-Fab-IgG--Fab-Fab (Light Chain)
DIQMNQSPSSLSASLGDTISITCRASQNINIWLSWYQQKPGNVPKLLIYKASNLHTGVPSRF
SGSGSGTDFTLIISSLQPEDIATYYCLQGQSYTRTFGGGTKLEIKTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 101
Annotated Amino Acid sequence of Synthetic anti-Salmonella LPS
Fab fragment of Fab-Fab-IgG-Fab-Fab (Light Chain)
DIQMNQSPSSLSASLGDTISITCRASQNINIWLSWYQQKPGNVPKLLIYKASNLHTGVPSRF
SGSGSGTDFTLIISSLQPEDIATYYCLQGQSYPRTFGGGTKLEIKTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 102
Annotated DNA sequence of anti-Gonorrhea (2C7) IgG (Heavy
Chain)
GAGGTTCAGCTGCAGCAGTCTGGACCTGAGCTGGTCAAGCCTGGCAGCAGCGTGAAGATCAG
CTGTAAAGGCAGCGGCTACACCTTCACCGACTACAACATGGAATGGGTCAAGCAGAGCCACG
GCAAGAGCCTGGAATGGATCGGCGTGATCAACCCCAACAACCGGTTCACCAGCTACAACCAG
AACTTCAGAGGCAAGGCCACACTGACCGTGGACAAGAGCAGCAGCACCGCCTACATGGATCT
GAGAAGCCTGACCAGCGAGGACAGCGCCGTGTATTTTTGTGCCGGCAGCCGGTGGTATCAGT
ACGACTATTGGGGCCAGGGCACAACCCTGACCGTTAGCTCTGCTAGCACCAAGGGCCCATCG
GTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAG
CAACACCAAGGTGGACAAGAAAGTTGAGCCCAAATCTTGTGACAAAACTCACACATGCCCAC
CGTGCCCAGCACCTGAACTCCTGGGGGGACCGTCAGTCTTCCTCTTCCCCCCAAAACCCAAG
GACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAA
AGCCGCGGGAGGAGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGCCCTCCCAGCCCC
CATCGAGAAAACCATCTCCAAAGCCAAAGGGCAGCCCCGAGAACCACAGGTGTACACCCTGC
CCCCATCCCGGGACGAGCTGACCAAGAACCAGGTCAGCCTGACCTGCCTGGTCAAAGGCTTC
TATCCCAGCGACATCGCCGTGGAGTGGGAGAGCAATGGGCAGCCGGAGAACAACTACAAGAC
CACGCCTCCCGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTAAA
SEQ ID NO: 103
Annotated Amino Acid sequence of anti-Gonorrhea (2C7) IgG
(Heavy Chain)
EVQLQQSGPELVKPGSSVKISCKGSGYTFTDYNMEWVKQSHGKSLEWIGVINPNNRFTSYNQ
NFRGKATLTVDKSSSTAYMDLRSLTSEDSAVYFCAGSRWYQYDYWGQGTTLTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLH
QDWINGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVFSCSVMHEALHN
HYTQKSLSLSPGK
SEQ ID NO: 104
Annotated Amino Acid sequence of Fab fragment of anti-
Gonorrhea (2C7) (Heavy Chain)
EVQLQQSGPELVKPGSSVKISCKGSGYTFTDYNMEWVKQSHGKSLEWIGVINPNNRFTSYNQ
NERGKATLTVDKSSSTAYMDLRSLTSEDSAVYFCAGSRWYQYDYWGQGTTLTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHT
SEQ ID NO: 105
Annotated Amino Acid sequence of Fc fragment of anti-Gonorrhea
(2C7) (Heavy Chain)
PCPAPELLGGPSVELFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 106
DNA sequence of anti-Gonorrhea (2C7) (Light Chain)
CAGGTGGTGGTCACACAAGAGAGCGCCCTGACAACAAGCCCTGGCGAGACAGTGACCCTGAC
CTGCAGATCTTCTACAGGCGCCGTGACCACCTCCAACTACGCCAATTGGGTGCAAGAGAAGC
CCGACCACCTGTTCACAGGCCTGATCGGCGGCATCAACAATAGAGCACCTGGCGTGCCAGCC
AGATTCAGCGGATCTCTGATCGGAGACAAGGCCGCACTGACAATCACAGGCGCCCAGACAGA
GGACGAGGCCATCTACTTTTGCGCCCTGTGGTACAGCAACCACTGGGTTTTCGGCGGAGGCA
CCAAGCTGACAGTTCTGGGCCAACCTAAGGCCGCTCCTAGCGTGACACTGTTCCCTCCAAGC
AGCGAAGAACTGCAGGCCAACAAGGCCACACTCGTGTGCCTGATCAGCGACTTTTATCCTGG
CGCCGTGACCGTGGCCTGGAAGGCTGATAGTTCTCCTGTGAAGGCCGGCGTGGAAACCACCA
CACCTAGCAAGCAGAGCAACAACAAATACGCCGCCAGCAGCTACCTGAGCCTGACACCTGAG
CAGTGGAAGTCCCACAGATCCTACAGCTGCCAAGTGACCCACGAGGGCAGCACCGTGGAAAA
AACAGTGGCCCCTACCGAGTGCAGC
SEQ ID NO: 107
Annotated Amino Acid sequence of anti-Gonorrhea (2C7) (Light
Chain)
QVVVTQESALTTSPGETVTLTCRSSTGAVTTSNYANWVQEKPDHLFTGLIGGINNRAPGVPA
RFSGSLIGDKAALTITGAQTEDEATYFCALWYSNHWVFGGGTKLTVLGQPKAAPSVTLFPPS
SEELQANKATLVCLISDFYPGAVTVAWKADSSPVKAGVETTTPSKQSNNKYAASSYLSLTPE
QWKSHRSYSCQVTHEGSTVEKTVAPTECS
SEQ ID NO: 108
Annotated Amino Acid sequence of Fab fragment of anti-
Gonorrhea (2C7) IgG (Light Chain)
QVVVTQESALTTSPGETVTLTCRSSTGAVTTSNYANWVQEKPDHLFTGLIGGINNRAPGVPA
RFSGSLIGDKAALTITGAQTEDEAIYFCALWYSNHWVFGGGTKLTVLGQPKAAPSVTLFPPS
SEELQANKATLVCLISDFYPGAVTVAWKADSSPVKAGVETTTPSKQSNNKYAASSYLSLTPE
QWKSHRSYSCQVTHEGSTVEKTVAPTECS
SEQ ID NO: 109
Annotated DNA sequence of Synthetic anti-Gonorrhea (2C7) Fab-
IgG (Heavy Chain)
GAGGTACAACTGCAACAGAGTGGCCCCGAGCTTGTGAAGCCAGGGTCCAGCGTGAAGATTTC
TTGCAAGGGAAGTGGGTACACGTTCACGGACTACAACATGGAGTGGGTGAAACAAAGTCACG
GTAAATCCTTGGAGTGGATCGGAGTTATCAACCCAAACAACCGATTTACTAGCTACAACCAG
AATTTCAGGGGGAAGGCAACACTCACCGTCGACAAATCCTCTTCTACGGCATATATGGATCT
CCGCTCACTTACTAGCGAGGACTCTGCAGTCTATTTTTGCGCGGGGAGCCGATGGTATCAAT
ACGACTATTGGGGTCAAGGTACAACGCTTACTGTTAGCTCAGCTAGCACCAAGGGCCCATCG
GTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAG
CAACACCAAGGTGGACAAGAAAGTTGAGCCCAAATCTTGTGACAAAACTCACACAGGTGGCG
GAGGATCTGGTGGAGGAGGGAGCGGAGGGGGTGGGTCCGGAGGGGGTGGTTCCGGAGGTGGT
GGATCAGGTGGCGGAGGAAGTGAGGTTCAGCTGCAGCAGTCTGGACCTGAGCTGGTCAAGCC
TGGCAGCAGCGTGAAGATCAGCTGTAAAGGCAGCGGCTACACCTTCACCGACTACAACATGG
AATGGGTCAAGCAGAGCCACGGCAAGAGCCTGGAATGGATCGGCGTGATCAACCCCAACAAC
CGGTTCACCAGCTACAACCAGAACTTCAGAGGCAAGGCCACACTGACCGTGGACAAGAGCAG
CAGCACCGCCTACATGGATCTGAGAAGCCTGACCAGCGAGGACAGCGCCGTGTATTTTTGTG
CCGGCAGCCGGTGGTATCAGTACGACTATTGGGGCCAGGGCACAACCCTGACCGTTAGCTCT
GCTAGCACCAAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAGGACTC
TACTCCCTCAGCAGCGTGGTGACCGTGCCCTCCAGCAGCTTGGGCACCCAGACCTACATCTG
CAACGTGAATCACAAGCCCAGCAACACCAAGGTGGACAAGAAAGTTGAGCCCAAATCTTGTG
ACAAAACTCACACATGCCCACCGTGCCCAGCACCTGAACTCCTGGGGGGACCGTCAGTCTTC
CTCTTCCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACGGCGTGG
AGGTGCATAATGCCAAGACAAAGCCGCGGGAGGAGCAGTACAACAGCACGTACCGTGTGGTC
AGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAG
AACCACAGGTGTACACCCTGCCCCCATCCCGGGACGAGCTGACCAAGAACCAGGTCAGCCTG
ACCTGCCTGGTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCTTCTTCCTCT
ACAGCAAGCTCACCGTGGACAAGAGGAGGTGGCAGCAGGGGAACGTCTTCTCATGCTCCGTG
ATGCATGAGGCTCTGCACAACCACTACACGCAGAAGAGCCTCTCCCTGTCTCCGGGTAAA
SEQ ID NO: 110
Annotated Amino Acid sequence of Synthetic anti-Gonorrhea
(2C7) Fab-IgG (Heavy Chain)
EVQLQQSGPELVKPGSSVKISCKGSGYTFTDYNMEWVKQSHGKSLEWIGVINPNNRFTSYNQ
NFRGKATLTVDKSSSTAYMDLRSLTSEDSAVYFCAGSRWYQYDYWGQGTTLTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTGGGGSGGGGSGGGGSGGGGSGGG
GSGGGGSEVQLQQSGPELVKPGSSVKISCKGSGYTFTDYNMEWVKQSHGKSLEWIGVINPNN
RFTSYNQNFRGKATLTVDKSSSTAYMDLRSLTSEDSAVYFCAGSRWYQYDYWGQGTTLTVSS
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVF
LFPPKPKDTLMISRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLETSRDELTKNQVSL
TCLVKGFYPSDIAVEWESNGQPENNYKTTPPVLDSDGSFFLYSKLTVDKSRWQQGNVFSCSV
MHEALHNHYTQKSLSLSPGK
SEQ ID NO: 111
Annotated Amino Acid sequence of Synthetic anti-Gonorrhea
(2C7) Fab fragment of Fab-IgG (Heavy Chain)
EVQLQQSGPELVKPGSSVKISCKGSGYTFTDYNMEWVKQSHGKSLEWIGVINPNNRFTSYNQ
NFRGKATLTVDKSSSTAYMDLRSLTSEDSAVYFCAGSRWYQYDYWGQGTTLTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHT
SEQ ID NO: 112
Annotated Amino Acid sequence of Synthetic anti-Gonorrhea
(2C7) Fc fragment of Fab-IgG (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 113
DNA sequence of Synthetic anti-Gonorrhea (2C7) Fab-IgG (Light
Chain)
CAGGTGGTGGTCACACAAGAGAGCGCCCTGACAACAAGCCCTGGCGAGACAGTGACCCTGAC
CTGCAGATCTTCTACAGGCGCCGTGACCACCTCCAACTACGCCAATTGGGTGCAAGAGAAGC
CCGACCACCTGTTCACAGGCCTGATCGGCGGCATCAACAATAGAGCACCTGGCGTGCCAGCC
AGATTCAGCGGATCTCTGATCGGAGACAAGGCCGCACTGACARTCACAGGCGCCCAGACAGA
GGACGAGGCCATCTACTTTTGCGCCCTGTGGTACAGCAACCACTGGGTTTTCGGCGGAGGCA
CCAAGCTGACAGTTCTGGGCCAACCTAAGGCCGCTCCTAGCGTGACACTGTTCCCTCCAAGC
AGCGAAGAACTGCAGGCCAACAAGGCCACACTCGTGTGCCTGATCAGCGACTTTTATCCTGG
CGCCGTGACCGTGGCCTGGAAGGCTGATAGTTCTCCTGTGAAGGCCGGCGTGGAAACCACCA
CACCTAGCAAGCAGAGCAACAACAAATACGCCGCCAGCAGCTACCTGAGCCTGACACCTGAG
CAGTGGAAGTCCCACAGATCCTACAGCTGCCAAGTGACCCACGAGGGCAGCACCGTGGAAAA
AACAGTGGCCCCTACCGAGTGCAGC
SEQ ID NO: 114
Annotated Amino Acid sequence of Synthetic anti-Gonorrhea
(2C7) Fab-IgG (Light Chain)
QVVVTQESALTTSPGETVTLTCRSSTGAVTTSNYANWVQEKPDHLFTGLIGGINNRAPGVPA
RFSGSLIGDKAALTITGAQTEDEAIYFCALWYSNHWVEGGGTKLTVLGQPKAAPSVTLFPPS
SEELQANKATLVCLISDFYPGAVTVAWKADSSPVKAGVETTTPSKQSNNKYAASSYLSLTPE
QWKSHRSYSCQVTHEGSTVEKTVAPTECS
SEQ ID NO: 115
Annotated Amino Acid sequence of Synthetic anti-Gonorrhea
(2C7) Fab fragment of Fab-IgG (Light Chain)
QVVVTQESALTTSPGETVTLTCRSSTGAVTTSNYANWVQEKPDHLFTGLIGGINNRAPGVPA
RFSGSLIGDKAALTITGAQTEDEAIYFCALWYSNHWVFGGGTKLTVLGQPKAAPSVTLEPPS
SEELQANKATINCLISDFYPGAVTVAWKADSSPVKAGVETTTPSKQSNNKYAASSYLSLTPE
QWKSHRSYSCQVTHEGSTVEKTVAPTECS
SEQ ID NO: 116
Annotated DNA sequence of Synthetic anti-Gonorrhea (2C7) IgG-
Fab (Heavy Chain)
GAGGTTCAGCTGCAGCAGTCTGGACCTGAGCTGGTCAAGCCTGGCAGCAGCGTGAAGATCAG
CTGTAAAGGCAGCGGCTACACCTTCACCGACTACAACATGGAATGGGTCAAGCAGAGCCACG
GCAAGAGCCTGGAATGGATCGGCGTGATCAACCCCAACAACCGGTTCACCAGCTACAACCAG
AACTTCAGAGGCAAGGCCACACTGACCGTGGACAAGAGCAGCAGCACCGCCTACATGGATCT
GAGAAGCCTGACCAGCGAGGACAGCGCCGTGTATTTTTGTGCCGGCAGCCGGTGGTATCAGT
ACGACTATTGGGGCCAGGGCACAACCCTGACCGTTAGCTCTGCTAGCACCAAGGGCCCATCG
GTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGCAGCGTGGTG
ACCGTGCCCTCCAGCAGCTTGGGCACCCAGACCTACATCTGCAACGTGAATCACAAGCCCAG
CAACACCAAGGTGGACAAGAAAGTTGAGCCCAAATCTTGTGACAAAACTCACACATGCCCAC
CGTGCCCAGCACCTGAACTCCTGGGGGGACCGTCAGTCTTCCTCTTCCCCCCAAAACCCAAG
GACACCCTCATGATCTCCCGGACCCCTGAGGTCACATGCGTGGTGGTGGACGTGAGCCACGA
AGACCCTGAGGTCAAGTTCAACTGGTACGTGGACGGCGTGGAGGTGCATAATGCCAAGACAA
AGCCGCGGGAGGAGCAGTACAACAGCACGTACCGTGTGGTCAGCGTCCTCACCGTCCTGCAC
CAGGACTGGCTGAATGGCAAGGAGTACAAGTGCAAGGTCTCCAACAAAGCCCTCCCAGCCCC
CATCGAGAAAACCATCTCCAAAGCCAAAGGGCAGCCCCGAGAACCACAGGTGTACACCCTGC
CCCCATCCCGGGACGAGCTGACCAAGAACCAGGTCAGCCTGACCTGCCTGGTCAAAGGCTTC
TATCCCAGCGACATCGCCGTGGAGTGGGAGAGCAATGGGCAGCCGGAGAACAACTACAAGAC
CACGCCTCCCGTGCTGGACTCCGACGGCTCCTTCTTCCTCTACAGCAAGCTCACCGTGGACA
AGAGCAGGTGGCAGCAGGGGAACGTCTTCTCATGCTCCGTGATGCATGAGGCTCTGCACAAC
CACTACACGCAGAAGAGCCTCTCCCTGTCTCCGGGTAAAGGATCCGGTGGCGGAGGATCTGG
TGGAGGAGGGAGCGGAGGGGGTGGGTCCGGAGGGGGTGGTTCCGGAGGTGGTGGATCAGGTG
GCGGAGGAAGTGAGGTACAACTGCAACAGAGTGGCCCCGAGCTTGTGAAGCCAGGGTCCAGC
GTGAAGATTTCTTGCAAGGGAAGTGGGTACACGTTCACGGACTACAACATGGAGTGGGTGAA
ACAAAGTCACGGTAAATCCTTGGAGTGGATCGGAGTTATCAACCCAAACAACCGATTTACTA
GCTACAACCAGAATTTCAGGGGGAAGGCAACACTCACCGTCGACAAATCCTCTTCTACGGCA
TATATGGATCTCCGCTCACTTACTAGCGAGGACTCTGCAGTCTATTTTTGCGCGGGGAGCCG
ATGGTATCAATACGACTATTGGGGTCAAGGTACAACGCTTACTGTTAGCTCAGCTAGCACCA
AGGGCCCATCGGTCTTCCCCCTGGCACCCTCCTCCAAGAGCACCTCTGGGGGCACAGCGGCC
CTGGGCTCCCTGGTCAAGGACTACTTCCCCGAACCGGTGACGGTGTCGTGGAACTCAGGCGC
CCTGACCAGCGGCGTGCACACCTTCCCGGCTGTCCTACAGTCCTCAGGACTCTACTCCCTCA
GCAGCGTGGTGACCGTGCCCTCCAGCAGCTTGGGCACCCAGACCTACATCTGCAACGTGAAT
CACAAGCCCAGCAACACCAAGGTGGACAAGAAAGTTGAGCCCAAATCTTGTGACAAAACTCA
CACA
SEQ ID NO: 117
Annotated Amino Acid sequence of Synthetic anti-Gonorrhea
(2C7) IgG-Fab (Heavy Chain)
EVQLQQSGPELVKPGSSVKISCKGSGYTFTDYNMEWVKQSHGKSLEWIGVINPNNRFTSYNQ
NFRGKATLTVDKSSSTAYMDLRSLTSEDSAVYFCAGSRWYQYDYWGQGTTLTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTCPPCPAPELLGGPSVFLFPPKPK
DTLMISRTPEVTCVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNVESCSVMHEALHN
HYTQKSLSLSPGKGSGGGGSGCCGSGGGGSGGGGSGGGGSGGGGSEVQLQQSGPELVKPGSS
VKISCKGSGYTFTDYNMEWVKQSHGKSLEWIGVINPNNRFTSYNQNFRGKATLTVDKSSSTA
YMDLRSLTSEDSAVYFCAGSRWYQYDYWGQGTTLTVSSASTKGPSVFPLAPSSKSTSGGTAA
LGCLVKDYFPEPVTVSWNSGALTSGVHTFPAVLQSSGLYSLSSVVTVPSSSLGTQTYICNVN
HKPSNTKVDKKVEPKSCDKTHT
SEQ ID NO: 118
Annotated Amino Acid sequence of Synthetic anti-Gonorrhea
(2C7) Fab fragment of IgG-Fab (Heavy Chain)
EVQLQQSGPELVKPGSSVKISCKGSGYTFTDYNMEWVKQSHGKSLEWIGVINPNNRFTSYNQ
NFRGKATLTVDKSSSTAYMDLRSLTSEDSAVYFCAGSRWYQYDYWGQGTTLTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHT
SEQ ID NO: 119
Annotated Amino Acid sequence of Synthetic anti-Gonorrhea
(2C7) Fc fragment of IgG-Fab (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPEWLDSDGSFFLYSKLTVD
KSRWQQGNVFSCSVMHEALHNHYTQKSLSLSPGK
SEQ ID NO: 120
DNA sequence of Synthetic anti-Gonorrhea (2C7) IgG-Fab (Light
Chain)
CAGGTGGTGGTCACACAAGAGAGCGCCCTGACAACAAGCCCTGGCGAGACAGTGACCCTGAC
CTGCAGATCTTCTACAGGCGCCGTGACCACCTCCAACTACGCCAATTGGGTGCAAGAGAAGC
CCGACCACCTGTTCACAGGCCTGATCGGCGGCATCAACAATAGAGCACCTGGCCTGCCAGCC
AGATTCAGCGGATCTCTGATCGGAGACAAGGCCGCACTGACAATCACAGGCGCCCAGACAGA
GGACGAGGCCATCTACTTTTGCGCCCTGTGGTACAGCAACCACTGGGTTTTCGGCGGAGGCA
CCAAGCTGACAGTTCTGGGCCAACCTAAGGCCGCTCCTAGCGTGACACTGTTCCCTCCAAGC
AGCGAAGAACTGCAGGCCAACAAGGCCACACTCGTGTGCCTGATCAGCGACTTTTATCCTGG
CGCCGTGACCGTGGCCTGGAAGGCTGATAGTTCTCCTGTGAAGGCCGGCGTGGAAACCACCA
CACCTAGCAAGCAGAGCAACAACAAATACGCCGCCAGCAGCTACCTGAGCCTGACACCTGAG
CAGTGGAAGTCCCACAGATCCTACAGCTGCCAAGTGACCCACGAGGGCAGCACCGTGGAAAA
AACAGTGGCCCCTACCGAGTGCAGC
SEQ ID NO: 121
Annotated Amino Acid sequence of Synthetic anti-Gonorrhea
(2C7) IgG-Fab (Light Chain)
QVVVTQESALTTSPGETVTLTCRSSTGAVTTSNYANWVQEKPDHLFTGLIGGINNRAPGVPA
RFSGSLIGDKAALTITGAQTEDEAIYFCALWYSNHWVFGGGTKLTVLGQPKAAPSVTLFPPS
SEELQANKATLVCLISDFYPGAVTVAWKADSSPVKAGVETTTPSKQSNNKYAASSYLSLTPE
QWKSHRSYSCQVTHEGSTVEKTVAPTECS
SEQ ID NO: 122
Annotated Amino Acid sequence of Synthetic anti-Gonorrhea
(2C7) Fab fragment of IgG-Fab (Light Chain)
QVVVTQESALTTSPGETVTLTCRSSTGAVTTSNYANWVQEKPDHLFTGLIGGINNRAPGVPA
RFSGSLIGQKAALTITGAQTEDEAIYFCALWYSNHWVFGGGTKLTVLGQPKAAPSVTLFETS
SEELQANKATINCLISDFYPGAVTVAWKADSSPVKAGVETTTPSKQSNNKYAASSYLSLTET
QNKSHRSYSCQVTHEGSTVEKTVAPTECS
SEQ ID NO: 123
Annotated DNA sequence of Synthetic anti-Gonorrhea (2C7) Fab-
IgG-Fab (Heavy Chain)
GAGGTACAACTGCAACAGAGTGGCCCCGAGCTTGTGAAGCCAGGGTCCAGCGTGAAGATTTC
TTGCAAGGGAAGTGGGTACACGTTCACGGACTACAACATGGAGTGGGTGAAACAAAGTCACG
GTAAATCCTTGGAGTGGATCGGAGTTATCAACCCAAACAACCGATTTACTAGCTACAACCAG
AATTTCAGGGGGAAGGCAACACTCACCGTCGACAAATCCTCTTCTACGGCATATATGGATCT
CCGCTCACTTACTAGCGAGGACTCTGCAGTCTATTTTTGCGCGGGGAGCCGATGGTATCAAT
ACGACTATTGGGGTCAAGGTACAACGCTTACTGTTAGCTCAGCTAGCACCAAGGGCCCATCG
GTCTTCCCCCTGGCACCCTCCTCCAAGAGCACCTCTGGGGGCACAGCGGCCCTGGGCTGCCT
GGTCAAGGACTACTTCCCCGAACCGGTGACGGTGTCGTGGAACTCAGGCGCCCTGACCAGCG
GCGTGCACACCTTCCCGGCTGTCCTACAGTCCTCAGGACTCTACTCCCTCAGGAGCGTGGTG
ACCGTGCCCTCCAGCAGCTTGGGCACCCAGACCTACATCTGCAACGTGAATCACAAGCCCAG
CAACACCAAGGTGGACAAGAAAGTTGAGCCCAAATCTTGTGACAAAACTCACACAGGTGGCG
GAGGATCTGGTGGAGGAGGGAGCGGAGGGGGTGGGTCCGGAGGGGGTGGTTCCGGAGGTGGT
GGATCAGGTGGCGGAGGAAGTGAGGTTCAGCTGCAGGAGTCTGGACCTGAGCTGGTCAAGCC
TGGCAGCAGCGTGAAGATCAGCTGTAAAGGCAGCGGCTACACCTTCACCGACTACAACATGG
AATGGGTCAAGCAGAGCCACGGCAAGAGCCTGGAATGGATCGGCGTGATCAACCCCAACAAC
CGGTTCACCAGCTACAACCAGAACTTCAGAGGCAAGGCCACACTGACCGTGGACAAGAGCAG
CAGCACCGCCTACATGGATCTGAGAAGCCTGACCAGCGAGGACAGCGCCGTGTATTTTTGTG
CCGGCAGCCGGTGCTATCAGTACGACTATTGGGGCCAGGGCACAACCCTGACCGTTAGCTCT
GCTAGCACCAAGGGCCCATCGGTCTTCCCCCTGGCACCCTCCTCCAAGAGCACCTCTGGGGG
CACAGCGGCCCTGGGCTGCCTGGTCAAGGACTACTTCCCCGAACCGGTGACGGTGTCGTGGA
ACTCAGGCGCCCTGACCAGCGGCGTGCACACCTTCCCGGCTGTCCTACAGTCCTCAGGACTC
TACTCCCTCAGGAGCGTGGTGACCGTGCCCTCCAGGAGCTTGGGCACCCAGACCTACATCTG
CAACGTGAATCACAAGCCCAGCAACACCAAGGTGGACAAGAAAGTTGAGCCCAAATCTTGTG
ACAAAACTCACACATGCCCACCGTGCCCAGCACCTGAACTCCTGGGGGGACCGTCAGTCTTC
CTCTTCCCCCCAAAACCCAAGGACACCCTCATGATCTCCCGGACCCCTGAGGTCACATGCGT
GGTGGTGGACGTGAGCCACGAAGACCCTGAGGTCAAGTTCAACTGGTACGTGGACGGCGTGG
AGGTGCATAATGCCAAGACAAAGCCGCGGGAGGAGGAGTACAACAGCACGTACCGTGTGGTC
AGCGTCCTCACCGTCCTGCACCAGGACTGGCTGAATGGCAAGGAGTACAAGTGCAAGGTCTC
CAACAAAGCCCTCCCAGCCCCCATCGAGAAAACCATCTCCAAAGCCAAAGGGCAGCCCCGAG
AACCACAGGTGTACACCCTGCCCCCATCCCGGGACGAGCTGACCAAGAACCAGGTCAGCCTG
ACCTGCCTGGTCAAAGGCTTCTATCCCAGCGACATCGCCGTGGAGTGGGAGAGCAATGGGCA
GCCGGAGAACAACTACAAGACCACGCCTCCCGTGCTGGACTCCGACGGCTCCTTCTTCCTCT
ACAGCAAGCTCACCGTGGACAAGAGGAGGTGGCAGGAGGGGAACGTCTTCTCATGCTCCGTG
ATGCATGAGGCTCTGCACAACCACTACACGCAGAAGAGCCTCTCCCTGTCTCCGGGTAAAGG
ATCCGGTGGCGGAGGATCTGGTGGAGGAGGGAGCGGAGGGGGTGGGTCCGGAGGGGGTGGTT
CCGGAGGTGGTGGATCAGGTGGCGGAGGAAGTGAGGTACAACTGCAACAGAGTGGCCCCGAG
CTTGTGAAGCCAGGGTCCAGCGTGAAGATTTCTTGCAAGGGAAGTGGGTACACGTTCACGGA
CTACAACATGGAGTGGGTGAAACAAAGTCACGGTAAATCCTTGGAGTGGATCGGAGTTATCA
ACCCAAACAACCGATTTACTAGCTACAACCAGAATTTCAGGGGGAAGGCAACACTCACCGTC
GACAAATCCTCTTCTACGGCATATATGGATCTCCGCTCACTTACTAGCGAGGACTCTGCAGT
CTATTTTTGCGCGGGGAGCCGATGGTATCAATACGACTATTGGGGTCAAGGTACAACGCTTA
CTGTTAGCTCAGCTAGCACCAAGGGCCCATCGGTCTTCCCCCTGGCACCCTCCTCCAAGAGC
ACCTCTGGGGGCACAGCGGCCCTGGGCTGCCTGGTCAAGGACTACTTCCCCGAACCGGTGAC
GGTGTCGTGGAACTCAGGCGCCCTGACCAGCGGCGTGCACACCTTCCCGGCTGTCCTACAGT
CCTCAGGACTCTACTCCCTCAGCAGCGTGGTGACCGTGCCCTCCAGGAGCTTGGGCACCCAG
ACCTACATCTGCAACGTGAATCACAAGCCCAGCAACACCAAGGTGGACAAGAAAGTTGAGCC
CAAATCTTGTGACAAAACTCACACA
SEQ ID NO: 124
Annotated Amino Acid sequence of Synthetic anti-Gonorrhea
(2C7) Fab-IgG-Fab (Heavy Chain)
EVQLQQSGPELVKPGSSVKISCKGSGYTFTDYNMEWVKQSHGKSLEWIGVINPNNRFTSYNQ
NFRGKATLTVDKSSSTAYMDLRSLTSEDSAVYFCAGSRWYQYDYWGQGTTLTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTGGGGSGGGGSGGGGSGGGGSGGG
GSGGGGSEVQLQQSGPELVKPGSSVKISCKGSGYTFTDYNMEWVKQSHGKSLEWIGVINPNN
RFTSYNQNFRGKATLTVDKSSSTAYMDLRSLTSEDSAVYFCAGSRWYQYDYWGQGTTLTVSS
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTCPPCPAPELLGGPSVF
LFETKPKDTLMISRTPEVTCVVVDVSHEDPEVKFNWYVDGVEVHNAKTKPREEQYNSTYRVV
SVLTVLHQDWINGKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKSRWQQGNVESCSV
MHEALHNHYTQKSLSLSPGKGSGGGGSGGGGSGGGGSGGGGSGGGGSGGGGSEVQLQQSGPE
LVKPGSSVKISCKGSGYTFTDYNMEWVKQSHGKSLEWIGVINPNNRFTSYNQNFRGKATLTV
DKSSSTAYMDLRSLTSEDSAVYFCAGSRWYQYDYWGQGTTLTVSSASTKGPSVFPLAPSSKS
TSGGTAALGCLVKDYFPEPVTVSWNSGALTSGVHTFPAVLQSSGLYSLSSVVTVPSSSLGTQ
TYICNVNHKPSNTKVDKKVEPKSCDKTHT
SEQ ID NO: 125
Annotated Amino Acid sequence of Synthetic anti-Gonorrhea
(2C7) Fab fragment of Fab-IgG-Fab (Heavy Chain)
EVQLQQSGPELVKPGSSVKISCKGSGYTFTDYNMEWVKQSHGKSLEWIGVINPNNRFTSYNQ
NFRGKATLTVDKSSSTAYMDLRSLTSEDSAVYFCAGSRWYQYDYWGQGTTLTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHT
SEQ ID NO: 126
Annotated Amino Acid sequence of Synthetic anti-Gonorrhea
(2C7) Fc fragment of Fab-IgG-Fab (Heavy Chain)
PCPAPELLGGPSVFLEPPKPECTLMISRTPEVTCVVVINSHEDPEVKFNWYVDGVEVHNAKT
KPREEQYNSTYRVVSVLTVLHQDWLNGKEYKCKVSNKALPAPTEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 127
DNA sequence of Synthetic anti-Gonorrhea (2C7) Fab-IgG-Fab
(Light Chain)
CAGGTGGTGGTCACACAAGAGAGCGCCCTGACAACAAGCCCTGGCGAGACAGTGACCCTGAC
CTGCAGATCTTCTACAGGCGCCGTGACCACCTCCAACTACGCCAATTGGGTGCAAGAGAAGC
CCGACCACCTGTTCACAGGCCTGATCGGCGGCATCAACAATAGAGCACCTGGCGTGCCAGCC
AGATTCAGCGGATCTCTGATCGGAGACAAGGCCGCACTGACAATCACAGGCGCCCAGACAGA
GGACGAGGCCATCTACTTTTGCGCCCTGTGGTACAGCAACCACTGGGTTTTCGGCGGAGGCA
CCAAGCTGACAGTTCTGGGCCAACCTAAGGCCGCTCCTAGCGTGACACTGTTCCCTCCAAGC
AGCGAAGAACTGCAGGCCAACAAGGCCACACTCGTGTGCCTGATCAGCGACTTTTATCCTGG
CGCCGTGACCGTGGCCTGGAAGGCTGATAGTTCTCCTGTGAAGGCCGGCGTGGAAACCACCA
CACCTAGCAAGCAGAGCAACAACAAATACGCCGCCAGCAGCTACCTGAGCCTGACACCTGAG
CAGTGGAAGTCCCACAGATCCTACAGCTGCCAAGTGACCCACGAGGGCAGCACCGTGGAAAA
AACAGTGGCCCCTACCGAGTGCAGC
SEQ ID NO: 128
Annotated Amino Acid sequence of Synthetic anti-Gonorrhea
(2C7) Fab-IgG-Fab (Light Chain)
QVVVTQESALTTSPGETVTLTCRSSTGAVTTSNYANWVQEKPDHLFTGLIGGINNRAPGVPA
RFSGSLIGDKAALTITGAQTEDEAIYFCALWYSNHWVFGGGTKLTVLGQPKAARSVTLFPPS
SEELQANKATLVCLISQFYPGAVTVAWKADSSPVKAGVETTTPSKQSNNKYAASSYLSLTPE
QWKSHRSYSCQVTHEGSTVEKTVAPTECS
SEQ ID NO: 129
Annotated Amino Acid sequence of Synthetic anti-Gonorrhea
(2C7) Fab fragment of Fab-IgG-Fab (Light Chain)
QVVVTQESALTTSPGETVTLTCRSSTGAVTTSNYANWVQEKPDHLFTGLIGGINNRAPGVPA
RFSGSLIGDKAALTITGAQTEDEATYFCALWYSNHWVEGGGTKLTVLGQPKAAPSVTLFPPS
SEELQANKATLVCLISDFYPGAVTVAWKADSSPVKAGVETTTPSKQSNNKYAASSYLSLTPE
QWKSHRSYSCQVTHEGSTVEKTVAPTECS
SEQ ID NO: 130
Forward primer
TAAGCAGGTACCGCCACCATGAAGTG
SEQ ID NO: 131
Reverse primer
TGCTTAGCTAGCTGGAGAAACTGTC
SEQ ID NO: 132
Annotated DNA sequence of anti-RSV (Respiratory syncytia1
virus) IgG (Heavy Chain)
CAAGTGACCCTGAGAGAGTCTGGCCCCGCTCTGGTTAAGCCCACACAGACCCTGACACTGAC
CTGCACCTTCAGCGGCTTTAGCCTGTCTACAGCCGGCATGAGCGTCGGCTGGATTAGACAGC
CTCCTGGCAAAGCCCTGGAATGGCTGGCCGACATTTGGTGGGACGACAAGAAGCACTACAAC
CCCAGCCTGAAGGACCGGCTGACCATCAGCAAGGACACCAGCAAGAACCAGGTGGTGCTGAA
AGTGACCAACATGGACCCTGCCGACACCGCCACCTACTACTGCGCCAGAGACATGATCTTCA
ACTTCTACTTCGACGTGTGGGGCCAGGGCACCACCGTGACAGTTAGCTCTGCCTCTACAAAG
GGCCCCAGCGTGTTCCCTCTGGCTCCTAGCAGCAAGTCTACAAGCGGAGGAACAGCCGCTCT
GGGCTGCCTGGTCAAGGATTACTTTCCCGAGCCTGTGACCGTGTCCTGGAATTCTGGCGCTC
TGACAAGCGGCGTGCACACCTTTCCAGCTGTGCTGCAAAGCAGCGGCCTGTACTCTCTGAGC
AGCGTGGTCACAGTGCCAAGCTCTAGCCTGGGCACCCAGACCTACATCTGCAATGTGAACCA
CAAGCCTAGCAACACCAAGGTGGACAAGAAGGTGGAACCCAAGAGCTGCGACAAGACCCACA
CCTGTCCTCCATGTCCTGCTCCAGAACTGCTCGGCGGACCTTCCGTGTTTCTGTTCCCTCCA
AAGCCTAAGGACACCCTGATGATCAGCAGAACCCCTGAAGTGACCTGCGTGGTGGTGGATGT
GTCCCACGAGGATCCCGAAGTGAAGTTCAATTGGTACGTGGACGGCGTGGAAGTGCACAACG
CCAAGACCAAGCCTAGAGAGGAACAGTACAACAGCACCTACAGAGTGGTGTCTGTGCTGACC
GTGCTGCACCAGGATTGGCTGAACGGCAAAGAGTACAAGTGCAAGGTGTCCAACAAGGCCCT
GCCTGCTCCTATCGAGAAAACCATCTCCAAGGCCAAGGGCCAGCCTAGGGAACCCCAGGTTT
ACACACTGCCTCCAAGCAGGGACGAGCTGACCAAGAATCAGGTGTCCCTGACCTGCCTCGTG
AAGGGCTTCTACCCTTCCGATATCGCCGTGGAATGGGAGAGCAATGGCCAGCCTGAGAACAA
CTACAAGACAACCCCTCCTGTGCTGGACAGCGACGGCTCATTCTTCCTGTACAGCAAGCTGA
CAGTGGACAAGTCCAGATGGCAGCAGGGCAACGTGTTCAGCTGCAGCGTGATGCACGAGGCC
CTGCACAACCACTACACCCAGAAGTCCCTGAGCCTGTCTCCAGGCAAG
SEQ ID NO: 133
Annotated Amino Acid sequence of anti-RSV (Respiratory
syncytial virus) IgG (Heavy Chain)
QVTLRESGPALVKPTQTLTLTCTFSGESLSTAGMSVGWIRQPPGKALEWLADIWWDDKKHYN
PSLKDRLTISKDTSKNQVVLKVTNMDPADTATYYCARDMIFNFYFDVWGQGTTVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEA
LHNHYTQKSLSLSPGK
SEQ ID NO: 134
Annotated Amino Amid sequence of Fab fragment of anti-RSV
(Respiratory syncytial virus) (Heavy Chain)
QVTLRESGPALVKPTQTLTLTCTFSGFSLSTAGMSVGWIRQPPGKALEWLADIWWDDKKHYN
PSLKDRLTISKDTSKNQVVLKVTNMDPADTATYYCARDMIFNFYFDVWGQGTTVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHT
SEQ ID NO: 135
Annotated Amino Acid sequence of Fc fragment of anti-RSV
(Respiratory syncytial virus) (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 136
DNA sequence of anti-RSV (Respiratory syncytial virus) (Light
Chain)
GACATCCAGATGACACAGAGCCCCAGCACACTGTCTGCCAGCGTGGGAGACAGAGTGACCAT
CACATGTAGCGCCAGCAGCAGAGTGGGCTACATGCACTGGTATCAGCAGAAGCCTGGCAAGG
CCCCTAAGCTGCTGATCTACGACACAAGCAAGCTGGCCTCTGGCGTGCCCAGCAGATTTTCT
GGCTCTGGCAGCGGCACCGAGTTCACCCTGACCATCTCTAGCCTGCAGCCTGACGACTTCGC
CACCTACTACTGCTTTCAAGGCAGCGGCTACCCCTTCACCTTTGGCGGCGGAACAAAGGTGG
AAATCAAGCGGACAGTGGCCGCTCCTAGCGTGTTCATCTTTCCACCTAGCGACGAGCAGCTG
AAGTCTGGCACAGCCTCTGTCGTGTGCCTGCTCAACAACTTCTACCCCAGAGAAGCCAAGGT
GCAGTGGAAGGTGGACAACGCCCTGCAGAGCGGCAATAGCCAAGAGAGCGTGACCGAGCAGG
ACAGCAAGGACTCTACCTACAGCCTGTCCTCCACACTGACCCTGAGCAAGGCCGACTACGAG
AAGCACAAAGTGTACGCCTGCGAAGTGACCCACCAGGGCCTTTCTAGCCCTGTGACCAAGAG
CTTCAACCGGGGCGAGTGC
SEQ ID NO: 137
Annotated Amino Acid sequence of anti-RSV (Respiratory
syncytial virus) (Light Chain)
DIQMTQSPSTLSASVGDRVTITCSASSRVGYMHWYQQKPGKAPKLLIYDTSKLASGVPSRFS
GSGSGTEFTLTISSLQPDDFATYYCFQGSGYPFTFGGGTKVEIKRTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 138
Annotated Amino Acid sequence of Fab fragment of anti-RSV
(Respiratory syncytial virus) IgG Might Chain)
DIQMTQSPSTLSASVGDRVTITCSASSRVGYMHWYQQKPGKAPKILIYDTSKLASGVPSRFS
GSGSGTEFTLTISSLQPDDFATYYCFQGSGYPFTFGGGTKVEIKRTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 139
Annotated DNA sequence of Synthetic anti-RSV (Respiratory
syncytial virus) Fab-IgG (Heavy Chain)
CAAGTGACCCTGAGAGAGTCTGGCCCCGCTCTGGTTAAGCCCACACAGACCCTGACACTGAC
CTGCACCTTCAGCGGCTTTAGCCTGTCTACAGCCGGCATGAGCGTCGGCTGGATTAGACAGC
CTCCTGGCAAAGCCCTGGAATGGCTGGCCGACATTTGGTGGGACGACAAGAAGCACTACAAC
CCCAGCCTGAAGGACCGGCTGACCATCAGCAAGGACACCAGCAAGAACCAGGTGGTGCTGAA
AGTGACCAACATGGACCCTGCCGACACCGCCACCTACTACTGCGCCAGAGACATGATCTTCA
ACTTCTACTTCGACGTGTGGGGCCAGGGCACCACCGTGACAGTTAGCTCTGCCTCTACAAAG
GGCCCCAGCGTGTTCCCTCTGGCTCCTAGCAGCAAGTCTACAAGCGGAGGAACAGCCGCTCT
GGGCTGCCTGGTCAAGGATTACTTTCCCGAGCCTGTGACCGTGTCCTGGAATTCTGGCGCTC
TGACAAGCGGCGTGCACACCTTTCCAGCTGTGCTGCAAAGCAGCGGCCTGTACTCTCTGAGC
AGCGTGGTCACAGTGCCAAGCTCTAGCCTGGGCACCCAGACCTACATCTGCAATGTGAACCA
CAAGCCTAGCAACACCAAGGTGGACAAGAAGGTGGAACCCAAGAGCTGCGACAAGACCCATA
CAGGCGGCGGAGGATCTGGCGGAGGTGGAAGCGGAGGCGGAGGAAGCGGTGGCGGCGGTAGT
GGCGGTGGTGGTTCAGGTGGTGGTGGCTCTCAAGTCACACTGAGAGAAAGCGGCCCTGCTCT
CGTGAAGCCTACTCAGACACTCACCCTGACCTGTACATTCTCTGGCTTCAGCCTGAGCACCG
CCGGCATGTCTGTTGGATGGATCAGACAACCACCAGGCAAGGCTCTCGAGTGGCTCGCTGAT
ATTTGGTGGGATGATAAGAAACATTATAACCCATCTCTCAAGGACCGCCTCACAATCTCCAA
GGATACCTCCAAGAATCAGGTCGTCCTCAAAGTCACGAATATGGATCCCGCCGATACGGCCA
CATATTACTGTGCCCGGGATATGATCTTTAATTTCTATTTTGATGTCTGGGGCCAAGGGACA
ACCGTCACCGTGTCTAGCGCCAGCACAAAGGGACCCTCCGTGTTTCCACTGGCACCCAGCTC
TAAGAGCACCTCTGGTGGAACAGCTGCCCTGGGATGTCTCGTGAAAGACTACTTCCCCGAAC
CAGTGACAGTCAGCTGGAACAGCGGAGCCCTGACTTCTGGGGTGCACACATTCCCTGCCGTC
CTGCAATCTTCTGGCCTGTACAGCCTGTCCAGCGTCGTGACCGTTCCTTCTAGCTCTCTGGG
AACACAGACATATATCTGTAATGTCAATCACAAACCCTCCAATACGAAGGTCGACAAAAAGG
TCGAGCCTAAGTCCTGTGATAAGACCCACACCTGTCCTCCATGTCCTGCTCCAGAACTGCTC
GGCGGACCTTCTGTGTTTCTGTTCCCTCCAAAGCCTAAGGACACCCTGATGATCAGCAGAAC
CCCTGAAGTGACCTGCGTGGTGGTCGATGTGTCCCACGAGGATCCCGAAGTGAAGTTCAATT
GGTACGTGGACGGCGTGGAAGTGCACAACGCCAAGACCAAGCCTAGAGAGGAACAGTACAAC
AGCACCTACAGAGTGGTGTCTGTGCTGACCGTGCTGCACCAGGATTGGCTGAACGGCAAAGA
GTACAAGTGCAAGGTGTCCAACAAGGCCCTGCCTGCTCCTATCGAGAAAACCATCTCTAAGG
CCAAGGGCCAGCCTCGCGAACCCCAGGTTTACACACTTCCACCAAGCCGGGACGAGCTGACA
AAAAACCAGGTGTCCCTGACATGCCTCGTGAAGGGCTTCTACCCCTCCGATATCGCCGTGGA
ATGGGAGAGCAATGGCCAGCCTGAGAACAACTACAAGACCACACCTCCTGTGCTGGACAGCG
ACGGCTCATTCTTCCTGTACTCCAAGCTGACAGTGGACAAGTCCAGATGGCAGCAGGGCAAC
GTGTTCAGCTGCAGCGTGATGCACGAGGCCCTGCACAACCACTACACCCAGAAAAGCCTGTC
TCTGAGCCCCGGCAAG
SEQ ID NO: 140
Annotated Amino Acid sequence of Synthetic anti-RSV
(Respiratory syncytial virus) IgG Fab-IgG (Heavy Chain)
QVTLRESGPALVKPTQTLTLTCTFSGFSLSTAGMSVGWIRQPPGKALEWLADIWWDDKKHYN
PSLKDRLTISKDTSKNQVVLKVTNMDPADTATYYCARDMIFNEYFDVWGQGTTVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTGGGGSGGGGSGGGGSGGGGS
GGGGSGGGGSQVTLRESGPALVKPTQTLTLTCTFSGFSLSTAGMSVGWIRQPPGKALEWLAD
IWWDDKKHYNPSLKDRLTISKDTSKNQVVLKVTNMDPADTATYYCARDMIFNFYFDVWGQGT
TVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCIWTHTCPPCPAPELL
GGPSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQGN
VFSCSVMHEALHNHYTQKSLSLSPGK
SEQ ID NO: 141
Annotated Amino Acid sequence of Synthetic anti-RSV
(Respiratory syncytial virus) Fab fragment of Fab-IgG (Heavy
Chain)
QVTLRESGPALVKPTQTLTLTCTFSGFSLSTAGMSVGWIRQPPGKALEWLADIWWDDKKHYN
PSLKDRLTISKDTSKNQVVLKVTNMDPADTATYYCARDMIFNFYFDVWGQGTTVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHT
SEQ ID NO: 142
Annotated Amino Acid sequence of Synthetic anti-RSV
(Respiratory syncytial virus) Fc fragment of Fab-IgG (Heavy
Chain)
PCPAPELLGGPSVFLIPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 143
DNA sequence of Synthetic anti-RSV (Respiratory syncytial
virus) Fab-IgG (Light Chain)
GACATCCAGATGACACAGAGCCCCAGCACACTGTCTGCCAGCGTGGGAGACAGAGTGACCAT
CACATGTAGCGCCAGCAGCAGAGTGGGCTACATGCACTGGTATCAGCAGAAGCCTGGCAAGG
CCCCTAAGCTGCTGATCTACGACACAAGCAAGCTGGCCTCTGGCGTGCCCAGCAGATTTTCT
GGCTCTGGCAGCGGCACCGAGTTCACCCTGACCATCTCTAGCCTGCAGCCTGACGACTTCGC
CACCTACTACTGCTTTCAAGGCAGCGGCTACCCCTTCACCTTTGGCGGCGGAACAAAGGTGG
AAATCAAGCGGACAGTGGCCGCTCCTAGCGTGTTCATCTTTCCACCTAGCGACGAGCAGCTG
AAGTCTGGCACAGCCTCTGTCGTGTGCCTGCTCAACAACTTCTACCCCAGAGAAGCCAAGGT
GCAGTGGAAGGTGGACAACGCCCTGCAGAGCGGCAATAGCCAAGAGAGCGTGACCGAGCAGG
ACAGCAAGGACTCTACCTACAGCCTGTCCTCCACACTGACCCTGAGCAAGGCCGACTACGAG
AAGCACAAAGTGTACGCCTGCGAAGTGACCCACCAGGGCCTTTCTAGCCCTGTGACCAAGAG
CTTCAACCGGGGCGAGTGC
SEQ ID NO: 144
Annotated Amino Acid sequence of Synthetic anti-RSV
(Respiratory syncytial virus) Fab-IgG (Light Chain)
DIQMTQSPSTLSASVGDRVTITCSASSRVGYMHWYQQKPGKAPKLLIYDTSKLASGVPSRFS
GSGSGTEFTLTISSLQPDDEATYYCFQGSGYPFTFGGGTKVEIKRTVAAPSVFIFPPSDEQL
KSGTASVVCLLNNFYPREAKVQWKVDNALQSGNSQESVTEQDSKDSTYSLSSTLTLSKADYE
KHKVYACEVTHQGLSSPVTKSFNRGEC
SEQ ID NO: 145
Annotated Amino Acid sequence of Synthetic anti-RSV
(Respiratory syncytial virus) Fab fragment of Fab-IgG (Light
Chain)
DIQMTQSPSTLSASVGDRVTITCSASSRVGYMHWYQQKPGKAPKLLITDTSKLASGVPSRFS
GSGSGTEFTLTISSLQPDDFATYYCFQGSGYPFTFGGGTKVEIKRTVAAPSVFIFPPSDEQL
KSGTASVVCLLNNFYPREAKVQWKVIDNALUGNSQESVTEQDSKDSTYSLSSTLTLSKADYE
KHKVYACEVTHQGLSSPVTKSFNRGEC
SEQ ID NO: 146
Annotated DNA sequence of Synthetic anti-RSV (Respiratory
syncytial virus) IgG-Fab (Heavy Chain)
CAAGTGACCCTGAGAGAGTCTGGCCCCGCTCTGGTTAAGCCCACACAGACCCTGACACTGAC
CTGCACCTTCAGCGGCTTTAGCCTGTCTACAGCCGGCATGAGCGTCGGCTGGATTAGACAGC
CTCCTGGCAAAGCCCTGGAATGGCTGGCCGACATTTGGTGGGACGACAAGAAGCACTACAAC
CCCAGCCTGAAGGACCGGCTGACCATCAGCAAGGACACCAGCAAGAACCAGGTGGTGCTGAA
AGTGACCAACATGGACCCTGCCGACACCGCCACCTACTACTGCGCCAGAGACATGATCTTCA
ACTTCTACTTCGACGTGTGGGGCCAGGGCACCACCGTGACAGTTAGCTCTGCCTCTACAAAG
GGCCCCAGCGTGTTCCCTCTGGCTCCTAGCAGCAAGTCTACAAGCGGAGGAACAGCCGCTCT
GGGCTGCCTGGTCAAGGATTACTTTCCCGAGCCTGTGACCGTGTCCTGGAATTCTGGCGCTC
TGACAAGCGGCGTGCACACCTTTCCAGCTGTGCTGCAAAGCAGCGGCCTGTACTCTCTGAGC
AGCGTGGTCACAGTGCCAAGCTCTAGCCTGGGCACCCAGACCTACATCTGCAATGTGAACCA
CAAGCCTAGCAACACCAAGGTGGACAAGAAGGTGGAACCCAAGAGCTGCGACAAGACCCACA
CCTGTCCTCCATGTCCTGCTCCAGAACTGCTCGGCGGACCTTCCGTGTTTCTGTTCCCTCCA
AAGCCTAAGGACACCCTGATGATCAGCAGAACCCCTGAAGTGACCTGCGTGGTGGTGGATGT
GTCCCACGAGGATCCCGAAGTGAAGTTCAATTGGTACGTGGACGGCGTGGAAGTGCACAACG
CCAAGACCAAGCCTAGAGAGGAACAGTACAACAGCACCTACAGAGTGGTGTCTGTGCTGACC
GTGCTGCACCAGGATTGGCTGAACGGCAAAGAGTACAAGTGCAAGGTGTCCAACAAGGCCCT
GCCTGCTCCTATCGAGAAAACCATCTCCAAGGCCAAGGGCCAGCCTAGGGAACCCCAGGTTT
ACACACTGCCTCCAAGCAGGGACGAGCTGACCAAGAATCAGGTGTCCCTGACCTGCCTCGTG
AAGGGCTTCTACCCTTCCGATATCGCCGTGGAATGGGAGAGCAATGGCCAGCCTGAGAACAA
CTACAAGACAACCCCTCCTGTGCTGGACAGCGACGGCTCATTCTTCCTGTACAGCAAGCTGA
CAGTGGACAAGTCCAGATGGCAGCAGGGCAACGTGTTCAGCTGCAGCGTGATGCACGAGGCC
CTGCACAACCACTACACCCAGAAGTCCCTGAGCCTGTCTCCAGGCAAAGGTGGCGGAGGATC
TGGCGGAGGTGGAAGCGGCGGAGGCGGTTCTGGTGGTGGCGGCTCTGGCGGCGGTGGTTCAG
GTGGCGGCGGTTCTCAAGTTACACTGAGAGAAAGCGGCCCAGCTCTCGTGAAGCCTACTCAG
ACACTCACCCTGACATGTACCTTCTCTGGCTTCAGCCTGAGCACCGCCGGCATGTCTGTTGG
ATGGATCAGACAACCACCAGGCAAGGCTCTCGAGTGGCTCGCTGATATTTGGTGGGATGATA
AGAAACATTATAACCCATCTCTCAAGGACCGCCTCACCATTTCCAAGGATACCTCCAAAAAT
CAGGTCGTGCTCAAAGTCACGAATATGGATCCCGCCGATACGGCCACATATTACTGTGCCCG
GGATATGATCTTTAATTTCTATTTTGATGTCTGGGGCCAAGGGACAACCGTCACCGTGTCTA
GCGCCAGCACAAAGGGACCCTCTGTGTTTCCACTGGCTCCCAGCTCTAAGAGCACCTCCGGT
GGAACAGCTGCCCTGGGATGTCTCGTGAAAGACTACTTCCCCGAACCAGTGACAGTCAGCTG
GAACAGCGGAGCCCTGACTAGTGGGGTGCACACATTCCCTGCCGTCCTGCAATCTAGCGGAC
TGTACAGCCTGTCCAGCGTCGTGACCGTGCCTTCTAGCTCTCTGGGAACACAGACATATATC
TGTAATGTCAATCACAAACCCTCCAATACGAAGGTCGACAAAAAGGTCGAGCCTAAGTCCTG
TGATAAGACGCACACA
SEQ ID NO: 147
Annotated Amino Acid sequence of Synthetic anti-RSV
(Respiratory syncytial virus) IgG-Fab (Heavy Chain)
QVTLRESGPALVKPTQTLTLTCTFSGFSLSTAGMSVGWIRQPPGKALEWLADIWWDDKKHYN
PSLKDRLTISKDTSKNQVVLKVTNMDPADTATYYCARDMIFNFYFDVWGQGTTVTVSSASTK
GPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVFPKSCDKTHTCPPCPAPELLGGPSVFLFPP
KPKDTLMISRTPEVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEA
LHNHYTQKSLSLSPGKGGGGSGGGGSGGGGSGGGGSGGGGSGGGGSQVTLRESGPALVKPTQ
TLTLTCTFSGFSLSTAGMSVGWIRQPPGKALEWLADIWWDDKKHYNPSLKDRLTISKDTSKN
QVVLKVTNMDPADTATYYCARDMIFNFYFDVWGQGTTVTVSSASTKGPSVFPLAPSSKSTSG
GTAALGCLVKDYFPEPVTVSWNSGALTSGVHTFPAVLQSSGLYSLSSVVTVPSSSLGTQTYI
CNVNHKPSNTKVDKKVEPKSCDKTHT
SEQ ID NO: 148
Annotated Amino Acid sequence of Synthetic anti-RSV
(Respiratory syncytial virus) Fab fragment of IgG-Fab (Heavy
Chain)
QVTLRESGPALVKPTQTLTLTCTFSGFSLSTAGMSVGWIRQPPGRALEWLADIWWDDKKHYN
PSLKDRLTISKDTSKNQVVLKVTNMDPADTATYYCARDMIFNFYFDVWGQGTTVTVSSASTK
GPSVFPLAPSSKSTSGGTAALGCLVKDYFPEPVTVSWNSGALTSCVHTFPAVLQSSGLYSLS
SVVTVPSSSLGTQTYICNVNHKPSNTKVDKKVEPKSCDKTHT
SEQ ID NO: 149
Annotated Amino Acid sequence of Synthetic anti-RSV
(Respiratory syncytial virus) Fc fragment of IgG-Fab (Heavy
Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 150
DNA sequence of Synthetic anti-RSV (Respiratory syncytial
virus) IgG-Fab (Light Chain)
GACATCCAGATGACACAGAGCCCCAGCACACTGTCTGCCAGCGTGGGAGACAGAGTGACCAT
CACATGTAGCGCCAGGAGCAGAGTGGGCTACATGCACTGGTATCAGCAGAAGCCTGGCAAGG
CCCCTAAGCTGCTGATCTACGACACAAGCAAGCTGGCCTCTGGCGTGCCCAGCAGATTTTCT
GGCTCTGGCAGCGGCACCGAGTTCACCCTGACCATCTCTAGCCTGCAGCCTGACGACTTCGC
CACCTACTACTGCTTTCAAGGCAGCGGCTACCCCTTCACCTTTGGCGGCGGAACAAAGGTGG
AAATCAAGCGGACAGTGGCCGCTCCTAGCGTGTTCATCTTTCCACCTAGCGACGAGCAGCTG
AAGTCTGGCACAGCCTCTGTCGTGTGCCTGCTCAACAACTTCTACCCCAGAGAAGCCAAGGT
GCAGTGGAAGGTGGACAACGCCCTGCAGAGCGGCAATAGCCAAGAGAGCGTGACCGAGCAGG
ACAGCAAGGACTCTACCTACAGCCTGTCCTCCACACTGACCCTGAGCAAGGCCGACTACGAG
AAGCACAAAGTGTACGCCTGCGAAGTGACCCACCAGGGCCTTTCTAGCCCTGTGACCAAGAG
CTTCAACCGGGGCGAGTGC
SEQ ID NO: 151
Annotated Amino Acid sequence of Synthetic anti-RSV
(Respiratory syncytial virus) IgG-Fab (Light Chain)
DIQMTQSPSTLSASVGDRVTITCSASSRVGYMHWYQQKPGKAPKLLIYETSKLASGVPSRFS
GSGSGTEFTLTISSLQPDDFATYYCFQGSGYPFTFGGGTKVEIKRTVAAPSVFIFPPSDEQL
KSGTASVVCLLNNEYPREAKVQWKVDNALQSGNSQESVTEQDSKQSTYSLSSTLTLSKADYE
KHKVYACEVTHQGLSSPVTKSFNRGEC
SEQ ID NO: 152
Annotated Amino Acid sequence of Synthetic anti-RSV
(Respiratory syncytial virus) Fab fragment of IgG-Fab (Light
Chain)
DIQMTQSPSTLSASVGDRVTITCSASSRVGYMHWYQQKPGKAPKLLTYDTSKLASCVPSRES
GSGSGTEFTLTISSLQPDDFATYYCFQGSGYPFTFGGGTKVEIKRTVAAPSVFIFPPSDEQL
KSGTASVVCLLNNFYPREAKVQWKVDNALQPGNSQESVTEQDSKDSTYSLSSTLTLSKADYE
KHKVYACEVTHQGLSSPVTKSFNRGEC
SEQ ID NO: 153
Annotated DNA sequence of anti-Gonorrhea (2C7) Fab-Fab-IgG-
Fab-Fab (Heavy Chain)
GAGGTTCAGCTGCAGCAGTCTGGACCTGAGCTGGTCAAGCCTGGCAGGAGCGTGAAGATCAG
CTGTAAAGGCAGCGGCTACACCTTCACCGACTACAACATGGAATGGGTCAAGCAGAGCCACG
GCAAGAGCCTGGAATGGATCGGCGTGATCAACCCCAACAACCGGTTCACCAGCTACAACCAG
AACTTCAGAGGCAAGGCCACACTGACCGTGGACAAGAGCAGCAGCACCGCCTACATGGATCT
GAGAAGCCTGACCAGCGAGGACAGCGCCGTGTATTTTTGTGCCGGCAGCCGGTGGTATCAGT
ACGACTATTGGGGCCAGGGCACAACCCTGACAGTGTCTAGCGCCTCTACAAAGGGCCCCAGC
GTTTTCCCACTGGCTCCTAGCAGCAAGAGCACATCTGGCGGAACAGCCGCTCTGGGCTGTCT
GGTCAAGGACTACTTTCCCGAGCCTGTGACCGTGTCCTGGAATTCTGGCGCTCTGACAAGCG
GCGTGCACACCTTTCCAGCTGTGCTGCAAAGCAGCGGCCTGTACTCTCTGAGCAGCGTGGTC
ACAGTGCCAAGCTCTAGCCTGGGCACCCAGACCTACATCTGCAATGTGAACCACAAGCCTAG
CAACACCAAGGTCGACAAGAAGGTGGAACCCAAGAGCTGCGACAAGACCCATACAGGCGGCG
GAGGAAGCGGAGGCGGAGGATCTGGTGGTGGTGGATCTGGCGGTGGCGGAAGCGGCGGAGGC
GGTTCTGGTGGCGGAGGTAGTGAAGTGCAGCTTCAGCAGAGCGGCCCAGAACTCGTGAAGCC
TGGAAGCTCTGTGAAGATCTCCTGCAAAGGCTCTGGGTACACGTTTACGGATTACAATATGG
AATGGGTTAAGCAATCCCACGGGAAGTCCCTCGAGTGGATTGGAGTGATCAATCCGAACAAT
CGCTTTACCTCCTACAATCAAAACTTCCGGGGCAAAGCTACCCTGACTGTGGATAAGTCCAG
CTCCACAGCCTATATGGACCTGCGGTCCCTGACCTCTGAGGACTCCGCTGTGTACTTCTGCG
CCGGCTCCAGATGGTATCAATATGATTACTGGGGACAGGGAACCACTCTGACTGTGTCCTCC
GCCAGCACAAAGGGACCCTCCGTGTTTCCTCTGGCTCCCAGCTCTAAGTCTACCAGCGGAGG
AACAGCTGCCCTGGGATGCCTCGTGAAAGACTACTTCCCTGAACCAGTGACAGTCAGCTGGA
ACAGCGGAGCCCTGACTTCTGGGGTGCACACATTCCCTGCCGTCCTGCAATCTTCTGGCCTG
TACAGCCTGTCCAGCGTCGTGACCGTTCCTTCTAGCTCTCTGGGAACACAGACATATATCTG
TAATGTCAATCACAAACCCTCCAATACGAAGGTGGACAAAAAGGTCGAGCCTAAGTCCTGTG
ATAAGACCCACACAGGTGGTGGTGGTAGTGGTGGCGGCGGTTCAGGCGGAGGTGGTAGTGGC
GGAGGTGGCAGCGGTGGTGGCGGTAGCGGAGGTGGTGGAAGTGAAGTTCAACTGCAACAGTC
AGGCCCCGAGCTTGTGAAGCCAGGCTCCTCCGTGAAAATCAGTTGCAAAGGATCCGGGTATA
CCTTCACAGACTATAATATGGAATGGGTCAAACAGTCTCACGGCAAAAGTCTTGAGTGGATA
GGTGTCATTAACCCGAACAACAGATTCACCTCTTATAATCAAAATTTCAGAGGGAAAGCCAC
GCTCACAGTCGACAAGTCCTCCAGCACTGCTTATATGGATCTCCGCAGCCTGACATCCGAGG
ATTCTGCCGTCTACTTCTGTGCAGGCAGTCGCTGGTATCAGTACGATTACTGGGGTCAAGGG
ACTACCCTGACCGTCAGCTCTGCTAGCACCAAGGGCCCATCGGTCTTCCCCCTGGCACCCTC
CTCCAAGAGCACCTCTGGGGGCACAGCGGCCCTGGGCTGCCTGGTCAAGGACTACTTCCCCG
AACCGGTGACGGTGTCGTGGAACTCAGGCGCCCTGACCAGCGGCGTGCACACCTTCCCGGCT
GTCCTACAGTCCTCAGGACTCTACTCCCTCAGCAGCGTGGTGACCGTGCCCTCCAGCAGCTT
GGGCACCCAGACCTACATCTGCAACGTGAATCACAAGCCCAGCAACACCAAGGTGGACAAGA
AAGTTGAGCCCAAATCTTGTGACAAAACTCACACATGCCCACCGTGCCCAGCACCTGAACTC
CTGGGGGGACCGTCAGTCTTCCTCTTCCCCCCAAAACCCAAGGACACCCTCATGATCTCCCG
GACCCCTGAGGTCACATGCGTGGTGGTGGACGTGAGCCACGAAGACCCTGAGGTCAAGTTCA
ACTGGTACGTGGACGGCGTGGAGGTGCATAATGCCAAGACAAAGCCGCGGGAGGAGCAGTAC
AACAGCACGTACCGTGTGGTCAGCGTCCTCACCGTCCTGCACCAGGACTGGCTGAATGGCAA
GGAGTACAAGTGCAAGGTCTCCAACAAAGCCCTCCCAGCCCCCATCGAGAAAACCATCTCCA
AAGCCAAAGGGCAGCCCCGAGAACCACAGGTGTACACCCTGCCCCCATCCCGGGACGAGCTG
ACCAAGAACCAGGTCAGCCTGACCTGCCTGGTCAAAGGCTTCTATCCCAGCGACATCGCCGT
GGAGTGGGAGAGCAATGGGCAGCCGGAGAACAACTACAAGACCACGCCTCCCGTGCTGGACT
CCGACGGCTCCTTCTTCCTCTACAGCAAGCTCACCGTGGACAAGAGCAGGTGGCAGCAGGGG
AACGTCTTCTCATGCTCCGTGATGCATGAGGCTCTGCACAACCACTACACGCAGAAGAGCCT
CTCCCTGTCTCCGGGTAAAGGATCCGGTGGTGGTGGTAGTGGTGGCGGCGGTTCAGGCGGAG
GTGGTAGTGGCGGAGGTGGCAGCGGTGGTGGCGGTAGCGGAGGTGGTGGAAGTGAGGTTCAG
CTGCAGCAGTCTGGACCTGAGCTGGTCAAGCCTGGCAGCAGCGTGAAGATCAGCTGTAAAGG
CAGCGGCTACACCTTCACCGACTACAACATGGAATGGGTCAAGCAGAGCCACGGCAAGAGCC
TGGAATGGATCGGCGTGATCAACCCCAACAACCGGTTCACCAGCTACAACCAGAACTTCAGA
GGCAAGGCCACACTGACCGTGGACAAGAGCAGCAGCACCGCCTACATGGATCTGAGAAGCCT
GACCAGCGAGGACAGCGCCGTGTATTTTTGTGCCGGCAGCCGGTGGTATCAGTACGACTATT
GGGGCCAGGGCACAACCCTGACAGTGTCTAGCGCCTCTACAAAGGGCCCCAGCGTTTTCCCA
CTGGCTCCTAGCAGCAAGAGCACATCTGGCGGAACAGCCGCTCTCGGCTGTCTGGTCAAGGA
CTACTTTCCCGAGCCTGTGACCGTGTCCTGGAATTCTGGCGCTCTGACAAGCGGCGTGCACA
CCTTTCCAGCTGTGCTGCAAAGCAGCGGCCTGTACTCTCTGAGCAGCGTGGTCACAGTGCCA
AGCTCTAGCCTGGGCACCCAGACCTACATCTGCAATGTGAACCACAAGCCTAGCAACACCAA
GGTCGACAAGAAGGTGGAACCCAAGAGCTGCGACAAGACCCATACAGGCGGCGGAGGAAGCG
GAGGCGGAGGATCTGGTGGTGGTGGATCTGGCGGTGGCGGAAGCGGCGGAGGCGGTTCTGGT
GGCGGAGGTAGTGAAGTGCAGCTTCAGCAGAGCGGCCCACAACTCGTGAAGCCTGGAAGCTC
TGTGAAGATCTCCTGCAAAGGCTCTGGGTACACGTTTACGGATTACAATATGGAATGGCTTA
AGCAATCCCACGGGAAGTCCCTCGAGTGGATTGGAGTGATCAATCCGAACAATCGCTTTACC
TCCTACAATCAAAACTTCCGGGGCAAAGCTACCCTGACTGTGGATAAGTCCAGCTCCACAGC
CTATATGGACCTGCGGTCCCTGACCTCTGAGGACTCCGCTGTGTACTTCTGCGCCGGCTCCA
GATGGTATCAATATGATTACTGGGGACAGGGAACCACTCTGACTGTGTCCTCCGCCAGCACA
AAGGGACCCTCCGTGTTTCCTCTCGCTCCCAGCTCTAAGTCTACCAGCGGAGGAACAGCTGC
CCTGGGATGCCTCGTCAAAGACTACTTCCCTGAACCAGTGACAGTCAGCTGGAACAGCGGAG
CCCTGACTTCTGGGGTGCACACATTCCCTGCCGTCCTGCAATCTTCTGGCCTGTACAGCCTG
TCCAGCGTCGTGACCGTTCCTTCTAGCTCTCTGGGAACACAGACATATATCTGTAATGTCAA
TCACAAACCCTCCAATACGAAGGTGGACAAAAAGGTCGAGCCTAAGTCCTGTGATAAGACCC
ACACA
SEQ ID NO: 154
Annotated Amino Acid sequence of anti-Gonorrhea (2C7) Fab-Fab-
IgG-Fab-Fab (Heavy Chain)
EVQLQQSGPELVKPGSSVKISCKGSGYTFTDYNMEWVKQSHGKSLEWIGVINPNNRFTSYNQ
NFRGKATLTVDKSSSTAYMDLRSLTSEDSAVYFCAGSRWYQYDYWGQGTTLTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHTGGGGSGGGGSGGGGSGGGGSGGG
GSGGGGSEVQLQQSGPELVKPGSSVKISCKGSGYTFTDYNMEWVKQSHGKSLEWIGVINPNN
RFTSYNQNFRGKATLTVDKSSSTAYMDLRSLTSEDSAVYFCAGSRWYQYDYWGQGTTLTVSS
ASTKGPSVFPLAPSSKSTSGGTAALGCLVKDYFPEPVTVSWNSGALTSGVHTFPAVLQSSGL
YSLSSVVTVPSSSLGTQTYICNVNHKPSNTKVDKKVEPKSCDKTHTGGGGSGGGGSGGGGSG
GGGSGGGGSGGGGSEVQLQQSGPELVKPGSSVKISCKGSGYTFTDYNMEWVKQSHGKSLEWI
GVINPNNRFTSYNQNFRGKATLTVDKSSSTAYMDLRSLTSEDSAVYFCAGSRWYQYDYWGQG
TTLTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAPEL
LGGPSVFLFETKPKDTLMISRTPEVTCVVVDVSHEDPEVKFNWYVDGVEVHNAKTKPREEQY
NSTYRVVSVLTVLHQDWLNCKEYKCKVSNKALPAPIEKTISKAKGQPREPQVYTLPPSRDEL
TKNQVSLTCLVKGFYPSDIAVEWESNGQPENNYKTTPPVLDSDCSFFLYSKLTVDKSRWQQG
NVFSCSVMHEALHNHYTQKSLSLSPGKGSGGGGSGGGGSGGGGSGGGGSGGGGSGGGGSEVQ
LQQSGPELVKPGSSVKISCKGSGYTFTDYNMEWVKQSHGKSLEWIGVINPNNRFTSYNQNFR
GKATLTVDKSSSTAYMDLRSLTSEDSAVYFCAGSRWYQYDYWGQGTTLTVSSASTKGPSVFP
LAPSSKSTSGGTAALGCLVKDYFPEPVTVSWNSGALTSGVHTFPAVLQSSGLYSLSSVVTVP
SSSLGTQTYICNVNHKPSNTKVDKKVEPKSCDKTHTGGGGSGGGGSGGGGSGGGGSGGGGSG
GGGSEVQLQQSGPELVKPGSSVKISCKGSGYTFTDYNMEWVKQSHGKSLEWIGVINPNNRFT
SYNQNFRGKATLTVDKSSSTAYMDLRSLTSEDSAVYFCAGSRWYQYDYWCQCTTLTVSSAST
KGPSVFPLAPSSKSTSGGTAALGCLVKDYFPEPVTVSWNSGALTSGVHTFPAVLQSSGLYSL
SSVVTVPSSSLGTQTYICNVNHKPSNTKVDKKVEPKSCDKTHT
SEQ ID NO: 155
Annotated Amino Acid sequence of Fab fragment of anti-
Gonorrhea (2C7) (Heavy Chain)
EVQLQQSGPELVKPGSSVKISCKGSGYTFTDYNMEWVKQSHGKSLEWIGVINPNNRFTSYNQ
NFRGKATLTVDKSSSTAYMDLRSLTSEDSAVYFCAGSRWYQYDYWGQGTTLTVSSASTKGPS
VFPLAPSSKSTSGGTAALGCLVKDYFPEPVTVSWNSGALTSGVHTFPAVLQSSGLYSLSSVV
TVPSSSLGTQTYICNVNHKPSNTKVDKKVEPKSCDKTHT
SEQ ID NO: 156
Annotated Amino Acid sequence of Fc fragment of anti-Gonorrhea
(2C7) (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
SEQ ID NO: 157
DNA sequence of anti-Gonorrhea (2C7) (Light Chain)
CAGGTGGTGGTCACACAAGAGAGCGCCCTGACAACAAGCCCTGGCGAGACAGTGACCCTGAC
CTGCAGATCTTCTACAGGCGCCGTGACCACCTCCAACTACGCCAATTGGGTGCAAGAGAAGC
CCGACCACCTGTTCACAGGCCTGATCGGCGGCATCAACAATAGAGCACCTGGCGTGCCAGCC
AGATTCAGCGGATCTCTGATCGGAGACAAGGCCGCACTGACAATCACAGGCGCCCAGACAGA
GGACGAGGCCATCTACTTTTGCGCCCTGTGGTACAGCAACCACTGGGTTTTCGGCGGAGGCA
CCAAGCTGACAGTTCTGGGCCAACCTAAGGCCGCTCCTAGCGTGACACTGTTCCCTCCAAGC
AGCGAAGAACTGCAGGCCAACAAGGCCACACTCGTGTGCCTGATCAGCGACTTTTATCCTGG
CGCCGTGACCGTGGCCTGGAAGGCTGATAGTTCTCCTGTGAAGGCCGGCGTGGAAACCACCA
CACCTAGCAAGGAGAGCAACAACAAATACGCCGCCAGCAGCTACCTGAGCCTGACACCTGAG
CAGTGGAAGTCCCACAGATCCTACAGCTGCCAAGTGACCCACGAGGGCAGCACCGTGGAAAA
AACAGTGGCCCCTACCGAGTGCAGC
SEQ ID NO: 158
Annotated Amino Acid sequence of anti-Gonorrhea (2C7) (Light
Chain)
QVVVTQESALTTSPGETVTLTCRSSTGAVTTSNYANWVQEMPDHLFTGLIGGINNRAPGVPA
RFSGSLIGDKAALTITGAQTEDEAIYFCALWYSNHWVEGGGTKLTVLGQPKAAPSVTLFPPS
SEELQANKATLVCLISDFYPGAVTVAWKADSSPVKAGVETTTPSKQSNNKYAASSYLSLTPE
QWKSHRSYSCQVTHEGSTVEKTVAPTECS
SEQ ID NO: 159
Annotated Amino Acid sequence of Fab fragment of anti-
Gonorrhea (2C7) IgG (Light Chain)
QVVVTQESALTTSPGETVTLTCRSSTGAVTTSNYANWVQEKPDHLFTGLIGGINNRAPGVPA
RFSGSLIGDKAALTITGAQTEDEAIYFCALWYSNHWVFGGGTKLTVLGQPKAAPSVTLEPPS
SEELQANKATLVCLISDFYPGAVTVAWKADSSPVKAGVETTTPSKQSNNKYAASSYLSLTPE
QWKSHRSYSCQVTHEGSTVEKTVAPTECS
SEQ ID NO: 160
Annotated DNA sequence of anti-klebsiella Fab-Fab-IgG-Fab-Fab
(Heavy Chain)
GAGGTGCAGCTGGTTGAATCTGGCGGAGGACTGGTTCAGCCTGGCGGATCTCTGAGACTGTC
TTGCGCCGCCAGCTTTAGCCTGACAAGCTACGCCGTGCACATCCACTGGGTTCGACAGGCCC
CTGGCAAAGGCCTTGAATGGGTTGCCAGAGTGATCTGGGCTGGCGGCATCACCCACTACAAT
AGCGCCCTGATGAGCAGATACGCCGACAGCGTGAAGGGCAGATTCACCATCAGCGCCGACAC
CAGCAAGAACACCGCCTACCTGCAGATGAACAGCCTGAGAGCCGAGGACACCGCCGTGTACT
ATTGCGCCAGAGGCAACTGGGCCTTCGACTATTGGGGACAGGGCACCCTGGTCACAGTGTCT
AGCGCCTCTACAAAGGGCCCCAGCGTTTTCCCACTGGCTCCTAGCAGCAAGAGCACATCTGG
TGGAACAGCCGCTCTGGGCTGCCTGGTCAAGGATTACTTTCCCGAGCCTGTGACCGTGTCCT
GGAATTCTGGCGCTCTGACAAGCGGCGTGCACACCTTTCCAGCTGTGCTGCAAAGCAGCGGC
CTGTACTCTCTGAGCAGCGTCGTGACAGTGCCAAGCAGCTCTCTGGGCACCCAGACCTACAT
CTGCAATGTGAACCACAAGCCTAGCAACACCAAGGTGGACAAGAAGGTGGAACCCAAGAGCT
GCGACAAGACCCATACAGGCGGAGGTGGAAGCGGAGGCGGAGGCTCTGGCGGCGGAGGAAGT
GGTGGCGGCGGTAGTGGCGGTGGTGGATCAGGTGGCGGAGGTTCTGAAGTCCAGCTGGTGGA
AAGTGGCGGCGGACTTGTGCAACCAGGTGGAAGTCTGAGGCTGAGCTGTGCCGCCTCTTTCA
GCCTGACCTCTTATGCCGTGCATATTCATTGGGTCCGCCAAGCTCCAGGCAAGGGCCTCGAG
TGGGTCGCACGAGTTATTTGGGCAGGCGGAATTACACACTATAACTCTGCCCTCATGTCCCG
CTACGCCGACTCTGTGAAAGGCCGGTTTACCATCTCCGCCGATACCTCCAAGAATACTGCCT
ATCTCCAAATGAACTCTCTGCGCGCCGAAGATACAGCCGTGTATTACTGTGCTCGCGGAAAT
TGGGCCTTTGATTACTGGGGCCAAGGCACACTGGTTACCGTCAGCTCTGCCAGCACAAAGGG
ACCCTCCGTGTTTCCTCTGGCTCCCAGCTCTAAGTCTACCAGCGGAGGAACAGCTGCCCTGG
GATGTCTCGTGAAAGACTACTTCCCCGAACCAGTGACAGTCAGCTGGAACAGCGGAGCCCTG
ACTTCTGGGGTGCACACATTCCCTGCCGTCCTGCAATCTTCTGGCCTGTACAGCCTGTCCAG
CGTGGTCACCGTTCCTAGCTCTAGCCTGGGAACACAGACATATATCTGTAATGTCAATCACA
AACCCTCCAATACGAAGGTCGACAAAAAGGTCGAGCCTAAGTCCTGTGATAAGACACACACT
GGCGGTGGCGGTTCAGGCGGAGGCGGAAGTGGCGGAGGCGGATCCGGCGGTGGTGGTAGTGG
TGGTGGCGGCAGCGGAGGCGGCGGATCTGAAGTTCAGCTTGTTGAGTCAGGTGGTGGCCTCG
TGCAACCTGGCGGAAGCCTTAGACTTTCCTGCGCCGCTTCATTCTCCCTGACCTCATACGCT
GTCCATATACACTGGGTCCGACAAGCACCCGGAAAAGGATTGGAGTGGGTTGCCCGGGTTAT
ATGGGCTGGTGGTATCACACATTATAACAGCGCTCTGATGTCTCGCTATGCCGATTCCGTCA
AGGGGCGCTTCACAATCTCTGCCGACACCTCTAAAAACACGGCTTACCTTCAAATGAATTCC
CTCCGCGCTGAGGATACCGCTGTCTACTACTGTGCACGCGGCAACTGGGCTTTCGACTACTG
GGGTCAAGGGACTCTCGTGACTGTGTCCTCTGCCTCTACAAAGGGCCCTAGTGTGTTCCCTC
TGGCTCCCAGCAGCAAGTCTACATCTGGCGGAACAGCCGCTCTGGGCTGCCTGGTCAAGGAT
TACTTTCCCGAGCCTGTGACCGTGTCCTGGAATTCTGGCGCTCTGACAAGCGGCGTGCACAC
CTTTCCAGCTGTGCTGCAAAGCAGCGGCCTGTACTCTCTGAGCAGCGTGGTCACAGTGCCTA
GCTCTAGCCTGGGCACCCAGACCTACATCTGCAATGTGAACCACAAGCCTAGCAACACCAAG
GTGGACAAGAAGGTGGAACCCAAGAGCTGCGACAAGACCCACACCTGTCCTCCATGTCCTGC
TCCAGAACTGCTCGGCGGACCTTCCGTGTTCCTGTTTCCTCCAAAGCCTAAGGACACCCTGA
TGATCAGCAGAACCCCTGAAGTGACCTGCGTGGTGGTGGATGTGTCCCACGAGGATCCCGAA
GTGAAGTTCAATTGGTACGTGGACGGCGTGGAAGTGCACAACGCCAAGACCAAGCCTAGAGA
GGAACAGTACAACAGCACCTACAGAGTGGTGTCCGTGCTGACCGTGCTGCACCAGGATTGGC
TGAACGGCAAAGAGTACAAGTGCAAGGTGTCCAACAAGGCCCTGCCTGCTCCTATCGAGAAA
ACCATCAGCAAGGCCAAGGGCCAGCCTAGGGAACCCCAGGTTTACACACTGCCTCCAAGCCG
GGAAGAGATGACCAAGAACCAGGTGTCCCTGACCTGCCTCGTGAAGGGCTTCTACCCTTCCG
ATATCGCCGTGGAATGGGAGAGCAATGGCCAGCCTGAGAACAACTACAAGACAACCCCTCCT
GTGCTGGACAGCGACGGCTCATTCTTCCTGTACAGCAAGCTGACAGTGGACAAGTCCAGATG
GCAGCAGGGCAACGTGTTCAGCTGCAGCGTGATGCACGAGGCCCTGCACAACCACTACACCC
AGAAGTCCCTGAGCCTGTCTCCTGGCAAAGGATCCGGCGGTGGCGGTTCAGGCGGAGGCGGA
AGTGGCGGAGGCGGATCCGGCGGTGGTGGTAGTGGTGGTGGCGGCAGCGGAGGCGGCGGATC
TGAGGTGCAGCTGGTTGAATCTGGCGGAGGACTGGTTCAGCCTGGCGGATCTCTGAGACTGT
CTTGCGCCGCCAGCTTTAGCCTGACAAGCTACGCCGTGCACATCCACTGGGTTCGACAGGCC
CCTGGCAAAGGCCTTGAATGGGTTGCCAGAGTGATCTGGGCTGGCGGCATCACCCACTACAA
TAGCGCCCTGATGAGGAGATACGCCGACAGCGTGAAGGGCAGATTCACCATCAGCGCCGACA
CCAGCAAGAACACCGCCTACCTGCAGATGAACAGCCTGAGAGCCGAGGACACCGCCGTGTAC
TATTGCGCCAGAGGCAACTGGGCCTTCGACTATTGGGGACAGGGCACCCTGGTCACAGTGTC
TAGCGCCTCTACAAAGGGCCCCAGCGTTTTCCCACTGGCTCCTAGCAGCAAGAGCACATCTG
GTGGAACAGCCGCTCTGGGCTGCCTGGTCAAGGATTACTTTCCCGAGCCTGTGACCGTGTCC
TGGAATTCTGGCGCTCTGACAAGCGGCGTGCACACCTTTCCAGCTGTGCTGCAAAGGAGCGG
CCTGTACTCTCTGAGCAGCGTCGTGACAGTGCCAAGCAGCTCTCTGGGCACCCAGACCTACA
TCTGCAATGTGAACCACAAGCCTAGCAACACCAAGGTGGACAAGAAGGTGGAACCCAAGAGC
TGCGACAAGACCCATACAGGCGGAGGTGGAAGCGGAGGCGGAGGCTCTGGCGGCGGAGGAAG
TGGTGGCGGCGGTAGTGGCGGTGGTGGATCAGGTGGCGGAGGTTCTGAAGTCCAGCTGGTGG
AAAGTGGCGGCGGACTTGTGCAACCAGGTGGAAGTCTGAGGCTGAGCTGTGCCGCCTCTTTC
AGCCTGACCTCTTATGCCGTGCATATTCATTGGGTCCGCCAAGCTCCAGGCAAGGGCCTCGA
GTGGGTCGCACGAGTTATTTGGGCAGGCGGAATTACACACTATAACTCTGCCCTCATGTCCC
GCTACGCCGACTCTGTGAAAGGCCGGTTTACCATCTCCGCCGATACCTCCAAGAATACTGCC
TATCTCCAAATGAACTCTCTGCGCGCCGAAGATACAGCCGTGTATTACTGTGCTCGCGGAAA
TTGGGCCTTTGATTACTGGGGCCAAGGCACACTGGTTACCGTCAGCTCTGCCAGCACAAAGG
GACCCTCCGTGTTTCCTCTGGCTCCCAGCTCTAAGTCTACCAGCGGAGGAACAGCTGCCCTG
GGATGTCTCGTGAAAGACTACTTCCCCGAACCAGTGACAGTCAGCTGGAACAGCGGAGCCCT
GACTTCTGGGGTGCACACATTCCCTGCCGTCCTGCAATCTTCTGGCCTGTACAGCCTGTCCA
GCGTGGTCACCGTTCCTAGCTCTAGCCTGGGAACACAGACATATATCTGTAATGTCAATCAC
AAACCCTCCAATACGAAGGTCGACAAAAAGGTCGAGCCTAAGTCCTGTGATAAGACACACAC
T
SEQ ID NO: 161
Annotated Amino Acid sequence of anti-klebsiella Fab-Fab-IgG-
Fab-Fab (Heavy Chain)
EVQLVESGGGLVQPGGSLRLSCAASFSLTSYAVHIHWVRQAPGKGLEWVARVIWAGGITHYN
SALMSRYADSVKGRFTISADTSKNTAYLQMNSLRAEDTAVYYCARGNWAFDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHTGGGGSGGGGSGGGGS
GGGGSGGGGSGGGGSEVQLVESGGGLVQPGGSLRLSCAASFSLTSYAVHIHWVRQAPGKGLE
WVARVIWAGGITHYNSALMSRYADSVKGRFTISADTSKNTAYLQMNSLRAEDTAVYYCARGN
WAFDYWGQGTLVTVSSASTKGPSVFPLAPSSKSTSGGTAALGCLVKDYETEPVTVSWNSGAL
TSGVHTFPAVLQSSGLYSLSSVVTVPSSSLGTQTYICNVNHKPSNTKVDKKVEPKSCDKTHT
GGGGSGGGGSGGGGSGGGGSGGGGSGGGGSEVQLVESGGGLVQPGGSLRLSCAASFSLTSYA
VHIHWVRQAPGKGLEWVARVIWAGGITHYNSALMSRYADSVKGRFTISADTSKNTAYLQMNS
LRAEDTAVYYCARGNWAFDYWGQGTLVTVSSASTKGPSVFPLAPSSKSTSGGTAALGCLVKD
YEPEPVTVSWNSGALTSGVHTFPAVDDSSGLYSLSSVVTVPSSSLGTQTYICNVNHKPSNTK
VDKKVEPKSCDKTHTCPPCPAPELLGGPSVFLYPPKPKDTLMISRTPEVTCVVVDVSHEDPE
VKFNWYVDGVEVHNAKTKPREEQYNSTYRVVSVLTVLHQDWLNGKEYaCKVSNKALPAPIEK
TISKAKGQPREPQVYTLPPSREEMTKNQVSLTCLVKGEYPSDIAVEWESNGQPENNYKTTPP
VLDSDGSFFLYSKLTVDKSRWQQGNVFSCSVMHEALHNHYTQKSLSLSPGKGSGGGGSGGGG
SGGGGSGGGGSGGGGSGGGGSEVQLVESGGGLVQPGGSLRLSCAASFSLTSYAVHIHWVRQA
PGKGLEWVARVIWAGGITHYNSALMSRYADSVKGRFTISADTSKNTAYLQMNSLRAEDTAVY
YCARGNWAFDYWGQGTLVTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKKVEPKS
CDKTHTGGGGSGGGGSGGGGSGGGGSGGGGSGGGGSEVQLVESGGGLVQPGGSLRLSCAASF
SLTSYAVHIHWVRQAPGKGLEWVARVIWAGGITHYNSALMSRYADSVKGRFTISADTSKNTA
YLQMNSLRAEDTAVYYCARGNWAFDYWGQGTLVTVSSASTKGPSVFPLAPSSKSTSGGTAAL
GCLVKDYFPEPVTVSWNSGALTSGVHTFPAVLQSSGLYSLSSVVTVPSSSLGTQTYICNVNH
KPSNTKVDKKVEPKSCDKTHT
SEQ ID NO: 162
Annotated Amino Acid sequence of Fab fragment of anti-
klebsiella (Heavy Chain)
EVQLVESGGGLVQPGGSLRLSCAASFSLTSYAVHIHWVRQAPGKGLEWVARVIWAGGITHYN
SALMSRYADSVKGRFTISADTSKNTAYLQMNSLRAEDTAVYYCARGNWAFDYWGQGTLVTVS
SASTKGPSVFPLAPSSKSTSGGTAALGCLVKDYFPEPVTVSWNSGALTSGVHTFPAVLQSSG
LYSLSSVVTVPSSSLGTQTYICNVNHKPSNTKVDKKVEPKSCDKTHT
SEQ ID NO: 163
Annotated Amino Acid sequence of Fc fragment of anti-
klebsiella (Heavy Chain)
PCPAPELLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSDGSFFLYSKLTVD
KSRWQQGNVFSCSVMHEALHNHYTUSLSLSPGK
SEQ ID NO: 164
DNA sequence of anti-klebsiella (Light Chain)
GACATCCAGATGACACAGAGCCCTAGCAGCCTGTCTGCCAGCGTGGGAGACAGAGTGACCAT
CACCTGTAGAGCCAGCAGCGCCAGATCCAGCGTGTCCTATATTCACGTGGCCTGGTATCAGC
AGAAGCCCGGCAAAGCCCCTAAGCTGCTGATCTACGACACCAGCAAACTGGCCAGCTTCCTG
TACAGCGGCGTGCCCTCTAGATTCAGCGGCAGCAGATCTGGCACCGACTTCACCCTGACCAT
AAGCAGCCTGCAGCCTGAGGACTTCGCCACCTACTACTGCTTTCAAGGCAGCGGCTACCGCT
ACACCTTTGGCCAGGGAACAAAGGTGGAAATCAAGAGAACAGTGGCCGCTCCTAGCGTGTTC
ATCTTCCCACCTTCCGACGAGCAGCTGAAGTCTGGCACAGCCTCTGTCGTGTGCCTGCTGAA
CAACTTCTACCCCAGAGAAGCCAAGGTGCAGTGGAAGGTGGACAACGCCCTGCAGAGCGGCA
ATAGCCAAGAGAGCGTGACCGAGCAGGACAGCAAGGACTCTACCTACAGCCTGAGCAGCACC
CTGACACTGAGCAAGGCCGACTACGAGAAGCACAAAGTGTACGCCTGCGAAGTGACCCACCA
GGGCCTTTCTAGCCCTGTGACCAAGAGCTTCAACCGGGGCGAATGT
SEQ ID NO: 165
Annotated Amino Acid sequence of anti-klebsiella (Light Chain)
DIQMTQSPSSLSASVGDRVTITCRASSARSSVSYIHVAWYQQKPGKAPKLLIYDTSKLASFL
YSGVPSRFSGSRSGTQFTLTISSLQPEDFATYYCFQGSGYPYTFGQGTKVEIKRTVAAPSVF
IFPPSDEQLKSGTASVVCLLNNEYPREAKVQWKVDNALQSGNSQESVTEQDSKDSTYSLSST
LTLSKADYEKHKVYACEVTHQGLSSPVTKSFNRGEC
SEQ ID NO: 166
Annotated Amino Amid sequence of Fab fragment of anti-
klebsiella Fab-Fab-IgG-Fab-Fab (Light Chain)
DIQMTQSPSSLSASVGDRVTITCRASSARSSVSYIHVAWYQQKPGKAPKLLIYDTSKLASEL
YSGVPSRFSGSRSGTDFTLTISSLQPEDFATYYCFQGSGYPYTEGQGTKVEIKRTVAAPSVF
IFPPSDEQLKSGTASVVCLLNNEYPREAKVQWKVDNALQSGNSQESVTEQDSKDSTYSLSST
LTLSKADYEKHKVYACEVTHQGLSSPVTKSFNRGEC

1-126. (canceled)

127. A synthetic binding agent for enhancing agglutination of a target having an epitope, the synthetic binding agent comprising: a human or humanized Immunoglobulin G (IgG) having a pair of Fab domains, wherein the human or humanized IgG is linked to one or more additional immunoglobulin fragment antigen binding (Fab) domains by an amino acid linker, wherein the one or more additional Fab domains and the IgG Fab domains all specifically bind to the epitope of the target, so that the synthetic binding agent binds to the target with high affinity and reduces an average mobility of the target in mucus to less than about 50% relative to its native mobility in mucus.

128. The synthetic binding agent of claim 127, wherein the one or more additional Fab domains comprises 2, 4, 6 or 8 additional Fab domains.

129. The synthetic binding agent of claim 127, wherein the target is sperm and all of the one or more Fab domains and the IgG Fab domains specifically bind to an epitope of CD52g.

130. The synthetic binding agent of claim 129, wherein the IgG Fab domains specifically bind to repeating poly-n-acetyllactosaminyl structures.

131. The synthetic binding agent of claim 129, wherein the one or more additional Fab domains each comprise: (i) a heavy chain (HC) with a variable region (VH) comprising complementarity determining regions (CDRs) having the amino acid sequences of: SEQ ID NO: 4; and/or (ii) a light chain (LC) with a variable region (VL) comprising complementarity determining regions (CDRs) having the amino acid sequence of: SEQ ID NO: 7.

132. The synthetic binding agent of claim 129, wherein the one or more additional Fab domains each comprise: (i) a heavy chain (HC) with a variable region (VH) comprising complementarity determining regions (CDRs) having an amino acid sequence that is between 100% and 80% identical to the amino acid sequence of: SEQ ID NO: 4; and/or (ii) a light chain (LC) with a variable region (VL) comprising complementarity determining regions (CDRs) having an amino acid sequence that is between 100% and 80% identical to the amino acid sequence of: SEQ ID NO: 7.

133. The synthetic binding agent of claim 127, wherein the target is Klebsiella bacillus.

134. The synthetic binding agent of claim 133, wherein the one or more additional Fab domains each comprise: (i) a heavy chain (HC) with a variable region (VH) comprising complementarity determining regions (CDRs) having the amino acid sequences of: SEQ ID NO: 41; and/or (ii) a light chain (LC) with a variable region (VL) comprising complementarity determining regions (CDRs) having the amino acid sequences of: SEQ ID NO: 44.

135. The synthetic binding agent of claim 133, wherein the one or more additional Fab domains each comprise; (i) a heavy chain (HC) with a variable region (VH) comprising complementarity determining regions (CDRs) having an amino acid sequence that is between 100% and 80% identical to the amino add sequence of: SEQ ID NO: 41; and/or (ii) a light chain (LC) with a variable region (VL) comprising complementarity determining regions (CDRs) having an amino acid sequence that is between 100% and 80% identical to the amino acid sequence of SEQ ID NO: 44.

136. The synthetic binding agent of claim 127, wherein the target is respiratory syncytial virus (RSV).

137. The synthetic binding agent of claim 136, wherein the one or more additional Fab domains each comprise: (I) a heavy chain (HC) with a variable region (VH) comprising complementarity determining regions (CDRs) having the amino acid sequences of: SEQ ID NO.: 134; and/or (ii) a light chain (LC) with a variable region (VL) comprising complementarity determining regions (CDRs) having the amino acid sequences of: SEQ ID NO: 138.

138. The synthetic binding agent of claim 136, wherein the one or more additional Fab domains each comprise: (1) a heavy chain (HC) with a variable region (VH) comprising complementarity determining regions (CDRs) having an amino acid sequence that is between 100% and 80% identical to the amino acid sequence of: SEQ ID NO: 134; and/or (ii) a light chain (LC) with a variable region (VL) comprising complementarity determining regions (CDRs) having an amino acid sequence that is between 100% and 80% identical to the amino acid sequence of: SEQ ID NO: 138.

139. The synthetic binding agent of claim 127, wherein the target is Neisseria gonorrhoeae.

140. The synthetic binding agent of claim 139, wherein the one or more additional Fab domains each comprise: (i) a heavy chain (HC) with a variable region (VH) comprising complementarity determining regions (CDRs) having the amino acid sequences of: SEQ ID NO: 104; and/or (ii) a light chain (LC) with a variable region (VL) comprising complementarity determining regions (CDRs) having the amino acid sequences of: SEQ ID NO: 107.

141. The synthetic binding agent of claim 139, wherein the one or more additional Fab domains each comprise: (i) a heavy chain (HC) with a variable region (VH) comprising complementarity determining regions (CDRs) having an amino acid sequence that is between 100% and 80% identical to the amino acid sequence of SEQ ID NO: 104; and/or (ii) a light chain (LC) with a variable region (VL) comprising complementarity determining regions (CDRs) having an amino acid sequence that is between 100% and 80% identical to the amino acid sequence of: SEQ ID NO: 107.

142. The synthetic binding agent of claim 127, wherein the one or more additional Fab domains is linked to a Fab domain of the pair of Fab domains of the IgG.

143. The synthetic binding agent of claim 127, wherein the one or more additional Fab domain is linked to an Fc region of the IgG.

144. The synthetic binding agent of claim 127, wherein the IgG comprises at least one Fc region that is a naturally occurring sequence.

145. The synthetic binding agent of claim 127, wherein the IgG comprises at least one Fc region comprising one or more mutations that enhance or decrease binding to Fc receptors.

146. The synthetic binding agent of claim 145, wherein the IgG has a peptide sequence that is at least 80% homologous with the sequence of SEQ ID No: 5, comprising one or more mutations that decrease binding to an Fc receptor selected from the group consisting of: L7V, L8A and P102G.

147. The synthetic binding agent of claim 127, wherein the one or more additional Fab domains are linked to the IgG via a flexible linker comprising an amino acid sequence comprising n pentapeptide repeats consisting of Glycine (G) and Serine (S), wherein n is between 3 and 8.

148. The synthetic binding agent of claim 127, wherein the IgG Fab domains have an amino acid sequence that is not identical to the one or more additional Fab domains.

149. A synthetic binding agent for inhibiting sperm mobility through mucus, comprising; a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to an epitope of CD52g, so that the synthetic binding agent reduces mobility of sperm in mucus to less than about 50% relative to its native mobility in mucus.

150. A synthetic binding agent for treating or preventing infection by a Klebsiella bacillus pathogen, the synthetic binding agent comprising; a human or humanized Immunoglobulin G (IgG) having a pair of Fab domains, wherein the human or humanized. IgG is linked to one or more additional immunoglobulin fragment antigen binding (Fab) domains, wherein the one or more additional Fab domains and the IgG Fab domains all specifically bind to an epitope specific to Klebsiella bacillus, so that the synthetic binding agent reduces mobility of Klebsiella bacillus in mucus.

151. A synthetic binding agent for treating or preventing infection by a respiratory syncytial virus (RSV), the synthetic binding agent comprising: a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to an epitope specific to RSV, so that the synthetic binding agent reduces mobility of RSV in mucus.

152. A synthetic binding agent for treating or preventing infection by a Neisseria gonorrhoeae, the synthetic binding agent comprising: a human or humanized Immunoglobulin G (IgG) having a pair of Fab domains, wherein the human or humanized IgG is linked to one or more additional immunoglobulin fragment antigen binding (Fab) domains, wherein the one or more additional Fab domains and the IgG Fab domains all specifically bind to an epitope specific to Neisseria gonorrhoeae, so that the synthetic binding agent reduces mobility of Neisseria gonorrhoeae in mucus.


SEQUENCE LISTING (155 sequences)

SEQ ID NO: 1 - protein, 12 aa, Homo sapiens
GQNDTSQTSSPS

SEQ ID NO: 2 - DNA, 1362 bp, Artificial Sequence
  Annotated DNA sequence of HCA-UNC (anti-CD52g) (Heavy Chain)
caggttcagctgcagcaatggggagccggactgctgaagcctagcgagacactgtctctgacctgtgccgtgtacggcggcagcttcagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgagaagcgtgacagccgccgataccgccgtgtactattgcgccagaggctttatggtccgaggcatcatgtggaactactactacatggacgtgtggggcaagggcaccaccgtgacagtttctccagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 3 - protein, 454 aa, Artificial Sequence
  Annotated Amino Acid sequence of HCA-UNC (anti-CD52g)(Heavy Chain)
QVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 232 aa, Artificial Sequence
  Annotated Amino Acid sequence of Fab fragment of HCA-UNC (anti-CD52g) (Heavy Chain)
QVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 5 - protein, 220 aa, Artificial Sequence
  Annotated Amino Acid sequence of Fc fragment of HCA-UNC (anti-CD52g) (Heavy Chain)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 642 bp, Artificial Sequence
  DNA sequence of HCA-UNC (anti-CD52g) (Light Chain)
agcagcgagctgacacaggatccagtggtgtctgtggccctgggccagacagtgcggattacttgtcagggcgacagcctgagaacctaccacgcctcttggtatcagcagaagcccagacaggcccctgtgctggtcatctacgacgagaacaacagacccagcggcatccccgatagattcagcggcagcacatctggcaataccgccagcctgacaatcactggcgcccaggctgaagatgaggccgactactactgcaacagcagagacagcagcggcaaccggctggtttttggcggaggcacaaagctgacagtgctgggccaacctaaggccgctcctagcgtgacactgttccctccaagcagcgaagaactgcaggccaacaaggccacactcgtgtgcctgatcagcgacttttatcctggcgccgtgaccgtggcctggaaggctgatagttctcctgtgaaggccggcgtggaaaccaccacacctagcaagcagagcaacaacaaatacgccgccagcagctacctgagcctgacacctgagcagtggaagtcccacagatcctacagctgccaagtgacccacgagggcagcaccgtggaaaaaacagtggcccctaccgagtgcagc

SEQ ID NO: 7 - protein, 214 aa, Artificial Sequence
  Annotated Amino Acid sequence of HCA-UNC (anti-CD52g) (Light Chain)
SSELTQDPVVSVALGQTVRITCQGDSLRTYHASWYQQKPRQAPVLVIYDENNRPSGIPDRFSGSTSGNTASLTITGAQAEDEADYYCNSRDSSGNRLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 8 - DNA, 2148 bp, Artificial Sequence
  Annotated DNA sequence of Synthetic anti-CD52g Fab-IgG (Heavy Chain)
caggttcagctgcagcaatggggagccggactgctgaagcctagcgagacactgtctctgacctgtgccgtgtacggcggcagcttcagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgagaagcgtgacagccgccgataccgccgtgtactattgcgccagaggctttatggtccgaggcatcatgtggaactactactacatggacgtgtggggcaagggcaccaccgtgacagtttctccagcctctaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacaggtggcggaggatctggcggaggggggagcggagggggtgggtccggcggggggggttccgggggtggtggatcaggcggcggaggaagtcaggttcagctgcagcaatggggagccggactgctgaagcctagcgagacactgtctctgacctgtgccgtgtacggcggcagcttcagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgagaagcgtgacagccgccgataccgccgtgtactattgcgccagaggctttatggtccgaggcatcatgtggaactactactacatggacgtgtggggcaagggcaccaccgtgacagtttctccagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 9 - protein, 716 aa, Artificial Sequence
  Annotated Amino Acid sequence of Synthetic anti-CD52g Fab-IgG (Heavy Chain)
QVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSQVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 232 aa, Artificial Sequence; identical to SEQ ID NO: 4
  Annotated Amino Acid sequence of Synthetic anti-CD52g Fab fragment of Fab-IgG (Heavy Chain)

SEQ ID NO: 11 - protein, 220 aa, Artificial Sequence; identical to SEQ ID NO: 5
  Annotated Amino Acid sequence of Synthetic anti-CD52g Fc fragment of Fab-IgG (Heavy Chain)

SEQ ID NO: 12 - DNA, 642 bp, Artificial Sequence; identical to SEQ ID NO: 6
  DNA sequence of Synthetic anti-CD52g Fab-IgG (Light Chain)

SEQ ID NO: 13 - protein, 214 aa, Artificial Sequence; identical to SEQ ID NO: 7
  Annotated Amino Acid sequence of Synthetic anti-CD52g Fab-IgG (Light Chain)

SEQ ID NO: 14 - DNA, 2154 bp, Artificial Sequence
  Annotated DNA sequence of Synthetic anti-CD52g IgG-Fab (Heavy Chain)
caggttcagctgcagcaatggggagccggactgctgaagcctagcgagacactgtctctgacctgtgccgtgtacggcggcagcttcagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgagaagcgtgacagccgccgataccgccgtgtactattgcgccagaggctttatggtccgaggcatcatgtggaactactactacatggacgtgtggggcaagggcaccaccgtgacagtttctccagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccggtggcggaggatctggcggaggggggagcggagggggtgggtccggcggggggggttccgggggtggtggatcaggcggcggaggaagtcaggttcagctgcagcaatggggagccggactgctgaagcctagcgagacactgtctctgacctgtgccgtgtacggcggcagcttcagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgagaagcgtgacagccgccgataccgccgtgtactattgcgccagaggctttatggtccgaggcatcatgtggaactactactacatggacgtgtggggcaagggcaccaccgtgacagtttctccagcctctaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacaca

SEQ ID NO: 15 - protein, 718 aa, Artificial Sequence
  Annotated Amino Acid sequence of Synthetic anti-CD52g IgG-Fab (Heavy Chain)
QVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSQVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 16 - protein, 232 aa, Artificial Sequence; identical to SEQ ID NO: 4
  Annotated Amino Acid sequence of Synthetic anti-CD52g Fab fragment of IgG-Fab (Heavy Chain)

SEQ ID NO: 17 - protein, 220 aa, Artificial Sequence; identical to SEQ ID NO: 5
  Annotated Amino Acid sequence of Synthetic anti-CD52g Fc fragment of IgG-Fab (Heavy Chain)

SEQ ID NO: 18 - DNA, 642 bp, Artificial Sequence; identical to SEQ ID NO: 6
  DNA sequence of Synthetic anti-CD52g IgG-Fab (Light Chain)

SEQ ID NO: 19 - protein, 214 aa, Artificial Sequence; identical to SEQ ID NO: 7
  Annotated Amino Acid sequence of Synthetic anti-CD52g IgG-Fab (Light Chain)

SEQ ID NO: 20 - DNA, 2940 bp, Artificial Sequence
  Annotated DNA sequence of Synthetic anti-CD52g Fab-IgG-Fab (Heavy Chain)
caggttcagctgcagcaatggggagccggactgctgaagcctagcgagacactgtctctgacctgtgccgtgtacggcggcagcttcagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgagaagcgtgacagccgccgataccgccgtgtactattgcgccagaggctttatggtccgaggcatcatgtggaactactactacatggacgtgtggggcaagggcaccaccgtgacagtttctccagcctctaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacaggtggcggaggatctggcggaggggggagcggagggggtgggtccggcggggggggttccgggggtggtggatcaggcggcggaggaagtcaggttcagctgcagcaatggggagccggactgctgaagcctagcgagacactgtctctgacctgtgccgtgtacggcggcagcttcagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgagaagcgtgacagccgccgataccgccgtgtactattgcgccagaggctttatggtccgaggcatcatgtggaactactactacatggacgtgtggggcaagggcaccaccgtgacagtttctccagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccggtggcggaggatctggcggaggggggagcggagggggtgggtccggcggggggggttccgggggtggtggatcaggcggcggaggaagtcaggttcagctgcagcaatggggagccggactgctgaagcctagcgagacactgtctctgacctgtgccgtgtacggcggcagcttcagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgagaagcgtgacagccgccgataccgccgtgtactattgcgccagaggctttatggtccgaggcatcatgtggaactactactacatggacgtgtggggcaagggcaccaccgtgacagtttctccagcctctaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacaca

SEQ ID NO: 21 - protein, 980 aa, Artificial Sequence
  Annotated Amino Acid sequence of Synthetic anti-CD52g Fab-IgG-Fab (Heavy Chain)
QVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSQVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSQVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 22 - protein, 232 aa, Artificial Sequence; identical to SEQ ID NO: 4
  Annotated Amino Acid sequence of Synthetic anti-CD52g Fab fragment of Fab-IgG-Fab (Heavy Chain)

SEQ ID NO: 23 - protein, 220 aa, Artificial Sequence; identical to SEQ ID NO: 5
  Annotated Amino Acid sequence of Synthetic anti-CD52g Fc fragment of Fab-IgG-Fab (Heavy Chain)

SEQ ID NO: 24 - DNA, 642 bp, Artificial Sequence; identical to SEQ ID NO: 6
  DNA sequence of Synthetic anti-CD52g Fab-IgG-Fab (Light Chain)

SEQ ID NO: 25 - protein, 214 aa, Artificial Sequence; identical to SEQ ID NO: 7
  Annotated Amino Acid sequence of Synthetic anti-CD52g Fab-IgG-Fab (Light Chain)

SEQ ID NO: 26 - DNA, 3726 bp, Artificial Sequence
  Annotated DNA sequence of Synthetic anti-CD52g Fab-IgG-Fab-Fab (Heavy Chain)
caggttcagctgcagcaatggggagccggactgctgaagcctagcgagacactgtctctgacctgtgccgtgtacggcggcagcttcagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgagaagcgtgacagccgccgataccgccgtgtactattgcgccagaggctttatggtccgaggcatcatgtggaactactactacatggacgtgtggggcaagggcaccaccgtgacagtttctccagcctctaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacaggtggcggaggatctggcggaggggggagcggagggggtgggtccggcggggggggttccgggggtggtggatcaggcggcggaggaagtcaggttcagctgcagcaatggggagccggactgctgaagcctagcgagacactgtctctgacctgtgccgtgtacggcggcagcttcagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgagaagcgtgacagccgccgataccgccgtgtactattgcgccagaggctttatggtccgaggcatcatgtggaactactactacatggacgtgtggggcaagggcaccaccgtgacagtttctccagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccggtggcggaggctctggtggaggaggcagtggcggaggcggttctggtggtggtggctctggtggcggcggttcaggcggtggcggatctcaagttcagctgcagcaatggggagccggcctgctgaagccttctgagacactgtctctgacctgcgccgtgtacggcggcagctttagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgcggagcgtgacagccgctgatacagccgtgtactactgcgccagaggctttatggtccgaggcatcatgtggaactactactacatggacgtgtggggcaagggcaccaccgtgacagtttctccagccagcacaaagggccccagcgttttcccactggctcctagcagcaagagcacatctggtggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccatacagggggcggtggaagcggaggcgggggtagcggtggtggcggcagcggcggaggcggatcagggggcggcggaagtggtggcggtggttctcaggttcaactccaacagtggggcgctggactgctgaaacctagcgaaaccctgagcctgacatgtgctgtgtatggcggctccttctccggctactattggagctggattcggcagccaccaggcaagggactcgagtggattggagagatcaaccactccggctccaccaattacaatccatctctgcggtcccgcgtgaccatctccgtggatacctctaagaatcagttctcactgaagctgagatccgtgaccgctgccgacactgccgtgtattattgtgcccggggattcatggttcgagggattatgtggaattactattatatggatgtctggggaaaagggacgaccgtgactgtgtcccctgcctctacaaagggaccctccgtgtttcctctggctcccagctctaagtctaccagcggaggaacagctgccctgggatgtctcgtgaaagactacttccccgaaccagtgacagtcagctggaacagcggagccctgacttctggggtgcacacattccctgccgtcctgcaatcttctggcctgtacagcctgtccagcgtcgtgaccgttccttctagctctctgggaacacagacatatatctgtaatgtcaatcacaaaccctccaatacgaaggtcgacaaaaaggtcgagcctaagtcctgtgataagacccacacc

SEQ ID NO: 27 - protein, 1242 aa, Artificial Sequence
  Annotated Amino Acid sequence of Synthetic anti-CD52g Fab-IgG-Fab-Fab (Heavy Chain)
QVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSQVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSQVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSQVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 28 - protein, 232 aa, Artificial Sequence; identical to SEQ ID NO: 4
  Annotated Amino Acid sequence of Synthetic anti-CD52g Fab fragment of Fab-IgG-Fab-Fab (Heavy Chain)

SEQ ID NO: 29 - protein, 220 aa, Artificial Sequence; identical to SEQ ID NO: 5
  Annotated Amino Acid sequence of Synthetic anti-CD52g Fc fragment of Fab-IgG-Fab-Fab (Heavy Chain)

SEQ ID NO: 30 - DNA, 642 bp, Artificial Sequence; identical to SEQ ID NO: 6
  DNA sequence of Synthetic anti-CD52g Fab-IgG-Fab-Fab (Light Chain)

SEQ ID NO: 31 - protein, 214 aa, Artificial Sequence; identical to SEQ ID NO: 7
  Annotated Amino Acid sequence of Synthetic anti-CD52g Fab-IgG-Fab-Fab (Light Chain)

SEQ ID NO: 32 - DNA, 4512 bp, Artificial Sequence
  Annotated DNA sequence of Synthetic anti-CD52g Fab-Fab-IgG-Fab-Fab (Heavy Chain)
caagttcagctgcagcaatggggagccggcctgctgaagccttctgagacactgtctctgacctgcgccgtgtacggcggcagctttagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgcggagcgtgacagccgctgatacagccgtgtactactgcgccagaggctttatggtccgaggcatcatgtggaactactactacatggacgtgtggggcaagggcaccaccgtgacagtttctccagccagcacaaagggccccagcgttttcccactggctcctagcagcaagagcacatctggtggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccatacagggggcggtggaagcggaggcgggggtagcggtggtggcggcagcggcggaggcggatcagggggcggcggaagtggtggcggtggttctcaggttcaactccaacagtggggcgctggactgctgaaacctagcgaaaccctgagcctgacatgtgctgtgtatggcggctccttctccggctactattggagctggattcggcagccaccaggcaagggactcgagtggattggagagatcaaccactccggctccaccaattacaatccatctctgcggtcccgcgtgaccatctccgtggatacctctaagaatcagttctcactgaagctgagatccgtgaccgctgccgacactgccgtgtattattgtgcccggggattcatggttcgagggattatgtggaattactattatatggatgtctggggaaaagggacgaccgtgactgtgtcccctgcctctacaaagggaccctccgtgtttcctctggctcccagctctaagtctaccagcggaggaacagctgccctgggatgtctcgtgaaagactacttccccgaaccagtgacagtcagctggaacagcggagccctgacttctggggtgcacacattccctgccgtcctgcaatcttctggcctgtacagcctgtccagcgtcgtgaccgttccttctagctctctgggaacacagacatatatctgtaatgtcaatcacaaaccctccaatacgaaggtcgacaaaaaggtcgagcctaagtcctgtgataagacccacaccggtggcggaggctctgggggaggaggcagtggcggaggcggttctggtggtggtggctctggtggcggcggttcaggcggtggcggatctcaggttcagctgcagcaatggggagccggactgctgaagcctagcgagacactgtctctgacctgtgccgtgtacggcggcagcttcagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgagaagcgtgacagccgccgataccgccgtgtactattgcgccagaggctttatggtccgaggcatcatgtggaactactactacatggacgtgtggggcaagggcaccaccgttacagtctcacctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccggtggcggaggctctggtggaggaggcagtggcggaggcggttctggtggtggtggctctggtggcggcggttcaggcggtggcggatctcaagttcagctgcagcaatggggagccggcctgctgaagccttctgagacactgtctctgacctgcgccgtgtacggcggcagctttagcggctactactggtcctggatcagacagcctcctggcaaaggcctggaatggatcggcgagatcaatcacagcggcagcaccaactacaaccccagcctgagaagcagagtgaccatcagcgtggacaccagcaagaaccagttcagcctgaagctgcggagcgtgacagccgctgatacagccgtgtactactgcgccagaggctttatggtccgaggcatcatgtggaactactactacatggacgtgtggggcaagggcaccaccgtgacagtttctccagccagcacaaagggccccagcgttttcccactggctcctagcagcaagagcacatctggtggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccatacagggggcggtggaagcggaggcgggggtagcggtggtggcggcagcggcggaggcggatcagggggcggcggaagtggtggcggtggttctcaggttcaactccaacagtggggcgctggactgctgaaacctagcgaaaccctgagcctgacatgtgctgtgtatggcggctccttctccggctactattggagctggattcggcagccaccaggcaagggactcgagtggattggagagatcaaccactccggctccaccaattacaatccatctctgcggtcccgcgtgaccatctccgtggatacctctaagaatcagttctcactgaagctgagatccgtgaccgctgccgacactgccgtgtattattgtgcccggggattcatggttcgagggattatgtggaattactattatatggatgtctggggaaaagggacgaccgtgactgtgtcccctgcctctacaaagggaccctccgtgtttcctctggctcccagctctaagtctaccagcggaggaacagctgccctgggatgtctcgtgaaagactacttccccgaaccagtgacagtcagctggaacagcggagccctgacttctggggtgcacacattccctgccgtcctgcaatcttctggcctgtacagcctgtccagcgtcgtgaccgttccttctagctctctgggaacacagacatatatctgtaatgtcaatcacaaaccctccaatacgaaggtcgacaaaaaggtcgagcctaagtcctgtgataagacccacacc

SEQ ID NO: 33 - protein, 1504 aa, Artificial Sequence
  Annotated Amino Acid sequence of Synthetic anti-CD52g Fab-Fab-IgG-Fab-Fab (Heavy Chain)
QVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSQVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSQVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSQVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSQVQLQQWGAGLLKPSETLSLTCAVYGGSFSGYYWSWIRQPPGKGLEWIGEINHSGSTNYNPSLRSRVTISVDTSKNQFSLKLRSVTAADTAVYYCARGFMVRGIMWNYYYMDVWGKGTTVTVSPASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 34 - DNA, 642 bp, Artificial Sequence; identical to SEQ ID NO: 6
  Annotated DNA sequence of Synthetic anti-CD52g Fab-Fab-IgG-Fab-Fab (Light Chain)

SEQ ID NO: 35 - protein, 214 aa, Artificial Sequence; identical to SEQ ID NO: 7
  Annotated Amino Acid sequence of Synthetic anti-CD52g Fab-Fab-IgG-Fab-Fab (Light Chain)

SEQ ID NO: 36 - protein, 232 aa, Artificial Sequence; identical to SEQ ID NO: 4
  Annotated Amino Acid sequence of Synthetic anti-CD52g Fab fragment of Fab-Fab-IgG-Fab-Fab (Heavy Chain)

SEQ ID NO: 37 - protein, 214 aa, Artificial Sequence; identical to SEQ ID NO: 7
  Annotated Amino Acid sequence of Synthetic anti-CD52g Fab fragment of Fab-Fab-IgG-Fab-Fab (Light Chain)

SEQ ID NO: 38 - protein, 220 aa, Artificial Sequence; identical to SEQ ID NO: 5
  Annotated Amino Acid sequence of Synthetic anti-CD52g Fc fragment of Fab-Fab-IgG-Fab-Fab (Heavy Chain)

SEQ ID NO: 39 - DNA, 1365 bp, Artificial Sequence
  Annotated DNA sequence of anti-klebsiella IgG (Heavy Chain)
gaggtgcagctggttgaatctggcggaggactggttcagcctggcggatctctgagactgtcttgcgccgccagctttagcctgacaagctacgccgtgcacatccactgggttcgacaggcccctggcaaaggccttgaatgggttgccagagtgatctgggctggcggcatcacccactacaatagcgccctgatgagcagatacgccgacagcgtgaagggcagattcaccatcagcgccgacaccagcaagaacaccgcctacctgcagatgaacagcctgagagccgaggacaccgccgtgtactattgcgccagaggcaactgggccttcgactattggggacagggcaccctggtcaccgttagctctgcctctacaaagggccctagtgtgttccctctggctcccagcagcaagtctacatctggcggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggcaaa

SEQ ID NO: 40 - protein, 455 aa, Artificial Sequence
  Annotated Amino Acid sequence of anti-klebsiella IgG (Heavy Chain)
EVQLVESGGGLVQPGGSLRLSCAASFSLTSYAVHIHWVRQAPGKGLEWVARVIWAGGITHYNSALMSRYADSVKGRFTISADTSKNTAYLQMNSLRAEDTAVYYCARGNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 233 aa, Artificial Sequence
  Annotated Amino Acid sequence of Fab fragment of anti-klebsiella (Heavy Chain)
EVQLVESGGGLVQPGGSLRLSCAASFSLTSYAVHIHWVRQAPGKGLEWVARVIWAGGITHYNSALMSRYADSVKGRFTISADTSKNTAYLQMNSLRAEDTAVYYCARGNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 42 - protein, 220 aa, Artificial Sequence
  Annotated Amino Acid sequence of Fc fragment of anti-klebsiella (Heavy Chain)
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 666 bp, Artificial Sequence
  DNA sequence of anti-klebsiella (Light Chain)
gacatccagatgacacagagccctagcagcctgtctgccagcgtgggagacagagtgaccatcacctgtagagccagcagcgccagatccagcgtgtcctatattcacgtggcctggtatcagcagaagcccggcaaagcccctaagctgctgatctacgacaccagcaaactggccagcttcctgtacagcggcgtgccctctagattcagcggcagcagatctggcaccgacttcaccctgaccataagcagcctgcagcctgaggacttcgccacctactactgctttcaaggcagcggctacccctacacctttggccagggaacaaaggtggaaatcaagagaacagtggccgctcctagcgtgttcatcttcccaccttccgacgagcagctgaagtctggcacagcctctgtcgtgtgcctgctgaacaacttctaccccagagaagccaaggtgcagtggaaggtggacaacgccctgcagagcggcaatagccaagagagcgtgaccgagcaggacagcaaggactctacctacagcctgagcagcaccctgacactgagcaaggccgactacgagaagcacaaagtgtacgcctgcgaagtgacccaccagggcctttctagccctgtgaccaagagcttcaaccggggcgaatgt

SEQ ID NO: 44 - protein, 222 aa, Artificial Sequence
  Annotated Amino Acid sequence of anti-klebsiella (Light Chain)
DIQMTQSPSSLSASVGDRVTITCRASSARSSVSYIHVAWYQQKPGKAPKLLIYDTSKLASFLYSGVPSRFSGSRSGTDFTLTISSLQPEDFATYYCFQGSGYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 222 aa, Artificial Sequence; identical to SEQ ID NO: 44
  Annotated Amino Acid sequence of Fab fragment of anti-klebsiella IgG (Light Chain)

SEQ ID NO: 46 - DNA, 2154 bp, Artificial Sequence
  Annotated DNA sequence of Synthetic anti-klebsiella Fab-IgG (Heavy Chain)
gaggtgcagttggttgaatccggtggcgggttggtccagcctggcgggagccttcggcttagttgtgccgcatcatttagcctgacatcttacgctgtccatatccattgggtgcggcaagcgcctggtaagggcctggaatgggtggcaagggtgatatgggcagggggtattacgcattacaactctgcattgatgagtcggtacgccgacagcgtcaaaggtcggttcaccatttctgccgatacctctaagaacacagcctacctccagatgaactcactgcgagcggaggacactgctgtgtactattgcgcccgcggcaattgggcatttgactactgggggcaaggtacactcgtaacggtctcatctgcctctacaaagggccctagtgtgttccctctggctcccagcagcaagtctacatctggcggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacaccggtggcggaggatctggtggaggagggagcggagggggtgggtccggagggggtggttccggaggtggtggatcaggtggcggaggaagtgaggtgcagctggttgaatctggcggaggactggttcagcctggcggatctctgagactgtcttgcgccgccagctttagcctgacaagctacgccgtgcacatccactgggttcgacaggcccctggcaaaggccttgaatgggttgccagagtgatctgggctggcggcatcacccactacaatagcgccctgatgagcagatacgccgacagcgtgaagggcagattcaccatcagcgccgacaccagcaagaacaccgcctacctgcagatgaacagcctgagagccgaggacaccgccgtgtactattgcgccagaggcaactgggccttcgactattggggacagggcaccctggtcaccgttagctctgcctctacaaagggccctagtgtgttccctctggctcccagcagcaagtctacatctggcggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggcaaa

SEQ ID NO: 47 - protein, 718 aa, Artificial Sequence
  Annotated Amino Acid sequence of Synthetic anti-klebsiella IgG Fab-IgG (Heavy Chain)
EVQLVESGGGLVQPGGSLRLSCAASFSLTSYAVHIHWVRQAPGKGLEWVARVIWAGGITHYNSALMSRYADSVKGRFTISADTSKNTAYLQMNSLRAEDTAVYYCARGNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSEVQLVESGGGLVQPGGSLRLSCAASFSLTSYAVHIHWVRQAPGKGLEWVARVIWAGGITHYNSALMSRYADSVKGRFTISADTSKNTAYLQMNSLRAEDTAVYYCARGNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 233 aa, Artificial Sequence; identical to SEQ ID NO: 41
  Annotated Amino Acid sequence of Synthetic anti-klebsiella Fab fragment of Fab-IgG (Heavy Chain)

SEQ ID NO: 49 - protein, 220 aa, Artificial Sequence; identical to SEQ ID NO: 42
  Annotated Amino Acid sequence of Synthetic anti-klebsiella Fc fragment of Fab-IgG (Heavy Chain)

SEQ ID NO: 50 - DNA, 666 bp, Artificial Sequence; identical to SEQ ID NO: 43
  DNA sequence of Synthetic anti-klebsiella Fab-IgG (Light Chain)

SEQ ID NO: 51 - protein, 222 aa, Artificial Sequence; identical to SEQ ID NO: 44
  Annotated Amino Acid sequence of Synthetic anti-klebsiella Fab-IgG (Light Chain)

SEQ ID NO: 52 - protein, 222 aa, Artificial Sequence; identical to SEQ ID NO: 44
  Annotated Amino Acid sequence of Synthetic anti-klebsiella Fab fragment of Fab-IgG (Light Chain)

SEQ ID NO: 53 - DNA, 2160 bp, Artificial Sequence
  Annotated DNA sequence of Synthetic anti-klebsiella IgG-Fab (Heavy Chain)
gaggtgcagctggttgaatctggcggaggactggttcagcctggcggatctctgagactgtcttgcgccgccagctttagcctgacaagctacgccgtgcacatccactgggttcgacaggcccctggcaaaggccttgaatgggttgccagagtgatctgggctggcggcatcacccactacaatagcgccctgatgagcagatacgccgacagcgtgaagggcagattcaccatcagcgccgacaccagcaagaacaccgcctacctgcagatgaacagcctgagagccgaggacaccgccgtgtactattgcgccagaggcaactgggccttcgactattggggacagggcaccctggtcaccgttagctctgcctctacaaagggccctagtgtgttccctctggctcccagcagcaagtctacatctggcggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggcaaaggatccggtggcggaggatctggtggaggagggagcggagggggtgggtccggagggggtggttccggaggtggtggatcaggtggcggaggaagtgaggtgcagttggttgaatccggtggcgggttggtccagcctggcgggagccttcggcttagttgtgccgcatcatttagcctgacatcttacgctgtccatatccattgggtgcggcaagcgcctggtaagggcctggaatgggtggcaagggtgatatgggcagggggtattacgcattacaactctgcattgatgagtcggtacgccgacagcgtcaaaggtcggttcaccatttctgccgatacctctaagaacacagcctacctccagatgaactcactgcgagcggaggacactgctgtgtactattgcgcccgcggcaattgggcatttgactactgggggcaaggtacactcgtaacggtctcatctgcctctacaaagggccctagtgtgttccctctggctcccagcagcaagtctacatctggcggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacc

SEQ ID NO: 54 - protein, 720 aa, Artificial Sequence
  Annotated Amino Acid sequence of Synthetic anti-klebsiella IgG-Fab (Heavy Chain)
EVQLVESGGGLVQPGGSLRLSCAASFSLTSYAVHIHWVRQAPGKGLEWVARVIWAGGITHYNSALMSRYADSVKGRFTISADTSKNTAYLQMNSLRAEDTAVYYCARGNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSEVQLVESGGGLVQPGGSLRLSCAASFSLTSYAVHIHWVRQAPGKGLEWVARVIWAGGITHYNSALMSRYADSVKGRFTISADTSKNTAYLQMNSLRAEDTAVYYCARGNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 55 - protein, 233 aa, Artificial Sequence; identical to SEQ ID NO: 41
  Annotated Amino Acid sequence of Synthetic anti-klebsiella Fab fragment of IgG-Fab (Heavy Chain)

SEQ ID NO: 56 - protein, 220 aa, Artificial Sequence; identical to SEQ ID NO: 42
  Annotated Amino Acid sequence of Synthetic anti-klebsiella Fc fragment of IgG-Fab (Heavy Chain)

SEQ ID NO: 57 - DNA, 666 bp, Artificial Sequence; identical to SEQ ID NO: 43
  DNA sequence of Synthetic anti-klebsiella IgG-Fab (Light Chain)

SEQ ID NO: 58 - protein, 222 aa, Artificial Sequence; identical to SEQ ID NO: 44
  Annotated Amino Acid sequence of Synthetic anti-klebsiella IgG-Fab (Light Chain)

SEQ ID NO: 59 - protein, 222 aa, Artificial Sequence; identical to SEQ ID NO: 44
  Annotated Amino Acid sequence of Synthetic anti-klebsiella Fab fragment of IgG-Fab (Light Chain)

SEQ ID NO: 60 - DNA, 2949 bp, Artificial Sequence
  Annotated DNA sequence of Synthetic anti-klebsiella Fab-IgG-Fab (Heavy Chain)
gaggtgcagttggttgaatccggtggcgggttggtccagcctggcgggagccttcggcttagttgtgccgcatcatttagcctgacatcttacgctgtccatatccattgggtgcggcaagcgcctggtaagggcctggaatgggtggcaagggtgatatgggcagggggtattacgcattacaactctgcattgatgagtcggtacgccgacagcgtcaaaggtcggttcaccatttctgccgatacctctaagaacacagcctacctccagatgaactcactgcgagcggaggacactgctgtgtactattgcgcccgcggcaattgggcatttgactactgggggcaaggtacactcgtaacggtctcatctgcctctacaaagggccctagtgtgttccctctggctcccagcagcaagtctacatctggcggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacaccggtggcggaggatctggtggaggagggagcggagggggtgggtccggagggggtggttccggaggtggtggatcaggtggcggaggaagtgaggtgcagctggttgaatctggcggaggactggttcagcctggcggatctctgagactgtcttgcgccgccagctttagcctgacaagctacgccgtgcacatccactgggttcgacaggcccctggcaaaggccttgaatgggttgccagagtgatctgggctggcggcatcacccactacaatagcgccctgatgagcagatacgccgacagcgtgaagggcagattcaccatcagcgccgacaccagcaagaacaccgcctacctgcagatgaacagcctgagagccgaggacaccgccgtgtactattgcgccagaggcaactgggccttcgactattggggacagggcaccctggtcaccgttagctctgcctctacaaagggccctagtgtgttccctctggctcccagcagcaagtctacatctggcggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgttcctgtttcctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtccgtgctgaccgtgctgcaccaggattggctgaacggcaaagagtacaagtgcaaggtgtccaacaaggccctgcctgctcctatcgagaaaaccatcagcaaggccaagggccagcctagggaaccccaggtttacacactgcctccaagccgggaagagatgaccaagaaccaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctcctggcaaaggatccggtggcggaggatctggtggaggagggagcggagggggtgggtccggagggggtggttccggaggtggtggatcaggtggcggaggaagtgaggtgcagttggttgaatccggtggcgggttggtccagcctggcgggagccttcggcttagttgtgccgcatcatttagcctgacatcttacgctgtccatatccattgggtgcggcaagcgcctggtaagggcctggaatgggtggcaagggtgatatgggcagggggtattacgcattacaactctgcattgatgagtcggtacgccgacagcgtcaaaggtcggttcaccatttctgccgatacctctaagaacacagcctacctccagatgaactcactgcgagcggaggacactgctgtgtactattgcgcccgcggcaattgggcatttgactactgggggcaaggtacactcgtaacggtctcatctgcctctacaaagggccctagtgtgttccctctggctcccagcagcaagtctacatctggcggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgcctagctctagcctgggcacccagacctacatctgcaatgtgaaccacaagcctagcaacaccaaggtggacaagaaggtggaacccaagagctgcgacaagacccacacc

SEQ ID NO: 61 - protein, 983 aa, Artificial Sequence
  Annotated Amino Acid sequence of Synthetic anti-klebsiella Fab-IgG-Fab (Heavy Chain)
EVQLVESGGGLVQPGGSLRLSCAASFSLTSYAVHIHWVRQAPGKGLEWVARVIWAGGITHYNSALMSRYADSVKGRFTISADTSKNTAYLQMNSLRAEDTAVYYCARGNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSEVQLVESGGGLVQPGGSLRLSCAASFSLTSYAVHIHWVRQAPGKGLEWVARVIWAGGITHYNSALMSRYADSVKGRFTISADTSKNTAYLQMNSLRAEDTAVYYCARGNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSEVQLVESGGGLVQPGGSLRLSCAASFSLTSYAVHIHWVRQAPGKGLEWVARVIWAGGITHYNSALMSRYADSVKGRFTISADTSKNTAYLQMNSLRAEDTAVYYCARGNWAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 62 - protein, 233 aa, Artificial Sequence; identical to SEQ ID NO: 41
  Annotated Amino Acid sequence of Synthetic anti-klebsiella Fab fragment of Fab-IgG-Fab (Heavy Chain)

SEQ ID NO: 63 - protein, 220 aa, Artificial Sequence; identical to SEQ ID NO: 42
  Annotated Amino Acid sequence of Synthetic anti-klebsiella Fc fragment of Fab-IgG-Fab (Heavy Chain)

SEQ ID NO: 64 - DNA, 666 bp, Artificial Sequence; identical to SEQ ID NO: 43
  DNA sequence of Synthetic anti-klebsiella Fab-IgG-Fab (Light Chain)

SEQ ID NO: 65 - protein, 222 aa, Artificial Sequence; identical to SEQ ID NO: 44
  Annotated Amino Acid sequence of Synthetic anti-klebsiella Fab-IgG-Fab (Light Chain)

SEQ ID NO: 66 - protein, 222 aa, Artificial Sequence; identical to SEQ ID NO: 44
  Annotated Amino Acid sequence of Synthetic anti-klebsiella Fab fragment of Fab-IgG-Fab (Light Chain)

SEQ ID NO: 67 - DNA, 1350 bp, Artificial Sequence
  Annotated DNA sequence of anti-Salmonella LPS IgG (Heavy Chain)
gaagtgaagctggtggaatctggcggcggactggttcaacctggcggatctctgtctctgagctgtgccgccagcggcttcaccttcagcgactactacatgacctgggtccgacaggcccctggaaaagctcctgaatggctggccctgatccggaacaagagaaacggcgataccgccgagtacagcgcctctgtgaagggcagattcaccatcagccgggactacagccggtccatcctgcaccttcagatgaacgccctgagaaccgaggactccgccacctactattgcgtgcgacaaggcagaggctacaccctggattattggggccagggcacaagcgtgacagtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 68 - protein, 450 aa, Artificial Sequence
  Annotated Amino Acid sequence of anti-Salmonella LPS IgG (Heavy Chain)
EVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 228 aa, Artificial Sequence
  Annotated Amino Acid sequence of Fab fragment of anti-Salmonella LPS IgG (Heavy Chain)
EVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 70 - protein, 219 aa, Artificial Sequence
  Annotated Amino Acid sequence of Fc fragment of anti-Salmonella LPS (Heavy Chain)
CPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - DNA, 639 bp, Artificial Sequence
  DNA sequence of anti-Salmonella LPS (Light Chain)
gacatccagatgaatcagagccccagcagcctgtctgccagcctgggagataccatcagcatcacctgtcgggccagccagaacatcaacatctggctgagctggtatcagcagaaacccggcaacgtgcccaagctgctgatctacaaggccagcaatctgcacaccggcgtgcccagcagattttctggctctggcagcggcaccgacttcaccctgatcatatctagcctgcagcctgaggatatcgccacctactactgcctgcaaggccagagctaccccagaacatttggcggaggcaccaagctggaaatcaagacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 72 - protein, 213 aa, Artificial Sequence
  Annotated Amino Acid sequence of anti-Salmonella LPS (Light Chain)
DIQMNQSPSSLSASLGDTISITCRASQNINIWLSWYQQKPGNVPKLLIYKASNLHTGVPSRFSGSGSGTDFTLIISSLQPEDIATYYCLQGQSYPRTFGGGTKLEIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 73 - protein, 213 aa, Artificial Sequence; identical to SEQ ID NO: 72
  Annotated Amino Acid sequence of Fab fragment of Synthetic anti-Salmonella LPS IgG (Light Chain)

SEQ ID NO: 74 - DNA, 2124 bp, Artificial Sequence
  Annotated DNA sequence of Synthetic anti-Salmonella LPS Fab-IgG (Heavy Chain)
gaagtgaagctggtggaatctggcggcggactggttcaacctggcggatctctgtctctgagctgtgccgccagcggcttcaccttcagcgactactacatgacctgggtccgacaggcccctggaaaagctcctgaatggctggccctgatccggaacaagagaaacggcgataccgccgagtacagcgcctctgtgaagggcagattcaccatcagccgggactacagccggtccatcctgcaccttcagatgaacgccctgagaaccgaggactccgccacctactattgcgtgcgacaaggcagaggctacaccctggattattggggccagggcacaagcgtgacagtgtctagcgccagcacaaagggcccctctgtttttccactggctcccagcagcaagagcacaagcggaggaacagctgccctgggatgcctcgtgaaggactacttccctgaaccagtgaccgtgtcctggaactctggcgctctgacttctggggtccacactttcccagctgtcctgcagtctagcggactgtactctctgagcagcgtggtcacagtgcctagctctagcctgggcacccagacctacatctgcaatgtgaaccacaagcctagcaacacgaaggtcgacaaaaaggtggaacccaagagctgcgacaagacccatacaggcggcggaggatctggcggaggtggaagcggaggcggaggaagcggtggcggcggtagtggcggtggtggttcaggcggtggcggatctgaagtgaaactggttgaaagcggcggaggcctggttcagccaggtggaagtctctctctgtcttgtgccgcctctggctttaccttctctgattactatatgacgtgggttcgccaagctcctggcaaggcaccagaatggctcgctctgattagaaacaagcggaatggcgacacagccgagtattccgccagcgtgaaaggccggttcaccatctccagagactactcccgcagcatcctgcatctgcaaatgaatgctctgcggaccgaggacagcgctacctattactgcgttaggcaaggccggggatacacactggactactggggacaaggcacctccgtgactgtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 75 - protein, 708 aa, Artificial Sequence
  Annotated Amino Acid sequence of Synthetic anti-Salmonella LPS IgG Fab-IgG (Heavy Chain)
EVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSEVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 76 - protein, 228 aa, Artificial Sequence; identical to SEQ ID NO: 69
  Annotated Amino Acid sequence of Synthetic anti-Salmonella LPS Fab fragment of Fab-IgG (Heavy Chain)

SEQ ID NO: 77 - protein, 220 aa, Artificial Sequence; identical to SEQ ID NO: 5
  Annotated Amino Acid sequence of Synthetic anti-Salmonella LPS Fc fragment of Fab-IgG (Heavy Chain)

SEQ ID NO: 78 - DNA, 639 bp, Artificial Sequence; identical to SEQ ID NO: 71
  DNA sequence of Synthetic anti-Salmonella LPS Fab-IgG (Light Chain)

SEQ ID NO: 79 - protein, 213 aa, Artificial Sequence; identical to SEQ ID NO: 72
  Annotated Amino Acid sequence of Synthetic anti-Salmonella LPS Fab-IgG (Light Chain)

SEQ ID NO: 80 - protein, 213 aa, Artificial Sequence; identical to SEQ ID NO: 72
  Annotated Amino Acid sequence of Synthetic anti-Salmonella LPS Fab fragment of Fab-IgG (Light Chain)

SEQ ID NO: 81 - DNA, 2130 bp, Artificial Sequence
  Annotated DNA sequence of Synthetic anti-Salmonella LPS IgG-Fab (Heavy Chain)
gaagtgaagctggtggaatctggcggcggactggttcaacctggcggatctctgtctctgagctgtgccgccagcggcttcaccttcagcgactactacatgacctgggtccgacaggcccctggaaaagctcctgaatggctggccctgatccggaacaagagaaacggcgataccgccgagtacagcgcctctgtgaagggcagattcaccatcagccgggactacagccggtccatcctgcaccttcagatgaacgccctgagaaccgaggactccgccacctactattgcgtgcgacaaggcagaggctacaccctggattattggggccagggcacaagcgtgacagtgtctagcgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccggcggaggcggatctggtggcggaggtagtggcggcggaggttcaggtggtggtggtagcggaggtggcggttctggcggtggtggatctgaagtgaagctggtggaatctggcggaggcctggttcaacctggcggatctctgtctctgagctgtgccgccagcggcttcaccttcagcgactactacatgacctgggtccgacaggcccctggaaaagctcctgaatggctggccctgatccggaacaagagaaacggcgataccgccgagtacagcgcctctgtgaagggcagattcaccatcagccgggactacagccggtccatcctgcaccttcagatgaacgccctgagaaccgaggatagcgccacctactactgcgtgcgacaaggcagaggctacaccctggattattggggccagggcacaagcgtgacagtgtctagcgcctctacaaagggccccagcgttttcccactggctcctagcagcaagagcacaagcggaggaacagccgctctgggctgtctggtcaaggactactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccacacc

SEQ ID NO: 82 - protein, 710 aa, Artificial Sequence
  Annotated Amino Acid sequence of Synthetic anti-Salmonella LPS IgG-Fab (Heavy Chain)
EVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSEVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 83 - protein, 228 aa, Artificial Sequence; identical to SEQ ID NO: 69
  Annotated Amino Acid sequence of Synthetic anti-Salmonella LPS Fab fragment of IgG-Fab (Heavy Chain)

SEQ ID NO: 84 - protein, 220 aa, Artificial Sequence; identical to SEQ ID NO: 5
  Annotated Amino Acid sequence of Synthetic anti-Salmonella LPS Fc fragment of IgG-Fab (Heavy Chain)

SEQ ID NO: 85 - DNA, 639 bp, Artificial Sequence; identical to SEQ ID NO: 71
  DNA sequence of Synthetic anti-Salmonella LPS IgG-Fab (Light Chain)

SEQ ID NO: 86 - protein, 213 aa, Artificial Sequence; identical to SEQ ID NO: 72
  Annotated Amino Acid sequence of Synthetic anti-Salmonella LPS IgG-Fab (Light Chain)

SEQ ID NO: 87 - protein, 213 aa, Artificial Sequence; identical to SEQ ID NO: 72
  Annotated Amino Acid sequence of Synthetic anti-Salmonella LPS Fab fragment of IgG-Fab (Light Chain)

SEQ ID NO: 88 - DNA, 2904 bp, Artificial Sequence
  Annotated DNA sequence of Synthetic anti-Salmonella LPS Fab-IgG-Fab (Heavy Chain)
gaagtgaagctggtggaatctggcggcggactggttcaacctggcggatctctgtctctgagctgtgccgccagcggcttcaccttcagcgactactacatgacctgggtccgacaggcccctggaaaagctcctgaatggctggccctgatccggaacaagagaaacggcgataccgccgagtacagcgcctctgtgaagggcagattcaccatcagccgggactacagccggtccatcctgcaccttcagatgaacgccctgagaaccgaggactccgccacctactattgcgtgcgacaaggcagaggctacaccctggattattggggccagggcacaagcgtgacagtgtctagcgccagcacaaagggcccctctgtttttccactggctcccagcagcaagagcacaagcggaggaacagctgccctgggatgcctcgtgaaggactacttccctgaaccagtgaccgtgtcctggaactctggcgctctgacttctggggtccacactttcccagctgtcctgcagtctagcggactgtactctctgagcagcgtggtcacagtgcctagctctagcctgggcacccagacctacatctgcaatgtgaaccacaagcctagcaacacgaaggtcgacaaaaaggtggaacccaagagctgcgacaagacccatacaggcggcggaggatctggcggaggtggaagcggaggcggaggaagcggtggcggcggtagtggcggtggtggttcaggcggtggcggatctgaagtgaaactggttgaaagcggcggaggcctggttcagccaggtggaagtctctctctgtcttgtgccgcctctggctttaccttctctgattactatatgacgtgggttcgccaagctcctggcaaggcaccagaatggctcgctctgattagaaacaagcggaatggcgacacagccgagtattccgccagcgtgaaaggccggttcaccatctccagagactactcccgcagcatcctgcatctgcaaatgaatgctctgcggaccgaggacagcgctacctattactgcgttaggcaaggccggggatacacactggactactggggacaaggcacctccgtgactgtgtcctccgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccggcggaggcggatctggtggcggaggtagtggcggcggaggttcaggtggtggtggtagcggaggtggcggttctggcggtggtggatctgaagtgaagctggtggaatctggcggaggcctggttcaacctggcggatctctgtctctgagctgtgccgccagcggcttcaccttcagcgactactacatgacctgggtccgacaggcccctggaaaagctcctgaatggctggccctgatccggaacaagagaaacggcgataccgccgagtacagcgcctctgtgaagggcagattcaccatcagccgggactacagccggtccatcctgcaccttcagatgaacgccctgagaaccgaggatagcgccacctactactgcgtgcgacaaggcagaggctacaccctggattattggggccagggcacaagcgtgacagtgtctagcgcctctacaaagggccccagcgttttcccactggctcctagcagcaagagcacaagcggaggaacagccgctctgggctgtctggtcaaggactactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccacacc

SEQ ID NO: 89 - protein, 968 aa, Artificial Sequence
  Annotated Amino Acid sequence of Synthetic anti-Salmonella LPS Fab-IgG-Fab (Heavy Chain)
EVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSEVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSEVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 90 - protein, 228 aa, Artificial Sequence; identical to SEQ ID NO: 69
  Annotated Amino Acid sequence of Synthetic anti-Salmonella LPS Fab fragment of Fab-IgG-Fab (Heavy Chain)

SEQ ID NO: 91 - protein, 220 aa, Artificial Sequence; identical to SEQ ID NO: 5
  Annotated Amino Acid sequence of Synthetic anti-Salmonella LPS Fc fragment of Fab-IgG-Fab (Heavy Chain)

SEQ ID NO: 92 - DNA, 639 bp, Artificial Sequence; identical to SEQ ID NO: 71
  DNA sequence of Synthetic anti-Salmonella LPS Fab-IgG-Fab (Light Chain)

SEQ ID NO: 93 - protein, 213 aa, Artificial Sequence; identical to SEQ ID NO: 72
  Annotated Amino Acid sequence of Synthetic anti-Salmonella LPS Fab-IgG-Fab (Light Chain)

SEQ ID NO: 94 - protein, 213 aa, Artificial Sequence; identical to SEQ ID NO: 72
  Annotated Amino Acid sequence of Synthetic anti-Salmonella LPS Fab fragment of Fab-IgG-Fab (Light Chain)

SEQ ID NO: 95 - DNA, 4452 bp, Artificial Sequence
  Annotated DNA sequence of Synthetic anti-Salmonella LPS Fab-Fab-IgG-Fab-Fab(Heavy Chain)
gaagtgaagctggtggaaagcggcggaggactggttcaacctggcggatctctgagcctgtcttgtgccgccagcggcttcaccttcagcgactactacatgacctgggtccgacaggcccctggaaaagctcctgaatggctggccctgatccggaacaagagaaacggcgataccgccgagtacagcgcctctgtgaagggcagattcaccatcagccgggactacagccggtccatcctgcaccttcagatgaacgccctgagaaccgaggactccgccacctactattgcgtgcgacaaggcagaggctacaccctggattattggggccagggcacaagcgtgacagtgtctagcgccagcacaaagggcccctctgtttttccactggctcccagcagcaagagcacaagcggaggaacagctgccctgggatgcctcgtgaaggactacttccctgaaccagtgaccgtgtcctggaactctggcgctctgacatctggggtgcacacattccctgctgtgctgcagagcagcggcctgtattctctgagcagcgtggtcacagtgcccagctctagtctgggcacccagacctacatctgcaatgtgaaccacaagcctagcaacacgaaggtcgacaaaaaggtggaacccaagagctgcgacaagacccatacaggcggaggcggatctggtggtggtggatctggcggtggcggttcaggtggcggcggtagcggaggtggtggtagtggtggtggcggctctgaagtgaaactcgtcgaatctggtggcggactggtgcagccaggtggaagtctgtctctgagctgtgccgcctctggctttaccttctctgattactatatgacgtgggttcgccaagctcctggcaaggcaccagaatggctcgctctgattagaaacaagcggaatggcgacacagccgagtattccgccagcgtgaaaggccggttcaccatctccagagactactcccgcagcatcctgcatctgcaaatgaatgctctgcggaccgaggacagcgctacctattactgcgttaggcaaggccggggatacacactggactactggggacaaggcacctccgtgactgtgtcctctgcctctaccaagggacccagcgtgttcccacttgcacctagcagcaagtctaccagcggcggaactgccgctctcggatgcctggtcaaagattatttccccgaacctgtcaccgtcagctggaatagcggagcccttaccagcggagtgcatactttccctgccgtcctccagtcatccgggctgtatagtctgtcctccgtggttaccgtgccaagcagctctctgggaacacagacatatatctgtaatgtcaatcacaaaccctccaacacaaaagtggacaaaaaagtcgagccgaagtcctgtgataagacacacactggcggcggaggttctggcggaggtggaagcggaggcggtggctcaggcggcggtggcagtggcggaggcggtagcggcggaggcggttctgaagttaagctggttgagtccggcggtggccttgtgcagcctggtggttctctctctctgtcctgtgctgcctccggattcaccttttccgattattacatgacatgggttcgacaagcaccagggaaagccccagagtggctggcactcatcagaaacaaacgcaacggggacaccgccgaatactctgccagtgtcaaaggcaggtttacaatcagcagggattactctcggagcattctccacctccaaatgaacgcactccgcacagaggatagcgccacttactactgtgtccggcaaggacggggctataccctcgattactggggtcaagggacatctgtgaccgtcagttctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccggcggaggcggatctggcggcggaggtagtggcggcggaggttcaggtggtggtggtagcggaggtggcggttctggcggtggtggaagcgaagtgaagctggtggaatctggcggaggcctggttcaacctggcggatctctgtctctgagctgtgccgccagcggcttcaccttcagcgactactacatgacctgggtccgacaggcccctggaaaagctcctgaatggctggccctgatccggaacaagagaaacggcgataccgccgagtacagcgcctctgtgaagggcagattcaccatcagccgggactacagccggtccatcctgcaccttcagatgaacgccctgagaaccgaggatagcgccacctactactgcgtgcgacaaggcagaggctacaccctggattattggggccagggcacaagcgtgacagtgtctagcgcctctacaaagggccccagcgttttcccactggctcctagcagcaagagcacaagcggaggaacagccgctctgggctgtctggtcaaggactactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccatacaggcggtggcggaagcggaggcggtggctcaggtggcggaggttctggcggaggcggcagtggtggtggtggcagtggtggcggaggatctgaagtcaaactggtcgaaagcggaggtggactggttcagccaggtggaagcctgtctctgtcttgtgccgcttccggctttaccttctctgattactatatgacgtgggttcgccaagctcctggcaaggcaccagaatggctcgctctgattagaaacaagcggaatggcgacacagccgagtattccgccagcgtgaaaggccggttcaccatctccagagactactcccgcagcatcctgcatctgcaaatgaatgctctgcggaccgaggactccgccacatattactgtgtcagacaaggccggggatacacactcgactactggggacagggaacctccgtgactgtgtcctctgccagcacaaaggggccctccgtgtttcctctggctccaagctccaagtctaccagcggtggaactgctgccctgggatgcctcgtgaaggattacttcccagaaccagtgacagtcagctggaacagcggagccctgacttctggggtgcacacattccctgccgtcctgcaatcttctggcctgtacagcctgtcctccgtcgtgaccgttccttctagctctctgggaacacagacatatatctgtaatgtcaatcacaaaccctccaatacgaaggtcgacaaaaaggtcgagcctaagtcctgtgataagacccacacc

SEQ ID NO: 96 - protein, 1484 aa, Artificial Sequence
  Annotated Amino Acid sequence of Synthetic anti-Salmonella LPS Fab-Fab-IgG-Fab-Fab (Heavy Chain)
EVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSEVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSEVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSEVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSEVKLVESGGGLVQPGGSLSLSCAASGFTFSDYYMTWVRQAPGKAPEWLALIRNKRNGDTAEYSASVKGRFTISRDYSRSILHLQMNALRTEDSATYYCVRQGRGYTLDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 97 - protein, 228 aa, Artificial Sequence; identical to SEQ ID NO: 69
  Annotated Amino Acid sequence of Synthetic anti-Salmonella LPS Fab fragment of Fab-Fab-IgG-Fab-Fab (Heavy Chain)

SEQ ID NO: 98 - protein, 220 aa, Artificial Sequence; identical to SEQ ID NO: 5
  Annotated Amino Acid sequence of Synthetic anti-Salmonella LPS Fc fragment of Fab-Fab-IgG-Fab-Fab (Heavy Chain)

SEQ ID NO: 99 - DNA, 639 bp, Artificial Sequence; identical to SEQ ID NO: 71
  DNA sequence of Synthetic anti-Salmonella LPS Fab-Fab-IgG-Fab-Fab (Light Chain)

SEQ ID NO: 100 - protein, 213 aa, Artificial Sequence; identical to SEQ ID NO: 72
  Annotated Amino Acid sequence of Synthetic anti-Salmonella LPS Fab-Fab-IgG-Fab-Fab (Light Chain)

SEQ ID NO: 101 - protein, 213 aa, Artificial Sequence; identical to SEQ ID NO: 72
  Annotated Amino Acid sequence of Synthetic anti-Salmonella LPS Fab fragment of Fab-Fab-IgG-Fab-Fab (Light Chain)

SEQ ID NO: 102 - DNA, 1341 bp, Artificial Sequence
  Annotated DNA sequence of anti-Gonorrhea (2C7) IgG (Heavy Chain)
gaggttcagctgcagcagtctggacctgagctggtcaagcctggcagcagcgtgaagatcagctgtaaaggcagcggctacaccttcaccgactacaacatggaatgggtcaagcagagccacggcaagagcctggaatggatcggcgtgatcaaccccaacaaccggttcaccagctacaaccagaacttcagaggcaaggccacactgaccgtggacaagagcagcagcaccgcctacatggatctgagaagcctgaccagcgaggacagcgccgtgtatttttgtgccggcagccggtggtatcagtacgactattggggccagggcacaaccctgaccgttagctctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 103 - protein, 447 aa, Artificial Sequence
  Annotated Amino Acid sequence of anti-Gonorrhea (2C7) IgG (Heavy Chain)
EVQLQQSGPELVKPGSSVKISCKGSGYTFTDYNMEWVKQSHGKSLEWIGVINPNNRFTSYNQNFRGKATLTVDKSSSTAYMDLRSLTSEDSAVYFCAGSRWYQYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 225 aa, Artificial Sequence
  Annotated Amino Acid sequence of Fab fragment of anti-Gonorrhea (2C7) (Heavy Chain)
EVQLQQSGPELVKPGSSVKISCKGSGYTFTDYNMEWVKQSHGKSLEWIGVINPNNRFTSYNQNFRGKATLTVDKSSSTAYMDLRSLTSEDSAVYFCAGSRWYQYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 105 - protein, 220 aa, Artificial Sequence; identical to SEQ ID NO: 5
  Annotated Amino Acid sequence of Fc fragment of anti-Gonorrhea (2C7) (Heavy Chain)

SEQ ID NO: 106 - DNA, 645 bp, Artificial Sequence
  DNA sequence of anti-Gonorrhea (2C7) (Light Chain)
caggtggtggtcacacaagagagcgccctgacaacaagccctggcgagacagtgaccctgacctgcagatcttctacaggcgccgtgaccacctccaactacgccaattgggtgcaagagaagcccgaccacctgttcacaggcctgatcggcggcatcaacaatagagcacctggcgtgccagccagattcagcggatctctgatcggagacaaggccgcactgacaatcacaggcgcccagacagaggacgaggccatctacttttgcgccctgtggtacagcaaccactgggttttcggcggaggcaccaagctgacagttctgggccaacctaaggccgctcctagcgtgacactgttccctccaagcagcgaagaactgcaggccaacaaggccacactcgtgtgcctgatcagcgacttttatcctggcgccgtgaccgtggcctggaaggctgatagttctcctgtgaaggccggcgtggaaaccaccacacctagcaagcagagcaacaacaaatacgccgccagcagctacctgagcctgacacctgagcagtggaagtcccacagatcctacagctgccaagtgacccacgagggcagcaccgtggaaaaaacagtggcccctaccgagtgcagc

SEQ ID NO: 107 - protein, 215 aa, Artificial Sequence
  Annotated Amino Acid sequence of anti-Gonorrhea (2C7) (Light Chain)
QVVVTQESALTTSPGETVTLTCRSSTGAVTTSNYANWVQEKPDHLFTGLIGGINNRAPGVPARFSGSLIGDKAALTITGAQTEDEAIYFCALWYSNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 108 - protein, 215 aa, Artificial Sequence; identical to SEQ ID NO: 107
  Annotated Amino Acid sequence of Fab fragment of anti-Gonorrhea (2C7) IgG (Light Chain)

SEQ ID NO: 109 - DNA, 2106 bp, Artificial Sequence
  Annotated DNA sequence of Synthetic anti-Gonorrhea (2C7) Fab-IgG (Heavy Chain)
gaggtacaactgcaacagagtggccccgagcttgtgaagccagggtccagcgtgaagatttcttgcaagggaagtgggtacacgttcacggactacaacatggagtgggtgaaacaaagtcacggtaaatccttggagtggatcggagttatcaacccaaacaaccgatttactagctacaaccagaatttcagggggaaggcaacactcaccgtcgacaaatcctcttctacggcatatatggatctccgctcacttactagcgaggactctgcagtctatttttgcgcggggagccgatggtatcaatacgactattggggtcaaggtacaacgcttactgttagctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacaggtggcggaggatctggtggaggagggagcggagggggtgggtccggagggggtggttccggaggtggtggatcaggtggcggaggaagtgaggttcagctgcagcagtctggacctgagctggtcaagcctggcagcagcgtgaagatcagctgtaaaggcagcggctacaccttcaccgactacaacatggaatgggtcaagcagagccacggcaagagcctggaatggatcggcgtgatcaaccccaacaaccggttcaccagctacaaccagaacttcagaggcaaggccacactgaccgtggacaagagcagcagcaccgcctacatggatctgagaagcctgaccagcgaggacagcgccgtgtatttttgtgccggcagccggtggtatcagtacgactattggggccagggcacaaccctgaccgttagctctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 110 - protein, 702 aa, Artificial Sequence
  Annotated Amino Acid sequence of Synthetic anti-Gonorrhea (2C7) IgG Fab-IgG (Heavy Chain)
EVQLQQSGPELVKPGSSVKISCKGSGYTFTDYNMEWVKQSHGKSLEWIGVINPNNRFTSYNQNFRGKATLTVDKSSSTAYMDLRSLTSEDSAVYFCAGSRWYQYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSEVQLQQSGPELVKPGSSVKISCKGSGYTFTDYNMEWVKQSHGKSLEWIGVINPNNRFTSYNQNFRGKATLTVDKSSSTAYMDLRSLTSEDSAVYFCAGSRWYQYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 225 aa, Artificial Sequence; identical to SEQ ID NO: 104
  Annotated Amino Acid sequence of Synthetic anti-Gonorrhea (2C7) Fab fragment of Fab-IgG (Heavy Chain)

SEQ ID NO: 112 - protein, 220 aa, Artificial Sequence; identical to SEQ ID NO: 5
  Annotated Amino Acid sequence of Synthetic anti-Gonorrhea (2C7) Fc fragment of Fab-IgG (Heavy Chain)

SEQ ID NO: 113 - DNA, 645 bp, Artificial Sequence; identical to SEQ ID NO: 106
  DNA sequence of Synthetic anti-Gonorrhea (2C7) Fab-IgG (Light Chain)

SEQ ID NO: 114 - protein, 215 aa, Artificial Sequence; identical to SEQ ID NO: 107
  Annotated Amino Acid sequence of Synthetic anti-Gonorrhea (2C7) Fab-IgG (Light Chain)

SEQ ID NO: 115 - protein, 215 aa, Artificial Sequence; identical to SEQ ID NO: 107
  Annotated Amino Acid sequence of Synthetic anti-Gonorrhea (2C7) Fab fragment of Fab-IgG (Light Chain)

SEQ ID NO: 116 - DNA, 2112 bp, Artificial Sequence
  Annotated DNA sequence of Synthetic anti-Gonorrhea (2C7) IgG-Fab (Heavy Chain)
gaggttcagctgcagcagtctggacctgagctggtcaagcctggcagcagcgtgaagatcagctgtaaaggcagcggctacaccttcaccgactacaacatggaatgggtcaagcagagccacggcaagagcctggaatggatcggcgtgatcaaccccaacaaccggttcaccagctacaaccagaacttcagaggcaaggccacactgaccgtggacaagagcagcagcaccgcctacatggatctgagaagcctgaccagcgaggacagcgccgtgtatttttgtgccggcagccggtggtatcagtacgactattggggccagggcacaaccctgaccgttagctctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccggtggcggaggatctggtggaggagggagcggagggggtgggtccggagggggtggttccggaggtggtggatcaggtggcggaggaagtgaggtacaactgcaacagagtggccccgagcttgtgaagccagggtccagcgtgaagatttcttgcaagggaagtgggtacacgttcacggactacaacatggagtgggtgaaacaaagtcacggtaaatccttggagtggatcggagttatcaacccaaacaaccgatttactagctacaaccagaatttcagggggaaggcaacactcaccgtcgacaaatcctcttctacggcatatatggatctccgctcacttactagcgaggactctgcagtctatttttgcgcggggagccgatggtatcaatacgactattggggtcaaggtacaacgcttactgttagctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacaca

SEQ ID NO: 117 - protein, 704 aa, Artificial Sequence
  Annotated Amino Acid sequence of Synthetic anti-Gonorrhea (2C7) IgG-Fab (Heavy Chain)
EVQLQQSGPELVKPGSSVKISCKGSGYTFTDYNMEWVKQSHGKSLEWIGVINPNNRFTSYNQNFRGKATLTVDKSSSTAYMDLRSLTSEDSAVYFCAGSRWYQYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSEVQLQQSGPELVKPGSSVKISCKGSGYTFTDYNMEWVKQSHGKSLEWIGVINPNNRFTSYNQNFRGKATLTVDKSSSTAYMDLRSLTSEDSAVYFCAGSRWYQYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 118 - protein, 225 aa, Artificial Sequence; identical to SEQ ID NO: 104
  Annotated Amino Acid sequence of Synthetic anti-Gonorrhea (2C7) Fab fragment of IgG-Fab (Heavy Chain)

SEQ ID NO: 119 - protein, 220 aa, Artificial Sequence; identical to SEQ ID NO: 5
  Annotated Amino Acid sequence of Synthetic anti-Gonorrhea (2C7) Fc fragment of IgG-Fab (Heavy Chain)

SEQ ID NO: 120 - DNA, 645 bp, Artificial Sequence; identical to SEQ ID NO: 106
  DNA sequence of Synthetic anti-Gonorrhea (2C7) IgG-Fab (Light Chain)

SEQ ID NO: 121 - protein, 215 aa, Artificial Sequence; identical to SEQ ID NO: 107
  Annotated Amino Acid sequence of Synthetic anti-Gonorrhea (2C7) IgG-Fab (Light Chain)

SEQ ID NO: 122 - protein, 215 aa, Artificial Sequence; identical to SEQ ID NO: 107
  Annotated Amino Acid sequence of Synthetic anti-Gonorrhea (2C7) Fab fragment of IgG-Fab (Light Chain)

SEQ ID NO: 123 - DNA, 2877 bp, Artificial Sequence
  Annotated DNA sequence of Synthetic anti-Gonorrhea (2C7) Fab-IgG-Fab (Heavy Chain)
gaggtacaactgcaacagagtggccccgagcttgtgaagccagggtccagcgtgaagatttcttgcaagggaagtgggtacacgttcacggactacaacatggagtgggtgaaacaaagtcacggtaaatccttggagtggatcggagttatcaacccaaacaaccgatttactagctacaaccagaatttcagggggaaggcaacactcaccgtcgacaaatcctcttctacggcatatatggatctccgctcacttactagcgaggactctgcagtctatttttgcgcggggagccgatggtatcaatacgactattggggtcaaggtacaacgcttactgttagctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacaggtggcggaggatctggtggaggagggagcggagggggtgggtccggagggggtggttccggaggtggtggatcaggtggcggaggaagtgaggttcagctgcagcagtctggacctgagctggtcaagcctggcagcagcgtgaagatcagctgtaaaggcagcggctacaccttcaccgactacaacatggaatgggtcaagcagagccacggcaagagcctggaatggatcggcgtgatcaaccccaacaaccggttcaccagctacaaccagaacttcagaggcaaggccacactgaccgtggacaagagcagcagcaccgcctacatggatctgagaagcctgaccagcgaggacagcgccgtgtatttttgtgccggcagccggtggtatcagtacgactattggggccagggcacaaccctgaccgttagctctgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggatccggtggcggaggatctggtggaggagggagcggagggggtgggtccggagggggtggttccggaggtggtggatcaggtggcggaggaagtgaggtacaactgcaacagagtggccccgagcttgtgaagccagggtccagcgtgaagatttcttgcaagggaagtgggtacacgttcacggactacaacatggagtgggtgaaacaaagtcacggtaaatccttggagtggatcggagttatcaacccaaacaaccgatttactagctacaaccagaatttcagggggaaggcaacactcaccgtcgacaaatcctcttctacggcatatatggatctccgctcacttactagcgaggactctgcagtctatttttgcgcggggagccgatggtatcaatacgactattggggtcaaggtacaacgcttactgttagctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacaca

SEQ ID NO: 124 - protein, 959 aa, Artificial Sequence
  Annotated Amino Acid sequence of Synthetic anti-Gonorrhea (2C7) Fab-IgG-Fab (Heavy Chain)
EVQLQQSGPELVKPGSSVKISCKGSGYTFTDYNMEWVKQSHGKSLEWIGVINPNNRFTSYNQNFRGKATLTVDKSSSTAYMDLRSLTSEDSAVYFCAGSRWYQYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSEVQLQQSGPELVKPGSSVKISCKGSGYTFTDYNMEWVKQSHGKSLEWIGVINPNNRFTSYNQNFRGKATLTVDKSSSTAYMDLRSLTSEDSAVYFCAGSRWYQYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSGGGGSGGGGSGGGGSEVQLQQSGPELVKPGSSVKISCKGSGYTFTDYNMEWVKQSHGKSLEWIGVINPNNRFTSYNQNFRGKATLTVDKSSSTAYMDLRSLTSEDSAVYFCAGSRWYQYDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 125 - protein, 225 aa, Artificial Sequence; identical to SEQ ID NO: 104
  Annotated Amino Acid sequence of Synthetic anti-Gonorrhea (2C7) Fab fragment of Fab-IgG-Fab (Heavy Chain)

SEQ ID NO: 126 - protein, 220 aa, Artificial Sequence; identical to SEQ ID NO: 5
  Annotated Amino Acid sequence of Synthetic anti-Gonorrhea (2C7) Fc fragment of Fab-IgG-Fab (Heavy Chain)

SEQ ID NO: 127 - DNA, 645 bp, Artificial Sequence; identical to SEQ ID NO: 106
  DNA sequence of Synthetic anti-Gonorrhea (2C7) Fab-IgG-Fab (Light Chain)

SEQ ID NO: 128 - protein, 215 aa, Artificial Sequence; identical to SEQ ID NO: 107
  Annotated Amino Acid sequence of Synthetic anti-Gonorrhea (2C7) Fab-IgG-Fab (Light Chain)

SEQ ID NO: 129 - protein, 215 aa, Artificial Sequence; identical to SEQ ID NO: 107
  Annotated Amino Acid sequence of Synthetic anti-Gonorrhea (2C7) Fab fragment of Fab-IgG-Fab (Light Chain)

SEQ ID NO: 130 - DNA, 26 bp, Artificial Sequence
  Forward primer
taagcaggtaccgccaccatgaagtg

SEQ ID NO: 131 - DNA, 25 bp, Artificial Sequence
  Reverse primer
tgcttagctagctggagaaactgtc

SEQ ID NO: 132 - DNA, 1350 bp, Artificial Sequence
  Annotated DNA sequence of anti-RSV (Respiratory syncytial virus) IgG (Heavy Chain)
caagtgaccctgagagagtctggccccgctctggttaagcccacacagaccctgacactgacctgcaccttcagcggctttagcctgtctacagccggcatgagcgtcggctggattagacagcctcctggcaaagccctggaatggctggccgacatttggtgggacgacaagaagcactacaaccccagcctgaaggaccggctgaccatcagcaaggacaccagcaagaaccaggtggtgctgaaagtgaccaacatggaccctgccgacaccgccacctactactgcgccagagacatgatcttcaacttctacttcgacgtgtggggccagggcaccaccgtgacagttagctctgcctctacaaagggccccagcgtgttccctctggctcctagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtctgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctccaggcaag

SEQ ID NO: 133 - protein, 450 aa, Artificial Sequence
  Annotated Amino Acid sequence of anti-RSV (Respiratory syncytial virus) IgG (Heavy Chain)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 228 aa, Artificial Sequence
  Annotated Amino Acid sequence of Fab fragment of anti-RSV (Respiratory syncytial virus) (Heavy Chain)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 135 - protein, 220 aa, Artificial Sequence; identical to SEQ ID NO: 5
  Annotated Amino Acid sequence of Fc fragment of anti-RSV (Respiratory syncytial virus) (Heavy Chain)

SEQ ID NO: 136 - DNA, 639 bp, Artificial Sequence
  DNA sequence of anti-RSV (Respiratory syncytial virus) (Light Chain)
gacatccagatgacacagagccccagcacactgtctgccagcgtgggagacagagtgaccatcacatgtagcgccagcagcagagtgggctacatgcactggtatcagcagaagcctggcaaggcccctaagctgctgatctacgacacaagcaagctggcctctggcgtgcccagcagattttctggctctggcagcggcaccgagttcaccctgaccatctctagcctgcagcctgacgacttcgccacctactactgctttcaaggcagcggctaccccttcacctttggcggcggaacaaaggtggaaatcaagcggacagtggccgctcctagcgtgttcatctttccacctagcgacgagcagctgaagtctggcacagcctctgtcgtgtgcctgctcaacaacttctaccccagagaagccaaggtgcagtggaaggtggacaacgccctgcagagcggcaatagccaagagagcgtgaccgagcaggacagcaaggactctacctacagcctgtcctccacactgaccctgagcaaggccgactacgagaagcacaaagtgtacgcctgcgaagtgacccaccagggcctttctagccctgtgaccaagagcttcaaccggggcgagtgc

SEQ ID NO: 137 - protein, 213 aa, Artificial Sequence
  Annotated Amino Acid sequence of anti-RSV (Respiratory syncytial virus) (Light Chain)
DIQMTQSPSTLSASVGDRVTITCSASSRVGYMHWYQQKPGKAPKLLIYDTSKLASGVPSRFSGSGSGTEFTLTISSLQPDDFATYYCFQGSGYPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 213 aa, Artificial Sequence; identical to SEQ ID NO: 137
  Annotated Amino Acid sequence of Fab fragment of anti-RSV (Respiratory syncytial virus) IgG (Light Chain)

SEQ ID NO: 139 - DNA, 2124 bp, Artificial Sequence
  Annotated DNA sequence of Synthetic anti-RSV (Respiratory syncytial virus) Fab-IgG (Heavy Chain)
caagtgaccctgagagagtctggccccgctctggttaagcccacacagaccctgacactgacctgcaccttcagcggctttagcctgtctacagccggcatgagcgtcggctggattagacagcctcctggcaaagccctggaatggctggccgacatttggtgggacgacaagaagcactacaaccccagcctgaaggaccggctgaccatcagcaaggacaccagcaagaaccaggtggtgctgaaagtgaccaacatggaccctgccgacaccgccacctactactgcgccagagacatgatcttcaacttctacttcgacgtgtggggccagggcaccaccgtgacagttagctctgcctctacaaagggccccagcgtgttccctctggctcctagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccatacaggcggcggaggatctggcggaggtggaagcggaggcggaggaagcggtggcggcggtagtggcggtggtggttcaggtggtggtggctctcaagtcacactgagagaaagcggccctgctctcgtgaagcctactcagacactcaccctgacctgtacattctctggcttcagcctgagcaccgccggcatgtctgttggatggatcagacaaccaccaggcaaggctctcgagtggctcgctgatatttggtgggatgataagaaacattataacccatctctcaaggaccgcctcacaatctccaaggatacctccaagaatcaggtcgtcctcaaagtcacgaatatggatcccgccgatacggccacatattactgtgcccgggatatgatctttaatttctattttgatgtctggggccaagggacaaccgtcaccgtgtctagcgccagcacaaagggaccctccgtgtttccactggcacccagctctaagagcacctctggtggaacagctgccctgggatgtctcgtgaaagactacttccccgaaccagtgacagtcagctggaacagcggagccctgacttctggggtgcacacattccctgccgtcctgcaatcttctggcctgtacagcctgtccagcgtcgtgaccgttccttctagctctctgggaacacagacatatatctgtaatgtcaatcacaaaccctccaatacgaaggtcgacaaaaaggtcgagcctaagtcctgtgataagacccacacctgtcctccatgtcctgctccagaactgctcggcggaccttctgtgtttctgttccctccaaagcctaaggacaccctgatgatcagcagaacccctgaagtgacctgcgtggtggtcgatgtgtcccacgaggatcccgaagtgaagttcaattggtacgtggacggcgtggaagtgcacaacgccaagaccaagcctagagaggaacagtacaacagcacctacagagtggtgtctgtgctgaccgtgctgcaccaggattggctgaacggcaaagagtacaagtgcaaggtgtccaacaaggccctgcctgctcctatcgagaaaaccatctctaaggccaagggccagcctcgcgaaccccaggtttacacacttccaccaagccgggacgagctgacaaaaaaccaggtgtccctgacatgcctcgtgaagggcttctacccctccgatatcgccgtggaatgggagagcaatggccagcctgagaacaactacaagaccacacctcctgtgctggacagcgacggctcattcttcctgtactccaagctgacagtggacaagtccagatggcagcagggcaacgtgttcagctgcagcgtgatgcacgaggccctgcacaaccactacacccagaaaagcctgtctctgagccccggcaag

SEQ ID NO: 140 - protein, 708 aa, Artificial Sequence
  Annotated Amino Acid sequence of Synthetic anti-RSV (Respiratory syncytial virus) IgG Fab-IgG (Heavy Chain)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSGGGGSGGGGSGGGGSGGGGSGGGGSQVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 228 aa, Artificial Sequence; identical to SEQ ID NO: 134
  Annotated Amino Acid sequence of Synthetic anti-RSV (Respiratory syncytial virus) Fab fragment of Fab-IgG (Heavy Chain)

SEQ ID NO: 142 - protein, 220 aa, Artificial Sequence; identical to SEQ ID NO: 5
  Annotated Amino Acid sequence of Synthetic anti-RSV (Respiratory syncytial virus) Fc fragment of Fab-IgG (Heavy Chain)

SEQ ID NO: 143 - DNA, 639 bp, Artificial Sequence; identical to SEQ ID NO: 136
  DNA sequence of Synthetic anti-RSV (Respiratory syncytial virus) Fab-IgG (Light Chain)

SEQ ID NO: 144 - protein, 213 aa, Artificial Sequence; identical to SEQ ID NO: 137
  Annotated Amino Acid sequence of Synthetic anti-RSV (Respiratory syncytial virus) Fab-IgG (Light Chain)

SEQ ID NO: 145 - protein, 213 aa, Artificial Sequence; identical to SEQ ID NO: 137
  Annotated Amino Acid sequence of Synthetic anti-RSV (Respiratory syncytial virus) Fab fragment of Fab-IgG (Light Chain)

SEQ ID NO: 146 - DNA, 2124 bp, Artificial Sequence
  Annotated DNA sequence of Synthetic anti-RSV (Respiratory syncytial virus) IgG-Fab (Heavy Chain)
caagtgaccctgagagagtctggccccgctctggttaagcccacacagaccctgacactgacctgcaccttcagcggctttagcctgtctacagccggcatgagcgtcggctggattagacagcctcctggcaaagccctggaatggctggccgacatttggtgggacgacaagaagcactacaaccccagcctgaaggaccggctgaccatcagcaaggacaccagcaagaaccaggtggtgctgaaagtgaccaacatggaccctgccgacaccgccacctactactgcgccagagacatgatcttcaacttctacttcgacgtgtggggccagggcaccaccgtgacagttagctctgcctctacaaagggccccagcgtgttccctctggctcctagcagcaagtctacaagcggaggaacagccgctctgggctgcctggtcaaggattactttcccgagcctgtgaccgtgtcctggaattctggcgctctgacaagcggcgtgcacacctttccagctgtgctgcaaagcagcggcctgtactctctgagcagcgtggtcacagtgccaagctctagcctgggcacccagacctacatctgcaatgtgaaccacaagcctagcaacaccaaggtggacaagaaggtggaacccaagagctgcgacaagacccacacctgtcctccatgtcctgctccagaactgctcggcggaccttccgtgtttctgttccctccaaagcctaaggacaccctgatgatcagcagaacccctgaagtgacctgcgtggtggtggatgtgtcccacgaggatcccgaagtgaagttcaattggtacgtggacggcgtggaagtgcacaacgccaagaccaagcctagagaggaacagtacaacagcacctacagagtggtgtctgtgctgaccgtgctgcaccaggattggctgaacggcaaagagtacaagtgcaaggtgtccaacaaggccctgcctgctcctatcgagaaaaccatctccaaggccaagggccagcctagggaaccccaggtttacacactgcctccaagcagggacgagctgaccaagaatcaggtgtccctgacctgcctcgtgaagggcttctacccttccgatatcgccgtggaatgggagagcaatggccagcctgagaacaactacaagacaacccctcctgtgctggacagcgacggctcattcttcctgtacagcaagctgacagtggacaagtccagatggcagcagggcaacgtgttcagctgcagcgtgatgcacgaggccctgcacaaccactacacccagaagtccctgagcctgtctccaggcaaaggtggcggaggatctggcggaggtggaagcggcggaggcggttctggtggtggcggctctggcggcggtggttcaggtggcggcggttctcaagttacactgagagaaagcggcccagctctcgtgaagcctactcagacactcaccctgacatgtaccttctctggcttcagcctgagcaccgccggcatgtctgttggatggatcagacaaccaccaggcaaggctctcgagtggctcgctgatatttggtgggatgataagaaacattataacccatctctcaaggaccgcctcaccatttccaaggatacctccaaaaatcaggtcgtgctcaaagtcacgaatatggatcccgccgatacggccacatattactgtgcccgggatatgatctttaatttctattttgatgtctggggccaagggacaaccgtcaccgtgtctagcgccagcacaaagggaccctctgtgtttccactggctcccagctctaagagcacctccggtggaacagctgccctgggatgtctcgtgaaagactacttccccgaaccagtgacagtcagctggaacagcggagccctgactagtggggtgcacacattccctgccgtcctgcaatctagcggactgtacagcctgtccagcgtcgtgaccgtgccttctagctctctgggaacacagacatatatctgtaatgtcaatcacaaaccctccaatacgaaggtcgacaaaaaggtcgagcctaagtcctgtgataagacgcacaca

SEQ ID NO: 147 - protein, 708 aa, Artificial Sequence
  Annotated Amino Acid sequence of Synthetic anti-RSV (Respiratory syncytial virus) IgG-Fab (Heavy Chain)
QVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSGGGGSGGGGSQVTLRESGPALVKPTQTLTLTCTFSGFSLSTAGMSVGWIRQPPGKALEWLADIWWDDKKHYNPSLKDRLTISKDTSKNQVVLKVTNMDPADTATYYCARDMIFNFYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 148 - protein, 228 aa, Artificial Sequence; identical to SEQ ID NO: 134
  Annotated Amino Acid sequence of Synthetic anti-RSV (Respiratory syncytial virus) Fab fragment of IgG-Fab (Heavy Chain)

SEQ ID NO: 149 - protein, 220 aa, Artificial Sequence; identical to SEQ ID NO: 5
  Annotated Amino Acid sequence of Synthetic anti-RSV (Respiratory syncytial virus) Fc fragment of IgG-Fab (Heavy Chain)

SEQ ID NO: 150 - DNA, 639 bp, Artificial Sequence; identical to SEQ ID NO: 136
  DNA sequence of Synthetic anti-RSV (Respiratory syncytial virus) IgG-Fab (Light Chain)

SEQ ID NO: 151 - protein, 213 aa, Artificial Sequence; identical to SEQ ID NO: 137
  Annotated Amino Acid sequence of Synthetic anti-RSV (Respiratory syncytial virus) IgG-Fab (Light Chain)

SEQ ID NO: 152 - protein, 213 aa, Artificial Sequence; identical to SEQ ID NO: 137
  Annotated Amino Acid sequence of Synthetic anti-RSV (Respiratory syncytial virus) Fab fragment of IgG-Fab (Light Chain)

SEQ ID NO: 153 - protein, 124 aa, Homo sapiens
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARGFMVRGIYYYYYGMDVWGKGTTVTVSP

SEQ ID NO: 154 - protein, 48 aa, Homo sapiens
SSELTQDPAVSVALGQTVRITCQGDSLRSYYASWYQQKPGQAPVLVIY

SEQ ID NO: 155 - protein, 57 aa, Homo sapiens
NRPSGIPDRFSGSSSGNTASLTITGAQAEDEADYYCNSRDSSGNHWVFGGGTKLTVL